(19) United States
(12) Patent Application Publication
(10) Pub. No.: US 2016/0331760 A1
(43) Pub. Date: Nov. 17, 2016

(54) INCREASING STORAGE OF VITAMIN A, VITAMIN D AND/OR LIPIDS

(71) Applicant: Salk Institute for Biological Studies, La Jolla, CA (US)

(72) Inventors: Ronald M. Evans, La Jolla, CA (US); Michael Downes, San Diego, CA (US); Mara Sherman, San Diego, CA (US); Ning Ding, Riverside, CA (US)

(73) Assignee: Salk Institute for Biological Studies, La Jolla, CA (US)

(21) Appl. No.: 15/226,052

(22) Filed: Aug. 2, 2016

Related U.S. Application Data
(63) Continuation of application No. PCT/US2015/015272, filed on Feb. 10, 2015.
(60) Provisional application No. 61/938,056, filed on Feb. 10, 2014.
(60) Provisional application No. 62/000,495, filed on May 19, 2014.

(51) Int. Cl. A61K 31/551; A61K 45/06; A61K 31/7068; A61K 9/14

(52) U.S. Cl. CPC A61K 31/551; A61K 9/14; A61K 45/06; A61K 31/7068

(57) ABSTRACT

The present disclosure provides compositions that include a nanoparticle and a compound that reduces the biological activity of one or more bromodomain and extra-terminal family member (BET) proteins (e.g., a bromodomain inhibitor), and methods of using such compounds to increase retention or storage of vitamin A, vitamin D, and/or lipids by a cell, such as an epithelial or stellate cell.

### CROSS REFERENCE TO RELATED APPLICATIONS

This application is a continuation of International Application No. PCT/US2015/015272, filed Feb. 10, 2015, which was published in English under PCT Article 21(2), which in turn claims the benefit of U.S. Provisional Application No. 61/938,056, filed Feb. 10, 2014, and U.S. Provisional Application No. 62/000,495, filed May 19, 2014, all incorporated herein in their entirety.

### ACKNOWLEDGEMENT OF GOVERNMENT SUPPORT

This invention was made with government support under Grant Nos. DK057978, DK090962, HL088093, HL105278, CA014195 and ES010337 awarded by The National Institutes of Health. The government has certain rights in the invention.

### FIELD

The present application provides compositions that include a nanoparticle and a compound that reduces the biological activity of one or more bromodomain and extra-terminal family member (BET) proteins, and methods of using such compounds to increase retention or storage of vitamin A, vitamin D, and/or lipids by a cell, such as an epithelial or stellate cell.

### BACKGROUND

Liver fibrosis and cirrhosis are serious clinical complications associated with a wide range of liver diseases including metabolic syndromes and cancer^{1,2}. However, no therapies have been approved by Food and Drug Administration (FDA) to-date³. While most anti-fibrotic strategies in development focus on cell-extrinsic molecules and cell membrane receptors, the contribution of cell-intrinsic pathways such as epigenetic pathways to liver fibrosis and their therapeutic potential remain poorly explored.

### SUMMARY

The present application provides therapeutic compositions, such as a composition that includes a nanoparticle and one or more compounds (such as 1, 2, 3, or 4 such compounds) that reduces the biological activity of one or more bromodomain and extra-terminal family member (BET) proteins. For example, compounds that reduce the biological activity of one or more BET proteins include those that promote or increase storage or retention of vitamin A, vitamin D and/or lipids by a cell, such as an epithelial or stellate cell. In one example, the at BET inhibitor reduces the biological activity of one or more BET proteins by at least 25% as compared to the biological activity in the absence of the BET inhibitor. In some examples, the BET inhibitor promotes or increases storage or retention of vitamin A, vitamin D and/or lipids by a cell, such as an epithelial or stellate cell, by at least 20% or at least 25% as compared to the storage or retention of vitamin A, vitamin D and/or lipids by a cell in the absence of the BET inhibitor. A specific example of a compound that reduces the biological activity of one or more BET proteins is JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate). In some examples, such a composition further includes a chemotherapeutic, a biologic (such as a therapeutic monoclonal antibody), a vitamin D receptor (VDR) agonist, or combinations thereof.

The disclosure also provides methods for increasing or retaining vitamin A, vitamin D, and/or lipid in a cell (such as an epithelial or stellate cell). Such methods can include contacting the cell with a therapeutically effective amount of the compositions disclosed herein, thereby increasing or retaining vitamin A, vitamin D, and/or lipid in the cell. In some examples, such a cell is in a subject, and the method includes administering a therapeutically effective amount of the composition to the subject, thereby increasing or retaining vitamin A, vitamin D, and/or lipid in the cells of the subject, such as epithelial or stellate cells in the subject. In some examples, such a subject has a disease of the liver, pancreas or kidney (or combinations thereof).

The foregoing and other objects and features of the disclosure will become more apparent from the following detailed description, which proceeds with reference to the accompanying figures.

### BRIEF DESCRIPTION OF THE DRAWINGS

FIGS. 1A-1D. BET expression in primary murine HSCs. mRNA-seq reads aligned to (A) Brd2, (B) Brd3, (C) Brd4 and (D) Brdt in primary murine HSCs at quiescent state (day 1).

FIGS. 2A-2H. BETs modulate pro-fibrotic super-enhancer activity in activated HSCs. A, COL1A1 expression in LX-2 cells treated with DMSO (vehicle) or BET inhibitor (JQ1, 500 nM) for 16 hr. B, BET expression shown by mRNA-seq reads aligned to BRD2, BRD3, BRD4 and BRDT in LX-2 cells. C, ChIP-qPCR at COL1A1 enhancer region in LX-2 cells treated with DMSO (vehicle) or JQ1 (500 nM, 16 hr). Data represents the mean±SEM of at least three independent experiments performed in triplicate. Asterisks denote statistically significant differences (Student's t-test, * p<0.05, ** p<0.01). D, De novo analysis of most enriched motifs located within 100 bp of BRD4 peaks in LX-2 cells (FDR=0.0001). E, Gene ontology (GO) analysis (MSigDB) of putative BRD4 target genes in LX-2 cells. F, Intensity plots showing hierarchical clustering of ChIP-fragment densities as a function of distance from the center of statistically significant H3K27ac peaks (FDR=0.0001) outside promoter regions (±2 kb of transcription start site). Intensity around position 0 of BRD2 (black), BRD3 (yellow) and BRD4 (red) indicates overlapping BET/H3K27ac sites with H3K27ac (green) acting as a positive control. G, Rank order of increased BRD4 fold enrichment at enhancer loci in LX-2 cells with super-enhancer defined as surpassing the inflection point. Representative fibrotic super-enhancers are indicated. H, Plots of BRD4 ChIP-seq signal intensity relative to the center of super-enhancers (SE) (500 most BRD4-loaded enhancers) and control enhancers (CE) (10,000 least BRD4-loaded enhancers) in LX-2 cells (±500 nM JQ1 for 16 hr).

FIGS. 3A and 3B. Involvement of BRD2/3/4 in mediating pro-fibrotic gene expression in activated human HSCs. A, COL1A1 and COL1A2, B, BRD2, BRD3 and BRD4 RT-qPCR analysis in control (siCNTL) or BET-specific (siBRD) siRNA-transfected LX-2 cells. Data represents the mean±SEM of at least three independent experiments performed in triplicate. Asterisks denote statistically significant differences (Student's t-test, * p<0.05, ** p<0.01, *** p<0.001).

FIG. 4. Gene expression analysis of anti-fibrotic properties of BET inhibitors in activated human HSCs. Heatmap of fold change of pro-fibrotic genes in LX-2 cells treated with three structurally distinct BET inhibitors, JQ1 (500 nM), I-BET (500 nM) and PFI-1 (500 nM) in LX-2 cells treated with or without TGFβ1 (5 ng/ml) for 16 hr. Gene expression levels in cells treated with vehicle (DMSO) only are arbitratively set as 1.00. Data represents the mean±SEM of at least three independent experiments performed in triplicate.

FIG. 5. Genomic colocalization of BETs in activated human HSCs. Intensity plots showing hierarchical clustering of ChIP-fragment densities as a function of distance from the center of statistically significant BRD4 binding peaks (FDR=0.0001). Intensity around position 0 of BRD2 and BRD3 indicates overlapping BET sites with BRD4 acting as a positive control.

FIGS. 6A-6D. BET inhibition perturbs transcriptional elongation in activated HSCs. A, Plots of BRD2, BRD3, BRD4 and Pol II ChIP-seq signal intensity relative to the center of their respective binding sites in LX-2 cells (±500 nM JQ1 for 16 hr). B, Intensity plots showing hierarchical clustering of ChIP-fragment densities as a function of distance from the center of statistically significant Pol II binding peaks (FDR=0.0001). Intensity around position 0 of BRD2, BRD3, and BRD4 indicates overlapping BETs/Pol II sites with Pol II acting as a positive control. c, Plots of CDK9, PAF1, Pol II S5p and Pol II S2p ChIP-seq signal intensity relative to the center of their respective binding sites in LX-2 cells (±500 nM JQ1 for 16 hr). D, Representative ChIP-seq reads aligned to COL1A1 and PDGFRB for BRD4, Pol II, Pol II S5p and Pol II S2p in LX-2 cells (±500 nM JQ1 for 16 hr). Super-enhancer (SE) regions are indicated.

FIG. 7. Diagram depicting in vitro HSC self-activation system.

FIGS. 8A-8G. BET inhibition blocks HSC activation into myofibroblasts. A, Selected heatmap of fold change of inducible genes in primary activated HSCs treated with DMSO (0.1%) or JQ1 (500 nM) at different time points (Day 3 and 6). Euclidean clustering of both rows and columns using log 2-transformed mRNA-seq expression data, n=3 per treatment group. Bullets (red) indicate key fibrosis marker genes: Col1a1, Col1a2, Acta2 and Des. b, Global analysis of gene expression showing activation-induced genes with time (red) and progressive suppression of this induction by JQ1. c, Volcano plot showing fold change (x axis) effect of JQ1 versus DMSO (shades of blue) on all genes upregulated at both time points (Day 3 and 6) versus Day 1 (shades of red). Progression from light to dark shading represents increasing time (Day 3 and 6). d, Gene ontology (GO) analysis (MSigDB) of activation-induced genes that were suppressed by JQ1. e, Representative images of primary HSCs at quiescent state (day 1: D1) and activated state (day 6: D6) treated with DMSO (0.1%) or JQ1 (500 nM) using different methods: bright field (top panel), Acta2 immunofluorescence staining (middle panel) and BODIPY staining (bottom panel). Scale bar, 50 μm. f, Expression of Acta2 in e was determined by western blot analysis. g, Quantitation of lipid-containing cells in e. Data represents the mean±SEM of at least three independent experiments. Asterisks denote statistically significant differences (Student's unpaired t-test, *** p<0.001).

FIG. 9. BET inhibition suppresses pro-fibrotic gene expression during HSC activation into myofibroblasts. Col1a1, Acta2, Timp1 and Des qRT-PCR analysis in primary murine HSCs treated with DMSO or JQ1 (500 nM) for indicated period. Data represents the mean±SEM of at least three independent experiments performed in triplicate. Asterisks denote statistically significant differences (Student's t-test, * p<0.05, ** p<0.01, *** p<0.001).

FIGS. 10A-10F. BET inhibition blocks proliferation underlying HSC activation into myofibroblasts. A, Anti-proliferative activity of JQ1 during HSC activation into myofibroblasts. B, Detection of apoptosis in JQ1-treated cells (500 nM) by TUNEL assay. C, Detection of cellular senenscence in JQ1-treated cells (500 nM) by β-galactosidase staining. D, BrdU incorporation assay in JQ1-treated cells (500 nM). E, Pdgfrb and Ccnd1, F, Ccnd2 and Myc RT-qPCR analysis in primary HSCs treated with DMSO or JQ1 (500 nM). Data represents the mean±SEM of at least three independent experiments performed in triplicate. Asterisks denote statistically significant differences (Student's t-test, * p<0.05, ** p<0.01, *** p<0.001). Scale bar, 50 μM.

FIGS. 11A-11F. Anti-proliferative properties of BET inhibitors in activated human HSCs. Anti-proliferative activity of A, JQ1 and B, I-BET-151 against LX-2 cells. C, BrdU incorporation assay in LX-2 cells treated with DMSO or JQ1/I-BET-151 (500 nM) for 72 hr. D, Detection of apoptosis in LX-2 cells treated with DMSO or JQ1/I-BET-151 (500 nM) for 72 hr by TUNEL assay. E, Detection of cellular senenscence in LX-2 cells treated with DMSO or JQ1/I-BET-151 (500 nM) for 72 hr by β-galactosidase staining. F, PDGFRB and CCND1 RT-qPCR analysis in LX-2 cells treated with DMSO or JQ1/I-BET-151 (500 nM) for 72 hr. Data represents the mean±SEM of at least three independent experiments performed in triplicate. Asterisks denote statistically significant differences (Student's t-test, * p<0.05, ** p<0.01, *** p<0.001). Scale bar, 50 μM.

FIGS. 12A-12C. Serum ALT and gene expression analysis in prophylactic model of liver fibrosis. A, Dosing regime in the preventive liver fibrosis model. B, Hepatic injury was measured by serum ALT. C, qRT-PCR measurement of hepatic gene expression levels of Col1a1, Acta2, Tgfβ1, and Timp1. Data represents the mean±SEM. Asterisks denote statistically significant differences (Student's t-test, * p<0.05, ** p<0.01, *** p<0.001).

FIGS. 13A-13G. Characterization of anti-fibrotic properties of BET inhibition in liver. A, Livers from 4-week-treated C57BL/6J mice (vehicle [corn oil plus 2-Hydroxypropyl-β-cyclodextrin (HP-β-CD), n=5], JQ1 [corn oil plus JQ1 50 mg/kg IP, n=5], carbon tetrachloride [CCl₄ 0.5 ml/kg plus HP-β-CD IP, n=10], and CCl₄ plus JQ1 [n=8]) stained with Sirius red (left) and hematoxylin and eosin (H&E, right). Scale bar, 250 μm. B, Selected heatmap of fold change of pro-fibrotic genes in liver samples described in A. Euclidean clustering of both rows and columns using log₂-transformed mRNA-seq expression data, n=3 per treatment group. C, Acta2 immunohistochemistry in liver samples shown in A. Fibrosis quantified by D H&E staining (Ishak score), E hydroxyproline content and F Sirius red staining. G, Quantification of Acta2 immunohistochemical staining in C. Data represents the mean±SEM. Asterisks denote statistically significant differences (Student's unpaired t-test, *** p<0.001).

FIGS. 14A-14J. Therapeutic effects of BET inhibition against liver fibrosis. A, Dosing regime in the therapeutic liver fibrosis model. B, Livers from 6-week-treated C57BL/6J mice (CCl₄ [n=10] and CCl₄ plus JQ1 [n=10]) stained with Sirius red (left) and hematoxylin and eosin (H&E, right). Scale bar, 250 μm. Fibrosis quantified by C H&E staining (Ishak score), D Sirius red staining and E hydroxyproline content. F, qRT-PCR measurement of hepatic gene expression levels of Col1a1 and Timp1. G, HSC activation was determined by Acta2 immunohistochemistry. H, Quantification of Acta2 immunohistochemical staining in G. I, qRT-PCR measurement of hepatic gene expression levels of Acta2. J, Model depicting proposed epigenetic control of liver fibrogenesis by BETs. Data represents the mean±SEM. Asterisks denote statistically significant differences (Student's unpaired t-test, ** p<0.01, *** p<0.001).

FIG. 15. Therapeutic effects of BET inhibition against pancreatic cancer cells in vitro. Cell lines shown were grown in the presence of 500 nM JQ1 for 72 hours in astromal or stromal culture conditions.

FIGS. 16A-16D. Therapeutic effects of BET inhibition against orthotopic allografts in vivo. Effect of JQ1 on orthotopic allografts of pancreatic cancer cells (A) BLL (B) pancreas weight, (C) phospho-H3⁺ nuclei per field of view (40×), and (D) CD45 and DAPI expression, following 14 days of treatment with 75 mg/kg JQ1 or vehicle alone.

### SEQUENCE LISTING

The nucleic acid sequences are shown using standard letter abbreviations for nucleotide bases, as defined in 37 C.F.R. 1.822. Only one strand of each nucleic acid sequence is shown, but the complementary strand is understood as included by any reference to the displayed strand. The sequence listing generated on Jul. 14, 2016 (5 kb) and submitted herewith is herein incorporated by reference.

SEQ ID NOS: 1 to 32 provide primer sequences used for QPCR.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All GenBank® Accession numbers referenced herein are incorporated by reference for the sequence available on Feb. 10, 2014. All references, including patents and patent applications, and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Suitable methods and materials for the practice or testing of the disclosure are described below. However, the provided materials, methods, and examples are illustrative only and are not intended to be limiting. Accordingly, except as otherwise noted, the methods and techniques of the present disclosure can be performed according to methods and materials similar or equivalent to those described and/or according to conventional methods well known in the art and as described in various general and more specific references that are cited and discussed throughout the present specification.

In order to facilitate review of the various embodiments of the disclosure, the following explanations of specific terms are provided:

Administration: The compositions provided herein can be delivered to a subject in need thereof using any method known in the art. Includes oral, nasal, rectal, vaginal, transdermal, and parenteral administration. Generally, parenteral formulations are those that are administered through any possible mode except ingestion. This term also refers to injections, whether administered intravenously, intrathecally, intramuscularly, intraperitoneally, intra-articularly, or subcutaneously, and various surface applications including intranasal, inhalational, intradermal, and topical application, for instance.

Bromodomain and extra-terminal family member (BET): A group of proteins that recognize acetylated lysines, such as those on N-terminal histone tails. Examples include bromodomain-containing protein 2 (Brd2), Brd3, Brd4 and bromodomain, testis-specific (Brdt).

Brd2 (OMIM 601540) is a putative nuclear transcriptional regulator and a member of a family of genes that are expressed during development. Brd2 sequences are publically available, for example from GenBank® sequence database (e.g., accession numbers NM_001113182.2 and NP_001106653). One of ordinary skill in the art can identify additional Brd2 nucleic acid and protein sequences, including Brd2 variants.

Brd3 (OMIM 601541), also known as RING3-like protein (RING3L), binds hyperacetylated chromatin and plays a role in the regulation of transcription. Brd3 sequences are publically available, for example from GenBank® sequence database (e.g., accession numbers NM_007371.3 and NP_031397.1). One of ordinary skill in the art can identify additional Brd3 nucleic acid and protein sequences, including Brd3 variants.

Brd4 (OMIM 608749) influences mitotic progress as it remains bound to transcriptional start sites of gene expressed during the M/G1 transition. Brd4 has been identified as a component of a recurrent chromosomal translocation in an aggressive form of human squamous carcinoma. Brd4 sequences are publically available, for example from GenBank® sequence database (e.g., accession numbers NM_058243.2, NM_014299.2, NP_055114.1, AAH35266.1, and NP_490597.1). One of ordinary skill in the art can identify additional Brd4 nucleic acid and protein sequences, including Brd4 variants.

Brdt (OMIM 602144) is a testis-specific chromatin protein that specifically binds histone H4 acetylated at ‘Lys-5’ and ‘Lys-8’ (H4K5ac and H4K8ac, respectively) and plays a role in spermatogenesis. Brdt sequences are publically available, for example from GenBank® sequence database (e.g., accession numbers AF019085 and AAB87862.1). One of ordinary skill in the art can identify additional Brdt nucleic acid and protein sequences, including Brdt variants.

Contact: To bring one agent into close proximity to another agent, thereby permitting the agents to interact. For example, a composition containing a BET inhibitor can be applied to a cell (for example in tissue culture), or administered to a subject, thereby permitting the BET inhibitor to interact with cells in vitro or in vivo.

Fibrosis: Refers to the formation or development of excess fibrous connective tissue in an organ or tissue as a reparative or reactive process, as opposed to a formation of fibrous tissue as a normal constituent of an organ or tissue. The term fibrosis includes at least liver/hepatic fibrosis, kidney/renal fibrosis, and pancreatic fibrosis. In particular examples the subjects treated herein have a fibrosis, such as a liver fibrosis.

Hepatic fibrosis is the accumulation of abnormal extracellular matrix (ECM) proteins and a resultant loss of liver function, and is an accompaniment of an inflammation-driven wound healing process triggered by chronic liver injury (Bataller & Brenner 2005 J Clin Invest., 115(2):209-18). The most common causes of liver injury that lead to fibrosis include chronic hepatitis C virus (HCV) infection, alcohol abuse, chronic hepatitis B infection (HBV) and increasingly, nonalcoholic steatohepatitis (NASH), which represents the hepatic metabolic consequence of rising obesity and associated insulin resistance in the setting of an increasingly sedentary lifestyle (Bataller & Brenner 2005 J Clin Invest., 115(2):209-18; Friedman 1999 Am J Med., 107(6B):27S-30S; Siegmund et al., 2005 Dig Dis., 23(3-4):264-74; Friedman & Bansal Hepatology., 43(2 Suppl 1):S82-8). The inflammatory process that results from hepatic injury triggers a variety of cellular responses that include cell repair, regeneration, increased extracellular matrix turnover, and ultimately, in some patients, significant fibrosis. Progressive fibrosis of the liver eventually can result in cirrhosis, loss of liver function (decompensated cirrhosis), portal hypertension, and hepatocelluar carcinoma (Bataller & Brenner 2005 J Clin Invest. 115(2):209-18; Friedman 2003 J. Hepatol. 38(Suppl. 1):S38-S53).

Without being bound by theory, hepatic fibrogenesis is thought to be the result of a wound healing process that occurs after continued liver injury in which parenchymal cells proliferate to replace necrotic or apoptotic cells. This process is associated with an inflammatory response and a limited deposition of ECM. If the hepatic injury persists, eventually hepatocytes are replaced by abundant ECM components, including fibrillar collagen. The distribution of this fibrous material within the lobular architecture of the liver depends on the origin of the liver injury. In chronic viral hepatitis and chronic cholestatic disorders, the fibrotic tissue is initially located around the portal tracts, while in alcohol-induced liver disease and NASH, it is found in the pericentral and perisinusoidal areas (Friedman 2003 J. Hepatol., 38(Suppl. 1):S38-S53; Popper & Uenfriend 1970. Am. J. Med., 49:707-721). As fibrotic liver diseases advance, the pathology progresses from isolated collagen bands to bridging fibrosis, and ultimately, established cirrhosis with regenerative nodules of hepatocytes encapsulated within type I collagen bands (Popper & Uenfriend 1970. Am. J. Med., 49:707-721).

Renal fibrosis causes significant morbidity and mortality as the primary acquired lesion leading to the need for dialysis or kidney transplantation. Renal fibrosis can occur in either the filtering or reabsorptive component of the nephron, the functional unit of the kidney. Experimental models have identified a number of factors that contribute to renal scarring, particularly derangements of physiology involved in the autoregulation of glomerular filtration. This in turn leads to replacement of normal structures with accumulated extracellular matrix (ECM). A spectrum of changes in the physiology of individual cells leads to the production of numerous peptide and non-peptide fibrogens that stimulate alterations in the balance between ECM synthesis and degradation to favor scarring. Almost all forms of end stage renal disease (ESRD) are characterized by significant renal fibrosis.

Fibrosis of the pancreas is a characteristic feature of chronic pancreatitis of various etiologies, and is caused by such processes as necrosis/apoptosis, inflammation, and duct obstruction. The initial event that induces fibrogenesis in the pancreas is an injury that may involve the interstitial mesenchymal cells, the duct cells and/or the acinar cells. Damage to any one of these tissue compartments of the pancreas is associated with cytokine-triggered transformation of resident fibroblasts/pancreatic stellate cells into myofibroblasts and the subsequent production and deposition of extracellular matrix. Depending on the site of injury in the pancreas and the involved tissue compartment, predominantly inter(peri)lobular fibrosis (as in alcoholic chronic pancreatitis), periductal fibrosis (as in hereditary pancreatitis), periductal and interlobular fibrosis (as in autoimmune pancreatitis) or diffuse inter- and intralobular fibrosis (as in obstructive chronic pancreatitis) develops.

Hepatic stellate cells (HSCs): Include pericytes found in the perisinusoidal space (a small area between the sinusoids and hepatocytes) of the liver. The hepatic stellate cell is the major cell type involved in liver fibrosis, which is the formation of scar tissue in response to liver damage. Stellate cells can be selectively stained with gold chloride, but their distinguishing feature in their quiescent (non-activated) state in routine histological preparations is the presence of multiple vitamin A-rich lipid droplets in their cytoplasm, which auto-fluoresce when exposed to ultraviolet (UV) light.

In the normal liver, stellate cells exist in a quiescent state. Quiescent stellate cells represent 5-8% of the total number of liver cells. Each cell has several long protrusions that extend from the cell body and wrap around the sinusoids. The lipid droplets in the cell body store vitamin A. Without being bound by theory, quiescent hepatic stellate cells are thought to play a role in physiological (normal) ECM production and turnover as well as acting as a liver-resident antigen-presenting cell, presenting lipid antigens to and stimulating proliferation of NKT cells.

When the liver is damaged, stellate cells can change into an activated state. The activated stellate cell is characterized by proliferation, contractility, and chemotaxis. The amount of stored vitamin A decreases progressively in liver injury. The activated stellate cell is also responsible for secreting excessive and pathological ECM components as well as reduced production of matrix degrading enzymes, which leads to fibrosis.

Isolated: An “isolated” biological component (such as a nucleic acid molecule, peptide, or cell) has been purified away from other biological components in a mixed sample (such as a cell extract). For example, an “isolated” peptide or nucleic acid molecule is a peptide or nucleic acid molecule that has been separated from the other components of a cell in which the peptide or nucleic acid molecule was present (such as an expression host cell for a recombinant peptide or nucleic acid molecule).

Pancreatic cancer: A malignant tumor within the pancreas. The prognosis is generally poor. About 95% of pancreatic cancers are adenocarcinomas (such as pancreatic ductal adenocarcinoma). The remaining 5% are tumors of the exocrine pancreas (for example, serous cystadenomas), acinar cell cancers, and pancreatic neuroendocrine tumors (such as insulinomas). An “insulinoma” is a cancer of the beta cells that retains the ability to secrete insulin. Patients with insulinomas usually develop neuroglycopenic symptoms. These include recurrent headache, lethargy, diplopia, and blurred vision, particularly with exercise or fasting. Severe hypoglycemia may result in seizures, coma and permanent neurological damage. Symptoms resulting from the catecholaminergic response to hypoglycemia (for example, tremulousness, palpitations, tachycardia, sweating, hunger, anxiety, nausea). A pancreatic adenocarciona occurs in the glandular tissue. Symptoms include abdominal pain, loss of appetite, weight loss, jaundice and painless extension of the gallbladder. In some examples, a pancreatic ductal adenocarcinoma is one having a Kras mutation.

Classical treatment for pancreatic cancer, including adenocarcinomas and insulinomas includes surgical resection (such as the Whipple procedure) and chemotherapy with agent such as fluorouracil, gemcitabine, and erlotinib.

Pancreatic stellate cells (PSCs): Myofibroblast-like cells, which like hepatic stellate cells can switch between the quiescent and activated phenotypes. PSCs reside in exocrine areas of the pancreas. The PSC is the major cell type involved in pancreatic fibrosis, which is the formation of scar tissue in response to damage to the pancreas. When activated, PSCs migrate to the injured location, and participate in tissue repair activities, secreting extracellular matrix (ECM) components. PSC are believed to play a role in the pathogenesis of pancreatitis and pancreatic cancer.

In healthy pancreas, quiescent PSCs store lipids, contain cytoplasmic lipid droplets, and express neural markers (such as nestin, desmin). In contrast, activated proliferative PSCs loose lipid droplets, are myofibroblast (CAF)-like (e.g., express alpha-SMA), and have increased ECM production.

A 3D model of a human pancreatic ductal adenocarcinoma with stromal features can be made using the methods of Weigelt et al. (Adv. Drug Deliv. Rev. 2014; 69-70:42-51).

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the compositions herein disclosed. For example a composition provided herein can be administered in the presence of on or more pharmaceutically acceptable carriers.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (for instance, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Embodiments of other pharmaceutical compositions can be prepared with conventional pharmaceutically acceptable carriers, adjuvants, and counter-ions, as would be known to those of skill in the art. The compositions in some embodiments are in the form of a unit dose in solid, semi-solid, and liquid dosage forms, such as tablets, pills, capsules, lozenges, powders, liquid solutions, or suspensions.

Subject: Living multi-cellular vertebrate organisms, a category that includes both human and non-human mammals. The methods and compositions disclosed herein have equal applications in medical and veterinary settings. Therefore, the general term “subject” is understood to include all animals, including, but not limited to, humans or veterinary subjects, such as other primates (including monkeys), dogs, cats, horses, and cows.

Therapeutically effective amount: An amount of a therapeutic agent (such as a composition provided herein that includes a BET inhibitor), alone or in combination with other agents sufficient to prevent advancement of a disease, to cause regression of the disease, or which is capable of relieving symptoms caused by the disease, such as a symptom associated with fibrosis of the liver, pancreas or kidney, for example fever, respiratory symptoms, fibrotic content, pain or swelling. In one example, a therapeutically effective amount is an amount of a composition provided herein that includes a BET inhibitor sufficient to reduce symptoms of fibrosis by at least 10%, at least 20%, at least 50%, at least 70%, or at least 90%.

Treating a disease: “Treatment” refers to a therapeutic intervention that ameliorates a sign or symptom of a disease or pathological condition (for instance, fibrosis) after it has begun to develop. “Prevention” refers to inhibiting the full development of a disease, for example in a person who is known to have a predisposition to a disease such as a person who has been or is at risk for developing fibrosis of the liver, pancreas or kidney.

Vitamin D: A group of fat-soluble secosteroid prohormones and hormones, the two major forms of which are vitamin D2 (ergocalciferol) and vitamin D3 (cholecalciferol), which are converted to 1α,25 dihydroxyvitamin D₃ (1,α25-(OH)₂-D3), also known as calcitriol, the physiologically active form of vitamin D.

Vitamin D agonist or analog: Any compound, synthetic or natural, that binds to and activates the vitamin D receptor, such as a VDR ligand (e.g., calcitriol), VDR agonist precursor, vitamin D analogs, vitamin D precursors. Specific, non-limiting examples of natural and synthetic vitamin D agonists and analogs include 1α,25(OH)₂D₃, calcipotriol, LG190090, LG9190119, LG190155, LG190176, and LG190178 (see, for instance, Boehm et al., (1999) Chemistry & Biology, 6:265-275); LY2108491, and LY2109866 (Ma et al., (2006)J Clin. Invest., 116:892-904); 2β-(3-Hydroxypropoxy)1α,25-Dihydroxyvitamin D₃ (ED-71) (Tsurukami et al., (1994) Calcif. Tiss. Int. 54:142-149); EB1089 (Pepper et al., (2003) Blood, 101:2454-2460); OCT(22-oxa-calcitrol) (Makibayashi et al., (2001) Am. J. Path., 158:1733-1741); (1αOH-2,19-nor-25hydroxyvitaminD₃) and (1,3-Deoxy-2-CHCH₂OH-19-nor-25-hydroxyvitaminD3) (Posner et al., (2005) Bioorganic & Medicinal Chemistry, 13:2959-2966) and any of the vitamin D analogs disclosed in Rey et al., (1999) J. Organic Chem., 64:3196-3206; and bile acid derivatives such as lithochoic acid (LCA) and ursodoxycholic acid (UDCA) (see, for instance, Nehring et al., (2007) PNAS, 104:10006-10009; Makishima et al., (2002) Science, 296:1313-1316; Copaci et al., (2005) Rom. J. Gastroenterol., 14:259-266). Each of these references is hereby incorporated by reference in its entirety.

Vitamin D precursor: Any compound capable of being converted to an agonist of the vitamin D receptor by an enzyme. In certain, non-limiting examples, that enzyme is CYP27B1. Specific, non-limiting examples of vitamin D precursors include vitamin D₃ (cholecalciferol), 25-hydroxy-vitamin D₃ (25-OH-D₃) (calcidiol), as well as vitamin D2 (ergocalciferol) and its precursors.

Vitamin D receptor (VDR): A member of the steroid hormone family of nuclear receptors. VDR possesses the common nuclear receptor structure, for example, is comprised of an N-terminal activation domain, a DNA-binding region (DBD) with two zinc finger domains, a hinge region and a ligand-binding domain (LBD). VDR activated gene transcription requires initial nuclear translocation via importin-α, heterodimerization with RXR, and binding to response elements present in target genes. VDR is known to regulate genes associated with the maintenance of calcium and phosphate homeostasis in the intestine and kidney. The signal initiated by VDR/RXR heterodimers is modulated by the association of co-activating or co-repressing proteins and also depends on other signaling partners in the nuclear compartment. The VDR/RXR heterodimer is non-permissive, in that the presence or absence of RXR ligands is not known to affect VDR responses.

Until recently the only known physiological ligand for VDR was 1α,25(OH)₂D3 (calcitriol). However, specific bile acids such as LCA and some derivatives (LCA-acetate, LCA-formate, 3-keto LCA) also can activate VDR.

### Overview

Fibrotic diseases contribute to as much as 45% of mortalities in developed countries, and thus contribute a huge health burden with few clinically available therapeutic options. While most anti-fibrotic strategies currently in development focus on cell-extrinsic molecules or autonomous receptors, the contribution of the human genome to organ fibrogenesis and its therapeutic potential remain unknown. The role of genetic enhancers in myofibroblasts, a cell type that dominates the pathogenesis and progression of tissue fibrosis, is examined herein. It is shown that bromodomain and extra-terminal family members (BETs), a group of epigenetic readers, are involved in super-enhancer-mediated pro-fibrotic gene expression in hepatic stellate cells (HSCs, a.k.a. lipid-containing liver-specific pericytes), which upon activation during liver fibrogenesis give rise to myofibroblasts. The data herein show BETs enrichment concentrated at hundreds of super-enhancers associated with genes involved in multiple pro-fibrotic pathways. This unique loading pattern serves as a molecular mechanism by which BETs coordinate pro-fibrotic gene expression in myofibroblasts. Strikingly, suppression of BET-enhancer interaction using small-molecule inhibitors such as JQ1 dramatically blocks activation of HSCs into myofibroblasts and significantly compromises the proliferation of activated HSCs. Furthermore, pharmacologic studies show that JQ1-mediated BET inhibition confers strong protective as well as therapeutic effects against liver fibrosis. In addition, it is shown that JQ1 reduces or inhibits growth of pancreatic cancer cell lines in vitro and in vivo. Since myofibroblasts are the final common pathological cell type underlying nearly all fibrotic disease, targeting pro-fibrotic super-enhancers in these cells through BET inhibition can have clinical benefits in patients with a broad spectrum of fibrotic complications.

In light of the enormous unfulfilled clinical need for anti-fibrotic therapies, these findings therefore not only identify BET-mediated super-enhancers as critical genomic regulators of fibrosis but also provide the first mechanistic insights into the intrinsic epigenetic vulnerabilities for fibrotic diseases that can be exploited for pharmacological intervention.

The role of epigenetic regulators in modulating pro-fibrotic response in hepatic stellate cells (HSCs), a key cellular player underlying the pathogenesis and progression of liver fibrosis^{1,4}, is demonstrated herein. It is shown that, a group of epigenetic readers, bromodomain and extra-terminal family members (BETs), are critical for pro-fibrotic gene expression during HSC activation. Suppression of BETs using small-molecule inhibitors such as JQ1 dramatically blocks transdifferentiation as well as proliferation of HSCs (as well as PSCs) during their activation into myofibroblasts in vitro. Pharmacological studies in the standard mouse model of liver fibrosis show that JQ1 confers strong protection against liver fibrosis. Notably, the compound even exhibits significant therapeutic effects as it slows the progression of the disease in the same animal model. Mechanistically, it is shown that BETs function as a platform for the recruitment of coregulatory complexes such as polymerase-associated factor complex (PAFc) and positive transcription elongation factor complex (P-TEFb) to facilitate transcriptional elongation of pro-fibrotic genes in activated HSCs. Similar results are shown for pancreatic stellate cells and cancers. These findings expose liver fibrosis/cirrhosis, as well as pancreatic fibrosis/cancer to their intrinsic epigenetic vulnerabilities that for the first time can be exploited for pharmacological intervention.

Activation of quiescent HSCs into myofibroblasts is the hallmark event in the pathogenesis of liver fibrosis^{1,4}. In addition to dramatic phenotypic changes, HSC activation is also accompanied by significant induction of pro-fibrotic gene expression². Therapeutic strategies that selectively target such a pathological gene expression program in HSCs therefore hold promise for anti-fibrotic therapies^{2,3}. In this regard, previous results demonstrated the genomic crosstalk between transcription factors mediates epigenetic attenuation of pro-fibrotic response in HSCs implicate chromatin-bound epigenetic regulatory machinery as novel drug targets for clinical management of liver fibrosis⁵. To explore such molecular conduits that can be exploited pharmaceutically, a subset of bromodomain and extra terminal family proteins (BETs: BRD2, BRD3, BRD4 and BRDT) were examined as: 1. These proteins are druggable targets and selective BET inhibitors that disrupt interaction between BETs and histones have been well characterized^{6,7}; 2. BETs are regulators of pathologenic gene expression programs which underlies cancerous^{8,9,10,11}, viral¹², pro-inflammatory¹³ and cardiac hypertrophic responses^{14,15}.

It is shown herein that BETs have an unexpected but critical role as epigenetic regulators of myofibroblast activation that is essential for fibrosis, such as liver, pancreatic, and kidney fibrosis. It is shown that BETs control HSC activation by governing super-enhancer activity that mediates transcriptional elongation of pro-fibrotic genes and pharmacological targeting of BETs leads to attenuation of liver fibrosis. The data provided herein establish the first example of intrinsic genomic/epigenetic susceptibilities that can be exploited pharmaceutically to ameliorate tissue fibrosis, for example to manage liver fibrosis, pancreatic fibrosis, kidney fibrosis, or pancreatic cancer.

### Compositions Containing Bromodomain Inhibitors

The present disclosure provides compositions that include a nanoparticle and one or more compounds that reduce the biological activity of one or more bromodomain and extra-terminal family member (BET) proteins. Such compositions can include additional agents, such as one or more pharmaceutically acceptable carriers, other therapeutic agents, or combinations thereof. In one example, the compositions further include a chemotherapeutic (such as gemcitabine), a biologic (such as a therapeutic antibody), a vitamin D receptor (VDR) agonist (such as vitamin D, a vitamin D precursor, a vitamin D analog, a vitamin D receptor ligand, a vitamin D receptor agonist precursor, or combinations thereof), or combinations thereof. Specific examples of VDR agonists that can be used include, but are not limited to: calcipotriol, 25-hydroxy-D₃ (25-OH-D₃) (calcidiol); vitamin D3 (cholecalciferol); vitamin D2 (ergocalciferol), 1,α25-dihydroxyvitamin D₃ (calcitriol), and combinations thereof. The disclose compositions can be used in the methods provided herein.

Examples of nanoparticles that can be used in the disclosed compositions include, but are not limited to those provided in US Publication Nos. 20130287688, 20130287857, 20100233251, 20100092425, 20120027808, 20080226739, and 20050215507 and U.S. Pat. Nos. 7,427,394, 8,343,497, 8,562,998, 7,550,441, 7,727,969, 8,343,498, and 8,277,812, all herein incorporated by reference. In some examples the nanoparticle is a lipid or polymeric nanoparticle. In a specific example, the nanoparticle includes a linear-dendritic hybrid polymer for encapsulating biologically active materials, comprising: a ligand for a predetermined target; a dendron; and a polyethylene glycol (PEG) chain linking the ligand to the dendron. In some examples, the nanoparticle is between about 0.1 nm and 5000 nm in diameter, such as 1-100 nm, 0.1-1 nm, 5-20 nm, 5-15 nm, 10-5,000 nm, 20-1,000 nm, 10-500 nm, 10-200 nm, 10-150 nm, 10-100 nm, 10-25 nm, 20-40 nm, or 10, 15, 20, 25, 35, 45, 50, 75, 100, 150 or 200 nm in diameter.

BET proteins whose function can be reduced or inhibited with the disclosed compositions include human bromodomain-containing protein 2 (Brd2), Brd3, Brd4 and/or Brdt. The BET family shares a common domain architecture feature two amino-terminal bromodomains that exhibit high levels of sequence conservation. The biological activity BET proteins that can be reduced or inhibited by the disclosed compositions can include the release of vitamin A from a cell, release of vitamin D from a cell, release of lipids from a cell, or combinations thereof. In some examples, the cell is a stellate cell (such as a pancreatic, kidney or hepatic stellate cell), an epithelial cell, or both. For example, in response to injury or stress, vitamins A and D and lipids can be released from an activated cell (such as an activated epithelial or stellate cell), which can result in other injury, such as fibrosis. Thus, in order to reduce these other injuries, such as fibrosis, the function of BET proteins can be reduced or inhibited to revert the cell to a quiescent state.

A compound that reduces the biological activity of a BET protein (e.g., a BET inhibitor or bromodomain inhibitor) need not completely inhibit BET protein activity. In some examples, such compounds reduce BET protein activity by at least 10%, at least 20%, at least 25%, at least 40%, at least 50%, at least 75%, at least 80%, at least 90%, at least 95%, or at least 99%. Thus in one example, a compound that reduces the biological activity of a BET protein can reduce the release of vitamin A from a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound. In one example, a compound that reduces the biological activity of a BET protein can reduce the release of vitamin D from a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound. In one example, a compound that reduces the biological activity of a BET protein can reduce the release of lipids from a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound.

In some examples, a compound that reduces the biological activity of a BET protein can increase the retention or storage of vitamin A by a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound. In one example, a compound that reduces the biological activity of a BET protein can increase the retention or storage of vitamin D by a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound. In one example, a compound that reduces the biological activity of a BET protein can increase the retention or storage of lipids by a cell (such as a stellate or epithelial cell) by at least 10%, at least 20%, at least 25%, at least 40%, at least 50%, at least 75%, at least 80%, at least 90%, at least 95%, or at least 99%, as compared to an absence of the compound.

Methods of measuring vitamin A, vitamin D, and lipid in a cell are known and are provided herein, and such assays can be used to determine if a compound reduces the biological activity of a BET protein and thus can be used in the compositions provided herein. Exemplary methods for measuring vitamin A in a cell are provided in Vogel et al. (J. Lipid Res. 41(6):882-93, 2000) and methods for measuring vitamin D in a cell are provided in Blum et al. (Endocrine. 33(1):90-4, 2008). In one example, the ability of a compound to revert a cell, such as a stellate cell, to a quiescent state can be determined by staining the cell in the presence and absence of the compound (for example before and after contact with the compound) with BODIPY®, a fluorescent dye that binds neutral lipid. Quiescent cells are characterized by cytoplasmic lipid droplets, which are lost in the activated cell state and accumulate upon treatment of activated cells with drugs such as a compound that reduces the biological activity of a BET protein and/or VDR ligands, which induce quiescence. Thus, treatment of activated cells followed by BODIPY® staining and fluorescence measurements can be used to identify compounds that reduce the biological activity of a BET protein which drive cells (such as stellate cells) toward quiescence.

Compounds that can reduce the biological activity of a BET protein (e.g., a BET inhibitor or bromodomain inhibitor) are known and are publicly available, and the disclosure is not limited to specific inhibitors. A specific example of a BET protein inhibitor is JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate) (see for example, Filippakopoulos et al., Nature 468:1067-73, 2010).

Another specific example of a BET protein inhibitor is LY294002 (2-Morpholin-4-yl-8-phenylchromen-4-one).

Other examples of compounds that reduce the biological activity of a BET protein include but are not limited to: I-BET151 (GSK12101151A) (Dawson et al., Nature 478: 529-533, 2011)

TEN-010 from Tensha Therapeutics, those listed in Muller and Knapp (Royal Soc. Chem. DOI: 10.1039/c3md00291h, 2014), and those available from APExBIO (Houston, Tex.), such as I-BET-762, which is also known as GSK525762.

I-BET-762 binds to the acetylated lysine recognition motifs on the bromodomain of BET proteins, thereby preventing the interaction between the BET proteins and acetylated histone peptide. Others include OTX-015 ((6S)-4-(4-chlorophenyl)-N-(4-hydroxyphenyl)-2,3,9-trimethyl-6H-thieno[3,2-f][1,2,4]triazolo[4,3-a][1,4]diazepine-6-acetamide)

### RVX-208 (2-(4-(2-hydroxyethoxy)-3,5-dimethylphenyl)-5,7-dimethoxyquinazolin-4(3H)-one)

Other exemplary BET inhibitors that can be used in the disclosed compositions and methods include those provided in Gallenkamp et al. (ChemMedChem 9:438-64, 2014). In some examples, a BET inhibitor is one not found in nature (e.g., is not naturally occurring). In some examples, a BET inhibitor is a small molecule inhibitor. In some examples, a BET inhibitor is not a protein or antibody.

### Methods of Using Compositions Containing Bromodomain Inhibitors

The present disclosure also provides methods of using the disclosed compositions that include a nanoparticle and a compound that reduces the biological activity of a BET protein to increase or retain vitamin A, vitamin D, and/or lipid in a cell, such as an epithelial or stellate cell. Thus, provided are methods that can be used to return an active stellate or epithelial cell to its quiescent state.

In some examples, the method includes contacting a therapeutically effective amount of the one or more of the disclosed compositions with a cell, such as an epithelial or stellate cell, such as an activated epithelial or stellate cell. Such a method can be used to increase or retain vitamin A, vitamin D, and/or lipid in the cell. In some examples, the cell is in a subject, and contacting includes administering a therapeutically effective amount of the composition to the subject, thereby increasing or retaining vitamin A, vitamin D, and/or lipid in the cells of the subject (such as epithelial and/or stellate cells, such as pancreatic stellate cells, liver stellate cells, and/or kidney stellate cells).

In some examples, the method increases the retention or storage of vitamin A by a cell (such as a stellate or epithelial cell) by at least 10%, at least 20%, at least 25%, at least 40%, at least 50%, at least 75%, at least 80%, at least 90%, or at least 95%, as compared to an absence of the treatment. In one example, the method increases the retention or storage of vitamin D by a cell (such as a stellate or epithelial cell) by at least 10%, at least 20%, at least 25%, at least 40%, at least 50%, at least 75%, at least 80%, at least 90%, at least 95%, or at least 99%, as compared to an absence of the treatment. In one example, the method increases the retention or storage of lipids by a cell (such as a stellate or epithelial cell) by at least 10%, at least 20%, at least 25%, at least 40%, at least 50%, at least 75%, at least 80%, at least 90%, at least 95%, or at least 99%, as compared to an absence of the treatment.

In some examples, the subject to be treated has a liver disease, such as one or more of alcohol liver disease, fatty liver disease, liver fibrosis/cirrhosis, biliary fibrosis/cirrhosis, liver cancer (such as hepatocellular carcinoma, cholangiocarcinoma, angiosarcoma, or hemangiosarcoma), hepatitis, sclerosing cholangitis, Budd-Chiari syndrome, jaundice, hemochromatosis, or Wilson's disease. In some examples, the subject to be treated has a pancreatic disease, such as pancreatic fibrosis, pancreatic ductal adenocarcinoma (PDA), or both. In some examples, the subject to be treated has a kidney disease, such as fibrosis of the kidney, renal cell carcinoma, or both.

Thus, in some examples, the disclosed methods decrease liver fibrosis by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the size, volume, and/or weight of a liver cancer by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the metastasis of a liver cancer by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment.

In some embodiments, the method can be used to treat pancreatic cancer. The pancreatic cancer can be a ductal adenocarcinoma. In one embodiment, a therapeutically effective amount reduces or inhibits further growth of a pancreatic adenocarcinoma, or reduces a sign or a symptom of the tumor, or reduces metatstasis. Site-specific administration of the disclosed compounds can be used, for instance by applying the compound from which a tumor has been removed, or a region suspected of being prone to tumor development. In some embodiments, sustained intra-tumoral (or near-tumoral) is used.

Thus, in some examples, the disclosed methods decrease pancreatic fibrosis by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the size, volume, and/or weight of a pancreatic cancer by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the metastasis of a pancreatic cancer by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment.

Most subjects diagnosed with pancreatic adenocarcinoma have a life expectancy of only a few months, even with some conventional treatments. The poor prognosis for these subjects is due to the metastasis of these tumors to distant sites early during the disease course, and the resistance of the disease to conventional chemotherapy and/or radiation therapy. For subjects with tumors located in the head and body of the pancreas, symptoms of disease are associated with compression of the bile duct, the pancreatic duct, the mesenteric and celiac nerves, and the duodenum; and these tumors may or may not cause the patient pain. For tumors located in the tail of the pancreas, subjects may have pain on the left side of the abdomen, but pain is generally associated with late stage disease. The disclosed methods can be used to treat any of these subjects. The disclosed methods can be combined with other chemotherapeutic agents or surgical resection for the treatment of pancreatic cancer, such as an adenocarcinoma.

In some embodiments, the subject shows symptoms of fibrosis of the liver, pancreas, or kidney. For example, the subject may be infected with hepatitis B or hepatitis C. In some examples, the administration of a therapeutic composition that includes a compound that reduces the biological activity of a BET protein reduces the symptoms of fibrosis. In some examples, the subject is at risk for developing fibrosis (e.g., is infected with hepatitis B or is an alcoholic or has other liver disease), and the therapeutic composition is administered prophylactically.

In some examples, the disclosed methods can be used to reduce one or more of fibrosis (for example by decreasing the fibrotic content of a fibrotic liver, kidney or pancreases), decrease tumor growth, size or volume, and metastatic lesions, as compared to no treatment with the disclosed compositions.

Thus, in some examples, the disclosed methods decrease kidney fibrosis by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the size, volume, and/or weight of a kidney cancer (such as RCC) by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment. In some examples, the disclosed methods decrease the metastasis of a kidney cancer (such as RCC) by at least 10%, at least 20%, at least 25%, at least 40%, at least 50%, at least 75%, at least 80%, at least 90%, at least 95%, at least 200%, at least 300%, at least 400%, or at least 500% as compared to an absence of the treatment.

In some examples, the disclosed methods are prophylactic. For example, the method can include administering subject at risk for developing fibrosis a therapeutic composition that includes a compound that reduces the biological activity of a BET protein. Such prophylactic administration can delay the onset of the symptoms of fibrosis of the liver, kidney or pancreas, such as a delay of at least 1 month, at least 2 months, at least 6 months, at least 1 year, at least 2 years or even at least 5 years. For example, prophylactic administration of a composition that includes a compound that reduces the biological activity of a BET protein can be used to prevent the onset of one or more symptoms or features of fibrosis. For example, as an organ undergoes fibrosis, the functional cellular mass of the organ is reduced as it is replaced by scar tissue (collagens and other abnormal matrix components). In addition, fibrosis causes architectural disorganization that can diminish function and lead to pathology, such as portal hypertension and increased risk of hepatocellular carcinoma in the case of the liver. Severe portal hypertension usually manifests as bleeding esophageal/gastric varices and/or ascities. In the kidney and pancreas the features of advanced fibrosis are renal failure and endocrine and/or exocrine pancreatic failure.

Monitoring Therapy

These actions of the compositions provided herein are, in certain embodiments, monitored by blood, serum and plasma markers of liver inflammation, injury, and fibrogenesis, including but not limited to; aspartate aminotransferase, alanine aminotransferase, gamma glutamyl transpeptidase, bilirubin, alpha-2 macroglobulin, haptoglobin, tissue inhibitor of metalloproteinase-1, hyaluronic acid, amino terminal propeptide of type III collagen and other collagen precursors and metabolites, platelet count, apolipoprotein A1, C-reactive protein and ferritin. These tests are used alone in some examples, whereas in other examples they are used in combination. Hepatic fibrosis may also be monitored by the technique of transient elastography (Fibroscan™). A further embodiment includes monitoring the impact of the treatments by direct examination of liver tissue obtained by liver biopsy.

The effects of the disclosed methods on diseases of the pancreas are monitored, in some embodiments, by blood, serum, plasma amylase, or lipase, as well as tests of pancreatic exocrine and endocrine function. In other embodiments, pancreatitis or pancreatic cancer is monitored by imaging techniques, including but not limited to radiological, nuclear medicine, ultrasound, and magnetic resonance.

The effects of the disclosed methods on diseases of the kidney are monitored, in some embodiments, by the measurement of blood, serum, or plasma urea or creatinine, or other tests of renal function, alone or in combination. Kidney disease is monitored, in some embodiments, by imaging techniques, including but not restricted to radiological, nuclear medicine, ultrasound, and magnetic resonance. In alternate embodiments, the impact of the treatments on the kidney is monitored by direct examination of tissue obtained by kidney biopsy.

Combination with Other Therapeutic Agents

The disclosed compositions can be used for treatment in combination with other therapeutic agents, such as VDR agonists, chemotherapies and biotherapies. In one example, the other therapeutic agents include one or more nuclear receptor ligands, including but not limited to ligands for peroxisome proliferator-activated receptor-gamma (PPAR-γ, NR1C3), peroxisome proliferator-activated receptor-alpha (PPAR-α, NR1C1) and peroxisome proliferator-activated receptor-delta (PPAR-δ, NR1C2), farnesoid x receptor (FXR, NR1H4), interferon-gamma (IFN-γ), angiotensin converting enzyme inhibitors, angiotensin II receptor antagonists, ursodeoxycholic acid (UDCA), curcumin, anti-oxidants including, but not limited to vitamin E, retinoids such as Vitamin A, and therapies that deliver proteases to the liver to degrade pathological ECM. In one example, the compositions are administered to a subject's previously administered TGF-β1, such as a mammalian (e.g., human or rodent) TGF-β1, sufficient to increase VDR expression (such as an increase of at least 3-fold or at least 5-fold).

Exemplary VDR Ligands and Agonists

In one example, the methods use the disclosed compositions in combination with VDR ligands or other VDR agonists that can bind to and activate the VDR, for example to prevent or attenuate the processes of injury, inflammation, and fibrogenesis in the liver, pancreas and/or kidney.

In some examples, 1α,25(OH)₂D₃ or a vitamin D precursor or analog is used as a VDR agonist. It is not necessary to use the most biologically active form of vitamin D to achieve a beneficial therapeutic effect. The naturally occurring ligand of the vitamin D receptor is calcitriol. In one embodiment, precursors of calcitriol (such as calcidiol) are administered to a subject, and are then converted within the target cell population to calcitriol.

In addition, HSCs express CYP24A1, a cytochrome P450 enzyme that terminates the biological effect of calcitriol by side chain hydroxylation. Thus, in one embodiment, a VDR ligand or other VDR agonist or agonist precursor that is resistant to deactivation by CYP24A1 is used to achieve more effective and longer lasting VDR activation in target cell populations. In specific examples, the VDR ligand is one that can be activated by CYP27B1 while being resistant to deactivation by CYP24A1. This permits VDR activation in target cell populations in the liver (for example, HSCs), pancreas and kidney, while minimizing undesirable systemic effects on calcium homeostasis.

A further embodiment is the use of a molecule that is a VDR agonist or precursor thereof that exhibits the property of high first-pass hepatic clearance due to extensive hepatic metabolism. A molecule with this property, when administered orally, is absorbed and transported to the liver via the portal vein. In the liver, the molecule activates VDR in cell populations such as hepatic stellate cells, Kupffer cells and sinusoidal endothelial cells while exhibiting minimal systemic effects on calcium homeostasis due to low systemic bioavailability.

Exemplary VDR agonists that can be used with the disclosed methods include those molecules that can activate the VDR. Methods of determining if an agent is a VDR agonist are routine. For example, induction of CYP24A1 expression can be measured in cells that expressing VDR contacted with the agent, wherein an increase in CYP24A1 expression (such as a 10- to 20-fold increase in expression) indicates that the agent is a VDR agonist. Other methods include transfected reporter gene constructs and FRET assays. In some example, binding of an agonist to a purified LBD is detected by measuring induced recruitment for coactivator peptides (e.g., LXXLL). For example VDR agonists can increase CYP24A1 expression in a VDR-expressing cell by at least 20%, at least 50%, at least 75%, at least 80%, at least 90% at least 100%, at least 200% or oven at least 1000% or more as compared to the absence of the agonist. VDR agonists include molecules that can bind to and activate the VDR, such as 1α,25(OH)₂-D3 and precursors and analogs thereof, VDR ligands, and VDR agonist precursors. The disclosure is not limited to particular vitamin D agonists. A variety of biologically active vitamin D agonists are contemplated. Exemplary agents are known in the art.

VDR agonists include vitamin D compounds, precursors and analogs thereof. Vitamin D compounds useful for the methods provided herein include, but are not limited to compounds which have at least one of the following features: the C-ring, D-ring and 3β-hydroxycyclohexane A-ring of vitamin D interconnected by the 5,7 diene double bond system of vitamin D together with any side chain attached to the D-ring (e.g., compounds with a ‘vitamin D nucleus’ and substituted or unsubstituted A-, C-, and D-rings interconnected by a 5,7 diene double bond system typical of vitamin D together with a side chain attached to the D-ring).

Vitamin D analogs include those nonsecosteroid compounds capable of mimicking various activities of the secosteroid calcitriol. Examples of such compounds include, but are not limited to, LG190090, LG190119, LG190155, LG190176, and LG1900178 (See, Boehm et al., Chemistry & Biology 6:265-275, 1999).

Vitamin D compounds includes those compounds includes those vitamin D compounds and vitamin D analogs which are biologically active in vivo, or are acted upon in a mammalian subject such that the compound becomes active in vivo. Examples of such compounds include, but are not limited to: vitamin D, calcitriol, and analogs thereof [e.g., 1α-hydroxyvitamin D₃ (1α-OH-D₃), 1α,25-dihydroxyvitamin D2 (1,25-(OH)₂D₂), 1α-hydroxyvitamin D₂ (1α-OH-D₂), 1α,25-(OH)₂-16-ene-D₃, 1α,25-(OH)₂-24-oxo-16-ene-D₃, 1α,24R(OH)₂-D₃, 1α,25(OH)₂-22-oxa-D₃, 20-epi-22-oxa-24a,24b,-dihomo-1α,25(OH)₂-D₃, 20-epi-22-oxa-24a,26a,27a,-trihomo-1α25(OH)₂-D₃, 20-epi-22-oxa-24homo-1α,25(OH)₂-D₃, 1,25-(OH)₂-16,23E-diene-26-trifluoro-19-nor-D₃, and nonsecosteroidal vitamin D mimics.

In one example, the VDR agonist is one or more of the following vitamin D, 1,α25 dihydroxyvitamin D3, 1α-hydroxyvitamin D₃, 1,25-dihydroxyvitamin D₂, 1α-hydroxyvitamin D₂, 1α,25-(OH)₂-16-ene-D₃, 1α,25-(OH)₂-24-oxo-16-ene-D₃, 1α,24R(OH)₂-D₃, 1α,25(OH)₂-22-oxa-D₃, 20-epi-22-oxa-24a,24b,-dihomo-1α,25(OH)₂-D₃, 20-epi-22-oxa-24a,26a,27a,-trihomo-1α25(OH)₂-D₃, 20-epi-22-oxa-24homo-1α,25(OH)₂-D3, and 1,25-(OH)₂-16,23E-diene-26-trifluoro-19-nor-D₃. In a preferred embodiment, the biologically active vitamin D compound is selected from 1,α25-dihydroxyvitamin D₃, 19-nor-1,25-dihydroxyvitamin D₂, 19-nor-1,25-dihydroxy-21-epi-vitamin D₃, 1,25-dihydroxy-24-homo-22-dehydro-22E-vitamin D₃, and 19-nor-1,25-dihydroxy-24-homo-22-dehydro-22E-vitamin D₃, and nonsecosteroidal vitamin D mimics. In an additional example, the biologically active VDR agonist is selected from the analogs represented by the following formula:

wherein X¹ and X² are each selected from the group consisting of hydrogen and acyl; wherein Y¹ and Y² can be H, or one can be O-aryl or O-alkyl while the other is hydrogen and can have a (3 or a. configuration, Z¹ and Z² are both H, or Z¹ and Z² taken together are CH₂; and wherein R is an alkyl, hydroxyalkyl or fluoroalkyl group, or R may represent the following side chain:

wherein (a) may have an S or R configuration and wherein R¹ represents hydrogen, hydroxy or O-acyl, R² and R³ are each selected from the group consisting of alkyl, hydroxyalkyl and fluoroalkyl, or, when taken together represent the group —(CH₂)m- where m is an integer having a value of from 2 to 5, R⁴ is selected from the group consisting of hydrogen, hydroxy, fluorine, O-acyl, alkyl, hydroxyalkyl and fluoroalkyl, R⁵ is selected from the group consisting of hydrogen, hydroxy, fluorine, alkyl, hydroxyalkyl and fluoroalkyl, or, R⁴ and R⁵ taken together represent double-bonded oxygen, R⁶ and R⁷ taken together form a carbon-carbon double bond and R⁸ may be H or CH₃, and wherein n is an integer having a value of from 1 to 5, and wherein the carbon at any one of positions 20, 22, or 23 in the side chain may be replaced by an O, S, or N atom.

In one example, the VDR agonists used in the methods provided herein do not cause symptoms of hypercalcemia when administered to a subject. In another example, the VDR agonists do not generate as much (i.e., a lesser degree) of a calcemic response as compared to calcitriol when administered to a subject. In one example, VDR agonists have low calcemic response characteristics as compared to calcitriol. In another embodiment, these compounds are selected from 1α,25-(OH)₂-24-epi-D₂, 1α,25-(OH)₂-24a-Homo-D₃, 1α,25-(OH)₂ 24a-Dihomo-D₃, 1α,25-(OH)₂-19-nor-D₃, and 20-epi-24-homo-1α,25-(OH)₂-D₃.

Other exemplary VDR agonists that can be used in the methods provided herein are provided in Table 1.

TABLE 1
1,25-(OH)₂D₃ and its synthetic analogs
(taken from Nagpal et al., Endocr. Rev. 2005; 26:662-687).
Vitamin D Analogs

| Compound | R |
|---|---|
| 1α,25-(OH)₂D₃ (Calcitriol) | |
| 1α,25-(OH)₂-22,24- diene-24a,26a,27a- trihomo-D₃ (EB 1089) | |
| 1α-(OH)D₃ (Alfacalcidol) | |
| 1α,25-(OH)₂-22- ene-25-oxa-D₃ (ZK 156718) | |
| 1α,24-(OH)₂-24- cyclopropyl-D₃ (Calcipotriol) | |
| 25-(4-methylthiazol- 2-yl)-calcipotriol (ZK 191732) | |
| 1α,25-(OH)₂-22- oxa-D₃ (Maxacalcitol) | |
| 1α,24R-(OH)₂D₃ (Tacalcitol) | |

```
  1α,25-(OH)₂D₃ (Calcitriol)
  ED-71 [1α,25-(OH)₂-2β-(3-hydroxypropyl)D₃)
“20-Epi Vitamin D Analogs”

```

| Compound | R |
|---|---|
| 20-epi-22-ethoxy-23- yne-24a,26a,27a- trihomo-1α,25- (OH)₂D₃ (CB 1093) | |
| 20-epi-1α,25- (OH)₂D₃ (KH 1060) | |

```
  1α-fluoro-25-(OH)-16,23E-diene-26,27-bishomo-20epi- cholecalciferol (Ro-26-6228, BXL-628, RS-980400)
  2-methylene-19-nor-(20S)-1α,25-(OH)₂D₃ (2MD)
```

In one example, therapeutically effective doses of vitamin D2 and D3 range, from about 50 IU to about 50,000 IU. In some embodiments, vitamin D2 and/or D3 is administered in an oral dose of, for example, less than about 75 IU, about 100 IU, about 250 IU, about 500 IU, about 750 IU, about 1,000 IU, about 1,500 IU, about 2,000 IU, about 2,500 IU, about 5,000 IU, about 7,500 IU, about 10,000 IU, about 15,000 IU, about 20,000 IU, about 25,000 IU, about 40,000 IU, or about 50,000 IU, or more. In other embodiments, calcitriol is administered in a dose of from 0.001 to 10 micrograms. For instance, calcitrol is administered, in some embodiments, in a dose of about 0.01 μg, about 0.05 μg, about 0.1 about 0.25 μg, about 0.5 μg, about 1 μg, about 5 μg, or about 10 μs. In some embodiments, larger doses of VDR agonists are administered via a delivery route that targets the organ of interest, for instance the liver, kidney or pancreas.

In certain embodiments, the VDR agonist is administered orally, for instance, in single or divided doses. For oral administration, the compositions are, for example, provided in the form of a tablet containing 1.0 to 1000 mg of the active ingredient, such as at least 75 IU, at least 100 IU, at least 250 IU, at least 500 IU, at least 750 IU, at least 800 IU, at least 1,000 IU, at least 1,500 IU, at least 2,000 IU, at least 2,500 IU, at least 5,000 IU, at least 7,500 IU, at least 10,000 IU, at least 15,000 IU, at least 20,000 IU, at least 25,000 IU, at least 40,000 IU, or 5 at least 0,000 IU per day, for example 50 IU to 2000 IU per day, 100 IU to 1000 IU per day, such as 800 IU per day, or more of the active ingredient for the symptomatic adjustment of the dosage to the subject being treated. An effective parenteral dose could be expected to be lower, for example in the range of about 0.001 μg to about 10 μg, depending on the compound.

In another embodiment, if the VDR agonist is not a 1α-hydroxy compound, a daily dose between 1.0 and 100 μg per day per 160 pound patient is administered, such as between 5.0 and 50 μg per day per 160 pound patient. In a different embodiment, if the biologically active vitamin D compound is a 1α-hydroxy compound, a daily dose of between 0.1 and 20 μg per day per 160 pound patient is administered, while a preferred dose is between 0.5 and 10μ per day per 160 pound patient. In a particular example, the dose is between 3-10 μg per day.

In one example, the VDR agonist is cholecalciferol or calcidiol. In some examples, a higher dose than usual is administered, but with less frequency, for example, 50,000 to 500,000 units weekly.

### Exemplary Chemotherapies and Biologic Therapies

The disclosed methods can use the disclosed compositions in combination with other therapeutic agents, such as chemotherapies and biotherapies. Chemotherapies and biotherapies can include anti-neoplastic chemotherapeutic agents, antibiotics, alkylating agents and antioxidants, kinase inhibitors, and other agents such as antibodies. Methods and therapeutic dosages of such agents are known to those skilled in the art, and can be determined by a skilled clinician. Other therapeutic agents, for example anti-tumor agents, that may or may not fall under one or more of the classifications below, also are suitable for administration in combination with the described BET inhibitors. Selection and therapeutic dosages of such agents are known to those skilled in the art, and can be determined by a skilled clinician.

In one example, a chemotherapy or biotherapy increases killing of cancer cells (or reduces their viability). Such killing need not result in 100% reduction of cancer cells; for example a cancer chemotherapy that results in reduction in the number of viable cancer cells by at least 10%, at least 20%, at least 30%, at least 40%, at least 50%, at least 75%, at least 90%, or at least 95% (for example as compared to no treatment with the cancer chemotherapy or bio-therapy) can be used in the methods provided herein. For example, the cancer chemotherapy or bio-therapy can reduce the growth of cancer cells by at least 10%, at least 20%, at least 30%, at least 40%, at least 50%, at least 75%, at least 90%, or at least 95% (for example as compared to no chemotherapy or bio-therapy).

Particular examples of chemotherapic agents that can be used include alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide); microtubule binding agents (such as paclitaxel, docetaxel, vinblastine, vindesine, vinorelbine (navelbine) vincristine, the epothilones, colchicine, dolastatin 15, nocodazole, podophyllotoxin, rhizoxin, and derivatives and analogs thereof), DNA intercalators or cross-linkers (such as cisplatin, carboplatin, oxaliplatin, mitomycins, such as mitomycin C, bleomycin, chlorambucil, cyclophosphamide, and derivatives and analogs thereof), DNA synthesis inhibitors (such as methotrexate, 5-fluoro-5′-deoxyuridine, 5-fluorouracil and analogs thereof); anthracycline family members (for example, daunorubicin, doxorubicin, epirubicin, idarubicin, mitoxantrone, and valrubicin); antimetabolites, such as cytotoxic/antitumor antibiotics, bleomycin, rifampicin, hydroxyurea, and mitomycin; topoisomerase inhibitors, such as topotecan and irinotecan; photosensitizers, such as aminolevulinic acid, methyl aminolevulinate, porfimer sodium, and verteporfin, enzymes, enzyme inhibitors (such as camptothecin, etoposide, formestane, trichostatin and derivatives and analogs thereof), kinase inhibitors (such as imatinib, gefitinib, and erolitinib), gene regulators (such as raloxifene, 5-azacytidine, 5-aza-2′-deoxycytidine, tamoxifen, 4-hydroxytamoxifen, mifepristone and derivatives and analogs thereof); and other agents, such as alitretinoin, altretamine, amsacrine, anagrelide, arsenic trioxide, asparaginase, axitinib, bexarotene, bevacizumab, bortezomib, celecoxib, denileukin diftitox, estramustine, hydroxycarbamide, lapatinib, pazopanib, pentostatin, masoprocol, mitotane, pegaspargase, tamoxifen, sorafenib, sunitinib, vemurafinib, vandetanib, and tretinoin.

In one example, a bio-therapy includes or consists of an antibody, such as a humanized antibody. Such antibodies can be polyclonal, monoclonal, or chimeric antibodies. As noted above, methods of making antibodies specific for a particular target is routine. In some example, the therapeutic antibody is conjugated to a toxin. Exemplary biotherapies include alemtuzumab, bevacizumab, cetuximab, gemtuzumab, rituximab, panitumumab, pertuzumab, and trastuzumab.

Other examples of bio-therapy include inhibitory nucleic acid molecules, such as an antisense oligonucleotide, a siRNA, a microRNA (miRNA), a shRNA or a ribozyme. Any type of antisense compound that specifically targets and regulates expression of a target nucleic acid is contemplated for use. An antisense compound is one which specifically hybridizes with and modulates expression of a target nucleic acid molecule. These compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound. In some examples, an antisense oligonucleotide is a single stranded antisense compound, such that when the antisense oligonucleotide hybridizes to a target mRNA, the duplex is recognized by RNaseH, resulting in cleavage of the mRNA. In other examples, a miRNA is a single-stranded RNA molecule of about 21-23 nucleotides that is at least partially complementary to an mRNA molecule that regulates gene expression through an RNAi pathway. In further examples, a shRNA is an RNA oligonucleotide that forms a tight hairpin, which is cleaved into siRNA. siRNA molecules are generally about 20-25 nucleotides in length and may have a two nucleotide overhang on the 3′ ends, or may be blunt ended. Generally, one strand of a siRNA is at least partially complementary to a target nucleic acid. Antisense compounds specifically targeting a gene can be prepared by designing compounds that are complementary to a target nucleotide sequence, such as a mRNA sequence. Antisense compounds need not be 100% complementary to the target nucleic acid molecule to specifically hybridize and regulate expression of the target. For example, the antisense compound, or antisense strand of the compound if a double-stranded compound, can be at least 75%, at least 80%, at least 85%, at least 90%, at least 95%, at least 99% or 100% complementary to a target nucleic acid sequence. Methods of screening antisense compounds for specificity are well known (see, for example, U.S. Publication No. 2003-0228689). In addition, methods of designing, preparing and using inhibitory nucleic acid molecules are within the abilities of one of skill in the art.

Administration of Therapeutic Agents

In some examples, the disclosed methods include providing a therapeutically effective amount of one or more of the disclosed compositions alone or in combination with another therapeutic agent, such as a VDR agonist, chemotherapy or biotherapy, to a subject. Methods and therapeutic dosages of such agents and treatments are known to those of ordinary skill in the art, and for example, can be determined by a skilled clinician. In some examples, the disclosed methods further include providing surgery and/or radiation therapy to the subject in combination with the treatments described herein (for example, sequentially, substantially simultaneously, or simultaneously). Administration can be accomplished by single or multiple doses. Methods and therapeutic dosages of such agents and treatments are known to those skilled in the art, and can be determined by a skilled clinician. The dose required will vary from subject to subject depending on the species, age, weight and general condition of the subject, the particular therapeutic agent being used and its mode of administration.

Therapeutic agents can be administered to a subject in need of treatment using any suitable means known in the art. Methods of administration include, but are not limited to, intradermal, transdermal, intramuscular, intraperitoneal, parenteral, intravenous, subcutaneous, vaginal, rectal, intranasal, inhalation, oral, or by gene gun. Intranasal administration refers to delivery of the compositions into the nose and nasal passages through one or both of the nares and can include delivery by a spraying mechanism or droplet mechanism, or through aerosolization of the therapeutic agent.

Administration of the therapeutic agents by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Therapeutic agents can be administered in any suitable manner, for example with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Therapeutic agents for oral administration include powders or granules, suspensions or solutions in water or non-aqueous media, capsules, sachets, or tablets. Thickeners, flavorings, diluents, emulsifiers, dispersing aids or binders may be desirable.

Therapeutic agents can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

In some examples, the dose of a composition that includes a BET inhibitor (such as one or more of those provided herein, for example JQ1, OTX-015, and/or I-BET-762) and a nanoparticle is about 1 mg to about 1000 mg, about 10 mg to about 500 mg, or about 50 mg to about 100 mg. In some examples, the dose of the composition is about 1 mg, about 10 mg, about 50 mg, about 100 mg, about 250 mg, about 500, about 700 mg, about 1000 mg, about 2000 mg, about 3000 mg, about 4000 mg, about 5000 mg, about 6000 mg, about 7000 mg, about 9000 mg or about 10,000 mg. In some embodiments, the dose of a the composition (such as one or more of those provided herein, for example JQ1, OTX-015, and/or I-BET-762) is about 1 mg/kg to about 1000 mg/kg, or about 5 mg/kg to about 500 mg/kg, about 10 mg/kg to about 100 mg/kg, about 50 mg/kg to 100 mg/kg, about 60 to about 80 mg/kg, or about 25 to about 50 mg/kg. In some examples, the dose of the composition is about 1 mg/kg, about 5 mg/kg, about 10 mg/kg, about 12.5 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 75 mg/kg, about 80 mg/kg or about 100 mg/kg. In some examples, the BET inhibitor (such as one or more of those provided herein, for example JQ1, OTX-015, and/or I-BET-762) in the composition is present at these levels. Thus, about 1 mg/kg to about 1000 mg/kg of a BET inhibitor can be administered (such as one or more of those provided herein, for example JQ1, OTX-015, and/or I-BET-762), for example about 5 mg/kg to about 500 mg/kg, about 10 mg/kg to about 100 mg/kg, about 10 mg/kg to about 80 mg/kg, about 50 mg/kg to 100 mg/kg, about 60 to about 80 mg/kg, about 40 mg/kg to about 80 mg/kg, or about 25 to about 50 mg/kg. In some examples, the dose of the BET inhibitor (such as one or more of those provided herein, for example JQ1, OTX-015, and/or I-BET-762) is about 1 mg/kg, about 5 mg/kg, about 10 mg/kg, about 12.5 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 75 mg/kg, about 80 mg/kg, or about 100 mg/kg. In some examples, the dose of the BET inhibitor (such as one or more of those provided herein, for example JQ1, OTX-015, and/or I-BET-762) is about 1 mg/day to about 1000 mg/day, such as about 5 mg/day to about 500 mg/day, about 10 mg/day to about 100 mg/day, about 1 mg/day, about 5 mg/day, about 10 mg/day, about 12.5 mg/day, about 15 mg/day, about 20 mg/day, about 25 mg/day, about 30 mg/day, about 35 mg/day, about 40 mg/day, about 45 mg/day, about 50 mg/day, about 60 mg/day, about 70 mg/day, about 75 mg/day, about 80 mg/day, or about 100 mg/day. In one example, the BET inhibitor is OTX-015 and is administered at a dose of 1 mg/day to 100 mg/day, such as 10 mg/day to 80 mg/day, for example 10 mg/day, 40 mg/day, or 80 mg/day. In one example, the BET inhibitor is I-BET-762 and is administered at a dose of 1 mg/day to 100 mg/day, such as 10 mg/day to 50 mg/day, 10 mg/day to 30 mg/day, for example 1 mg/day, 10 mg/day, or 30 mg/day. It will be appreciated that these dosages are examples only, and an appropriate dose can be determined by one of ordinary skill in the art using only routine experimentation. In some examples, the composition is administered orally with water.

### Example 1

### Materials and Methods

### Cell Culture and qRT-PCR

Primary HSCs were isolated from 16-week old male C57BL/6J mice by in situ pronase, collagenase perfusion and single-step Histogenz gradient as previously reported. LX-2 cells, a generous gift from Professor Scott Friedman, Mount Sinai School of Medicine, New York, N.Y., were cultured as described previously²⁰. For quantitative RT-PCR (qRT-PCR), total RNA was purified following TRIzol extraction and treated with DNaseI (Life Technologies). Complementary DNA synthesis was carried out with iScript RT Supermix (Bio-Rad). Quantitative PCR was performed in technical triplicates using SYBR Green reagent (Bio-Rad). The relative standard curve method was used for quantitation (Bio-Rad). Expression levels were calculated by normalization to either Gapdh (mouse) or U36B4 (human) quantities. The sequences of primers are listed in Table 2.

TABLE 2
Primer seqs for QPCR
RT-qPCR Primers

| | | | SEQ ID |
|---|---|---|---|
| Gene Name | Species | Sequence | NO: |
| Gapdh | mouse | TCAACAGCAACTCCCACTCTTCCA | 1 |
| | | TTGTCATTGAGAGCAATGCCAGCC | 2 |
| Vdr | mouse | GCTGAACCTCCATGAGGAAG | 3 |
| | | GGATCATCTTGGCGTAGAGC | 4 |
| Cyp24a1 | mouse | GACCGCAAACAGCTTGATGTGGAT | 5 |
| | | ATATTCCTCACATCTTCCGCCCGT | 6 |
| Col1a1 | mouse | ACTGCAACATGGAGACAGGTCAGA | 7 |
| | | ATCGGTCATGCTCTCTCCAAACCA | 8 |
| Tgfβ1 | mouse | TTTGGAGCCTGGACACACAGTACA | 9 |
| | | TGTGTTGGTTGTAGAGGGCAAGGA | 10 |
| Timp1 | mouse | GGTGTGCACAGTGTTTCCCTGTTT | 11 |
| | | TCCGTCCACAAACAGTGAGTGTCA | 12 |
| U36B4 | human | GCAGTGATGTAAAATTTCTTGG | 13 |
| | | AAAGCTCGGTTTTACTCTTCACA | 14 |
| VDR | human | CTGTGGCAACCAAGACTACA | 15 |
| | | CCCACCTGGAACTTGATGAG | 16 |
| CYP24A1 | human | CCTGCTGCAGATTCTCTGGAA | 17 |
| | | AGGGTGTCGTGCTGTTTCTTG | 18 |
| SMAD2 | human | ACCGAAGGCAGACGGTAACAAGTA | 19 |
| | | GACATGCTTGAGCAACGCACTGAA | 20 |
| SMAD3 | human | ATGTCAACAGGAATGCAGCAGTGG | 21 |
| | | ATAGCGCTGGTTACAGTTGGGAGA | 22 |
| Co11A1 | human | CGGTGTGACTCGTGCAGC | 23 |
| | | ACAGCCGCTTCACCTACAGC | 24 |
| Co11A2 | human | TCAAACTGGCTGCCAGCAT | 25 |
| | | CAAGAAACACGTCTGGCTAGG | 26 |
| TGFβ1 | human | CGCTAAGGCGAAAGCCCTCAATTT | 27 |
| | | ACAATTCCTGGCGATACCTCAGCA | 28 |
| TIMP1 | human | TCTGCAATTCCGACCTCGTCATCA | 29 |
| | | AAGGTGGTCTGGTTGACTTCTGGT | 30 |

ChIP-qPCR Primers

| Gene Name | Species | Sequence | |
|---|---|---|---|
| Col1A1 | human | CATTCCCAGCTCCCCTCTCT | 31 |
| | | AGTCTACGTGGCAGGCAAGG | 32 |

### RNA-seq and Data Analysis

HSCs isolated from mouse livers were cultured on plastic for 24 hours (day 1) prior to DMSO or JQ1 treatment for two (day 3) or five (day 6) additional days, with biological duplicates for all treatments. Total RNA was isolated using Trizol (Invitrogen) and the RNeasy mini kit (Qiagen). RNA purity and integrity were confirmed using an Agilent Bioanalyzer. Libraries were prepared from 100 ng total RNA (TrueSeq v2, Illumina) and singled-ended sequencing performed on the Illumina HiSeq 2000, using bar-coded multiplexing and a 100 bp read length, yielding a median of 34.1M reads per sample. Read alignment and junction finding was accomplished using STAR²⁹ and differential gene expression with Cuffdiff 2³⁰, utilizing UCSC mm9 as the reference sequence.

Transfection of siRNAs

Transfection was carried out at a concentration 10 nM of indicated siRNAs (Dharmacon) using RNAiMax transfection reagent (Invitrogen). Transfected cells were cultured without perturbation for at least 72 hours prior to terminal assays.

### CCl₄ Model of Liver Injury and Fibrosis

For preventive study, 6 week-old male C57BL/6J mice were IP injected with 0.5 ml/kg body weight CCl₄ (1:50 v/v in corn oil from Sigma) or corn oil three times a week for 4 weeks. JQ1 (50 mg/kg body weight) or vehicle (10% 2-Hydroxypropyl-β-cyclodextrin [HP-β-CD] from Sigma) was administered by IP injection 5 times a week, commencing 20 days after the first dose of CCl₄ or corn oil. The animals were terminated 72 hours after the final CCl₄ injection and whole livers were collected for histological, cytological, biochemical and molecular analyses. For therapeutic study, 6 week-old male C57BL/6J mice were first IP injected with 0.5 ml/kg body weight CCl₄ three times a week for 4 weeks to establish liver fibrosis. The same study group were then continuously IP injected with 0.5 ml/kg body weight CCl₄ three times a week for another 8 weeks with JQ1 (50 mg/kg body weight) or vehicle (10% HP-β-CD) co-administration by IP injection 5 times a week, commencing 40 days after liver fibrosis was initialized.

### Fibrotic Score and Quantification Hepatic Collagen and Hydroxyproline Content

5 μm sections of formalin-fixed liver were stained following standard hematoxylin-eosin (H&E) and Sirius Red methods and reviewed by a pathologist who was blinded to the experimental conditions. Fibrosis was scored using the Ishak modified histological activity index (HAI) scoring system. Liver fibrosis was also quantified using Image J software on 10 non-contiguous Sirius Red stained sections. Kidney fibrosis was quantified using hydroxyproline assay. All images were obtained using a high-resolution Leica DFC420 digital camera mounted on an Olympus microscope equipped with ×4/0.13, ×10/0.30, ×20/0.50 and ×40/0.75 UplanFL N plan objective lenses and processed with the Leica Application Suite. Tissue hydroxyproline content was measured using a kit from Biovision (K555-100).

### Serum Alanine Aminotransferase (ALT) Assay

The ALT activity in mice serum was measured using a kit from Thermo Scientific (TR71121).

### Cell Viability Assay

Primary HSCs or LX-2 cells were seeded onto 96-well tissue culture plates. 24 hours later, cells were treated with JQ1 at a series of concentrations for indicated periods prior to luciferase-based cell viability assay using CellTiter Glo kit (Promega, G7571).

### Cell Proliferation Assay

Primary HSCs or LX-2 cells cultured on 4-well chamber slides were treated with BrdU (3 μg/ml) for 4 hours followed by immunostaining using Alexa Fluor 488 conjugated mouse monoclonal BrdU antibody (Life Sciences, B35130). BrdU-positive cells were counted under fluorescent microscope (Olympus, IX51).

### TUNEL Assay

Primary HSCs or LX-2 cells cultured on 4-well chamber slides (Nalge Nunc, 154917) were subjected for TUNEL assay using DeadEnd™ Fluorometric TUNEL System (Promega, G3250).

### Cellular Senescence Staining

Primary HSCs or LX-2 cells cultured on 4-well chamber slides (Nalge Nunc, 154917) in the presence of DMSO (0.1%) or JQ1 (500 nM) were stained for senescence using a β-Galactosidase Staining Kit (Cell Signaling, 9860s).

### Lipid Droplet Accumulation Assay

Primary murine HSCs were seeded onto chamber slides (Nunc). After overnight attachment, cells were treated with DMSO or 500 nM JQ1 for another 5 days. Media was aspirated, cells were washed twice with PBS (Gibco) and fixed in 10% buffered formalin at room temperature for 15 minutes. Fixative was removed and cells were washed three times with PBS. Cells were then stained with 1 μg/ml 4,4-Difluoro-1,3,5,7,8-Pentamethyl-4-Bora-3a,4a-Diaza-s-Indacene (BODIPY 493/503, Molecular Probes) for 1 hour at room temperature, protected from light. Dye was removed and cells were washed three times with PBS, then mounted using Vectastain mounting medium (Vector Labs). Fluorescence was visualized through the GFP filter on a Leica DM5000B fluorescent microscope. Nuclear counterstaining was performed using DAPI (Vector).

### Immunocytochemistry

Primary murine HSCs were seeded onto chamber slides (Nunc). After overnight attachment, cells were treated with DMSO or 500 nM JQ1 for another 5 days. Media was aspirated, cells were washed twice with PBS (Gibco) and fixed in 10% buffered formalin at room temperature for 15 minutes. Fixative was removed and cells were washed three times with PBS. The slides were then blocked with 3% BSA and incubated with rabbit anti-ACTA2 antibody (at 1:100 dilution; Abcam) overnight at 4° C. After washing, the slides were incubated for 1 hour with Alexa Fluor546-labeled donkey anti-rabbit IgG antibody (at 1:200 dilution; Invitrogen). Finally, the slides were analyzed for fluorescence using an all-in-one type fluorescent microscope (BioZero BZ-9000; Keyence, Osaka, Japan). Nuclear counterstaining was performed using DAPI (Vector).

### Immunohistochemistry

Liver samples were deparaffinized and rehydrated in PBS. Following antigen retrieval with the target retrieval solution (Dako, Glostrup, Denmark), endogenous peroxidase activity was blocked by incubation with 0.3% hydrogen peroxide. After immersion in diluted normal rabbit serum, the sections were sequentially incubated with rabbit anti-ACTA2 antibody (at 1:2000 dilution; Abcam) 1 hour at 25° C. and biotinylated anti-rabbit IgG secondary antibody, followed by biotinylated enzyme-conjugated avidin. The color was developed by incubating the slides for several minutes with diaminobenzidine (Dojindo, Kumamoto, Japan). Counterstaining was performed using Haematoxylin (Sigma).

### Chromatin Immunoprecipitation

LX-2 cells were treated with DMSO (0.1%) or JQ1 (500 nM) for 16 hours. Cells were then harvested for ChIP assay. The experimental procedure for ChIP was as previously described. Briefly, after fixation, nuclei from LX-2 cells were isolated, lysed and sheared with a Diagenode Bioruptor to yield DNA fragment sizes of 200-1000 base pairs followed by immunoprecipitation using antibodies listed below: BRD2 (Bethyl, A302-583A) (2), BRD3 (Bethyl, A310-859A) (1), BRD4 (Bethyl, A301-985A) (1), PAF1 (Bethyl, A300-173A) (2), CDK9 (Santa Cruz, sc-484) (D0913), Pol II (Santa Cruz, sc-899) (C1413), Pol II S2p (Abcam, ab5095) (GR104063-1) and Pol II S5p (Abacm, ab5131) (GR104067-1).

ChIP-Seq and mRNA-Seq Data Analysis

The procedure was as previously described^{5,31}. Briefly, short DNA reads were demultiplexed using the Illumina CASAVA v1.8.2. Reads were aligned against the human hg18 reference genome (NCBI Build 36.1) using the Bowtie aligner allowing up to 2 mismatches in the read. Only tags that map uniquely to the genome were considered for further analysis. Subsequent peak calling and motif analysis were conducted using HOMER, a software suite for ChIP-Seq analysis. The methods for HOMER, which are described below, have been implemented and are freely available on the internet (biowhat.ucsd.edu/homer/). One tag from each unique position was considered to eliminate peaks resulting from clonal amplification of fragments during the ChIP-Seq protocol. Peaks were identified by searching for clusters of tags within a sliding 200 bp window, requiring adjacent clusters to be at least 1 kb away from each other. The threshold for the number of tags that determine a valid peak was selected for a false discovery rate of <0.01, as empirically determined by repeating the peak finding procedure using randomized tag positions. Peaks are required to have at least 4-fold more tags (normalized to total count) than input or IgG control samples and 4-fold more tags relative to the local background region (10 kb) to avoid identifying regions with genomic duplications or non-localized binding. Peaks are annotated to gene products by identifying the nearest RefSeq transcriptional start site. Visualization of ChIP-Seq results was achieved by uploading custom tracks onto the UCSC genome browser.

### Example 2

### BET Inhibition Blocks HSC Activation In Vitro

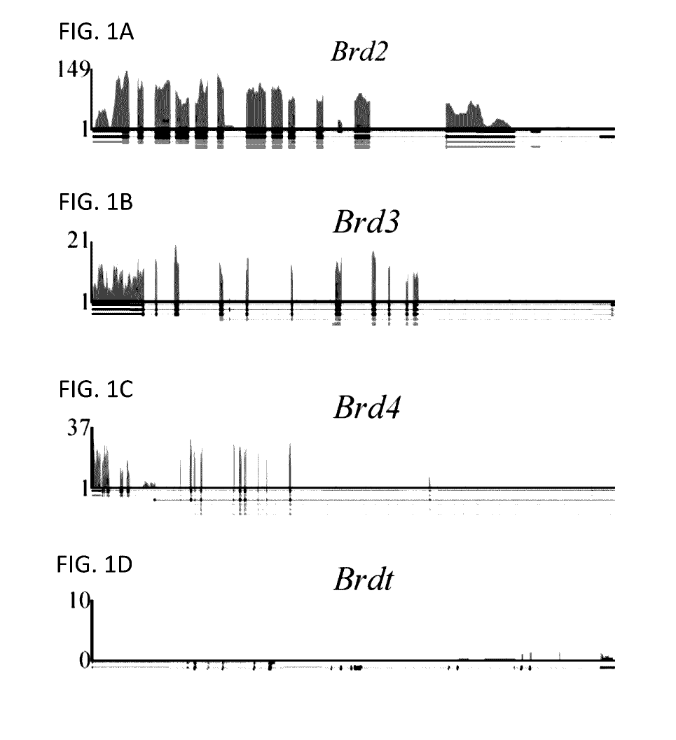

Activation of myofibroblasts is a hallmark of the pathogenesis and progression of tissue fibrosis⁴. The ability to pharmacologically target HSCs enables analysis of the activation mechanism and the ability to create a new type of anti-fibrotic therapy. Considering their critical role in modulating disease-relevant enhancer activity (Lee & Young, Cell. 152, 1237-1251. (2013), it was determined whether epigenetic pathways can be targeted to attenuate the fibrotic response in myofibroblasts. Through a directed chemical screen to identify such small molecules, it was observed that JQ1¹⁰, a highly selective bromodomain protein (BET) inhibitor, produced a 4.5 fold repression of COL1A1, in LX-2 cells, a well-established human activated HSC cell line (FIG. 1A), indicating a possible pro-fibrotic function for BETs.

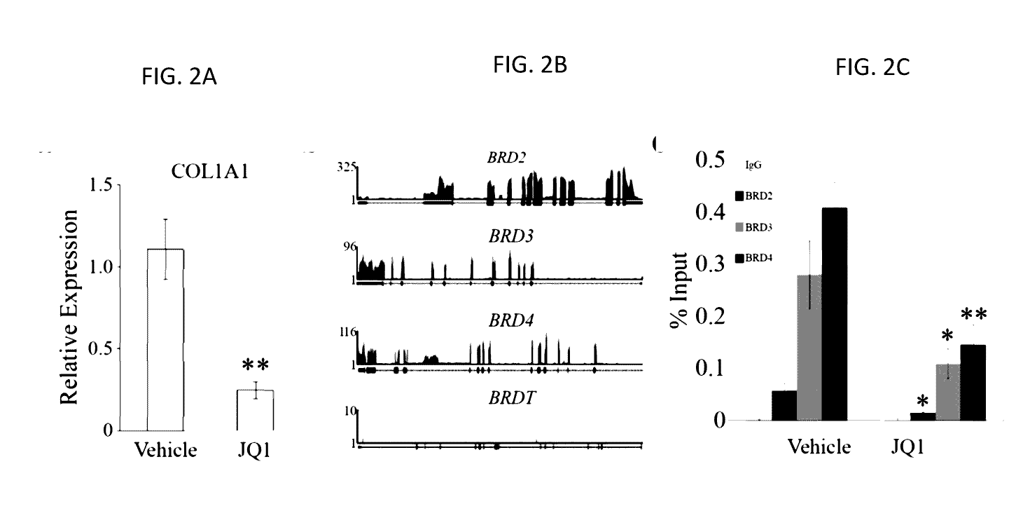

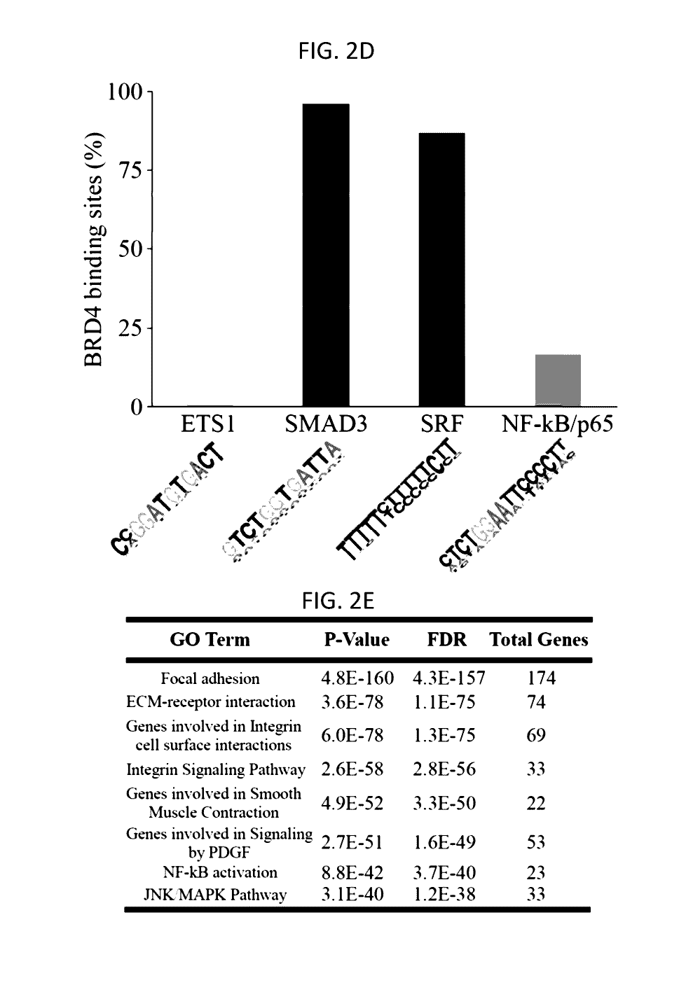

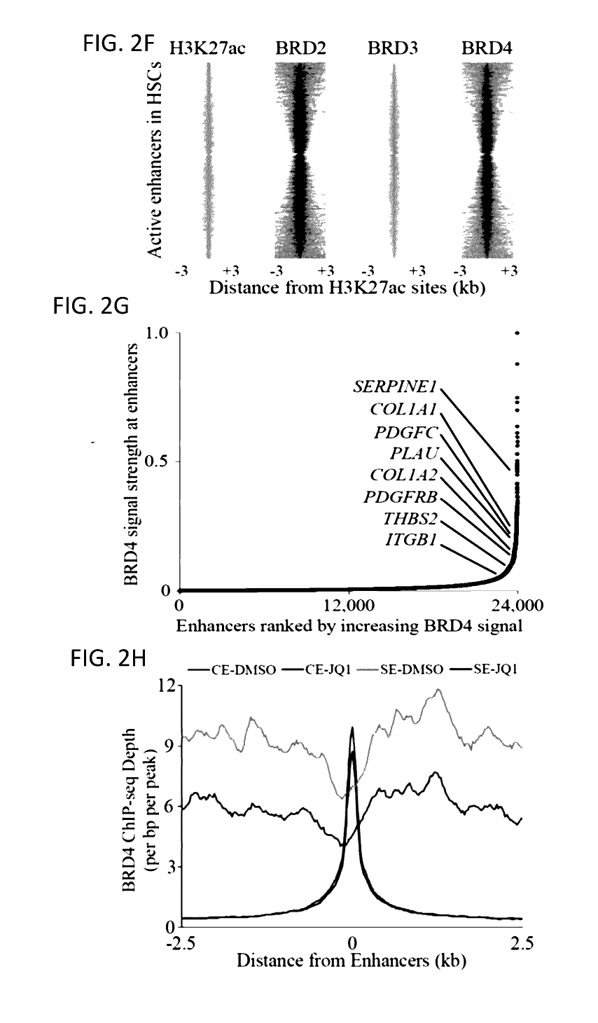

In support of this observation, RNAseq analysis of BET family members (Brd2, Brd3, Brd4 and Brdt) confirmed that Brd2/3/4 are all highly expressed in both LX-2 cell and primary HSCs while Brdt expression was not detected (FIGS. 1A-1D and FIG. 2B).

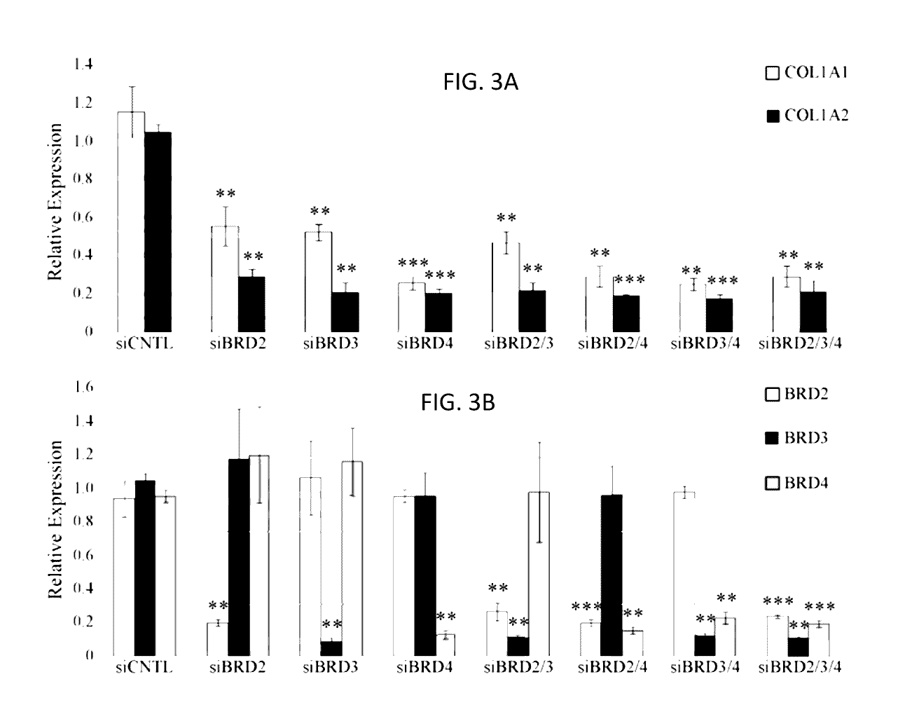

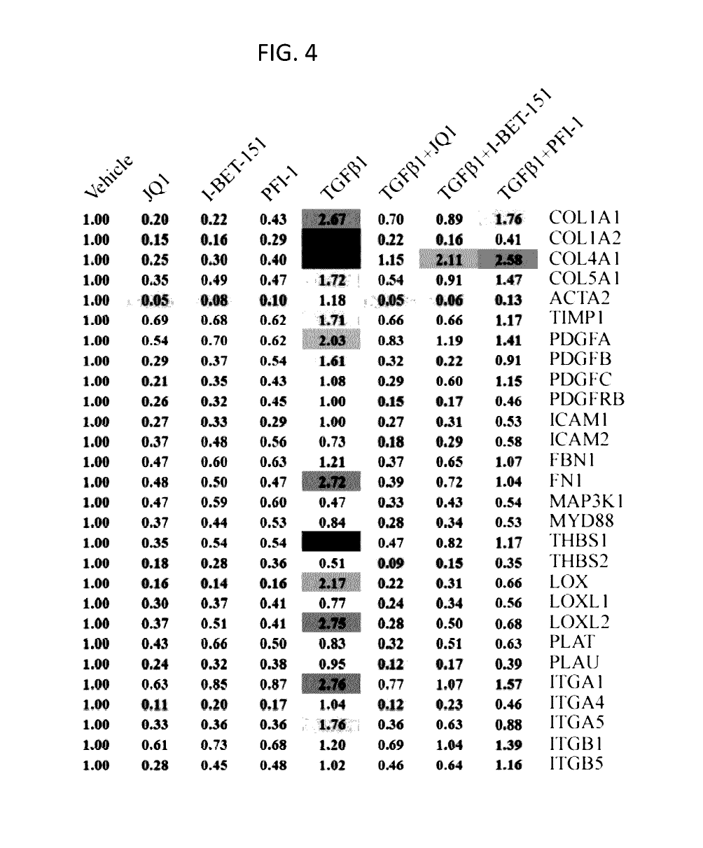

Furthermore, chromatin immunoprecipitation (ChIP) studies demonstrated that BETs bind to the COL1A1 enhancer locus and this occupancy is significantly diminished by JQ1 treatment (FIG. 2C), pointing to a direct role of BETs in modulating pro-fibrotic gene expression. Next, by coupling qRT-PCR analysis with RNA interference (RNAi), it was observed that loss of each BET compromised pro-fibrotic gene expression. No synergistic anti-fibrotic effects were observed when multiple BETs were simultaneously depleted (FIGS. 3A and 3B), indicating that BRD2/3/4 are all involved in mediating pro-fibrotic gene expression in activated HSCs, likely through a multisubunit complex¹¹. Consistent with these findings, two structurally distinct BET inhibitors (I-BET-151^{11,13} and PFI-1¹⁹) possess comparable inhibitory effects to JQ1 on the expression of a wide range of pro-fibrotic genes at equimolar doses in the absence or presence of TGFβ₁, a master pro-fibrotic cytokine (FIG. 4), revealing the ability of these inhibitors to exert a system wide suppression of fibrotic gene expression.

### Example 3

### BETs Modulate Pro-Fibrotic Super-Enhancer Activity in Activated HSCs

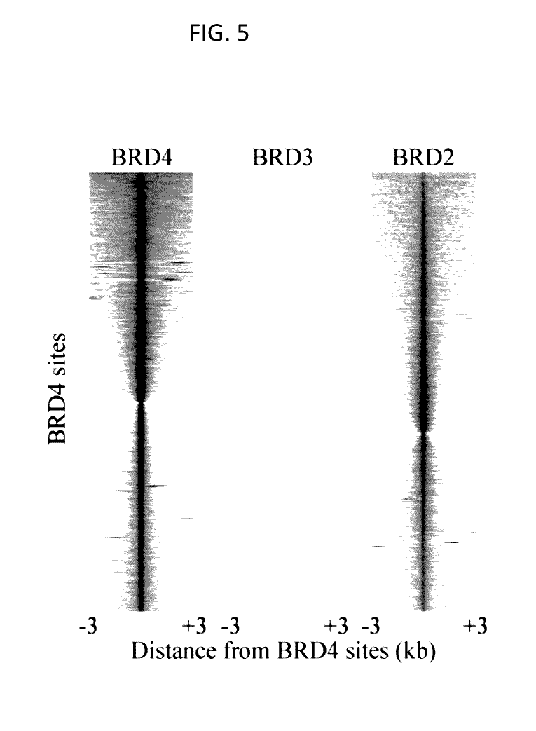

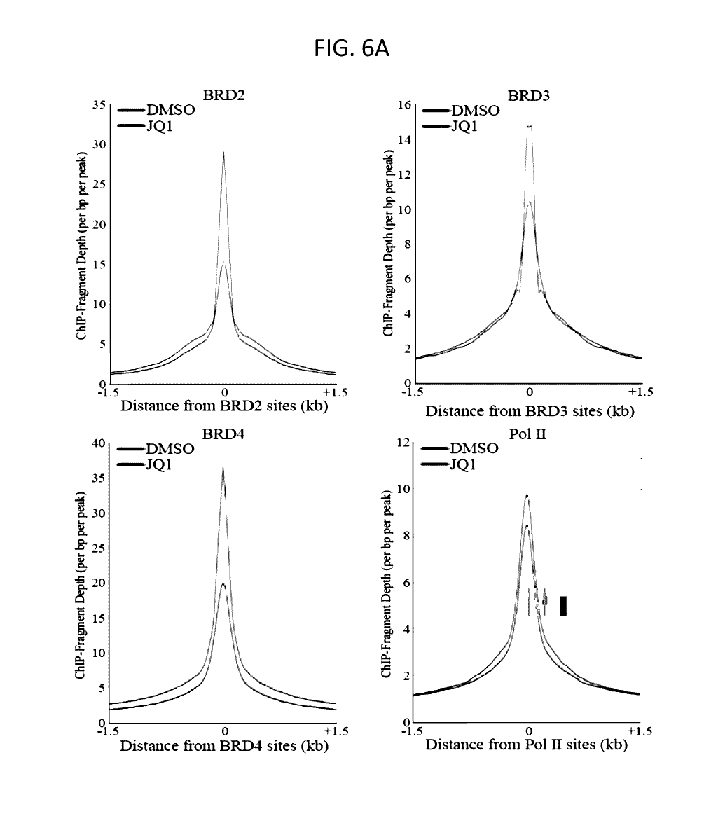

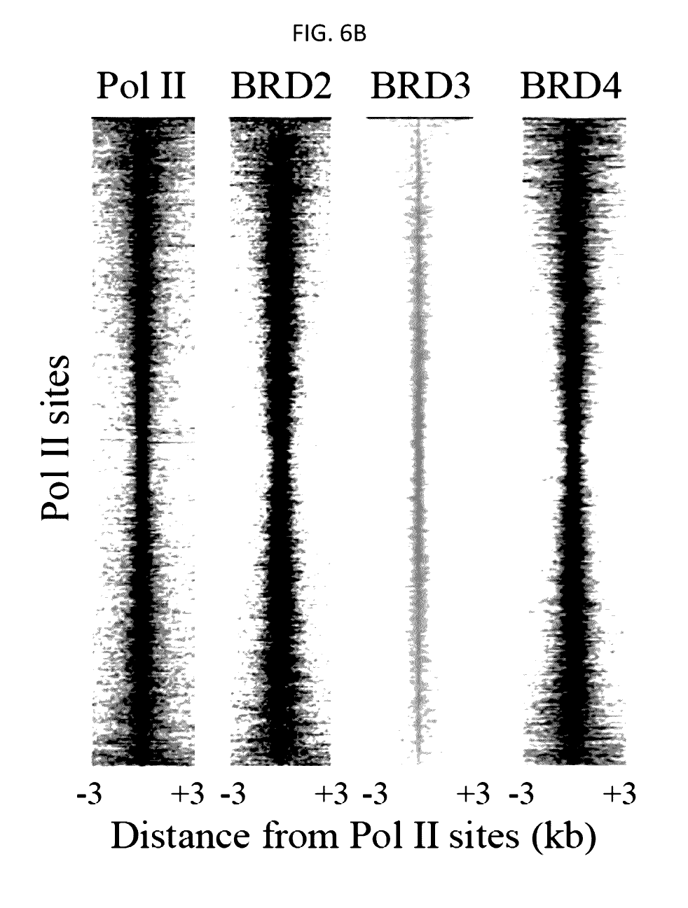

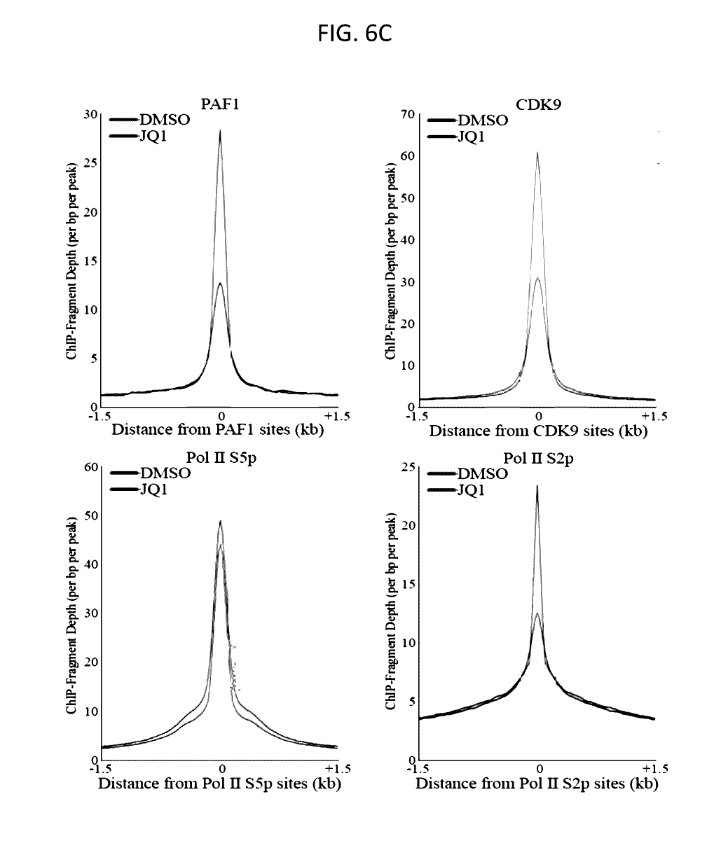

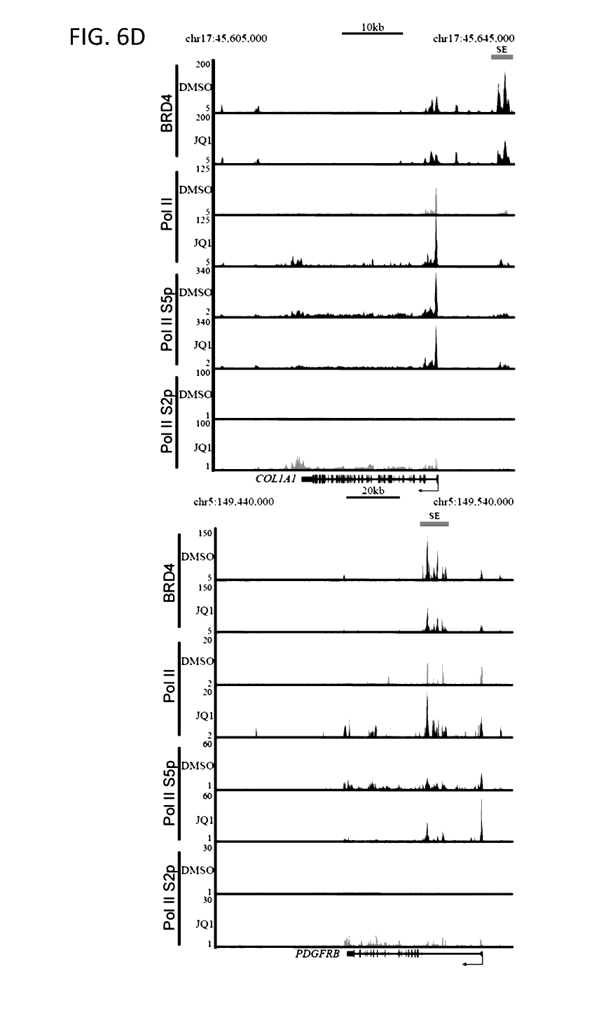

BETs are chromatin regulators⁷. To determine if they facilitate pro-fibrotic gene expression through their ability to co-activate multiple transcriptional pathways from regulatory enhancer elements, ChIP coupled with deep sequencing (ChIP-Seq) was performed to determine the global binding sites of BRD2/3/4 in LX-2 cells in the absence or presence of JQ1. The resulting cistromes revealed that BETs are largely co-localized in the genome (FIG. 5) and that JQ1 treatment dramatically reduces the occupancy of BETs on chromatin (FIG. 6A). Using BRD4 as a representative factor, it was observed that BETs are selectively loaded onto genomic regions highly enriched in binding motifs of prominent pro-fibrotic transcription factors including ETS1²³, SRF²⁴, SMAD3²⁶ and NF-κB²⁷ (FIG. 2D). In addition, gene ontology (GO) analysis of putative target genes confirmed that BETs target almost all of the well-characterized pro-fibrotic pathways including focal adhesion, ECM-receptor interaction, integrin signaling, smooth muscle contraction, PDGF signaling, NF-κB signaling and JNK/MAPK signaling² (FIG. 2E). Collectively, these results are consistent with the gene expression profiling data (FIG. 4) and indicate direct coordination of BETs with pro-fibrotic transcription factors.

To identify the molecular mechanism linking BETs to the fibrotic gene network, the genomic distribution of BET binding sites was examined relative to H3K27ac an epigenetic marker of active enhancers (FIG. 2F). This shows that all three BET proteins are localized to active enhancers. In contrast to BRD-2 and -3, BRD4 has been reported to bind to large enhancer clusters known as super-enhancers and to mark genes linked to cell identity and cancer^{28,32}. By extension, as activated stellate cells acquire a fibroblast identity and begin to proliferate, it was determined whether the pro-fibrotic actions of BETs are mediated through such super-enhancer activity. Preferential BRD4 loading was observed in a small subset (˜3%) of enhancers, whose genomic regions are considerably larger (>20 KB) than typical enhancers (FIGS. 2G and 2H), (defined as super-enhancers). Notably, fibrosis marker genes such as COL1A1 and PDGFRB are associated with BRD4-loaded super-enhancers (FIG. 2G). In addition, BRD4 occupancy on these super-enhancer regions is more sensitive to JQ1-induced reduction than control enhancer regions (FIG. 2H), indicating that BET inhibition may preferentially modulate pro-fibrotic gene expression through super-enhancers.

### Example 4

### BET Inhibition Perturbs Transcriptional Elongation in Activated HSCs

However, the pro-fibrotic function of BETs cannot be solely attributed to super-enhancers, as JQ1 causes a broader anti-fibrotic effect than super-enhancers alone could explain. The close localization of BETs to Pol II sites on the genome brings up the possibility that BETs might also target transcriptional elongation (FIG. 6B). It was observed that BET inhibition displaced BET-interacting transcriptional elongation cofactors such as PAF1 and CDK9 (part of PAF and P-TEFb complex, respectively) from chromatin (FIG. 6C, top 2 panels). Moreover, a specific decrease in the elongation-specific serine 2 phosphorylated form (S2p) of RNA Pol II recruitment was observed upon JQ1 treatment with little change to the initiation-specific serine 5 Pol II phospho-form (S5p) (FIG. 6C, bottom 2 panels), indicating that transcriptional elongation might be perturbed by JQ1. Taken together, it was concluded that combinatorial regulation of pro-fibrotic super-enhancers and transcriptional elongation serves as a major molecular mechanism through which BETs facilitate pro-fibrotic gene expression in activated human HSCs. The impact of blocking this pathway is exemplified at two key fibrosis marker genes; COL1A1 and PDGFRB (FIG. 6D).

### Example 5

### BET Inhibition Blocks HSC Activation into Myofibroblasts

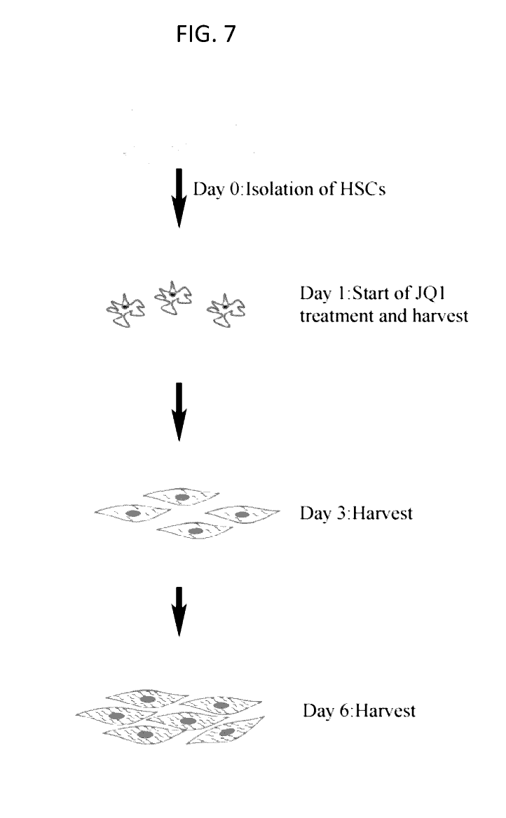

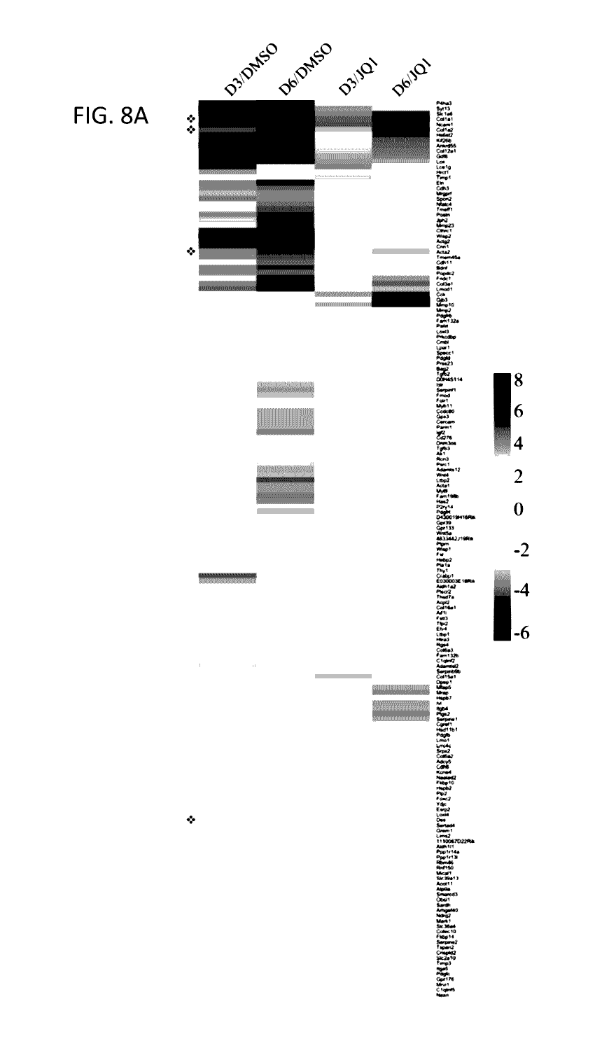

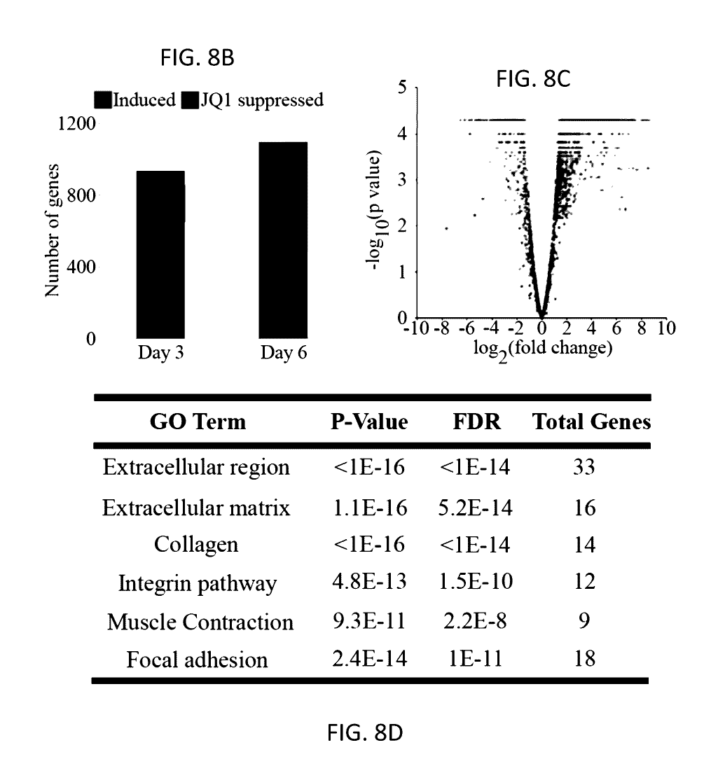

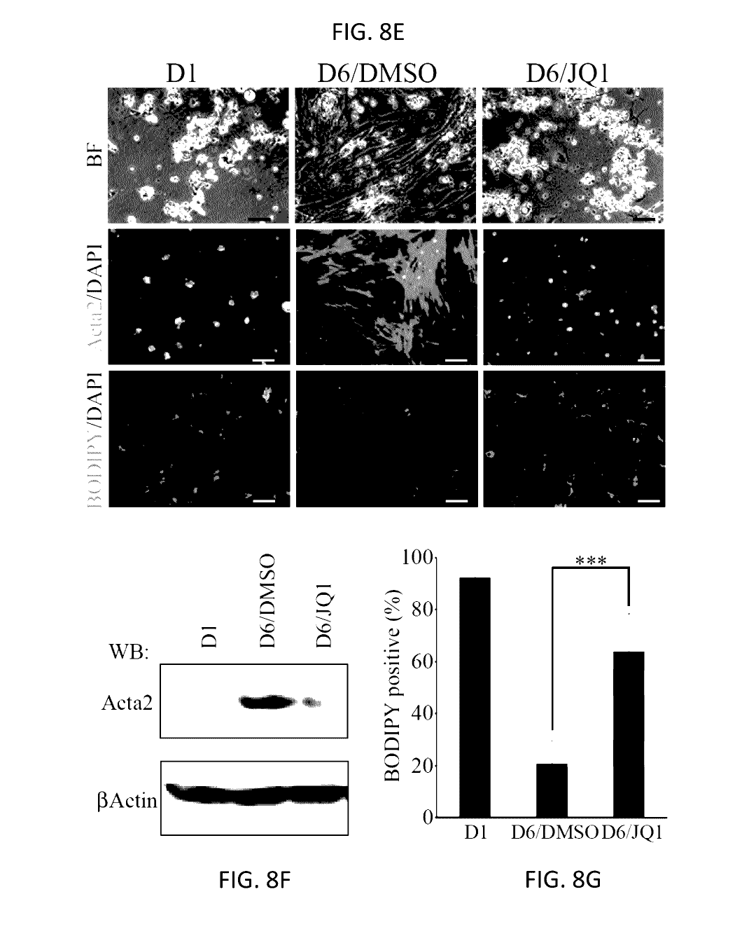

The intrinsic capability of BET-loaded enhancers to control pro-fibrotic gene expression prompted investigation of their regulatory potential in myofibroblast activation using integrative chemical genetics and functional genomics in a well-established HSC-to-myofibroblast self-activation system (FIG. 7), which recapitulates the key cellular (wound healing) event typified in the pathogenesis and progression of liver fibrosis¹⁶⁻¹⁸. Specifically, mRNA-Seq analysis of gene expression was examined in primary HSCs in the quiescent state (day 1, baseline) and during self-activation into myofibroblasts (days 3 and 6) in the absence or presence of JQ1. Assessment of differentially expressed transcripts revealed more than 900 genes that are significantly upregulated during HSC activation; among these more than 400 are suppressed by JQ1 (FIGS. 8B and 8C and Table 3). Table 3 provides a comparison of quiescent stellate cells (day 1) and fully activated stellate cells (day 6) in the presence or absence of JQ1, demonstrating that JQ1 inhibits/blocks the activation of stellate cells. Global transcriptome is similar to Day 1.

TABLE 3

| gene | sample_1 | sample_2 | status | value_1 | value_2 | log2(fold_change) | gene | sample_1 | sample_2 | value_1 | value_2 | log2(fold_change) |
|---|---|---|---|---|---|---|---|---|---|---|---|---|
| Kcnmb1 | Day_1 | Day_6 | OK | 0.30897 | 6.65507 | 4.42893 | Kcnmb1 | Day_6 | Day_6_JQ1 | 6.65507 | 0.01726 | −8.5908 |
| Gjb3 | Day_1 | Day_6 | OK | 4.00269 | 16.27 | 2.02317 | Gjb3 | Day_6 | Day_6_JQ1 | 16.27 | 0.08229 | −7.6272 |
| Trpc6 | Day_1 | Day_6 | OK | 0.67698 | 8.93446 | 3.72219 | Trpc6 | Day_6 | Day_6_JQ1 | 8.93446 | 0.05189 | −7.4279 |
| Tnfsf15 | Day_1 | Day_6 | OK | 0.38808 | 12.4033 | 4.99824 | Tnfsf15 | Day_6 | Day_6_JQ1 | 12.4033 | 0.07599 | −7.3507 |
| Cck | Day_1 | Day_6 | OK | 4.48985 | 38.3603 | 3.09487 | Cck | Day_6 | Day_6_JQ1 | 38.3603 | 0.31393 | −6.933 |
| Ccl12 | Day_1 | Day_6 | OK | 5.43655 | 25.1223 | 2.20821 | Ccl12 | Day_6 | Day_6_JQ1 | 25.1223 | 0.27482 | −6.5143 |
| Mfap5 | Day_1 | Day_6 | OK | 5.8228 | 91.8746 | 3.97988 | Mfap5 | Day_6 | Day_6_JQ1 | 91.8746 | 1.01061 | −6.5064 |
| Mfap4 | Day_1 | Day_6 | OK | 1.21483 | 24.431 | 4.32989 | Mfap4 | Day_6 | Day_6_JQ1 | 24.431 | 0.29945 | −6.3503 |
| Stra6 | Day_1 | Day_6 | OK | 0.4777 | 6.06103 | 3.6654 | Stra6 | Day_6 | Day_6_JQ1 | 6.06103 | 0.07484 | −6.3397 |
| Eln | Day_1 | Day_6 | OK | 0.34427 | 43.7918 | 6.99097 | Eln | Day_6 | Day_6_JQ1 | 43.7918 | 0.60766 | −6.1713 |
| Tnfsf8 | Day_1 | Day_6 | OK | 0.30072 | 9.15093 | 4.92745 | Tnfsf8 | Day_6 | Day_6_JQ1 | 9.15093 | 0.15809 | −5.8552 |
| Wisp2 | Day_1 | Day_6 | OK | 0.21048 | 76.9329 | 8.51375 | Wisp2 | Day_6 | Day_6_JQ1 | 76.9329 | 1.36854 | −5.8129 |
| Ivl | Day_1 | Day_6 | OK | 3.31204 | 27.4019 | 3.04849 | Ivl | Day_6 | Day_6_JQ1 | 27.4019 | 0.50562 | −5.7601 |
| Gjb5 | Day_1 | Day_6 | OK | 0.54971 | 6.43761 | 3.54978 | Gjb5 | Day_6 | Day_6_JQ1 | 6.43761 | 0.13875 | −5.5359 |
| Mrap | Day_1 | Day_6 | OK | 1.73744 | 10.651 | 2.61595 | Mrap | Day_6 | Day_6_JQ1 | 10.651 | 0.25485 | −5.3852 |
| Hspb7 | Day_1 | Day_6 | OK | 1.17365 | 12.0343 | 3.35808 | Hspb7 | Day_6 | Day_6_JQ1 | 12.0343 | 0.29864 | −5.3326 |
| Has2 | Day_1 | Day_6 | OK | 2.65793 | 71.064 | 4.74074 | Has2 | Day_6 | Day_6_JQ1 | 71.064 | 1.81183 | −5.2936 |
| Sorcs2 | Day_1 | Day_6 | OK | 0.44394 | 6.09526 | 3.77924 | Sorcs2 | Day_6 | Day_6_JQ1 | 6.09526 | 0.15878 | −5.2626 |
| Pdgfrl | Day_1 | Day_6 | OK | 0.88414 | 16.6573 | 4.23574 | Pdgfrl | Day_6 | Day_6_JQ1 | 16.6573 | 0.4741 | −5.1348 |
| Col15a1 | Day_1 | Day_6 | OK | 0.76257 | 9.82232 | 3.68712 | Col15a1 | Day_6 | Day_6_JQ1 | 9.82232 | 0.28324 | −5.116 |
| Pstpip1 | Day_1 | Day_6 | OK | 1.29243 | 8.98887 | 2.79805 | Pstpip1 | Day_6 | Day_6_JQ1 | 8.98887 | 0.26072 | −5.1076 |
| Cthrc1 | Day_1 | Day_6 | OK | 0.91908 | 134.517 | 7.19338 | Cthrc1 | Day_6 | Day_6_JQ1 | 134.517 | 4.10448 | −5.0344 |
| Fam198b | Day_1 | Day_6 | OK | 1.97589 | 62.2354 | 4.97716 | Fam198b | Day_6 | Day_6_JQ1 | 62.2354 | 1.90697 | −5.0284 |
| Cnn1 | Day_1 | Day_6 | OK | 0.45223 | 172.057 | 8.57162 | Cnn1 | Day_6 | Day_6_JQ1 | 172.057 | 5.62822 | −4.9341 |
| Ltbp2 | Day_1 | Day_6 | OK | 3.15905 | 142.507 | 5.4954 | Ltbp2 | Day_6 | Day_6_JQ1 | 142.507 | 4.7344 | −4.9117 |
| P2ry14 | Day_1 | Day_6 | OK | 11.7032 | 185.476 | 3.98626 | P2ry14 | Day_6 | Day_6_JQ1 | 185.476 | 6.94616 | −4.7389 |
| Ptgs2 | Day_1 | Day_6 | OK | 16.9785 | 57.0483 | 1.74847 | Ptgs2 | Day_6 | Day_6_JQ1 | 57.0483 | 2.22088 | −4.683 |
| Tnfsf18 | Day_1 | Day_6 | OK | 1.6144 | 6.1463 | 1.92872 | Tnfsf18 | Day_6 | Day_6_JQ1 | 6.1463 | 0.24 | −4.6786 |
| Actg2 | Day_1 | Day_6 | OK | 0.76427 | 248.058 | 8.34237 | Actg2 | Day_6 | Day_6_JQ1 | 248.058 | 9.74735 | −4.6695 |
| Dpep1 | Day_1 | Day_6 | OK | 1.09917 | 14.2067 | 3.69209 | Dpep1 | Day_6 | Day_6_JQ1 | 14.2067 | 0.63569 | −4.4821 |
| AA467197 | Day_1 | Day_6 | OK | 8.07806 | 40.0088 | 2.30824 | AA467197 | Day_6 | Day_6_JQ1 | 40.0088 | 1.79182 | −4.4808 |
| Gbx2 | Day_1 | Day_6 | OK | 0 | 8.30408 | inf | Gbx2 | Day_6 | Day_6_JQ1 | 8.30408 | 0.43306 | −4.2612 |
| Pdlim3 | Day_1 | Day_6 | OK | 1.82008 | 10.5905 | 2.54069 | Pdlim3 | Day_6 | Day_6_JQ1 | 10.5905 | 0.59527 | −4.1531 |
| Tmem200a | Day_1 | Day_6 | OK | 0.52758 | 5.49736 | 3.38129 | Tmem200a | Day_6 | Day_6_JQ1 | 5.49736 | 0.31922 | −4.1061 |
| Wisp1 | Day_1 | Day_6 | OK | 4.93029 | 72.6617 | 3.88145 | Wisp1 | Day_6 | Day_6_JQ1 | 72.6617 | 4.26216 | −4.0915 |
| Fxyd6 | Day_1 | Day_6 | OK | 0.39557 | 16.7433 | 5.40351 | Fxyd6 | Day_6 | Day_6_JQ1 | 16.7433 | 0.99208 | −4.077 |
| Saa3 | Day_1 | Day_6 | OK | 16.8818 | 179.773 | 3.41263 | Saa3 | Day_6 | Day_6_JQ1 | 179.773 | 10.7546 | −4.0632 |
| Slc16a3 | Day_1 | Day_6 | OK | 21.0056 | 170.37 | 3.01982 | Slc16a3 | Day_6 | Day_6_JQ1 | 170.37 | 10.7025 | −3.9927 |
| Rasl12 | Day_1 | Day_6 | OK | 1.15539 | 4.62854 | 2.00218 | Rasl12 | Day_6 | Day_6_JQ1 | 4.62854 | 0.3063 | −3.9176 |
| Pla1a | Day_1 | Day_6 | OK | 2.19123 | 30.8506 | 3.81548 | Pla1a | Day_6 | Day_6_JQ1 | 30.8506 | 2.04702 | −3.9137 |
| Dkk2 | Day_1 | Day_6 | OK | 0.21569 | 13.426 | 5.95996 | Dkk2 | Day_6 | Day_6_JQ1 | 13.426 | 0.91797 | −3.8705 |
| AI427809 | Day_1 | Day_6 | OK | 1.31804 | 5.43643 | 2.04427 | AI427809 | Day_6 | Day_6_JQ1 | 5.43643 | 0.37822 | −3.8454 |
| Des | Day_1 | Day_6 | OK | 37.9047 | 203.65 | 2.42564 | Des | Day_6 | Day_6_JQ1 | 203.65 | 14.4745 | −3.8145 |
| Pappa | Day_1 | Day_6 | OK | 0.27925 | 9.27675 | 5.05401 | Pappa | Day_6 | Day_6_JQ1 | 9.27675 | 0.6606 | −3.8118 |
| Elfn1 | Day_1 | Day_6 | OK | 0.94662 | 5.70819 | 2.59217 | Elfn1 | Day_6 | Day_6_JQ1 | 5.70819 | 0.41406 | −3.7851 |
| Tmeff1 | Day_1 | Day_6 | OK | 1.44924 | 70.9082 | 5.61258 | Tmeff1 | Day_6 | Day_6_JQ1 | 70.9082 | 5.18135 | −3.7746 |
| Bcat1 | Day_1 | Day_6 | OK | 4.60208 | 15.8837 | 1.78719 | Bcat1 | Day_6 | Day_6_JQ1 | 15.8837 | 1.17141 | −3.7612 |
| Thy1 | Day_1 | Day_6 | OK | 3.00349 | 29.6049 | 3.30112 | Thy1 | Day_6 | Day_6_JQ1 | 29.6049 | 2.22239 | −3.7357 |
| Htra3 | Day_1 | Day_6 | OK | 1.90975 | 25.1293 | 3.71791 | Htra3 | Day_6 | Day_6_JQ1 | 25.1293 | 1.89579 | −3.7285 |
| Col11a1 | Day_1 | Day_6 | OK | 0.03228 | 56.9722 | 10.7852 | Col11a1 | Day_6 | Day_6_JQ1 | 56.9722 | 4.30333 | −3.7267 |
| Srpx2 | Day_1 | Day_6 | OK | 9.81776 | 39.4625 | 2.00701 | Srpx2 | Day_6 | Day_6_JQ1 | 39.4625 | 2.99797 | −3.7184 |
| Pdgfc | Day_1 | Day_6 | OK | 3.58118 | 20.0741 | 2.48683 | Pdgfc | Day_6 | Day_6_JQ1 | 20.0741 | 1.52698 | −3.7166 |
| Serpinb9b | Day_1 | Day_6 | OK | 4.55261 | 54.2365 | 3.5745 | Serpinb9b | Day_6 | Day_6_JQ1 | 54.2365 | 4.13566 | −3.7131 |
| C1qtnf2 | Day_1 | Day_6 | OK | 1.67462 | 16.4148 | 3.29309 | C1qtnf2 | Day_6 | Day_6_JQ1 | 16.4148 | 1.26244 | −3.7007 |
| Fst | Day_1 | Day_6 | OK | 3.89679 | 39.6029 | 3.34525 | Fst | Day_6 | Day_6_JQ1 | 39.6029 | 3.07866 | −3.6852 |
| Col7a1 | Day_1 | Day_6 | OK | 0.10363 | 6.90127 | 6.0573 | Col7a1 | Day_6 | Day_6_JQ1 | 6.90127 | 0.54438 | −3.6642 |
| Olfm2 | Day_1 | Day_6 | OK | 0.19663 | 10.5489 | 5.74549 | Olfm2 | Day_6 | Day_6_JQ1 | 10.5489 | 0.83718 | −3.6554 |
| Tll1 | Day_1 | Day_6 | OK | 0.37774 | 4.91621 | 3.70209 | Tll1 | Day_6 | Day_6_JQ1 | 4.91621 | 0.39156 | −3.6502 |
| Cdh3 | Day_1 | Day_6 | OK | 0.44919 | 17.9348 | 5.31928 | Cdh3 | Day_6 | Day_6_JQ1 | 17.9348 | 1.47683 | −3.6022 |
| Kctd15 | Day_1 | Day_6 | OK | 3.33369 | 13.3028 | 1.99654 | Kctd15 | Day_6 | Day_6_JQ1 | 13.3028 | 1.12409 | −3.5649 |
| 4930583H14Rik | Day_1 | Day_6 | OK | 0.33238 | 7.74985 | 4.54327 | 4930583H14Rik | Day_6 | Day_6_JQ1 | 7.74985 | 0.65724 | −3.5597 |
| Acta1 | Day_1 | Day_6 | OK | 2.78597 | 61.4835 | 4.46395 | Acta1 | Day_6 | Day_6_JQ1 | 61.4835 | 5.29945 | −3.5363 |
| Angpt4 | Day_1 | Day_6 | OK | 0 | 25.6326 | inf | Angpt4 | Day_6 | Day_6_JQ1 | 25.6326 | 2.21625 | −3.5318 |
| Hhipl1 | Day_1 | Day_6 | OK | 0.47984 | 5.88806 | 3.61718 | Hhipl1 | Day_6 | Day_6_JQ1 | 5.88806 | 0.51696 | −3.5097 |
| Zfp827 | Day_1 | Day_6 | OK | 1.07952 | 5.14206 | 2.25195 | Zfp827 | Day_6 | Day_6_JQ1 | 5.14206 | 0.4625 | −3.4748 |
| Adamtsl2 | Day_1 | Day_6 | OK | 0.7674 | 7.30924 | 3.25167 | Adamtsl2 | Day_6 | Day_6_JQ1 | 7.30924 | 0.67456 | −3.4377 |
| Hebp2 | Day_1 | Day_6 | OK | 0.8872 | 8.17359 | 3.20364 | Hebp2 | Day_6 | Day_6_JQ1 | 8.17359 | 0.76842 | −3.411 |
| Alox5ap | Day_1 | Day_6 | OK | 3.39874 | 19.1261 | 2.49247 | Alox5ap | Day_6 | Day_6_JQ1 | 19.1261 | 1.8396 | −3.3781 |
| Rgs4 | Day_1 | Day_6 | OK | 1.81829 | 24.1832 | 3.73335 | Rgs4 | Day_6 | Day_6_JQ1 | 24.1832 | 2.34489 | −3.3664 |
| Hsd11b1 | Day_1 | Day_6 | OK | 13.9492 | 38.1155 | 1.4502 | Hsd11b1 | Day_6 | Day_6_JQ1 | 38.1155 | 3.83872 | −3.3117 |
| Col6a3 | Day_1 | Day_6 | OK | 4.98244 | 71.0539 | 3.83399 | Col6a3 | Day_6 | Day_6_JQ1 | 71.0539 | 7.23443 | −3.296 |
| Postn | Day_1 | Day_6 | OK | 10.945 | 570.293 | 5.70336 | Postn | Day_6 | Day_6_JQ1 | 570.293 | 58.0905 | −3.2953 |
| Prkar1b | Day_1 | Day_6 | OK | 0.90454 | 10.5692 | 3.54653 | Prkar1b | Day_6 | Day_6_JQ1 | 10.5692 | 1.1011 | −3.2629 |
| Fam132b | Day_1 | Day_6 | OK | 0.40533 | 5.78404 | 3.8349 | Fam132b | Day_6 | Day_6_JQ1 | 5.78404 | 0.61357 | −3.2368 |
| Naalad2 | Day_1 | Day_6 | OK | 1.48606 | 8.60497 | 2.53368 | Naalad2 | Day_6 | Day_6_JQ1 | 8.60497 | 0.97386 | −3.1434 |
| Def6 | Day_1 | Day_6 | OK | 0.9963 | 7.6116 | 2.93355 | Def6 | Day_6 | Day_6_JQ1 | 7.6116 | 0.86236 | −3.1418 |
| Esrp2 | Day_1 | Day_6 | OK | 1.89409 | 6.55651 | 1.79142 | Esrp2 | Day_6 | Day_6_JQ1 | 6.55651 | 0.74594 | −3.1358 |
| Sertad4 | Day_1 | Day_6 | OK | 1.27593 | 6.00615 | 2.23489 | Sertad4 | Day_6 | Day_6_JQ1 | 6.00615 | 0.69036 | −3.121 |
| Fkbp10 | Day_1 | Day_6 | OK | 36.6459 | 236.986 | 2.69308 | Fkbp10 | Day_6 | Day_6_JQ1 | 236.986 | 27.4534 | −3.1097 |
| Chst11 | Day_1 | Day_6 | OK | 62.2241 | 192.286 | 1.62771 | Chst11 | Day_6 | Day_6_JQ1 | 192.286 | 22.6881 | −3.0832 |
| Rap1gap2 | Day_1 | Day_6 | OK | 5.10773 | 21.5087 | 2.07417 | Rap1gap2 | Day_6 | Day_6_JQ1 | 21.5087 | 2.58013 | −3.0594 |
| Ak4 | Day_1 | Day_6 | OK | 5.33475 | 18.3772 | 1.78442 | Ak4 | Day_6 | Day_6_JQ1 | 18.3772 | 2.20735 | −3.0575 |
| Nfatc4 | Day_1 | Day_6 | OK | 0.54257 | 18.5966 | 5.09908 | Nfatc4 | Day_6 | Day_6_JQ1 | 18.5966 | 2.23537 | −3.0565 |
| F3 | Day_1 | Day_6 | OK | 112.086 | 269.445 | 1.26538 | F3 | Day_6 | Day_6_JQ1 | 269.445 | 32.9312 | −3.0325 |
| C1qtnf5 | Day_1 | Day_6 | OK | 6.8122 | 43.3705 | 2.67052 | C1qtnf5 | Day_6 | Day_6_JQ1 | 43.3705 | 5.30288 | −3.0319 |
| Gm12824 | Day_1 | Day_6 | OK | 1.21985 | 6.62967 | 2.44223 | Gm12824 | Day_6 | Day_6_JQ1 | 6.62967 | 0.81073 | −3.0317 |
| P4ha3 | Day_1 | Day_6 | OK | 0.10271 | 10.8738 | 6.72615 | P4ha3 | Day_6 | Day_6_JQ1 | 10.8738 | 1.33454 | −3.0265 |
| Tnfsf13b | Day_1 | Day_6 | OK | 1.36718 | 9.03107 | 2.72369 | Tnfsf13b | Day_6 | Day_6_JQ1 | 9.03107 | 1.11905 | −3.0126 |
| Tfpi2 | Day_1 | Day_6 | OK | 5.6267 | 35.9556 | 2.67586 | Tfpi2 | Day_6 | Day_6_JQ1 | 35.9556 | 4.48596 | −3.0027 |
| Chd3 | Day_1 | Day_6 | OK | 8.95681 | 34.7271 | 1.955 | Chd3 | Day_6 | Day_6_JQ1 | 34.7271 | 4.38612 | −2.985 |
| Npr3 | Day_1 | Day_6 | OK | 25.0281 | 65.9709 | 1.39828 | Npr3 | Day_6 | Day_6_JQ1 | 65.9709 | 8.40845 | −2.9719 |
| Etv4 | Day_1 | Day_6 | OK | 1.10429 | 8.66792 | 2.97257 | Etv4 | Day_6 | Day_6_JQ1 | 8.66792 | 1.11615 | −2.9572 |
| Wntl1 | Day_1 | Day_6 | OK | 0.96983 | 10.057 | 3.37434 | Wntl1 | Day_6 | Day_6_JQ1 | 10.057 | 1.29943 | −2.9523 |
| Gpr176 | Day_1 | Day_6 | OK | 9.52687 | 41.5675 | 2.12538 | Gpr176 | Day_6 | Day_6_JQ1 | 41.5675 | 5.41282 | −2.941 |
| Ptprn | Day_1 | Day_6 | OK | 2.93552 | 39.7337 | 3.75867 | Ptprn | Day_6 | Day_6_JQ1 | 39.7337 | 5.20356 | −2.9328 |
| Col6a2 | Day_1 | Day_6 | OK | 8.0342 | 48.06 | 2.58061 | Col6a2 | Day_6 | Day_6_JQ1 | 48.06 | 6.3149 | −2.928 |
| Jph2 | Day_1 | Day_6 | OK | 0.40085 | 26.1105 | 6.02541 | Jph2 | Day_6 | Day_6_JQ1 | 26.1105 | 3.45063 | −2.9197 |
| Mmp23 | Day_1 | Day_6 | OK | 0.88911 | 45.1918 | 5.66756 | Mmp23 | Day_6 | Day_6_JQ1 | 45.1918 | 6.01408 | −2.9097 |
| Prelid2 | Day_1 | Day_6 | OK | 3.22952 | 12.662 | 1.97111 | Prelid2 | Day_6 | Day_6_JQ1 | 12.662 | 1.70999 | −2.8884 |
| Hspb2 | Day_1 | Day_6 | OK | 8.35596 | 49.9611 | 2.57993 | Hspb2 | Day_6 | Day_6_JQ1 | 49.9611 | 6.82616 | −2.8717 |
| Spon2 | Day_1 | Day_6 | OK | 3.51573 | 89.5714 | 4.67114 | Spon2 | Day_6 | Day_6_JQ1 | 89.5714 | 12.3682 | −2.8564 |
| Wbscr17 | Day_1 | Day_6 | OK | 0.08779 | 5.23502 | 5.89807 | Wbscr17 | Day_6 | Day_6_JQ1 | 5.23502 | 0.72559 | −2.851 |
| Cdh6 | Day_1 | Day_6 | OK | 1.10656 | 5.39051 | 2.28434 | Cdh6 | Day_6 | Day_6_JQ1 | 5.39051 | 0.76401 | −2.8188 |
| Dok5 | Day_1 | Day_6 | OK | 2.08648 | 8.28634 | 1.98967 | Dok5 | Day_6 | Day_6_JQ1 | 8.28634 | 1.20334 | −2.7837 |
| Aoc3 | Day_1 | Day_6 | OK | 0.12163 | 5.25859 | 5.43408 | Aoc3 | Day_6 | Day_6_JQ1 | 5.25859 | 0.7708 | −2.7703 |
| Lmod1 | Day_1 | Day_6 | OK | 0.29531 | 37.7126 | 6.99668 | Lmod1 | Day_6 | Day_6_JQ1 | 37.7126 | 5.53332 | −2.7688 |
| Atp1a3 | Day_1 | Day_6 | OK | 0.57206 | 7.12661 | 3.63898 | Atp1a3 | Day_6 | Day_6_JQ1 | 7.12661 | 1.07031 | −2.7352 |
| Fibin | Day_1 | Day_6 | OK | 0.36507 | 12.1912 | 5.06151 | Fibin | Day_6 | Day_6_JQ1 | 12.1912 | 1.84731 | −2.7224 |
| Lox | Day_1 | Day_6 | OK | 4.72165 | 675.941 | 7.16146 | Lox | Day_6 | Day_6_JQ1 | 675.941 | 102.497 | −2.7213 |
| Fblim1 | Day_1 | Day_6 | OK | 35.3398 | 80.287 | 1.18387 | Fblim1 | Day_6 | Day_6_JQ1 | 80.287 | 12.2302 | −2.7147 |
| Mrgprf | Day_1 | Day_6 | OK | 0.34823 | 8.94379 | 4.68278 | Mrgprf | Day_6 | Day_6_JQ1 | 8.94379 | 1.3677 | −2.7091 |
| Loxl4 | Day_1 | Day_6 | OK | 12.2323 | 40.4193 | 1.72435 | Loxl4 | Day_6 | Day_6_JQ1 | 40.4193 | 6.18137 | −2.7091 |
| 4833442J19Rik | Day_1 | Day_6 | OK | 2.82494 | 38.3446 | 3.76273 | 4833442J19Rik | Day_6 | Day_6_JQ1 | 38.3446 | 5.92769 | −2.6935 |
| D430019H16Rik | Day_1 | Day_6 | OK | 1.43387 | 12.678 | 3.14434 | D430019H16Rik | Day_6 | Day_6_JQ1 | 12.678 | 1.97148 | −2.685 |
| Syt13 | Day_1 | Day_6 | OK | 0.43667 | 40.2229 | 6.52533 | Syt13 | Day_6 | Day_6_JQ1 | 40.2229 | 6.25723 | −2.6844 |
| Ltbp1 | Day_1 | Day_6 | OK | 4.02311 | 24.4677 | 2.6045 | Ltbp1 | Day_6 | Day_6_JQ1 | 24.4677 | 3.83254 | −2.6745 |
| Fkbp11 | Day_1 | Day_6 | OK | 13.9457 | 44.328 | 1.6684 | Fkbp11 | Day_6 | Day_6_JQ1 | 44.328 | 6.95477 | −2.6721 |
| Csdc2 | Day_1 | Day_6 | OK | 0.29105 | 5.77018 | 4.30929 | Csdc2 | Day_6 | Day_6_JQ1 | 5.77018 | 0.91303 | −2.6599 |
| Mgp | Day_1 | Day_6 | OK | 50.0853 | 141.183 | 1.49511 | Mgp | Day_6 | Day_6_JQ1 | 141.183 | 22.5571 | −2.6459 |
| Myl9 | Day_1 | Day_6 | OK | 37.364 | 865.564 | 4.53392 | Myl9 | Day_6 | Day_6_JQ1 | 865.564 | 142.669 | −2.601 |
| Serpinf1 | Day_1 | Day_6 | OK | 17.3031 | 395.807 | 4.5157 | Serpinf1 | Day_6 | Day_6_JQ1 | 395.807 | 66.5572 | −2.5721 |
| Adcy5 | Day_1 | Day_6 | OK | 1.30694 | 5.80427 | 2.15092 | Adcy5 | Day_6 | Day_6_JQ1 | 5.80427 | 0.98012 | −2.5661 |
| Serinc2 | Day_1 | Day_6 | OK | 6.09221 | 21.0809 | 1.7909 | Serinc2 | Day_6 | Day_6_JQ1 | 21.0809 | 3.59613 | −2.5514 |
| Adcy1 | Day_1 | Day_6 | OK | 0.09389 | 4.7329 | 5.65566 | Adcy1 | Day_6 | Day_6_JQ1 | 4.7329 | 0.80919 | −2.5482 |
| Arhgef6 | Day_1 | Day_6 | OK | 5.27131 | 11.5472 | 1.13131 | Arhgef6 | Day_6 | Day_6_JQ1 | 11.5472 | 2.02497 | −2.5116 |
| Itga5 | Day_1 | Day_6 | OK | 68.3133 | 251.416 | 1.87984 | Itga5 | Day_6 | Day_6_JQ1 | 251.416 | 44.6427 | −2.4936 |
| Folr1 | Day_1 | Day_6 | OK | 0.73472 | 10.6375 | 3.85581 | Folr1 | Day_6 | Day_6_JQ1 | 10.6375 | 1.90454 | −2.4816 |
| Fmod | Day_1 | Day_6 | OK | 0.70518 | 13.1548 | 4.22145 | Fmod | Day_6 | Day_6_JQ1 | 13.1548 | 2.36858 | −2.4735 |
| Crispld2 | Day_1 | Day_6 | OK | 3.9184 | 27.2778 | 2.79939 | Crispld2 | Day_6 | Day_6_JQ1 | 27.2778 | 4.91183 | −2.4734 |
| Cplx2 | Day_1 | Day_6 | OK | 4.88988 | 18.6259 | 1.92944 | Cplx2 | Day_6 | Day_6_JQ1 | 18.6259 | 3.35819 | −2.4716 |
| Crlf1 | Day_1 | Day_6 | OK | 1.28239 | 147.398 | 6.84474 | Crlf1 | Day_6 | Day_6_JQ1 | 147.398 | 26.6612 | −2.4669 |
| Srpx | Day_1 | Day_6 | OK | 0.14839 | 14.497 | 6.61021 | Srpx | Day_6 | Day_6_JQ1 | 14.497 | 2.6404 | −2.4569 |
| Pygl | Day_1 | Day_6 | OK | 3.01315 | 17.9554 | 2.57507 | Pygl | Day_6 | Day_6_JQ1 | 17.9554 | 3.27475 | −2.455 |
| Sh3gl3 | Day_1 | Day_6 | OK | 0.14259 | 5.97316 | 5.38858 | Sh3gl3 | Day_6 | Day_6_JQ1 | 5.97316 | 1.08955 | −2.4548 |
| Thbs3 | Day_1 | Day_6 | OK | 0.43435 | 7.78988 | 4.16468 | Thbs3 | Day_6 | Day_6_JQ1 | 7.78988 | 1.4249 | −2.4507 |
| Gpr133 | Day_1 | Day_6 | OK | 1.60624 | 20.8713 | 3.69976 | Gpr133 | Day_6 | Day_6_JQ1 | 20.8713 | 3.84236 | −2.4415 |
| Kcne4 | Day_1 | Day_6 | OK | 11.2086 | 46.0538 | 2.03871 | Kcne4 | Day_6 | Day_6_JQ1 | 46.0538 | 8.54726 | −2.4298 |
| Sparcl1 | Day_1 | Day_6 | OK | 8.57813 | 29.8971 | 1.80127 | Sparcl1 | Day_6 | Day_6_JQ1 | 29.8971 | 5.62864 | −2.4092 |
| Fzd6 | Day_1 | Day_6 | OK | 4.45863 | 12.0533 | 1.43476 | Fzd6 | Day_6 | Day_6_JQ1 | 12.0533 | 2.32764 | −2.3725 |
| Foxc2 | Day_1 | Day_6 | OK | 1.84116 | 7.4612 | 2.01879 | Foxc2 | Day_6 | Day_6_JQ1 | 7.4612 | 1.45305 | −2.3603 |
| Podn | Day_1 | Day_6 | OK | 1.82972 | 7.42598 | 2.02096 | Podn | Day_6 | Day_6_JQ1 | 7.42598 | 1.45845 | −2.3482 |
| Ydjc | Day_1 | Day_6 | OK | 2.38189 | 9.6914 | 2.0246 | Ydjc | Day_6 | Day_6_JQ1 | 9.6914 | 1.90466 | −2.3472 |
| Myh11 | Day_1 | Day_6 | OK | 2.33747 | 32.1738 | 3.78287 | Myh11 | Day_6 | Day_6_JQ1 | 32.1738 | 6.32838 | −2.346 |
| Ncam1 | Day_1 | Day_6 | OK | 0.15518 | 40.77 | 8.03741 | Ncam1 | Day_6 | Day_6_JQ1 | 40.77 | 8.07272 | −2.3364 |
| Mrvi1 | Day_1 | Day_6 | OK | 3.35937 | 13.0175 | 1.95419 | Mrvi1 | Day_6 | Day_6_JQ1 | 13.0175 | 2.60224 | −2.3226 |
| Tsku | Day_1 | Day_6 | OK | 39.2352 | 95.2113 | 1.27899 | Tsku | Day_6 | Day_6_JQ1 | 95.2113 | 19.2147 | −2.3089 |
| Phyhd1 | Day_1 | Day_6 | OK | 5.06758 | 17.5053 | 1.78842 | Phyhd1 | Day_6 | Day_6_JQ1 | 17.5053 | 3.55162 | −2.3012 |
| Scel | Day_1 | Day_6 | OK | 0.38079 | 17.6919 | 5.53793 | Scel | Day_6 | Day_6_JQ1 | 17.6919 | 3.6703 | −2.2691 |
| Fam38b | Day_1 | Day_6 | OK | 2.03096 | 16.2499 | 3.0002 | Fam38b | Day_6 | Day_6_JQ1 | 16.2499 | 3.39 | −2.2611 |
| Col12a1 | Day_1 | Day_6 | OK | 1.06926 | 180.423 | 7.39862 | Col12a1 | Day_6 | Day_6_JQ1 | 180.423 | 37.8465 | −2.2532 |
| Igfbp2 | Day_1 | Day_6 | OK | 0.8821 | 12.1193 | 3.78021 | Igfbp2 | Day_6 | Day_6_JQ1 | 12.1193 | 2.5845 | −2.2293 |
| Fstl3 | Day_1 | Day_6 | OK | 11.9449 | 69.6853 | 2.54446 | Fstl3 | Day_6 | Day_6_JQ1 | 69.6853 | 14.8692 | −2.2285 |
| Nexn | Day_1 | Day_6 | OK | 6.2418 | 29.6814 | 2.24952 | Nexn | Day_6 | Day_6_JQ1 | 29.6814 | 6.34291 | −2.2263 |
| Emp2 | Day_1 | Day_6 | OK | 17.5438 | 41.6354 | 1.24685 | Emp2 | Day_6 | Day_6_JQ1 | 41.6354 | 8.90301 | −2.2254 |
| Aif11 | Day_1 | Day_6 | OK | 0.76716 | 5.6445 | 2.87925 | Aif11 | Day_6 | Day_6_JQ1 | 5.6445 | 1.20792 | −2.2243 |
| Psrc1 | Day_1 | Day_6 | OK | 1.816 | 31.7836 | 4.12945 | Psrc1 | Day_6 | Day_6_JQ1 | 31.7836 | 6.87429 | −2.209 |
| Slc2a10 | Day_1 | Day_6 | OK | 1.77128 | 9.44286 | 2.41443 | Slc2a10 | Day_6 | Day_6_JQ1 | 9.44286 | 2.06196 | −2.1952 |
| Adcy7 | Day_1 | Day_6 | OK | 5.54115 | 15.7463 | 1.50676 | Adcy7 | Day_6 | Day_6_JQ1 | 15.7463 | 3.43874 | −2.1951 |
| Dpysl3 | Day_1 | Day_6 | OK | 14.2184 | 65.1443 | 2.19588 | Dpysl3 | Day_6 | Day_6_JQ1 | 65.1443 | 14.2712 | −2.1905 |
| Fndc1 | Day_1 | Day_6 | OK | 0.67409 | 68.9277 | 6.67601 | Fndc1 | Day_6 | Day_6_JQ1 | 68.9277 | 15.189 | −2.1821 |
| Cacng7 | Day_1 | Day_6 | OK | 1.04882 | 10.1921 | 3.28062 | Cacng7 | Day_6 | Day_6_JQ1 | 10.1921 | 2.27612 | −2.1628 |
| Pter | Day_1 | Day_6 | OK | 2.11164 | 7.79658 | 1.88448 | Pter | Day_6 | Day_6_JQ1 | 7.79658 | 1.74261 | −2.1616 |
| Fndc4 | Day_1 | Day_6 | OK | 1.28275 | 5.52496 | 2.10672 | Fndc4 | Day_6 | Day_6_JQ1 | 5.52496 | 1.24382 | −2.1512 |
| Zbtb8b | Day_1 | Day_6 | OK | 0.07223 | 8.93825 | 6.95124 | Zbtb8b | Day_6 | Day_6_JQ1 | 8.93825 | 2.02273 | −2.1437 |
| Tspan2 | Day_1 | Day_6 | OK | 2.46328 | 12.1915 | 2.30722 | Tspan2 | Day_6 | Day_6_JQ1 | 12.1915 | 2.76172 | −2.1422 |
| Wnt5a | Day_1 | Day_6 | OK | 3.03982 | 32.9179 | 3.43681 | Wnt5a | Day_6 | Day_6_JQ1 | 32.9179 | 7.52414 | −2.1293 |
| Fah | Day_1 | Day_6 | OK | 1.2575 | 7.83959 | 2.64022 | Fah | Day_6 | Day_6_JQ1 | 7.83959 | 1.79329 | −2.1282 |
| Serpine2 | Day_1 | Day_6 | OK | 5.72009 | 34.0957 | 2.57548 | Serpine2 | Day_6 | Day_6_JQ1 | 34.0957 | 7.82469 | −2.1235 |
| Vegfa | Day_1 | Day_6 | OK | 56.766 | 120.097 | 1.0811 | Vegfa | Day_6 | Day_6_JQ1 | 120.097 | 27.576 | −2.1227 |
| Adamts12 | Day_1 | Day_6 | OK | 3.50135 | 74.0615 | 4.40274 | Adamts12 | Day_6 | Day_6_JQ1 | 74.0615 | 17.1447 | −2.111 |
| Plscr2 | Day_1 | Day_6 | OK | 2.96386 | 32.5691 | 3.45795 | Plscr2 | Day_6 | Day_6_JQ1 | 32.5691 | 7.54439 | −2.11 |
| Hk2 | Day_1 | Day_6 | OK | 15.6611 | 41.6616 | 1.41153 | Hk2 | Day_6 | Day_6_JQ1 | 41.6616 | 9.85122 | −2.0803 |
| A430110N23Rik | Day_1 | Day_6 | OK | 1.29228 | 6.44734 | 2.31878 | A430110N23Rik | Day_6 | Day_6_JQ1 | 6.44734 | 1.52901 | −2.0761 |
| Rab15 | Day_1 | Day_6 | OK | 1.13696 | 6.27629 | 2.46473 | Rab15 | Day_6 | Day_6_JQ1 | 6.27629 | 1.49816 | −2.0667 |
| Spp1 | Day_1 | Day_6 | OK | 199.456 | 943.878 | 2.24253 | Spp1 | Day_6 | Day_6_JQ1 | 943.878 | 226.746 | −2.0575 |
| Plp2 | Day_1 | Day_6 | OK | 24.7083 | 69.2544 | 1.48691 | Plp2 | Day_6 | Day_6_JQ1 | 69.2544 | 16.6661 | −2.055 |
| Foxf2 | Day_1 | Day_6 | OK | 0.46119 | 6.84136 | 3.89084 | Foxf2 | Day_6 | Day_6_JQ1 | 6.84136 | 1.6602 | −2.0429 |
| Rab7l1 | Day_1 | Day_6 | OK | 9.89624 | 24.1496 | 1.28705 | Rab7l1 | Day_6 | Day_6_JQ1 | 24.1496 | 5.90148 | −2.0329 |
| Gpc1 | Day_1 | Day_6 | OK | 20.1767 | 41.9106 | 1.05463 | Gpc1 | Day_6 | Day_6_JQ1 | 41.9106 | 10.285 | −2.0268 |
| Vash2 | Day_1 | Day_6 | OK | 1.09787 | 5.45022 | 2.31161 | Vash2 | Day_6 | Day_6_JQ1 | 5.45022 | 1.34279 | −2.0211 |
| Kif26b | Day_1 | Day_6 | OK | 0.07128 | 12.7898 | 7.48735 | Kif26b | Day_6 | Day_6_JQ1 | 12.7898 | 3.16995 | −2.0125 |
| Sgce | Day_1 | Day_6 | OK | 10.0374 | 31.2083 | 1.63655 | Sgce | Day_6 | Day_6_JQ1 | 31.2083 | 7.75142 | −2.0094 |
| Gpr39 | Day_1 | Day_6 | OK | 14.6805 | 88.6895 | 2.59486 | Gpr39 | Day_6 | Day_6_JQ1 | 88.6895 | 22.0499 | −2.008 |
| Igf2 | Day_1 | Day_6 | OK | 0.47733 | 13.0449 | 4.77237 | Igf2 | Day_6 | Day_6_JQ1 | 13.0449 | 3.24902 | −2.0054 |
| Aldh1l2 | Day_1 | Day_6 | OK | 4.42269 | 30.1463 | 2.76899 | Aldh1l2 | Day_6 | Day_6_JQ1 | 30.1463 | 7.69789 | −1.9695 |
| Iglon5 | Day_1 | Day_6 | OK | 1.78061 | 8.38123 | 2.23479 | Iglon5 | Day_6 | Day_6_JQ1 | 8.38123 | 2.14801 | −1.9642 |
| Tbxa2r | Day_1 | Day_6 | OK | 0.89782 | 6.20309 | 2.78848 | Tbxa2r | Day_6 | Day_6_JQ1 | 6.20309 | 1.60344 | −1.9518 |
| B3galnt1 | Day_1 | Day_6 | OK | 2.89948 | 10.2307 | 1.81904 | B3galnt1 | Day_6 | Day_6_JQ1 | 10.2307 | 2.65254 | −1.9475 |
| Wnt4 | Day_1 | Day_6 | OK | 3.71533 | 71.2032 | 4.26038 | Wnt4 | Day_6 | Day_6_JQ1 | 71.2032 | 18.5297 | −1.9421 |
| Rftn2 | Day_1 | Day_6 | OK | 2.01233 | 5.26739 | 1.38822 | Rftn2 | Day_6 | Day_6_JQ1 | 5.26739 | 1.38734 | −1.9248 |
| Lims2 | Day_1 | Day_6 | OK | 8.20797 | 45.5241 | 2.47153 | Lims2 | Day_6 | Day_6_JQ1 | 45.5241 | 12.2591 | −1.8928 |
| Plac8 | Day_1 | Day_6 | OK | 154.788 | 383.958 | 1.31066 | Plac8 | Day_6 | Day_6_JQ1 | 383.958 | 104.172 | −1.882 |
| Bdnf | Day_1 | Day_6 | OK | 0.32522 | 16.8931 | 5.69887 | Bdnf | Day_6 | Day_6_JQ1 | 16.8931 | 4.6088 | −1.874 |
| Casp12 | Day_1 | Day_6 | OK | 3.64638 | 14.6069 | 2.00211 | Casp12 | Day_6 | Day_6_JQ1 | 14.6069 | 4.00015 | −1.8685 |
| Fam19a5 | Day_1 | Day_6 | OK | 0.27883 | 4.90769 | 4.1376 | Fam19a5 | Day_6 | Day_6_JQ1 | 4.90769 | 1.34431 | −1.8682 |
| Gys1 | Day_1 | Day_6 | OK | 15.2212 | 41.9131 | 1.46132 | Gys1 | Day_6 | Day_6_JQ1 | 41.9131 | 11.4841 | −1.8678 |
| Adam8 | Day_1 | Day_6 | OK | 19.3616 | 43.2975 | 1.16109 | Adam8 | Day_6 | Day_6_JQ1 | 43.2975 | 11.9385 | −1.8587 |
| Ptpla | Day_1 | Day_6 | OK | 14.7256 | 45.1915 | 1.61773 | Ptpla | Day_6 | Day_6_JQ1 | 45.1915 | 12.4632 | −1.8584 |
| Sh3kbp1 | Day_1 | Day_6 | OK | 4.0983 | 36.39 | 3.15045 | Sh3kbp1 | Day_6 | Day_6_JQ1 | 36.39 | 10.1373 | −1.8439 |
| Ddah1 | Day_1 | Day_6 | OK | 17.6408 | 37.5608 | 1.09031 | Ddah1 | Day_6 | Day_6_JQ1 | 37.5608 | 10.4898 | −1.8403 |
| Ankrd55 | Day_1 | Day_6 | OK | 0.18997 | 26.7203 | 7.13603 | Ankrd55 | Day_6 | Day_6_JQ1 | 26.7203 | 7.51879 | −1.8294 |
| Trim46 | Day_1 | Day_6 | OK | 1.78764 | 6.22108 | 1.79911 | Trim46 | Day_6 | Day_6_JQ1 | 6.22108 | 1.75814 | −1.8231 |
| 9930013L23Rik | Day_1 | Day_6 | OK | 6.8121 | 110.741 | 4.02294 | 9930013L23Rik | Day_6 | Day_6_JQ1 | 110.741 | 31.3095 | −1.8225 |
| Cd276 | Day_1 | Day_6 | OK | 1.35074 | 19.9855 | 3.88714 | Cd276 | Day_6 | Day_6_JQ1 | 19.9855 | 5.67351 | −1.8166 |
| Speg | Day_1 | Day_6 | OK | 7.3792 | 26.0645 | 1.82055 | Speg | Day_6 | Day_6_JQ1 | 26.0645 | 7.40942 | −1.8147 |
| Aldh111 | Day_1 | Day_6 | OK | 1.26164 | 5.37875 | 2.09197 | Aldh111 | Day_6 | Day_6_JQ1 | 5.37875 | 1.53574 | −1.8083 |
| Avpr1a | Day_1 | Day_6 | OK | 0.15285 | 5.73321 | 5.22916 | Avpr1a | Day_6 | Day_6_JQ1 | 5.73321 | 1.65135 | −1.7957 |
| Timp1 | Day_1 | Day_6 | OK | 68.2229 | 1027.64 | 3.91293 | Timp1 | Day_6 | Day_6_JQ1 | 1027.64 | 296.521 | −1.7931 |
| Ppp1r14a | Day_1 | Day_6 | OK | 17.7118 | 69.878 | 1.98012 | Ppp1r14a | Day_6 | Day_6_JQ1 | 69.878 | 20.201 | −1.7904 |
| Aldh1a2 | Day_1 | Day_6 | OK | 7.73533 | 65.9266 | 3.09133 | Aldh1a2 | Day_6 | Day_6_JQ1 | 65.9266 | 19.1605 | −1.7827 |
| Obsl1 | Day_1 | Day_6 | OK | 2.80737 | 10.6278 | 1.92055 | Obsl1 | Day_6 | Day_6_JQ1 | 10.6278 | 3.10798 | −1.7738 |
| Pmepa1 | Day_1 | Day_6 | OK | 36.2919 | 99.1242 | 1.44959 | Pmepa1 | Day_6 | Day_6_JQ1 | 99.1242 | 29.4329 | −1.7518 |
| Acpl2 | Day_1 | Day_6 | OK | 1.95323 | 24.1273 | 3.62673 | Acpl2 | Day_6 | Day_6_JQ1 | 24.1273 | 7.20488 | −1.7436 |
| Dmpk | Day_1 | Day_6 | OK | 10.9112 | 88.3241 | 3.017 | Dmpk | Day_6 | Day_6_JQ1 | 88.3241 | 26.4788 | −1.738 |
| Pdlim4 | Day_1 | Day_6 | OK | 4.66132 | 15.8892 | 1.76924 | Pdlim4 | Day_6 | Day_6_JQ1 | 15.8892 | 4.7862 | −1.7311 |
| Sardh | Day_1 | Day_6 | OK | 2.69386 | 10.5599 | 1.97085 | Sardh | Day_6 | Day_6_JQ1 | 10.5599 | 3.19278 | −1.7257 |
| Ndrg2 | Day_1 | Day_6 | OK | 12.5875 | 41.6291 | 1.7256 | Ndrg2 | Day_6 | Day_6_JQ1 | 41.6291 | 12.6911 | −1.7138 |
| Col16a1 | Day_1 | Day_6 | OK | 1.58376 | 16.4853 | 3.37976 | Col16a1 | Day_6 | Day_6_JQ1 | 16.4853 | 5.06354 | −1.703 |
| Gria4 | Day_1 | Day_6 | OK | 0.10915 | 8.46008 | 6.27636 | Gria4 | Day_6 | Day_6_JQ1 | 8.46008 | 2.60027 | −1.702 |
| Srgap3 | Day_1 | Day_6 | OK | 0.56476 | 4.51645 | 2.99949 | Srgap3 | Day_6 | Day_6_JQ1 | 4.51645 | 1.38937 | −1.7008 |
| Loxl3 | Day_1 | Day_6 | OK | 5.18778 | 39.0539 | 2.91228 | Loxl3 | Day_6 | Day_6_JQ1 | 39.0539 | 12.0376 | −1.6979 |
| Popdc2 | Day_1 | Day_6 | OK | 0.36904 | 13.8107 | 5.22586 | Popdc2 | Day_6 | Day_6_JQ1 | 13.8107 | 4.26297 | −1.6959 |
| Cdh11 | Day_1 | Day_6 | OK | 1.45907 | 56.5065 | 5.2753 | Cdh11 | Day_6 | Day_6_JQ1 | 56.5065 | 17.4722 | −1.6934 |
| Bnip3 | Day_1 | Day_6 | OK | 25.2823 | 60.3077 | 1.25422 | Bnip3 | Day_6 | Day_6_JQ1 | 60.3077 | 18.6877 | −1.6903 |
| Acot11 | Day_1 | Day_6 | OK | 1.66525 | 5.30358 | 1.67122 | Acot11 | Day_6 | Day_6_JQ1 | 5.30358 | 1.6538 | −1.6812 |
| Fap | Day_1 | Day_6 | OK | 0.38807 | 5.10834 | 3.71848 | Fap | Day_6 | Day_6_JQ1 | 5.10834 | 1.60084 | −1.674 |
| Pde1a | Day_1 | Day_6 | OK | 11.3714 | 29.5722 | 1.37884 | Pde1a | Day_6 | Day_6_JQ1 | 29.5722 | 9.28199 | −1.6717 |
| Fam132a | Day_1 | Day_6 | OK | 2.25595 | 13.7405 | 2.60663 | Fam132a | Day_6 | Day_6_JQ1 | 13.7405 | 4.33062 | −1.6658 |
| Tmem45a | Day_1 | Day_6 | OK | 1.4933 | 68.1356 | 5.51183 | Tmem45a | Day_6 | Day_6_JQ1 | 68.1356 | 21.4757 | −1.6657 |
| Akap5 | Day_1 | Day_6 | OK | 0.0923 | 4.63302 | 5.64948 | Akap5 | Day_6 | Day_6_JQ1 | 4.63302 | 1.47826 | −1.6481 |
| Pcsk5 | Day_1 | Day_6 | OK | 0.27905 | 15.0557 | 5.75364 | Pcsk5 | Day_6 | Day_6_JQ1 | 15.0557 | 4.87984 | −1.6254 |
| Col1a1 | Day_1 | Day_6 | OK | 8.99318 | 2559.42 | 8.15277 | Col1a1 | Day_6 | Day_6_JQ1 | 2559.42 | 830.694 | −1.6234 |
| Gdf6 | Day_1 | Day_6 | OK | 0.79228 | 80.0405 | 6.65858 | Gdf6 | Day_6 | Day_6_JQ1 | 80.0405 | 25.9944 | −1.6225 |
| Nts | Day_1 | Day_6 | OK | 0.82385 | 9.86908 | 3.58247 | Nts | Day_6 | Day_6_JQ1 | 9.86908 | 3.20989 | −1.6204 |
| 1110067D22Rik | Day_1 | Day_6 | OK | 5.49736 | 23.6605 | 2.10567 | 1110067D22Rik | Day_6 | Day_6_JQ1 | 23.6605 | 7.6991 | −1.6197 |
| Lrrn2 | Day_1 | Day_6 | OK | 0.15891 | 5.54336 | 5.12449 | Lrrn2 | Day_6 | Day_6_JQ1 | 5.54336 | 1.8093 | −1.6153 |
| Hrct1 | Day_1 | Day_6 | OK | 0.63877 | 9.85494 | 3.94747 | Hrct1 | Day_6 | Day_6_JQ1 | 9.85494 | 3.22849 | −1.61 |
| Shisa3 | Day_1 | Day_6 | OK | 0.7263 | 13.1189 | 4.17495 | Shisa3 | Day_6 | Day_6_JQ1 | 13.1189 | 4.34506 | −1.5942 |
| Bag2 | Day_1 | Day_6 | OK | 4.877 | 43.7103 | 3.16391 | Bag2 | Day_6 | Day_6_JQ1 | 43.7103 | 14.4878 | −1.5931 |
| Tcea3 | Day_1 | Day_6 | OK | 0.65267 | 5.86891 | 3.16868 | Tcea3 | Day_6 | Day_6_JQ1 | 5.86891 | 1.94665 | −1.5921 |
| Ppapdc1b | Day_1 | Day_6 | OK | 15.5022 | 39.9374 | 1.36526 | Ppapdc1b | Day_6 | Day_6_JQ1 | 39.9374 | 13.4067 | −1.5748 |
| Pfkfb4 | Day_1 | Day_6 | OK | 2.71872 | 7.0037 | 1.36519 | Pfkfb4 | Day_6 | Day_6_JQ1 | 7.0037 | 2.359 | −1.5699 |
| Cercam | Day_1 | Day_6 | OK | 1.45447 | 28.9258 | 4.31378 | Cercam | Day_6 | Day_6_JQ1 | 28.9258 | 9.77516 | −1.5652 |
| Hs6st2 | Day_1 | Day_6 | OK | 0.46726 | 68.95 | 7.20519 | Hs6st2 | Day_6 | Day_6_JQ1 | 68.95 | 23.4423 | −1.5564 |
| Tmem14a | Day_1 | Day_6 | OK | 1.22257 | 6.27333 | 2.35931 | Tmem14a | Day_6 | Day_6_JQ1 | 6.27333 | 2.14481 | −1.5484 |
| Palld | Day_1 | Day_6 | OK | 8.68722 | 50.7402 | 2.54616 | Palld | Day_6 | Day_6_JQ1 | 50.7402 | 17.5752 | −1.5296 |
| Fam162a | Day_1 | Day_6 | OK | 73.9909 | 229.685 | 1.63424 | Fam162a | Day_6 | Day_6_JQ1 | 229.685 | 79.8975 | −1.5234 |
| Gpx7 | Day_1 | Day_6 | OK | 10.6539 | 63.6378 | 2.57851 | Gpx7 | Day_6 | Day_6_JQ1 | 63.6378 | 22.4891 | −1.5007 |
| P4ha2 | Day_1 | Day_6 | OK | 14.122 | 92.0875 | 2.70506 | P4ha2 | Day_6 | Day_6_JQ1 | 92.0875 | 32.777 | −1.4903 |
| Ppp1r13l | Day_1 | Day_6 | OK | 4.40351 | 15.0987 | 1.7777 | Ppp1r13l | Day_6 | Day_6_JQ1 | 15.0987 | 5.37842 | −1.4892 |
| Gpr161 | Day_1 | Day_6 | OK | 1.50608 | 5.66372 | 1.91095 | Gpr161 | Day_6 | Day_6_JQ1 | 5.66372 | 2.03169 | −1.4791 |
| Acta2 | Day_1 | Day_6 | OK | 45.8516 | 2402.37 | 5.71134 | Acta2 | Day_6 | Day_6_JQ1 | 2402.37 | 862.735 | −1.4775 |
| Lrrc27 | Day_1 | Day_6 | OK | 4.0541 | 13.0163 | 1.68286 | Lrrc27 | Day_6 | Day_6_JQ1 | 13.0163 | 4.68285 | −1.4749 |
| Bmper | Day_1 | Day_6 | OK | 6.65935 | 40.2027 | 2.59384 | Bmper | Day_6 | Day_6_JQ1 | 40.2027 | 14.4843 | −1.4728 |
| Mical2 | Day_1 | Day_6 | OK | 30.0672 | 84.7219 | 1.49455 | Mical2 | Day_6 | Day_6_JQ1 | 84.7219 | 30.5383 | −1.4721 |
| Tpi1 | Day_1 | Day_6 | OK | 129.064 | 418.772 | 1.69808 | Tpi1 | Day_6 | Day_6_JQ1 | 418.772 | 151.67 | −1.4652 |
| Trim16 | Day_1 | Day_6 | OK | 6.27334 | 13.8067 | 1.13807 | Trim16 | Day_6 | Day_6_JQ1 | 13.8067 | 5.01456 | −1.4612 |
| Mical1 | Day_1 | Day_6 | OK | 6.02089 | 24.9254 | 2.04957 | Mical1 | Day_6 | Day_6_JQ1 | 24.9254 | 9.05465 | −1.4609 |
| 6030419C18Rik | Day_1 | Day_6 | OK | 1.14586 | 17.6127 | 3.94212 | 6030419C18Rik | Day_6 | Day_6_JQ1 | 17.6127 | 6.41445 | −1.4572 |
| Dock5 | Day_1 | Day_6 | OK | 2.60342 | 9.98509 | 1.93937 | Dock5 | Day_6 | Day_6_JQ1 | 9.98509 | 3.64425 | −1.4542 |
| Alpl | Day_1 | Day_6 | OK | 9.27242 | 29.2545 | 1.65764 | Alpl | Day_6 | Day_6_JQ1 | 29.2545 | 10.6985 | −1.4513 |
| Slc38a1 | Day_1 | Day_6 | OK | 4.82912 | 15.8376 | 1.71352 | Slc38a1 | Day_6 | Day_6_JQ1 | 15.8376 | 5.82226 | −1.4437 |
| A130022J15Rik | Day_1 | Day_6 | OK | 5.76633 | 14.3808 | 1.31842 | A130022J15Rik | Day_6 | Day_6_JQ1 | 14.3808 | 5.29253 | −1.4421 |
| 2610029I01Rik | Day_1 | Day_6 | OK | 5.47637 | 16.0859 | 1.5545 | 2610029I01Rik | Day_6 | Day_6_JQ1 | 16.0859 | 5.92945 | −1.4398 |
| Thsd7a | Day_1 | Day_6 | OK | 0.8397 | 9.10897 | 3.43935 | Thsd7a | Day_6 | Day_6_JQ1 | 9.10897 | 3.36217 | −1.4379 |
| Fuca2 | Day_1 | Day_6 | OK | 8.19198 | 19.4095 | 1.24448 | Fuca2 | Day_6 | Day_6_JQ1 | 19.4095 | 7.1839 | −1.4339 |
| Leprel4 | Day_1 | Day_6 | OK | 11.7809 | 77.0854 | 2.71001 | Leprel4 | Day_6 | Day_6_JQ1 | 77.0854 | 28.5678 | −1.4321 |
| Limk2 | Day_1 | Day_6 | OK | 12.9407 | 29.7553 | 1.20123 | Limk2 | Day_6 | Day_6_JQ1 | 29.7553 | 11.0379 | −1.4307 |
| Prkag2 | Day_1 | Day_6 | OK | 3.73635 | 19.385 | 2.37524 | Prkag2 | Day_6 | Day_6_JQ1 | 19.385 | 7.31482 | −1.4061 |
| 2010011I20Rik | Day_1 | Day_6 | OK | 8.14544 | 31.9271 | 1.97072 | 2010011I20Rik | Day_6 | Day_6_JQ1 | 31.9271 | 12.0649 | −1.404 |
| Grem1 | Day_1 | Day_6 | OK | 5.51939 | 32.789 | 2.57063 | Grem1 | Day_6 | Day_6_JQ1 | 32.789 | 12.4243 | −1.4001 |
| Parm1 | Day_1 | Day_6 | OK | 2.27917 | 48.0525 | 4.39803 | Parm1 | Day_6 | Day_6_JQ1 | 48.0525 | 18.3367 | −1.3899 |
| Prrx2 | Day_1 | Day_6 | OK | 1.07176 | 10.4227 | 3.28168 | Prrx2 | Day_6 | Day_6_JQ1 | 10.4227 | 4.00366 | −1.3803 |
| Pacsin3 | Day_1 | Day_6 | OK | 4.47631 | 12.0027 | 1.42298 | Pacsin3 | Day_6 | Day_6_JQ1 | 12.0027 | 4.62741 | −1.3751 |
| Pgam1 | Day_1 | Day_6 | OK | 21.3537 | 65.3347 | 1.61336 | Pgam1 | Day_6 | Day_6_JQ1 | 65.3347 | 25.4014 | −1.3629 |
| Smarcd3 | Day_1 | Day_6 | OK | 4.30324 | 15.3482 | 1.83457 | Smarcd3 | Day_6 | Day_6_JQ1 | 15.3482 | 5.97342 | −1.3614 |
| Samd14 | Day_1 | Day_6 | OK | 1.83341 | 8.4334 | 2.20159 | Samd14 | Day_6 | Day_6_JQ1 | 8.4334 | 3.31384 | −1.3476 |
| Zdhhc2 | Day_1 | Day_6 | OK | 1.20497 | 8.92704 | 2.88919 | Zdhhc2 | Day_6 | Day_6_JQ1 | 8.92704 | 3.51017 | −1.3466 |
| D18Ertd653e | Day_1 | Day_6 | OK | 3.18521 | 12.258 | 1.94427 | D18Ertd653e | Day_6 | Day_6_JQ1 | 12.258 | 4.82101 | −1.3463 |
| Tgfb3 | Day_1 | Day_6 | OK | 8.63214 | 104.925 | 3.6035 | Tgfb3 | Day_6 | Day_6_JQ1 | 104.925 | 41.3316 | −1.3441 |
| Pcolce2 | Day_1 | Day_6 | OK | 10.309 | 26.9038 | 1.3839 | Pcolce2 | Day_6 | Day_6_JQ1 | 26.9038 | 10.6303 | −1.3396 |
| Ccdc8 | Day_1 | Day_6 | OK | 3.2331 | 11.5983 | 1.84292 | Ccdc8 | Day_6 | Day_6_JQ1 | 11.5983 | 4.58871 | −1.3378 |
| Atp9a | Day_1 | Day_6 | OK | 7.24735 | 20.4394 | 1.49583 | Atp9a | Day_6 | Day_6_JQ1 | 20.4394 | 8.10449 | −1.3346 |
| Gsn | Day_1 | Day_6 | OK | 33.981 | 135.742 | 1.99806 | Gsn | Day_6 | Day_6_JQ1 | 135.742 | 53.8689 | −1.3333 |
| Lmcd1 | Day_1 | Day_6 | OK | 19.3376 | 56.9504 | 1.5583 | Lmcd1 | Day_6 | Day_6_JQ1 | 56.9504 | 22.7407 | −1.3244 |
| Grb14 | Day_1 | Day_6 | OK | 0.15109 | 5.8885 | 5.28443 | Grb14 | Day_6 | Day_6_JQ1 | 5.8885 | 2.35486 | −1.3223 |
| Fgf2 | Day_1 | Day_6 | OK | 22.2416 | 77.3565 | 1.79826 | Fgf2 | Day_6 | Day_6_JQ1 | 77.3565 | 30.9424 | −1.3219 |
| Cryab | Day_1 | Day_6 | OK | 202.441 | 485.019 | 1.26054 | Cryab | Day_6 | Day_6_JQ1 | 485.019 | 194.462 | −1.3186 |
| Mgll | Day_1 | Day_6 | OK | 6.92622 | 15.8873 | 1.19774 | Mgll | Day_6 | Day_6_JQ1 | 15.8873 | 6.38862 | −1.3143 |
| Txndc15 | Day_1 | Day_6 | OK | 22.7825 | 55.5454 | 1.28574 | Txndc15 | Day_6 | Day_6_JQ1 | 55.5454 | 22.6625 | −1.2934 |
| C1s | Day_1 | Day_6 | OK | 9.29901 | 57.7096 | 2.63366 | C1s | Day_6 | Day_6_JQ1 | 57.7096 | 23.7532 | −1.2807 |
| D0H4S114 | Day_1 | Day_6 | OK | 3.56187 | 48.611 | 3.77058 | D0H4S114 | Day_6 | Day_6_JQ1 | 48.611 | 20.0603 | −1.2769 |
| Lrrn4cl | Day_1 | Day_6 | OK | 2.0859 | 6.79235 | 1.70324 | Lrrn4cl | Day_6 | Day_6_JQ1 | 6.79235 | 2.82782 | −1.2642 |
| Colec10 | Day_1 | Day_6 | OK | 1.55681 | 7.57159 | 2.282 | Colec10 | Day_6 | Day_6_JQ1 | 7.57159 | 3.15752 | −1.2618 |
| Arhgef40 | Day_1 | Day_6 | OK | 7.73592 | 22.7295 | 1.55492 | Arhgef40 | Day_6 | Day_6_JQ1 | 22.7295 | 9.51754 | −1.2559 |
| Creld1 | Day_1 | Day_6 | OK | 13.2336 | 36.9446 | 1.48116 | Creld1 | Day_6 | Day_6_JQ1 | 36.9446 | 15.4782 | −1.2551 |
| C1rl | Day_1 | Day_6 | OK | 1.06166 | 5.07307 | 2.25654 | C1rl | Day_6 | Day_6_JQ1 | 5.07307 | 2.13718 | −1.2472 |
| Slc39a13 | Day_1 | Day_6 | OK | 17.0097 | 62.5275 | 1.87813 | Slc39a13 | Day_6 | Day_6_JQ1 | 62.5275 | 26.4452 | −1.2415 |
| Tspan17 | Day_1 | Day_6 | OK | 5.9937 | 22.3346 | 1.89776 | Tspan17 | Day_6 | Day_6_JQ1 | 22.3346 | 9.45011 | −1.2409 |
| Ak1 | Day_1 | Day_6 | OK | 8.67228 | 107.924 | 3.63745 | Ak1 | Day_6 | Day_6_JQ1 | 107.924 | 45.9168 | −1.2329 |
| Dnm3os | Day_1 | Day_6 | OK | 0.71877 | 9.02295 | 3.64999 | Dnm3os | Day_6 | Day_6_JQ1 | 9.02295 | 3.84658 | −1.23 |
| Enpp3 | Day_1 | Day_6 | OK | 0.76849 | 6.93159 | 3.17308 | Enpp3 | Day_6 | Day_6_JQ1 | 6.93159 | 2.96186 | −1.2267 |
| Tgfb2 | Day_1 | Day_6 | OK | 2.30559 | 23.0676 | 3.32266 | Tgfb2 | Day_6 | Day_6_JQ1 | 23.0676 | 9.85858 | −1.2264 |
| Stbd1 | Day_1 | Day_6 | OK | 26.3563 | 111.959 | 2.08675 | Stbd1 | Day_6 | Day_6_JQ1 | 111.959 | 48.409 | −1.2096 |
| Rbm46 | Day_1 | Day_6 | OK | 2.61214 | 8.05482 | 1.62462 | Rbm46 | Day_6 | Day_6_JQ1 | 8.05482 | 3.53377 | −1.1887 |
| Hspb8 | Day_1 | Day_6 | OK | 30.7671 | 110.374 | 1.84294 | Hspb8 | Day_6 | Day_6_JQ1 | 110.374 | 48.5135 | −1.1859 |
| Cdc14b | Day_1 | Day_6 | OK | 2.5023 | 6.47418 | 1.37144 | Cdc14b | Day_6 | Day_6_JQ1 | 6.47418 | 2.86398 | −1.1767 |
| Prkcdbp | Day_1 | Day_6 | OK | 13.0952 | 68.1118 | 2.37886 | Prkcdbp | Day_6 | Day_6_JQ1 | 68.1118 | 30.1978 | −1.1735 |
| Mr1 | Day_1 | Day_6 | OK | 3.51043 | 12.5345 | 1.83618 | Mr1 | Day_6 | Day_6_JQ1 | 12.5345 | 5.56987 | −1.1702 |
| Rarg | Day_1 | Day_6 | OK | 14.2647 | 39.2734 | 1.4611 | Rarg | Day_6 | Day_6_JQ1 | 39.2734 | 17.4545 | −1.17 |
| Nudt6 | Day_1 | Day_6 | OK | 18.6938 | 66.686 | 1.83483 | Nudt6 | Day_6 | Day_6_JQ1 | 66.686 | 29.706 | −1.1666 |
| Adam19 | Day_1 | Day_6 | OK | 22.1947 | 121.076 | 2.44763 | Adam19 | Day_6 | Day_6_JQ1 | 121.076 | 54.8988 | −1.1411 |
| Tiam1 | Day_1 | Day_6 | OK | 0.71062 | 5.51715 | 2.95678 | Tiam1 | Day_6 | Day_6_JQ1 | 5.51715 | 2.52853 | −1.1256 |
| Fez1 | Day_1 | Day_6 | OK | 1.06222 | 6.55469 | 2.62544 | Fez1 | Day_6 | Day_6_JQ1 | 6.55469 | 3.033 | −1.1118 |
| Spsb1 | Day_1 | Day_6 | OK | 23.9615 | 73.6595 | 1.62016 | Spsb1 | Day_6 | Day_6_JQ1 | 73.6595 | 34.2732 | −1.1038 |
| Tceal8 | Day_1 | Day_6 | OK | 28.2285 | 93.7038 | 1.73096 | Tceal8 | Day_6 | Day_6_JQ1 | 93.7038 | 43.7417 | −1.0991 |
| Serf1 | Day_1 | Day_6 | OK | 8.22668 | 27.5993 | 1.74625 | Serf1 | Day_6 | Day_6_JQ1 | 27.5993 | 12.9215 | −1.0949 |
| Rcn3 | Day_1 | Day_6 | OK | 26.0723 | 344.846 | 3.72536 | Rcn3 | Day_6 | Day_6_JQ1 | 344.846 | 162.127 | −1.0888 |
| Mark1 | Day_1 | Day_6 | OK | 5.43527 | 24.108 | 2.14909 | Mark1 | Day_6 | Day_6_JQ1 | 24.108 | 11.3381 | −1.0883 |
| Olfml2b | Day_1 | Day_6 | OK | 1.42306 | 40.4936 | 4.83063 | Olfm12b | Day_6 | Day_6_JQ1 | 40.4936 | 19.0933 | −1.0846 |
| Adamts10 | Day_1 | Day_6 | OK | 3.01995 | 22.1077 | 2.87195 | Adamts10 | Day_6 | Day_6_JQ1 | 22.1077 | 10.4283 | −1.0841 |
| Cmbl | Day_1 | Day_6 | OK | 1.84816 | 14.4733 | 2.96923 | Cmbl | Day_6 | Day_6_JQ1 | 14.4733 | 6.84861 | −1.0795 |
| Islr | Day_1 | Day_6 | OK | 1.13495 | 20.3801 | 4.16645 | Islr | Day_6 | Day_6_JQ1 | 20.3801 | 9.71723 | −1.0685 |
| Ctsk | Day_1 | Day_6 | OK | 37.3865 | 79.3053 | 1.0849 | Ctsk | Day_6 | Day_6_JQ1 | 79.3053 | 37.8835 | −1.0659 |
| Cdkn2a | Day_1 | Day_6 | OK | 3.64104 | 44.4489 | 3.60973 | Cdkn2a | Day_6 | Day_6_JQ1 | 44.4489 | 21.4445 | −1.0515 |
| Ccdc80 | Day_1 | Day_6 | OK | 20.9979 | 423.888 | 4.33537 | Ccdc80 | Day_6 | Day_6_JQ1 | 423.888 | 205.926 | −1.0416 |
| Rnf150 | Day_1 | Day_6 | OK | 3.12739 | 10.6853 | 1.7726 | Rnf150 | Day_6 | Day_6_JQ1 | 10.6853 | 5.20097 | −1.0388 |
| Pfkp | Day_1 | Day_6 | OK | 11.4982 | 39.8468 | 1.79306 | Pfkp | Day_6 | Day_6_JQ1 | 39.8468 | 19.5069 | −1.0305 |
| Ripk3 | Day_1 | Day_6 | OK | 15.6438 | 54.4914 | 1.80044 | Ripk3 | Day_6 | Day_6_JQ1 | 54.4914 | 26.7225 | −1.028 |
| Kalrn | Day_1 | Day_6 | OK | 2.61523 | 11.4268 | 2.12741 | Kalrn | Day_6 | Day_6_JQ1 | 11.4268 | 5.60976 | −1.0264 |
| Lpar1 | Day_1 | Day_6 | OK | 2.16438 | 16.1507 | 2.89957 | Lpar1 | Day_6 | Day_6_JQ1 | 16.1507 | 7.93879 | −1.0246 |
| Ndrg4 | Day_1 | Day_6 | OK | 1.96491 | 22.7804 | 3.53526 | Ndrg4 | Day_6 | Day_6_JQ1 | 22.7804 | 11.1993 | −1.0244 |
| Paqr7 | Day_1 | Day_6 | OK | 10.6383 | 27.0186 | 1.34468 | Paqr7 | Day_6 | Day_6_JQ1 | 27.0186 | 13.3093 | −1.0215 |
| Gatsl3 | Day_1 | Day_6 | OK | 4.02765 | 15.7693 | 1.96911 | Gatsl3 | Day_6 | Day_6_JQ1 | 15.7693 | 7.79422 | −1.0166 |
| Bmp1 | Day_1 | Day_6 | OK | 14.4285 | 196.598 | 3.76826 | Bmp1 | Day_6 | Day_6_JQ1 | 196.598 | 97.3794 | −1.0136 |
| B230120H23Rik | Day_1 | Day_6 | OK | 10.3473 | 22.904 | 1.14635 | B230120H23Rik | Day_6 | Day_6_JQ1 | 22.904 | 11.3632 | −1.0112 |
| E130203B14Rik | Day_1 | Day_6 | OK | 2.235 | 7.29324 | 1.70628 | E130203B14Rik | Day_6 | Day_6_JQ1 | 7.29324 | 3.62279 | −1.0095 |
| Fbln2 | Day_1 | Day_6 | OK | 28.5536 | 941.151 | 5.04268 | Fbln2 | Day_6 | Day_6_JQ1 | 941.151 | 468.044 | −1.0078 |
| Rcn1 | Day_1 | Day_6 | OK | 61.2239 | 130.531 | 1.09222 | Rcn1 | Day_6 | Day_6_JQ1 | 130.531 | 64.9546 | −1.0069 |
| Vldlr | Day_1 | Day_6 | OK | 1.88636 | 9.73966 | 2.36826 | Vldlr | Day_6 | Day_6_JQ1 | 9.73966 | 4.85613 | −1.0041 |
| Aug-01 | Day_1 | Day_6 | OK | 2.05768 | 26.8582 | 3.70627 | Aug-01 | Day_6 | Day_6_JQ1 | 26.8582 | 13.5276 | −0.9895 |
| Pmaip1 | Day_1 | Day_6 | OK | 10.4375 | 24.4661 | 1.229 | Pmaip1 | Day_6 | Day_6_JQ1 | 24.4661 | 12.3575 | −0.9854 |
| Pck2 | Day_1 | Day_6 | OK | 23.3315 | 55.7193 | 1.2559 | Pck2 | Day_6 | Day_6_JQ1 | 55.7193 | 28.1623 | −0.9844 |
| Mgmt | Day_1 | Day_6 | OK | 6.75372 | 33.5483 | 2.31249 | Mgmt | Day_6 | Day_6_JQ1 | 33.5483 | 16.9626 | −0.9839 |
| Tagln | Day_1 | Day_6 | OK | 306.108 | 1946.13 | 2.6685 | Tagln | Day_6 | Day_6_JQ1 | 1946.13 | 987.996 | −0.978 |
| Slc38a4 | Day_1 | Day_6 | OK | 1.51389 | 7.39574 | 2.28844 | Slc38a4 | Day_6 | Day_6_JQ1 | 7.39574 | 3.75506 | −0.9779 |
| Mdk | Day_1 | Day_6 | OK | 3.15138 | 16.3487 | 2.37513 | Mdk | Day_6 | Day_6_JQ1 | 16.3487 | 8.31166 | −0.976 |
| Bgn | Day_1 | Day_6 | OK | 290.092 | 2577.12 | 3.15118 | Bgn | Day_6 | Day_6_JQ1 | 2577.12 | 1311.2 | −0.9749 |
| Prepl | Day_1 | Day_6 | OK | 6.8776 | 18.1879 | 1.403 | Prepl | Day_6 | Day_6_JQ1 | 18.1879 | 9.26061 | −0.9738 |
| Col1a2 | Day_1 | Day_6 | OK | 9.44635 | 1443.81 | 7.25591 | Col1a2 | Day_6 | Day_6_JQ1 | 1443.81 | 736.183 | −0.9717 |
| Specc1 | Day_1 | Day_6 | OK | 4.50547 | 35.8604 | 2.99264 | Specc1 | Day_6 | Day_6_JQ1 | 35.8604 | 18.3367 | −0.9677 |
| Nynrin | Day_1 | Day_6 | OK | 2.77465 | 10.4252 | 1.90971 | Nynrin | Day_6 | Day_6_JQ1 | 10.4252 | 5.34951 | −0.9626 |
| Fkbp14 | Day_1 | Day_6 | OK | 7.35399 | 33.8756 | 2.20365 | Fkbp14 | Day_6 | Day_6_JQ1 | 33.8756 | 17.3854 | −0.9624 |
| Mfap2 | Day_1 | Day_6 | OK | 0.26808 | 9.53014 | 5.15174 | Mfap2 | Day_6 | Day_6_JQ1 | 9.53014 | 4.89339 | −0.9617 |
| Enah | Day_1 | Day_6 | OK | 6.56254 | 21.2241 | 1.69338 | Enah | Day_6 | Day_6_JQ1 | 21.2241 | 10.9113 | −0.9599 |
| Pvrl1 | Day_1 | Day_6 | OK | 5.78202 | 16.8959 | 1.54703 | Pvrl1 | Day_6 | Day_6_JQ1 | 16.8959 | 8.70747 | −0.9563 |
| Col3a1 | Day_1 | Day_6 | OK | 21.5668 | 1590.04 | 6.20411 | Col3a1 | Day_6 | Day_6_JQ1 | 1590.04 | 820.017 | −0.9553 |
| Prss23 | Day_1 | Day_6 | OK | 39.1859 | 294.857 | 2.91161 | Prss23 | Day_6 | Day_6_JQ1 | 294.857 | 152.592 | −0.9503 |
| Ltbp3 | Day_1 | Day_6 | OK | 6.55732 | 50.2028 | 2.93659 | Ltbp3 | Day_6 | Day_6_JQ1 | 50.2028 | 26.195 | −0.9385 |
| Tubb3 | Day_1 | Day_6 | OK | 7.47406 | 28.7314 | 1.94266 | Tubb3 | Day_6 | Day_6_JQ1 | 28.7314 | 15.0331 | −0.9345 |
| Mtap1a | Day_1 | Day_6 | OK | 2.05121 | 13.9425 | 2.76494 | Mtap1a | Day_6 | Day_6_JQ1 | 13.9425 | 7.30521 | −0.9325 |
| Ammecr1 | Day_1 | Day_6 | OK | 4.64328 | 15.2271 | 1.71342 | Ammecr1 | Day_6 | Day_6_JQ1 | 15.2271 | 8.01456 | −0.9259 |
| Gpx3 | Day_1 | Day_6 | OK | 8.47716 | 180.305 | 4.41071 | Gpx3 | Day_6 | Day_6_JQ1 | 180.305 | 94.97 | −0.9249 |
| Dap | Day_1 | Day_6 | OK | 44.4796 | 130.333 | 1.55099 | Dap | Day_6 | Day_6_JQ1 | 130.333 | 68.7322 | −0.9231 |
| Lrch2 | Day_1 | Day_6 | OK | 0.40642 | 12.5655 | 4.95034 | Lrch2 | Day_6 | Day_6_JQ1 | 12.5655 | 6.6562 | −0.9167 |
| Pdgfd | Day_1 | Day_6 | OK | 1.8886 | 13.4798 | 2.83541 | Pdgfd | Day_6 | Day_6_JQ1 | 13.4798 | 7.154 | −0.914 |
| Tbc1d8b | Day_1 | Day_6 | OK | 4.68401 | 12.4987 | 1.41596 | Tbc1d8b | Day_6 | Day_6_JQ1 | 12.4987 | 6.64364 | −0.9117 |
| Mtap6 | Day_1 | Day_6 | OK | 1.22695 | 28.6868 | 4.54724 | Mtap6 | Day_6 | Day_6_JQ1 | 28.6868 | 15.2963 | −0.9072 |
| Ptgs1 | Day_1 | Day_6 | OK | 33.0869 | 73.2427 | 1.14643 | Ptgs1 | Day_6 | Day_6_JQ1 | 73.2427 | 39.0747 | −0.9064 |
| Tmem97 | Day_1 | Day_6 | OK | 13.2278 | 36.1148 | 1.44902 | Tmem97 | Day_6 | Day_6_JQ1 | 36.1148 | 19.3422 | −0.9008 |
| Nlrx1 | Day_1 | Day_6 | OK | 1.86983 | 6.40945 | 1.77729 | Nlrx1 | Day_6 | Day_6_JQ1 | 6.40945 | 3.43477 | −0.9 |
| Cnn2 | Day_1 | Day_6 | OK | 124.001 | 415.117 | 1.74317 | Cnn2 | Day_6 | Day_6_JQ1 | 415.117 | 222.556 | −0.8993 |
| Nnmt | Day_1 | Day_6 | OK | 14.4082 | 56.0599 | 1.96008 | Nnmt | Day_6 | Day_6_JQ1 | 56.0599 | 30.0607 | −0.8991 |
| LOC100616095 | Day_1 | Day_6 | OK | 3.77973 | 26.0616 | 2.78557 | LOC100616095 | Day_6 | Day_6_JQ1 | 26.0616 | 13.992 | −0.8973 |
| Rian | Day_1 | Day_6 | OK | 0.37241 | 43.3546 | 6.86314 | Rian | Day_6 | Day_6_JQ1 | 43.3546 | 23.3478 | −0.8929 |
| 3110003A17Rik | Day_1 | Day_6 | OK | 55.7976 | 193.911 | 1.79712 | 3110003A17Rik | Day_6 | Day_6_JQ1 | 193.911 | 104.778 | −0.8881 |
| Gm5424 | Day_1 | Day_6 | OK | 1.21301 | 9.84164 | 3.02031 | Gm5424 | Day_6 | Day_6_JQ1 | 9.84164 | 5.33112 | −0.8845 |
| Sec23a | Day_1 | Day_6 | OK | 13.3774 | 53.1413 | 1.99004 | Sec23a | Day_6 | Day_6_JQ1 | 53.1413 | 28.8101 | −0.8833 |
| Nav2 | Day_1 | Day_6 | OK | 4.39739 | 9.77964 | 1.15313 | Nav2 | Day_6 | Day_6_JQ1 | 9.77964 | 5.31105 | −0.8808 |
| Meg3 | Day_1 | Day_6 | OK | 0.29619 | 27.9244 | 6.55886 | Meg3 | Day_6 | Day_6_JQ1 | 27.9244 | 15.1775 | −0.8796 |
| Col5a2 | Day_1 | Day_6 | OK | 18.2095 | 684.349 | 5.23197 | Col5a2 | Day_6 | Day_6_JQ1 | 684.349 | 372.729 | −0.8766 |
| Setbp1 | Day_1 | Day_6 | OK | 0.59949 | 4.45298 | 2.89296 | Setbp1 | Day_6 | Day_6_JQ1 | 4.45298 | 2.43409 | −0.8714 |
| Pla2g4a | Day_1 | Day_6 | OK | 10.7703 | 27.1316 | 1.33292 | Pla2g4a | Day_6 | Day_6_JQ1 | 27.1316 | 14.8571 | −0.8688 |
| Thbs2 | Day_1 | Day_6 | OK | 1.63805 | 81.5588 | 5.63779 | Thbs2 | Day_6 | Day_6_JQ1 | 81.5588 | 44.7287 | −0.8666 |
| Gas6 | Day_1 | Day_6 | OK | 14.1036 | 75.0981 | 2.41272 | Gas6 | Day_6 | Day_6_JQ1 | 75.0981 | 41.2636 | −0.8639 |
| Cdkn2b | Day_1 | Day_6 | OK | 17.4134 | 75.8805 | 2.12353 | Cdkn2b | Day_6 | Day_6_JQ1 | 75.8805 | 41.7085 | −0.8634 |
| Plod3 | Day_1 | Day_6 | OK | 39.5344 | 86.5209 | 1.12994 | Plod3 | Day_6 | Day_6_JQ1 | 86.5209 | 47.8896 | −0.8533 |
| Shroom3 | Day_1 | Day_6 | OK | 3.81196 | 10.367 | 1.4434 | Shroom3 | Day_6 | Day_6_JQ1 | 10.367 | 5.75741 | −0.8485 |
| Fam114a1 | Day_1 | Day_6 | OK | 11.6447 | 53.5642 | 2.2016 | Fam114a1 | Day_6 | Day_6_JQ1 | 53.5642 | 29.7898 | −0.8465 |
| Lpp | Day_1 | Day_6 | OK | 16.9227 | 44.7173 | 1.40187 | Lpp | Day_6 | Day_6_JQ1 | 44.7173 | 24.9042 | −0.8444 |
| Gpx8 | Day_1 | Day_6 | OK | 52.5363 | 198.065 | 1.91459 | Gpx8 | Day_6 | Day_6_JQ1 | 198.065 | 110.547 | −0.8413 |
| Ckap4 | Day_1 | Day_6 | OK | 51.401 | 263.99 | 2.36062 | Ckap4 | Day_6 | Day_6_JQ1 | 263.99 | 147.656 | −0.8382 |
| Rftn1 | Day_1 | Day_6 | OK | 16.0428 | 36.8013 | 1.19784 | Rftn1 | Day_6 | Day_6_JQ1 | 36.8013 | 20.5933 | −0.8376 |
| Ndn | Day_1 | Day_6 | OK | 14.1983 | 59.3502 | 2.06353 | Ndn | Day_6 | Day_6_JQ1 | 59.3502 | 33.2623 | −0.8354 |
| Rhoq | Day_1 | Day_6 | OK | 20.5798 | 64.9006 | 1.657 | Rhoq | Day_6 | Day_6_JQ1 | 64.9006 | 36.4737 | −0.8314 |
| Zranb3 | Day_1 | Day_6 | OK | 1.10734 | 4.63291 | 2.06481 | Zranb3 | Day_6 | Day_6_JQ1 | 4.63291 | 2.60803 | −0.829 |
| Cdc42bpa | Day_1 | Day_6 | OK | 14.0755 | 29.3325 | 1.05932 | Cdc42bpa | Day_6 | Day_6_JQ1 | 29.3325 | 16.5997 | −0.8213 |
| Fhl2 | Day_1 | Day_6 | OK | 15.6516 | 70.995 | 2.18141 | Fhl2 | Day_6 | Day_6_JQ1 | 70.995 | 40.1985 | −0.8206 |
| Pcolce | Day_1 | Day_6 | OK | 9.25823 | 182.278 | 4.29926 | Pcolce | Day_6 | Day_6_JQ1 | 182.278 | 103.689 | −0.8139 |
| Kif1a | Day_1 | Day_6 | OK | 0.36072 | 11.5345 | 4.99894 | Kif1a | Day_6 | Day_6_JQ1 | 11.5345 | 6.56999 | −0.812 |
| Serping1 | Day_1 | Day_6 | OK | 58.4117 | 210.044 | 1.84636 | Serping1 | Day_6 | Day_6_JQ1 | 210.044 | 119.681 | −0.8115 |
| Pkig | Day_1 | Day_6 | OK | 32.3964 | 73.1928 | 1.17587 | Pkig | Day_6 | Day_6_JQ1 | 73.1928 | 41.8645 | −0.806 |
| Ogdhl | Day_1 | Day_6 | OK | 0.97396 | 8.25111 | 3.08266 | Ogdhl | Day_6 | Day_6_JQ1 | 8.25111 | 4.72569 | −0.8041 |
| Col5a1 | Day_1 | Day_6 | OK | 14.676 | 185.302 | 3.65835 | Col5a1 | Day_6 | Day_6_JQ1 | 185.302 | 106.143 | −0.8039 |
| Mpp2 | Day_1 | Day_6 | OK | 1.11266 | 8.09833 | 2.86361 | Mpp2 | Day_6 | Day_6_JQ1 | 8.09833 | 4.63887 | −0.8039 |
| Atp8b2 | Day_1 | Day_6 | OK | 12.9888 | 33.9847 | 1.38761 | Atp8b2 | Day_6 | Day_6_JQ1 | 33.9847 | 19.4721 | −0.8035 |
| Adora2b | Day_1 | Day_6 | OK | 9.93474 | 22.4847 | 1.17839 | Adora2b | Day_6 | Day_6_JQ1 | 22.4847 | 12.9207 | −0.7993 |
| Gria3 | Day_1 | Day_6 | OK | 0.21903 | 8.31167 | 5.24595 | Gria3 | Day_6 | Day_6_JQ1 | 8.31167 | 4.78031 | −0.798 |
| Mboat7 | Day_1 | Day_6 | OK | 20.2091 | 49.7606 | 1.3 | Mboat7 | Day_6 | Day_6_JQ1 | 49.7606 | 28.6438 | −0.7968 |
| Cpe | Day_1 | Day_6 | OK | 24.5792 | 95.1186 | 1.95229 | Cpe | Day_6 | Day_6_JQ1 | 95.1186 | 54.9734 | −0.791 |
| 9030617O03Rik | Day_1 | Day_6 | OK | 5.78774 | 15.9871 | 1.46584 | 9030617O03Rik | Day_6 | Day_6_JQ1 | 15.9871 | 9.24851 | −0.7896 |
| Cpz | Day_1 | Day_6 | OK | 0.61816 | 8.368 | 3.75882 | Cpz | Day_6 | Day_6_JQ1 | 8.368 | 4.84264 | −0.7891 |
| Nradd | Day_1 | Day_6 | OK | 8.12753 | 26.7197 | 1.71702 | Nradd | Day_6 | Day_6_JQ1 | 26.7197 | 15.4656 | −0.7888 |
| Col8a1 | Day_1 | Day_6 | OK | 1.89849 | 120.141 | 5.98374 | Col8a1 | Day_6 | Day_6_JQ1 | 120.141 | 69.7251 | −0.785 |
| Lrrn4 | Day_1 | Day_6 | OK | 2.95918 | 20.1313 | 2.76617 | Lrrn4 | Day_6 | Day_6_JQ1 | 20.1313 | 11.7356 | −0.7786 |
| Pdia5 | Day_1 | Day_6 | OK | 9.9452 | 35.5037 | 1.8359 | Pdia5 | Day_6 | Day_6_JQ1 | 35.5037 | 20.7466 | −0.7751 |
| Igfbp3 | Day_1 | Day_6 | OK | 48.6506 | 824.196 | 4.08246 | Igfbp3 | Day_6 | Day_6_JQ1 | 824.196 | 483.66 | −0.769 |
| Tpm2 | Day_1 | Day_6 | OK | 10.7459 | 195.392 | 4.18452 | Tpm2 | Day_6 | Day_6_JQ1 | 195.392 | 114.662 | −0.769 |
| Chst14 | Day_1 | Day_6 | OK | 15.3299 | 32.1154 | 1.06692 | Chst14 | Day_6 | Day_6_JQ1 | 32.1154 | 18.8651 | −0.7675 |
| Ak3 | Day_1 | Day_6 | OK | 17.6426 | 47.2653 | 1.42172 | Ak3 | Day_6 | Day_6_JQ1 | 47.2653 | 27.7827 | −0.7666 |
| Tmem9 | Day_1 | Day_6 | OK | 10.8063 | 34.7986 | 1.68715 | Tmem9 | Day_6 | Day_6_JQ1 | 34.7986 | 20.5522 | −0.7597 |
| Fzd2 | Day_1 | Day_6 | OK | 0.38216 | 27.8682 | 6.1883 | Fzd2 | Day_6 | Day_6_JQ1 | 27.8682 | 16.4688 | −0.7589 |
| Pgm2 | Day_1 | Day_6 | OK | 24.9 | 62.1789 | 1.32028 | Pgm2 | Day_6 | Day_6_JQ1 | 62.1789 | 36.9612 | −0.7504 |
| Cav1 | Day_1 | Day_6 | OK | 12.689 | 29.4129 | 1.21287 | Cav1 | Day_6 | Day_6_JQ1 | 29.4129 | 17.5128 | −0.748 |
| Snurf | Day_1 | Day_6 | OK | 2.28542 | 11.2505 | 2.29946 | Snurf | Day_6 | Day_6_JQ1 | 11.2505 | 6.70769 | −0.7461 |
| Ptgfrn | Day_1 | Day_6 | OK | 9.0478 | 40.4837 | 2.1617 | Ptgfrn | Day_6 | Day_6_JQ1 | 40.4837 | 24.2749 | −0.7379 |
| Ass1 | Day_1 | Day_6 | OK | 8.59192 | 68.6003 | 2.99716 | Ass1 | Day_6 | Day_6_JQ1 | 68.6003 | 41.1545 | −0.7372 |
| Praf2 | Day_1 | Day_6 | OK | 39.4096 | 84.7571 | 1.10479 | Praf2 | Day_6 | Day_6_JQ1 | 84.7571 | 50.8557 | −0.7369 |
| Dst | Day_1 | Day_6 | OK | 10.5552 | 28.2022 | 1.41786 | Dst | Day_6 | Day_6_JQ1 | 28.2022 | 16.9506 | −0.7345 |
| Isoc2b | Day_1 | Day_6 | OK | 1.44931 | 6.75202 | 2.21995 | Isoc2b | Day_6 | Day_6_JQ1 | 6.75202 | 4.05902 | −0.7342 |
| Flnc | Day_1 | Day_6 | OK | 2.55444 | 40.2597 | 3.97826 | Flnc | Day_6 | Day_6_JQ1 | 40.2597 | 24.3492 | −0.7255 |
| Evc2 | Day_1 | Day_6 | OK | 5.1578 | 12.1294 | 1.23368 | Evc2 | Day_6 | Day_6_JQ1 | 12.1294 | 7.33697 | −0.7253 |
| Pde4dip | Day_1 | Day_6 | OK | 12.2212 | 24.5957 | 1.00902 | Pde4dip | Day_6 | Day_6_JQ1 | 24.5957 | 14.9114 | −0.722 |
| AI597468 | Day_1 | Day_6 | OK | 8.51985 | 32.9859 | 1.95295 | AI597468 | Day_6 | Day_6_JQ1 | 32.9859 | 20.0206 | −0.7204 |
| Cygb | Day_1 | Day_6 | OK | 12.0769 | 85.3948 | 2.82189 | Cygb | Day_6 | Day_6_JQ1 | 85.3948 | 51.8956 | −0.7185 |
| Pgcp | Day_1 | Day_6 | OK | 5.16972 | 33.7973 | 2.70875 | Pgcp | Day_6 | Day_6_JQ1 | 33.7973 | 20.5624 | −0.7169 |
| Dcaf12l1 | Day_1 | Day_6 | OK | 0.33377 | 5.67289 | 4.08714 | Dcaf12l1 | Day_6 | Day_6_JQ1 | 5.67289 | 3.45623 | −0.7149 |
| Nudt18 | Day_1 | Day_6 | OK | 3.39992 | 10.6469 | 1.64687 | Nudt18 | Day_6 | Day_6_JQ1 | 10.6469 | 6.50093 | −0.7117 |
| Fam115c | Day_1 | Day_6 | OK | 2.43033 | 7.53152 | 1.63179 | Fam115c | Day_6 | Day_6_JQ1 | 7.53152 | 4.61053 | −0.708 |
| Rasl11a | Day_1 | Day_6 | OK | 9.01536 | 111.081 | 3.62309 | Rasl11a | Day_6 | Day_6_JQ1 | 111.081 | 68.0496 | −0.707 |
| Dlg4 | Day_1 | Day_6 | OK | 5.89503 | 14.8274 | 1.33069 | Dlg4 | Day_6 | Day_6_JQ1 | 14.8274 | 9.10081 | −0.7042 |
| Fgf18 | Day_1 | Day_6 | OK | 1.05033 | 17.0736 | 4.02285 | Fgf18 | Day_6 | Day_6_JQ1 | 17.0736 | 10.4873 | −0.7031 |
| Mmgt2 | Day_1 | Day_6 | OK | 7.02204 | 16.9398 | 1.27045 | Mmgt2 | Day_6 | Day_6_JQ1 | 16.9398 | 10.425 | −0.7004 |
| Csrp1 | Day_1 | Day_6 | OK | 164.676 | 478.807 | 1.53981 | Csrp1 | Day_6 | Day_6_JQ1 | 478.807 | 294.801 | −0.6997 |
| Scd1 | Day_1 | Day_6 | OK | 12.2292 | 37.8217 | 1.62889 | Scd1 | Day_6 | Day_6_JQ1 | 37.8217 | 23.3211 | −0.6976 |
| Rasl11b | Day_1 | Day_6 | OK | 1.8916 | 44.1692 | 4.54536 | Rasl11b | Day_6 | Day_6_JQ1 | 44.1692 | 27.3606 | −0.6909 |
| Npr2 | Day_1 | Day_6 | OK | 4.09305 | 16.4152 | 2.00378 | Npr2 | Day_6 | Day_6_JQ1 | 16.4152 | 10.1828 | −0.6889 |
| Gabra3 | Day_1 | Day_6 | OK | 1.87806 | 23.8369 | 3.66588 | Gabra3 | Day_6 | Day_6_JQ1 | 23.8369 | 14.8418 | −0.6835 |
| Aga | Day_1 | Day_6 | OK | 18.206 | 67.3834 | 1.88798 | Aga | Day_6 | Day_6_JQ1 | 67.3834 | 42.0303 | −0.681 |
| Adam12 | Day_1 | Day_6 | OK | 6.15715 | 35.4191 | 2.5242 | Adam12 | Day_6 | Day_6_JQ1 | 35.4191 | 22.1402 | −0.6779 |
| Ppp2r5b | Day_1 | Day_6 | OK | 12.2241 | 24.914 | 1.02723 | Ppp2r5b | Day_6 | Day_6_JQ1 | 24.914 | 15.5804 | −0.6772 |
| Dnajb4 | Day_1 | Day_6 | OK | 33.9307 | 70.8231 | 1.06163 | Dnajb4 | Day_6 | Day_6_JQ1 | 70.8231 | 44.3814 | −0.6743 |
| C1qtnf6 | Day_1 | Day_6 | OK | 14.7405 | 93.4598 | 2.66456 | C1qtnf6 | Day_6 | Day_6_JQ1 | 93.4598 | 58.5752 | −0.6741 |
| Btg2 | Day_1 | Day_6 | OK | 19.899 | 61.6259 | 1.63084 | Btg2 | Day_6 | Day_6_JQ1 | 61.6259 | 38.7075 | −0.6709 |
| Nuak1 | Day_1 | Day_6 | OK | 4.24632 | 20.0655 | 2.24043 | Nuak1 | Day_6 | Day_6_JQ1 | 20.0655 | 12.6565 | −0.6648 |
| Mmp2 | Day_1 | Day_6 | OK | 16.8598 | 116.377 | 2.78715 | Mmp2 | Day_6 | Day_6_JQ1 | 116.377 | 73.6315 | −0.6604 |
| Grhpr | Day_1 | Day_6 | OK | 7.60535 | 21.8483 | 1.52244 | Grhpr | Day_6 | Day_6_JQ1 | 21.8483 | 13.8722 | −0.6553 |
| Col6a1 | Day_1 | Day_6 | OK | 11.0949 | 74.6784 | 2.75079 | Col6a1 | Day_6 | Day_6_JQ1 | 74.6784 | 47.4496 | −0.6543 |
| Steap2 | Day_1 | Day_6 | OK | 11.7631 | 27.4067 | 1.22026 | Steap2 | Day_6 | Day_6_JQ1 | 27.4067 | 17.4478 | −0.6515 |
| Ufsp2 | Day_1 | Day_6 | OK | 15.2292 | 33.4708 | 1.13606 | Ufsp2 | Day_6 | Day_6_JQ1 | 33.4708 | 21.3133 | −0.6512 |
| C1qtnf1 | Day_1 | Day_6 | OK | 13.3548 | 29.9333 | 1.16439 | C1qtnf1 | Day_6 | Day_6_JQ1 | 29.9333 | 19.1118 | −0.6473 |
| Ccdc109b | Day_1 | Day_6 | OK | 4.49649 | 14.3985 | 1.67905 | Ccdc109b | Day_6 | Day_6_JQ1 | 14.3985 | 9.23248 | −0.6411 |
| Lars2 | Day_1 | Day_6 | OK | 9.54309 | 19.6633 | 1.04298 | Lars2 | Day_6 | Day_6_JQ1 | 19.6633 | 12.6226 | −0.6395 |
| Cyp51 | Day_1 | Day_6 | OK | 8.86094 | 20.9057 | 1.23836 | Cyp51 | Day_6 | Day_6_JQ1 | 20.9057 | 13.4382 | −0.6376 |
| Lrig3 | Day_1 | Day_6 | OK | 2.55847 | 6.54864 | 1.35591 | Lrig3 | Day_6 | Day_6_JQ1 | 6.54864 | 4.21785 | −0.6347 |
| Zfp651 | Day_1 | Day_6 | OK | 1.17488 | 8.70948 | 2.89007 | Zfp651 | Day_6 | Day_6_JQ1 | 8.70948 | 5.6272 | −0.6302 |
| Reep5 | Day_1 | Day_6 | OK | 33.4079 | 65.9211 | 0.98055 | Reep5 | Day_6 | Day_6_JQ1 | 65.9211 | 42.7167 | −0.6259 |
| Fam149a | Day_1 | Day_6 | OK | 2.88227 | 6.85652 | 1.25027 | Fam149a | Day_6 | Day_6_JQ1 | 6.85652 | 4.45213 | −0.623 |
| Ypel5 | Day_1 | Day_6 | OK | 19.9197 | 52.56 | 1.39977 | Ypel5 | Day_6 | Day_6_JQ1 | 52.56 | 34.2738 | −0.6169 |
| Samd4 | Day_1 | Day_6 | OK | 10.9712 | 23.3172 | 1.08767 | Samd4 | Day_6 | Day_6_JQ1 | 23.3172 | 15.2321 | −0.6143 |
| Efemp2 | Day_1 | Day_6 | OK | 38.2828 | 259.832 | 2.76281 | Efemp2 | Day_6 | Day_6_JQ1 | 259.832 | 169.818 | −0.6136 |
| Anxa6 | Day_1 | Day_6 | OK | 71.4672 | 186.357 | 1.38271 | Anxa6 | Day_6 | Day_6_JQ1 | 186.357 | 122.139 | −0.6095 |
| Il1rap | Day_1 | Day_6 | OK | 9.60564 | 31.4831 | 1.71262 | Il1rap | Day_6 | Day_6_JQ1 | 31.4831 | 20.7058 | −0.6045 |
| 6330406I15Rik | Day_1 | Day_6 | OK | 4.85402 | 46.2832 | 3.25324 | 6330406I15Rik | Day_6 | Day_6_JQ1 | 46.2832 | 30.5279 | −0.6004 |
| Epdr1 | Day_1 | Day_6 | OK | 2.14348 | 11.0552 | 2.36669 | Epdr1 | Day_6 | Day_6_JQ1 | 11.0552 | 7.33627 | −0.5916 |
| Maged2 | Day_1 | Day_6 | OK | 44.253 | 91.2266 | 1.04368 | Maged2 | Day_6 | Day_6_JQ1 | 91.2266 | 60.8426 | −0.5844 |
| Paqr8 | Day_1 | Day_6 | OK | 3.81619 | 11.1195 | 1.54289 | Paqr8 | Day_6 | Day_6_JQ1 | 11.1195 | 7.42445 | −0.5827 |
| Zmat3 | Day_1 | Day_6 | OK | 11.5787 | 27.2408 | 1.23429 | Zmat3 | Day_6 | Day_6_JQ1 | 27.2408 | 18.2014 | −0.5817 |
| Ptger3 | Day_1 | Day_6 | OK | 0.93314 | 9.0659 | 3.28028 | Ptger3 | Day_6 | Day_6_JQ1 | 9.0659 | 6.06417 | −0.5801 |
| Lama2 | Day_1 | Day_6 | OK | 0.2719 | 13.845 | 5.67015 | Lama2 | Day_6 | Day_6_JQ1 | 13.845 | 9.26282 | −0.5798 |
| Mlec | Day_1 | Day_6 | OK | 29.2922 | 137.144 | 2.2271 | Mlec | Day_6 | Day_6_JQ1 | 137.144 | 92.0676 | −0.5749 |
| Eda2r | Day_1 | Day_6 | OK | 16.7524 | 34.2319 | 1.03097 | Eda2r | Day_6 | Day_6_JQ1 | 34.2319 | 23.0105 | −0.5731 |
| Slc24a3 | Day_1 | Day_6 | OK | 0.57811 | 4.60078 | 2.99246 | Slc24a3 | Day_6 | Day_6_JQ1 | 4.60078 | 3.11126 | −0.5644 |
| Fam189b | Day_1 | Day_6 | OK | 1.53073 | 10.9765 | 2.84212 | Fam189b | Day_6 | Day_6_JQ1 | 10.9765 | 7.43119 | −0.5628 |
| Psd3 | Day_1 | Day_6 | OK | 2.34709 | 8.23777 | 1.81138 | Psd3 | Day_6 | Day_6_JQ1 | 8.23777 | 5.57858 | −0.5624 |
| Ccdc104 | Day_1 | Day_6 | OK | 11.0369 | 29.0477 | 1.39609 | Ccdc104 | Day_6 | Day_6_JQ1 | 29.0477 | 19.7014 | −0.5601 |
| Ttc19 | Day_1 | Day_6 | OK | 7.19358 | 16.1487 | 1.16663 | Ttc19 | Day_6 | Day_6_JQ1 | 16.1487 | 10.9985 | −0.5541 |
| Tmem176a | Day_1 | Day_6 | OK | 33.8485 | 200.304 | 2.56503 | Tmem176a | Day_6 | Day_6_JQ1 | 200.304 | 136.733 | −0.5508 |
| Ptk2b | Day_1 | Day_6 | OK | 7.68773 | 25.1016 | 1.70715 | Ptk2b | Day_6 | Day_6_JQ1 | 25.1016 | 17.1497 | −0.5496 |
| Prnp | Day_1 | Day_6 | OK | 42.1317 | 297.574 | 2.82027 | Prnp | Day_6 | Day_6_JQ1 | 297.574 | 204.182 | −0.5434 |
| Slc7a6 | Day_1 | Day_6 | OK | 14.5017 | 29.5582 | 1.02734 | Slc7a6 | Day_6 | Day_6_JQ1 | 29.5582 | 20.2814 | −0.5434 |
| Igsf8 | Day_1 | Day_6 | OK | 21.8083 | 64.9591 | 1.57465 | Igsf8 | Day_6 | Day_6_JQ1 | 64.9591 | 44.7273 | −0.5384 |
| Inpp5a | Day_1 | Day_6 | OK | 22.8022 | 48.444 | 1.08714 | Inpp5a | Day_6 | Day_6_JQ1 | 48.444 | 33.3765 | −0.5375 |
| Sepw1 | Day_1 | Day_6 | OK | 57.6685 | 138.168 | 1.26057 | Sepw1 | Day_6 | Day_6_JQ1 | 138.168 | 95.2318 | −0.5369 |
| Qsox1 | Day_1 | Day_6 | OK | 50.1162 | 132.76 | 1.40547 | Qsox1 | Day_6 | Day_6_JQ1 | 132.76 | 91.7197 | −0.5335 |
| Arhgef10 | Day_1 | Day_6 | OK | 3.22378 | 8.62791 | 1.42026 | Arhgef10 | Day_6 | Day_6_JQ1 | 8.62791 | 5.98746 | −0.5271 |
| Tmem38a | Day_1 | Day_6 | OK | 3.37058 | 14.2445 | 2.07933 | Tmem38a | Day_6 | Day_6_JQ1 | 14.2445 | 9.93403 | −0.52 |
| Chst12 | Day_1 | Day_6 | OK | 10.3748 | 63.2077 | 2.60702 | Chst12 | Day_6 | Day_6_JQ1 | 63.2077 | 44.2429 | −0.5147 |
| Ror1 | Day_1 | Day_6 | OK | 0.70862 | 8.75807 | 3.62753 | Ror1 | Day_6 | Day_6_JQ1 | 8.75807 | 6.1328 | −0.5141 |
| Runx2 | Day_1 | Day_6 | OK | 1.35528 | 5.34228 | 1.97887 | Runx2 | Day_6 | Day_6_JQ1 | 5.34228 | 3.74129 | −0.5139 |
| Leprel1 | Day_1 | Day_6 | OK | 2.21261 | 6.90255 | 1.64138 | Leprel1 | Day_6 | Day_6_JQ1 | 6.90255 | 4.83998 | −0.5121 |
| Kank1 | Day_1 | Day_6 | OK | 6.13079 | 22.6011 | 1.88225 | Kank1 | Day_6 | Day_6_JQ1 | 22.6011 | 15.8777 | −0.5094 |
| Ccbe1 | Day_1 | Day_6 | OK | 11.362 | 37.2914 | 1.71463 | Ccbe1 | Day_6 | Day_6_JQ1 | 37.2914 | 26.2027 | −0.5091 |
| Whrn | Day_1 | Day_6 | OK | 1.74993 | 7.09694 | 2.0199 | Whrn | Day_6 | Day_6_JQ1 | 7.09694 | 4.99295 | −0.5073 |
| Man2b2 | Day_1 | Day_6 | OK | 18.1191 | 38.3648 | 1.08227 | Man2b2 | Day_6 | Day_6_JQ1 | 38.3648 | 26.9926 | −0.5072 |
| Ccnd2 | Day_1 | Day_6 | OK | 44.5804 | 203.751 | 2.19233 | Ccnd2 | Day_6 | Day_6_JQ1 | 203.751 | 143.49 | −0.5059 |
| Extl3 | Day_1 | Day_6 | OK | 20.832 | 50.5513 | 1.27895 | Extl3 | Day_6 | Day_6_JQ1 | 50.5513 | 35.6167 | −0.5052 |
| Boc | Day_1 | Day_6 | OK | 2.21053 | 20.1023 | 3.1849 | Boc | Day_6 | Day_6_JQ1 | 20.1023 | 14.1708 | −0.5044 |
| Gm14420 | Day_1 | Day_6 | OK | 9.50633 | 19.4871 | 1.03555 | Gm14420 | Day_6 | Day_6_JQ1 | 19.4871 | 13.7546 | −0.5026 |
| Sfrp1 | Day_1 | Day_6 | OK | 0.69921 | 47.0461 | 6.07221 | Sfrp1 | Day_6 | Day_6_JQ1 | 47.0461 | 33.2277 | −0.5017 |
| Prrc1 | Day_1 | Day_6 | OK | 11.6753 | 28.1642 | 1.27041 | Prrc1 | Day_6 | Day_6_JQ1 | 28.1642 | 19.9334 | −0.4987 |
| Prdx2 | Day_1 | Day_6 | OK | 114.011 | 236.922 | 1.05523 | Prdx2 | Day_6 | Day_6_JQ1 | 236.922 | 167.804 | −0.4976 |
| P4ha1 | Day_1 | Day_6 | OK | 35.9648 | 170.271 | 2.24318 | P4ha1 | Day_6 | Day_6_JQ1 | 170.271 | 120.896 | −0.4941 |
| Fbxw9 | Day_1 | Day_6 | OK | 8.52034 | 20.0939 | 1.23778 | Fbxw9 | Day_6 | Day_6_JQ1 | 20.0939 | 14.3107 | −0.4897 |
| Sat2 | Day_1 | Day_6 | OK | 1.0606 | 7.36815 | 2.79642 | Sat2 | Day_6 | Day_6_JQ1 | 7.36815 | 5.26042 | −0.4861 |
| Tubb2b | Day_1 | Day_6 | OK | 3.81711 | 26.3717 | 2.78843 | Tubb2b | Day_6 | Day_6_JQ1 | 26.3717 | 18.8633 | −0.4834 |
| Plscr4 | Day_1 | Day_6 | OK | 4.1982 | 11.4122 | 1.44274 | Plscr4 | Day_6 | Day_6_JQ1 | 11.4122 | 8.1813 | −0.4802 |
| Tpm1 | Day_1 | Day_6 | OK | 433.11 | 2009.87 | 2.21429 | Tpm1 | Day_6 | Day_6_JQ1 | 2009.87 | 1441.58 | −0.4794 |
| Dzip11 | Day_1 | Day_6 | OK | 3.20928 | 15.091 | 2.23336 | Dzip11 | Day_6 | Day_6_JQ1 | 15.091 | 10.8578 | −0.475 |
| Fam110c | Day_1 | Day_6 | OK | 2.6577 | 18.6224 | 2.80879 | Fam110c | Day_6 | Day_6_JQ1 | 18.6224 | 13.4372 | −0.4708 |
| Arl5a | Day_1 | Day_6 | OK | 20.8911 | 51.9075 | 1.31306 | Arl5a | Day_6 | Day_6_JQ1 | 51.9075 | 37.4802 | −0.4698 |
| Fkbp7 | Day_1 | Day_6 | OK | 8.37738 | 55.5496 | 2.72921 | Fkbp7 | Day_6 | Day_6_JQ1 | 55.5496 | 40.2493 | −0.4648 |
| Tm7sf3 | Day_1 | Day_6 | OK | 11.2316 | 24.6658 | 1.13495 | Tm7sf3 | Day_6 | Day_6_JQ1 | 24.6658 | 17.8846 | −0.4638 |
| Lgals1 | Day_1 | Day_6 | OK | 303.437 | 2017.95 | 2.73342 | Lgals1 | Day_6 | Day_6_JQ1 | 2017.95 | 1464.5 | −0.4625 |
| Myo1d | Day_1 | Day_6 | OK | 13.6087 | 30.9567 | 1.18572 | Myo1d | Day_6 | Day_6_JQ1 | 30.9567 | 22.5024 | −0.4602 |
| Mylk | Day_1 | Day_6 | OK | 18.0269 | 85.2267 | 2.24116 | Mylk | Day_6 | Day_6_JQ1 | 85.2267 | 62.0714 | −0.4574 |
| Col4a5 | Day_1 | Day_6 | OK | 0.32814 | 49.5103 | 7.23727 | Col4a5 | Day_6 | Day_6_JQ1 | 49.5103 | 36.069 | −0.457 |
| Stk39 | Day_1 | Day_6 | OK | 8.49219 | 23.3251 | 1.45767 | Stk39 | Day_6 | Day_6_JQ1 | 23.3251 | 17.0092 | −0.4556 |
| Lclat1 | Day_1 | Day_6 | OK | 8.76769 | 28.3218 | 1.69165 | Lclat1 | Day_6 | Day_6_JQ1 | 28.3218 | 20.6695 | −0.4544 |
| Pla2g15 | Day_1 | Day_6 | OK | 13.7039 | 33.1654 | 1.2751 | Pla2g15 | Day_6 | Day_6_JQ1 | 33.1654 | 24.3031 | −0.4485 |
| Zyx | Day_1 | Day_6 | OK | 91.5579 | 252.291 | 1.46233 | Zyx | Day_6 | Day_6_JQ1 | 252.291 | 185.005 | −0.4475 |
| Snx30 | Day_1 | Day_6 | OK | 5.84014 | 17.8788 | 1.61418 | Snx30 | Day_6 | Day_6_JQ1 | 17.8788 | 13.1255 | −0.4459 |
| Slit3 | Day_1 | Day_6 | OK | 0.66385 | 53.5246 | 6.3332 | Slit3 | Day_6 | Day_6_JQ1 | 53.5246 | 39.3977 | −0.4421 |
| Pppde2 | Day_1 | Day_6 | OK | 13.242 | 27.8192 | 1.07096 | Pppde2 | Day_6 | Day_6_JQ1 | 27.8192 | 20.4808 | −0.4418 |
| Ccng1 | Day_1 | Day_6 | OK | 54.3723 | 221.671 | 2.02748 | Ccng1 | Day_6 | Day_6_JQ1 | 221.671 | 163.356 | −0.4404 |
| Gpc4 | Day_1 | Day_6 | OK | 20.283 | 127.853 | 2.65615 | Gpc4 | Day_6 | Day_6_JQ1 | 127.853 | 94.2819 | −0.4394 |
| Mpzl1 | Day_1 | Day_6 | OK | 54.4981 | 119.398 | 1.1315 | Mpzl1 | Day_6 | Day_6_JQ1 | 119.398 | 88.2456 | −0.4362 |
| B4galt2 | Day_1 | Day_6 | OK | 8.94465 | 20.5014 | 1.19663 | B4galt2 | Day_6 | Day_6_JQ1 | 20.5014 | 15.1944 | −0.4322 |
| Ikbip | Day_1 | Day_6 | OK | 16.5246 | 92.222 | 2.48049 | Ikbip | Day_6 | Day_6_JQ1 | 92.222 | 68.3911 | −0.4313 |
| Prickle1 | Day_1 | Day_6 | OK | 3.46818 | 19.1964 | 2.46858 | Prickle1 | Day_6 | Day_6_JQ1 | 19.1964 | 14.268 | −0.428 |
| Sulf1 | Day_1 | Day_6 | OK | 1.78992 | 72.367 | 5.33736 | Sulf1 | Day_6 | Day_6_JQ1 | 72.367 | 53.8118 | −0.4274 |
| Pmp22 | Day_1 | Day_6 | OK | 19.4383 | 115.54 | 2.57141 | Pmp22 | Day_6 | Day_6_JQ1 | 115.54 | 85.9788 | −0.4263 |
| Plekhg3 | Day_1 | Day_6 | OK | 9.30938 | 24.416 | 1.39107 | Plekhg3 | Day_6 | Day_6_JQ1 | 24.416 | 18.1798 | −0.4255 |
| Jub | Day_1 | Day_6 | OK | 12.0748 | 31.5215 | 1.38433 | Jub | Day_6 | Day_6_JQ1 | 31.5215 | 23.4821 | −0.4248 |
| Snai1 | Day_1 | Day_6 | OK | 19.1001 | 47.6351 | 1.31844 | Snai1 | Day_6 | Day_6_JQ1 | 47.6351 | 35.4904 | −0.4246 |
| Tead3 | Day_1 | Day_6 | OK | 3.8944 | 26.2136 | 2.75084 | Tead3 | Day_6 | Day_6_JQ1 | 26.2136 | 19.5802 | −0.4209 |
| Sepx1 | Day_1 | Day_6 | OK | 45.8203 | 124.306 | 1.43983 | Sepx1 | Day_6 | Day_6_JQ1 | 124.306 | 93.1036 | −0.417 |
| Skp2 | Day_1 | Day_6 | OK | 2.38322 | 6.65052 | 1.48055 | Skp2 | Day_6 | Day_6_JQ1 | 6.65052 | 4.99666 | −0.4125 |
| Dhrs1 | Day_1 | Day_6 | OK | 17.041 | 39.9393 | 1.2288 | Dhrs1 | Day_6 | Day_6_JQ1 | 39.9393 | 30.0462 | −0.4106 |
| Wee1 | Day_1 | Day_6 | OK | 3.13386 | 10.6298 | 1.7621 | Wee1 | Day_6 | Day_6_JQ1 | 10.6298 | 8.03107 | −0.4044 |
| Selm | Day_1 | Day_6 | OK | 12.9543 | 106.477 | 3.03904 | Selm | Day_6 | Day_6_JQ1 | 106.477 | 80.5867 | −0.4019 |
| Cxx1c | Day_1 | Day_6 | OK | 5.25969 | 17.8552 | 1.76329 | Cxx1c | Day_6 | Day_6_JQ1 | 17.8552 | 13.5431 | −0.3988 |
| Trnp1 | Day_1 | Day_6 | OK | 0.62651 | 17.8785 | 4.83475 | Trnp1 | Day_6 | Day_6_JQ1 | 17.8785 | 13.5695 | −0.3979 |
| Arhgef25 | Day_1 | Day_6 | OK | 4.27284 | 34.2187 | 3.00152 | Arhgef25 | Day_6 | Day_6_JQ1 | 34.2187 | 25.9937 | −0.3966 |
| Hspb1 | Day_1 | Day_6 | OK | 29.0798 | 136.989 | 2.23597 | Hspb1 | Day_6 | Day_6_JQ1 | 136.989 | 104.396 | −0.392 |
| Trf | Day_1 | Day_6 | OK | 38.8043 | 96.3728 | 1.31241 | Trf | Day_6 | Day_6_JQ1 | 96.3728 | 73.4499 | −0.3919 |
| Wnt9a | Day_1 | Day_6 | OK | 0.73548 | 10.0572 | 3.77339 | Wnt9a | Day_6 | Day_6_JQ1 | 10.0572 | 7.67099 | −0.3907 |
| Leprel2 | Day_1 | Day_6 | OK | 11.4425 | 41.7163 | 1.8662 | Leprel2 | Day_6 | Day_6_JQ1 | 41.7163 | 31.892 | −0.3874 |
| Rhobtb3 | Day_1 | Day_6 | OK | 3.08153 | 17.8477 | 2.53402 | Rhobtb3 | Day_6 | Day_6_JQ1 | 17.8477 | 13.6471 | −0.3871 |
| Pon3 | Day_1 | Day_6 | OK | 8.85769 | 25.541 | 1.52781 | Pon3 | Day_6 | Day_6_JQ1 | 25.541 | 19.5497 | −0.3857 |
| Gpr180 | Day_1 | Day_6 | OK | 7.25687 | 19.5037 | 1.42633 | Gpr180 | Day_6 | Day_6_JQ1 | 19.5037 | 14.9448 | −0.3841 |
| Phldb2 | Day_1 | Day_6 | OK | 52.6953 | 194.592 | 1.88471 | Phldb2 | Day_6 | Day_6_JQ1 | 194.592 | 149.373 | −0.3815 |
| Apbb1ip | Day_1 | Day_6 | OK | 6.60214 | 24.1309 | 1.86988 | Apbb1ip | Day_6 | Day_6_JQ1 | 24.1309 | 18.5518 | −0.3793 |
| Celf2 | Day_1 | Day_6 | OK | 5.56333 | 14.497 | 1.38173 | Celf2 | Day_6 | Day_6_JQ1 | 14.497 | 11.1573 | −0.3778 |
| Rprd1a | Day_1 | Day_6 | OK | 3.00748 | 9.24257 | 1.61974 | Rprd1a | Day_6 | Day_6_JQ1 | 9.24257 | 7.11815 | −0.3768 |
| Phlda3 | Day_1 | Day_6 | OK | 72.1029 | 171.697 | 1.25174 | Phlda3 | Day_6 | Day_6_JQ1 | 171.697 | 132.249 | −0.3766 |
| Tlcd2 | Day_1 | Day_6 | OK | 2.51994 | 9.99628 | 1.988 | Tlcd2 | Day_6 | Day_6_JQ1 | 9.99628 | 7.71719 | −0.3733 |
| Kazald1 | Day_1 | Day_6 | OK | 0.3637 | 5.83154 | 4.00307 | Kazald1 | Day_6 | Day_6_JQ1 | 5.83154 | 4.52184 | −0.367 |
| Unc119 | Day_1 | Day_6 | OK | 6.2424 | 19.7412 | 1.66104 | Unc119 | Day_6 | Day_6_JQ1 | 19.7412 | 15.3231 | −0.3655 |
| Slc35a2 | Day_1 | Day_6 | OK | 14.3641 | 29.7477 | 1.05031 | Slc35a2 | Day_6 | Day_6_JQ1 | 29.7477 | 23.1121 | −0.3641 |
| Loxl1 | Day_1 | Day_6 | OK | 5.75125 | 100.404 | 4.1258 | Loxl1 | Day_6 | Day_6_JQ1 | 100.404 | 78.0679 | −0.363 |
| Cnrip1 | Day_1 | Day_6 | OK | 7.83535 | 19.4611 | 1.31252 | Cnrip1 | Day_6 | Day_6_JQ1 | 19.4611 | 15.1406 | −0.3622 |
| Klhl26 | Day_1 | Day_6 | OK | 8.85583 | 32.8741 | 1.89225 | Klhl26 | Day_6 | Day_6_JQ1 | 32.8741 | 25.6223 | −0.3596 |
| Rab3d | Day_1 | Day_6 | OK | 2.8875 | 11.5153 | 1.99566 | Rab3d | Day_6 | Day_6_JQ1 | 11.5153 | 8.98978 | −0.3572 |
| Numbl | Day_1 | Day_6 | OK | 10.1493 | 28.7535 | 1.50236 | Numbl | Day_6 | Day_6_JQ1 | 28.7535 | 22.4916 | −0.3544 |
| Loxl2 | Day_1 | Day_6 | OK | 56.0395 | 853.575 | 3.929 | Loxl2 | Day_6 | Day_6_JQ1 | 853.575 | 668.398 | −0.3528 |
| Dnpep | Day_1 | Day_6 | OK | 24.0294 | 52.3474 | 1.12332 | Dnpep | Day_6 | Day_6_JQ1 | 52.3474 | 41.0591 | −0.3504 |
| Csrp2 | Day_1 | Day_6 | OK | 56.5385 | 174.574 | 1.62654 | Csrp2 | Day_6 | Day_6_JQ1 | 174.574 | 137.115 | −0.3485 |
| Ryk | Day_1 | Day_6 | OK | 30.8186 | 70.3751 | 1.19126 | Ryk | Day_6 | Day_6_JQ1 | 70.3751 | 55.3732 | −0.3459 |
| Serpinh1 | Day_1 | Day_6 | OK | 366.754 | 1503.65 | 2.03558 | Serpinh1 | Day_6 | Day_6_JQ1 | 1503.65 | 1190.39 | −0.337 |
| Cul7 | Day_1 | Day_6 | OK | 8.9693 | 24.0995 | 1.42594 | Cul7 | Day_6 | Day_6_JQ1 | 24.0995 | 19.1665 | −0.3304 |
| Dynlrb1 | Day_1 | Day_6 | OK | 97.9371 | 204.187 | 1.05996 | Dynlrb1 | Day_6 | Day_6_JQ1 | 204.187 | 162.462 | −0.3298 |
| Snta1 | Day_1 | Day_6 | OK | 5.27672 | 31.6861 | 2.58614 | Snta1 | Day_6 | Day_6_JQ1 | 31.6861 | 25.2902 | −0.3253 |
| Kif21b | Day_1 | Day_6 | OK | 3.18346 | 10.1954 | 1.67926 | Kif21b | Day_6 | Day_6_JQ1 | 10.1954 | 8.14503 | −0.3239 |
| Kdm5b | Day_1 | Day_6 | OK | 10.9542 | 21.6857 | 0.98526 | Kdm5b | Day_6 | Day_6_JQ1 | 21.6857 | 17.3352 | −0.323 |
| Rnft1 | Day_1 | Day_6 | OK | 10.7549 | 22.2204 | 1.04689 | Rnft1 | Day_6 | Day_6_JQ1 | 22.2204 | 17.8003 | −0.32 |
| Ptplad2 | Day_1 | Day_6 | OK | 1.30319 | 10.1093 | 2.95556 | Ptplad2 | Day_6 | Day_6_JQ1 | 10.1093 | 8.10647 | −0.3185 |
| Lepre1 | Day_1 | Day_6 | OK | 16.4769 | 76.52 | 2.21539 | Lepre1 | Day_6 | Day_6_JQ1 | 76.52 | 61.4792 | −0.3157 |
| Tubb4 | Day_1 | Day_6 | OK | 1.67932 | 15.0361 | 3.16248 | Tubb4 | Day_6 | Day_6_JQ1 | 15.0361 | 12.0841 | −0.3153 |
| Mageh1 | Day_1 | Day_6 | OK | 12.4048 | 28.3847 | 1.19421 | Mageh1 | Day_6 | Day_6_JQ1 | 28.3847 | 22.8542 | −0.3127 |
| Ext1 | Day_1 | Day_6 | OK | 46.0682 | 99.6865 | 1.11363 | Ext1 | Day_6 | Day_6_JQ1 | 99.6865 | 80.2965 | −0.3121 |
| Gmpr | Day_1 | Day_6 | OK | 1.77651 | 9.9335 | 2.48325 | Gmpr | Day_6 | Day_6_JQ1 | 9.9335 | 8.02637 | −0.3076 |
| Pmm1 | Day_1 | Day_6 | OK | 24.4142 | 67.9028 | 1.47575 | Pmm1 | Day_6 | Day_6_JQ1 | 67.9028 | 54.8681 | −0.3075 |
| Pvrl2 | Day_1 | Day_6 | OK | 41.2626 | 101.202 | 1.29433 | Pvrl2 | Day_6 | Day_6_JQ1 | 101.202 | 81.9103 | −0.3051 |
| Slc44a2 | Day_1 | Day_6 | OK | 22.266 | 73.2362 | 1.71771 | Slc44a2 | Day_6 | Day_6_JQ1 | 73.2362 | 59.6308 | −0.2965 |
| Hspg2 | Day_1 | Day_6 | OK | 36.301 | 129.016 | 1.82946 | Hspg2 | Day_6 | Day_6_JQ1 | 129.016 | 105.328 | −0.2927 |
| Tmem119 | Day_1 | Day_6 | OK | 0.42055 | 47.2545 | 6.81204 | Tmem119 | Day_6 | Day_6_JQ1 | 47.2545 | 38.7358 | −0.2868 |
| Arsb | Day_1 | Day_6 | OK | 4.28362 | 20.4163 | 2.25282 | Arsb | Day_6 | Day_6_JQ1 | 20.4163 | 16.7663 | −0.2842 |
| Chsy3 | Day_1 | Day_6 | OK | 1.13591 | 5.06223 | 2.15593 | Chsy3 | Day_6 | Day_6_JQ1 | 5.06223 | 4.1726 | −0.2788 |
| Myl6 | Day_1 | Day_6 | OK | 285.402 | 643.656 | 1.17329 | Myl6 | Day_6 | Day_6_JQ1 | 643.656 | 531.936 | −0.275 |
| Ube2c | Day_1 | Day_6 | OK | 0.51889 | 37.0286 | 6.15708 | Ube2c | Day_6 | Day_6_JQ1 | 37.0286 | 30.6226 | −0.274 |
| Nid2 | Day_1 | Day_6 | OK | 3.4608 | 157.278 | 5.50607 | Nid2 | Day_6 | Day_6_JQ1 | 157.278 | 130.153 | −0.2731 |
| Frzb | Day_1 | Day_6 | OK | 23.4761 | 174.416 | 2.89327 | Frzb | Day_6 | Day_6_JQ1 | 174.416 | 144.416 | −0.2723 |
| Hoxb2 | Day_1 | Day_6 | OK | 11.8855 | 40.6056 | 1.77248 | Hoxb2 | Day_6 | Day_6_JQ1 | 40.6056 | 33.6747 | −0.27 |
| Chpf | Day_1 | Day_6 | OK | 28.5346 | 61.536 | 1.10872 | Chpf | Day_6 | Day_6_JQ1 | 61.536 | 51.0364 | −0.2699 |
| D4Bwg0951e | Day_1 | Day_6 | OK | 8.80885 | 36.3191 | 2.0437 | D4Bwg0951e | Day_6 | Day_6_JQ1 | 36.3191 | 30.19 | −0.2667 |
| Wfs1 | Day_1 | Day_6 | OK | 13.8056 | 27.591 | 0.99894 | Wfs1 | Day_6 | Day_6_JQ1 | 27.591 | 22.9762 | −0.2641 |
| Gm2a | Day_1 | Day_6 | OK | 9.1895 | 26.8765 | 1.54828 | Gm2a | Day_6 | Day_6_JQ1 | 26.8765 | 22.5206 | −0.2551 |
| Enpp5 | Day_1 | Day_6 | OK | 10.1414 | 30.4943 | 1.58828 | Enpp5 | Day_6 | Day_6_JQ1 | 30.4943 | 25.6268 | −0.2509 |
| Fam20a | Day_1 | Day_6 | OK | 4.94325 | 13.301 | 1.428 | Fam20a | Day_6 | Day_6_JQ1 | 13.301 | 11.2001 | −0.248 |
| S100a1 | Day_1 | Day_6 | OK | 29.6451 | 83.4459 | 1.49305 | S100a1 | Day_6 | Day_6_JQ1 | 83.4459 | 70.3658 | −0.246 |
| Setd7 | Day_1 | Day_6 | OK | 13.3383 | 38.7467 | 1.5385 | Setd7 | Day_6 | Day_6_JQ1 | 38.7467 | 32.7601 | −0.2421 |
| Rab34 | Day_1 | Day_6 | OK | 24.5114 | 65.2073 | 1.41158 | Rab34 | Day_6 | Day_6_JQ1 | 65.2073 | 55.2701 | −0.2385 |
| Copz2 | Day_1 | Day_6 | OK | 6.0688 | 55.4477 | 3.19165 | Copz2 | Day_6 | Day_6_JQ1 | 55.4477 | 47.0355 | −0.2374 |
| Myof | Day_1 | Day_6 | OK | 23.3658 | 49.6115 | 1.08627 | Myof | Day_6 | Day_6_JQ1 | 49.6115 | 42.2087 | −0.2331 |
| Polr3k | Day_1 | Day_6 | OK | 12.5938 | 26.8584 | 1.09266 | Polr3k | Day_6 | Day_6_JQ1 | 26.8584 | 22.8928 | −0.2305 |
| Apbb1 | Day_1 | Day_6 | OK | 1.37506 | 8.5134 | 2.63024 | Apbb1 | Day_6 | Day_6_JQ1 | 8.5134 | 7.27324 | −0.2271 |
| Myh10 | Day_1 | Day_6 | OK | 4.04707 | 40.2732 | 3.31487 | Myh10 | Day_6 | Day_6_JQ1 | 40.2732 | 34.498 | −0.2233 |
| Txndc5 | Day_1 | Day_6 | OK | 98.7357 | 227.24 | 1.20257 | Txndc5 | Day_6 | Day_6_JQ1 | 227.24 | 194.663 | −0.2232 |
| Ttc13 | Day_1 | Day_6 | OK | 9.71841 | 23.4296 | 1.26954 | Ttc13 | Day_6 | Day_6_JQ1 | 23.4296 | 20.1057 | −0.2207 |
| Tspan6 | Day_1 | Day_6 | OK | 7.06913 | 24.7264 | 1.80645 | Tspan6 | Day_6 | Day_6_JQ1 | 24.7264 | 21.2613 | −0.2178 |
| 2610018G03Rik | Day_1 | Day_6 | OK | 1.13088 | 9.02877 | 2.99709 | 2610018G03Rik | Day_6 | Day_6_JQ1 | 9.02877 | 7.78311 | −0.2142 |
| Gadd45g | Day_1 | Day_6 | OK | 40.6258 | 175.723 | 2.11283 | Gadd45g | Day_6 | Day_6_JQ1 | 175.723 | 151.82 | −0.2109 |
| Dhcr24 | Day_1 | Day_6 | OK | 6.768 | 20.0719 | 1.56837 | Dhcr24 | Day_6 | Day_6_JQ1 | 20.0719 | 17.3636 | −0.2091 |
| Kctd11 | Day_1 | Day_6 | OK | 18.7911 | 46.5122 | 1.30756 | Kctd11 | Day_6 | Day_6_JQ1 | 46.5122 | 40.3428 | −0.2053 |
| 2810055F11Rik | Day_1 | Day_6 | OK | 2.26706 | 10.7979 | 2.25185 | 2810055F11Rik | Day_6 | Day_6_JQ1 | 10.7979 | 9.37162 | −0.2044 |
| Pgrmc1 | Day_1 | Day_6 | OK | 57.1341 | 127.525 | 1.15836 | Pgrmc1 | Day_6 | Day_6_JQ1 | 127.525 | 110.711 | −0.204 |
| Dynlt3 | Day_1 | Day_6 | OK | 37.9254 | 85.2897 | 1.16921 | Dynlt3 | Day_6 | Day_6_JQ1 | 85.2897 | 74.0523 | −0.2038 |
| Cfh | Day_1 | Day_6 | OK | 16.8775 | 38.9763 | 1.2075 | Cfh | Day_6 | Day_6_JQ1 | 38.9763 | 33.8709 | −0.2025 |
| Ankrd44 | Day_1 | Day_6 | OK | 3.37512 | 9.10799 | 1.4322 | Ankrd44 | Day_6 | Day_6_JQ1 | 9.10799 | 7.9209 | −0.2015 |
| Sidt2 | Day_1 | Day_6 | OK | 33.853 | 98.3841 | 1.53914 | Sidt2 | Day_6 | Day_6_JQ1 | 98.3841 | 85.8124 | −0.1972 |
| Shisa4 | Day_1 | Day_6 | OK | 2.49743 | 33.7732 | 3.75736 | Shisa4 | Day_6 | Day_6_JQ1 | 33.7732 | 29.5421 | −0.1931 |
| Pde5a | Day_1 | Day_6 | OK | 1.51664 | 4.93208 | 1.70132 | Pde5a | Day_6 | Day_6_JQ1 | 4.93208 | 4.31494 | −0.1929 |
| Agl | Day_1 | Day_6 | OK | 3.56566 | 9.88516 | 1.4711 | Agl | Day_6 | Day_6_JQ1 | 9.88516 | 8.66673 | −0.1898 |
| 2700094K13Rik | Day_1 | Day_6 | OK | 16.6861 | 60.2615 | 1.85259 | 2700094K13Rik | Day_6 | Day_6_JQ1 | 60.2615 | 52.964 | −0.1862 |
| Cdc42bpg | Day_1 | Day_6 | OK | 5.16646 | 10.974 | 1.08684 | Cdc42bpg | Day_6 | Day_6_JQ1 | 10.974 | 9.64703 | −0.1859 |
| Ephb2 | Day_1 | Day_6 | OK | 1.42999 | 13.9864 | 3.28994 | Ephb2 | Day_6 | Day_6_JQ1 | 13.9864 | 12.3025 | −0.1851 |
| Maged1 | Day_1 | Day_6 | OK | 53.0852 | 238.382 | 2.16689 | Maged1 | Day_6 | Day_6_JQ1 | 238.382 | 209.848 | −0.1839 |
| Pbxip1 | Day_1 | Day_6 | OK | 18.1505 | 75.8901 | 2.0639 | Pbxip1 | Day_6 | Day_6_JQ1 | 75.8901 | 66.9739 | −0.1803 |
| Tmem176b | Day_1 | Day_6 | OK | 118.739 | 262.485 | 1.14444 | Tmem176b | Day_6 | Day_6_JQ1 | 262.485 | 232.156 | −0.1771 |
| Arhgap1 | Day_1 | Day_6 | OK | 46.2898 | 112.935 | 1.28672 | Arhgap1 | Day_6 | Day_6_JQ1 | 112.935 | 99.931 | −0.1765 |
| Ccdc122 | Day_1 | Day_6 | OK | 1.53542 | 6.20333 | 2.01441 | Ccdc122 | Day_6 | Day_6_JQ1 | 6.20333 | 5.50132 | −0.1733 |
| St6galnac6 | Day_1 | Day_6 | OK | 5.83072 | 23.8899 | 2.03466 | St6galnac6 | Day_6 | Day_6_JQ1 | 23.8899 | 21.1878 | −0.1732 |
| Mxra8 | Day_1 | Day_6 | OK | 30.147 | 141.996 | 2.23576 | Mxra8 | Day_6 | Day_6_JQ1 | 141.996 | 126.008 | −0.1723 |
| Ppic | Day_1 | Day_6 | OK | 80.8178 | 201.267 | 1.31637 | Ppic | Day_6 | Day_6_JQ1 | 201.267 | 178.683 | −0.1717 |
| Unc5b | Day_1 | Day_6 | OK | 56.3547 | 159.522 | 1.50114 | Unc5b | Day_6 | Day_6_JQ1 | 159.522 | 142.372 | −0.1641 |
| Oxct1 | Day_1 | Day_6 | OK | 14.1517 | 52.4194 | 1.88912 | Oxct1 | Day_6 | Day_6_JQ1 | 52.4194 | 46.8229 | −0.1629 |
| Wbp5 | Day_1 | Day_6 | OK | 144.674 | 376.359 | 1.3793 | Wbp5 | Day_6 | Day_6_JQ1 | 376.359 | 336.924 | −0.1597 |
| Ccdc102a | Day_1 | Day_6 | OK | 4.30774 | 13.5472 | 1.65299 | Ccdc102a | Day_6 | Day_6_JQ1 | 13.5472 | 12.129 | −0.1595 |
| Ogn | Day_1 | Day_6 | OK | 1.01907 | 76.3737 | 6.22776 | Ogn | Day_6 | Day_6_JQ1 | 76.3737 | 68.5101 | −0.1568 |
| Tln2 | Day_1 | Day_6 | OK | 3.36354 | 12.0135 | 1.8366 | Tln2 | Day_6 | Day_6_JQ1 | 12.0135 | 10.7793 | −0.1564 |
| Rab11fip5 | Day_1 | Day_6 | OK | 4.56532 | 20.7213 | 2.18232 | Rab11fip5 | Day_6 | Day_6_JQ1 | 20.7213 | 18.6008 | −0.1557 |
| St3gal2 | Day_1 | Day_6 | OK | 14.4615 | 30.0861 | 1.05688 | St3gal2 | Day_6 | Day_6_JQ1 | 30.0861 | 27.017 | −0.1552 |
| Fam26e | Day_1 | Day_6 | OK | 1.32909 | 12.9921 | 3.28912 | Fam26e | Day_6 | Day_6_JQ1 | 12.9921 | 11.6803 | −0.1536 |
| Tulp4 | Day_1 | Day_6 | OK | 8.35347 | 17.3692 | 1.05608 | Tulp4 | Day_6 | Day_6_JQ1 | 17.3692 | 15.6577 | −0.1497 |
| Cacna2d1 | Day_1 | Day_6 | OK | 3.70837 | 8.4428 | 1.18694 | Cacna2d1 | Day_6 | Day_6_JQ1 | 8.4428 | 7.61452 | −0.149 |
| Lamb1 | Day_1 | Day_6 | OK | 48.8535 | 129.207 | 1.40315 | Lamb1 | Day_6 | Day_6_JQ1 | 129.207 | 116.83 | −0.1453 |
| Kcnab1 | Day_1 | Day_6 | OK | 0.94661 | 6.60917 | 2.80363 | Kcnab1 | Day_6 | Day_6_JQ1 | 6.60917 | 5.98662 | −0.1427 |
| Synpo | Day_1 | Day_6 | OK | 44.7792 | 141.007 | 1.65487 | Synpo | Day_6 | Day_6_JQ1 | 141.007 | 128.3 | −0.1362 |
| Pdrg1 | Day_1 | Day_6 | OK | 39.8852 | 84.8667 | 1.08934 | Pdrg1 | Day_6 | Day_6_JQ1 | 84.8667 | 77.2251 | −0.1361 |
| Parva | Day_1 | Day_6 | OK | 39.5406 | 92.0928 | 1.21975 | Parva | Day_6 | Day_6_JQ1 | 92.0928 | 84.643 | −0.1217 |
| Gpam | Day_1 | Day_6 | OK | 5.93206 | 14.3291 | 1.27235 | Gpam | Day_6 | Day_6_JQ1 | 14.3291 | 13.2457 | −0.1134 |
| Ston1 | Day_1 | Day_6 | OK | 33.7801 | 87.115 | 1.36675 | Ston1 | Day_6 | Day_6_JQ1 | 87.115 | 80.759 | −0.1093 |
| Plcxd2 | Day_1 | Day_6 | OK | 2.57192 | 8.94656 | 1.79849 | Plcxd2 | Day_6 | Day_6_JQ1 | 8.94656 | 8.32094 | −0.1046 |
| Gpr153 | Day_1 | Day_6 | OK | 2.36492 | 16.4929 | 2.80199 | Gpr153 | Day_6 | Day_6_JQ1 | 16.4929 | 15.34 | −0.1046 |
| Pgp | Day_1 | Day_6 | OK | 23.4153 | 59.6272 | 1.34852 | Pgp | Day_6 | Day_6_JQ1 | 59.6272 | 55.4841 | −0.1039 |
| Hspb6 | Day_1 | Day_6 | OK | 2.00988 | 16.1812 | 3.00913 | Hspb6 | Day_6 | Day_6_JQ1 | 16.1812 | 15.1361 | −0.0963 |
| Sparc | Day_1 | Day_6 | OK | 381.767 | 4211.88 | 3.4637 | Sparc | Day_6 | Day_6_JQ1 | 4211.88 | 3945.98 | −0.0941 |
| Tubb2a | Day_1 | Day_6 | OK | 30.0454 | 60.4675 | 1.00902 | Tubb2a | Day_6 | Day_6_JQ1 | 60.4675 | 56.747 | −0.0916 |
| Gxylt2 | Day_1 | Day_6 | OK | 0.51552 | 8.53633 | 4.04951 | Gxylt2 | Day_6 | Day_6_JQ1 | 8.53633 | 8.03083 | −0.0881 |
| Clip3 | Day_1 | Day_6 | OK | 3.38137 | 27.0623 | 3.0006 | Clip3 | Day_6 | Day_6_JQ1 | 27.0623 | 25.4738 | −0.0873 |
| Ccne1 | Day_1 | Day_6 | OK | 1.35536 | 14.6308 | 3.43226 | Ccne1 | Day_6 | Day_6_JQ1 | 14.6308 | 13.7889 | −0.0855 |
| Rrm1 | Day_1 | Day_6 | OK | 12.5491 | 40.2147 | 1.68013 | Rrm1 | Day_6 | Day_6_JQ1 | 40.2147 | 37.9825 | −0.0824 |
| Efna4 | Day_1 | Day_6 | OK | 0.83029 | 6.4056 | 2.94765 | Efna4 | Day_6 | Day_6_JQ1 | 6.4056 | 6.05329 | −0.0816 |
| Cxxc5 | Day_1 | Day_6 | OK | 6.25036 | 30.2026 | 2.27266 | Cxxc5 | Day_6 | Day_6_JQ1 | 30.2026 | 28.6295 | −0.0772 |
| Fstl1 | Day_1 | Day_6 | OK | 154.992 | 929.991 | 2.58503 | Fstl1 | Day_6 | Day_6_JQ1 | 929.991 | 883.839 | −0.0734 |
| Nxn | Day_1 | Day_6 | OK | 15.6072 | 77.5795 | 2.31347 | Nxn | Day_6 | Day_6_JQ1 | 77.5795 | 73.7521 | −0.073 |
| Rbms3 | Day_1 | Day_6 | OK | 5.38255 | 15.2578 | 1.50318 | Rbms3 | Day_6 | Day_6_JQ1 | 15.2578 | 14.5408 | −0.0694 |
| Rsu1 | Day_1 | Day_6 | OK | 57.1146 | 121.192 | 1.08537 | Rsu1 | Day_6 | Day_6_JQ1 | 121.192 | 115.627 | −0.0678 |
| Pdlim5 | Day_1 | Day_6 | OK | 48.7308 | 126.229 | 1.37314 | Pdlim5 | Day_6 | Day_6_JQ1 | 126.229 | 120.722 | −0.0644 |
| Bsg | Day_1 | Day_6 | OK | 233.846 | 585.917 | 1.32514 | Bsg | Day_6 | Day_6_JQ1 | 585.917 | 560.767 | −0.0633 |
| Pdk3 | Day_1 | Day_6 | OK | 6.62895 | 18.569 | 1.48604 | Pdk3 | Day_6 | Day_6_JQ1 | 18.569 | 17.7734 | −0.0632 |
| Sepn1 | Day_1 | Day_6 | OK | 24.6136 | 55.4235 | 1.17104 | Sepn1 | Day_6 | Day_6_JQ1 | 55.4235 | 53.0761 | −0.0624 |
| Zfp449 | Day_1 | Day_6 | OK | 1.84026 | 5.93833 | 1.69015 | Zfp449 | Day_6 | Day_6_JQ1 | 5.93833 | 5.68898 | −0.0619 |
| Ngfrap1 | Day_1 | Day_6 | OK | 26.5178 | 204.667 | 2.94824 | Ngfrap1 | Day_6 | Day_6_JQ1 | 204.667 | 196.152 | −0.0613 |
| Prdx4 | Day_1 | Day_6 | OK | 36.6971 | 94.12 | 1.35883 | Prdx4 | Day_6 | Day_6_JQ1 | 94.12 | 90.3086 | −0.0596 |
| Aes | Day_1 | Day_6 | OK | 94.7644 | 196.876 | 1.05487 | Aes | Day_6 | Day_6_JQ1 | 196.876 | 188.935 | −0.0594 |
| Pdzrn3 | Day_1 | Day_6 | OK | 7.57927 | 36.9262 | 2.28451 | Pdzrn3 | Day_6 | Day_6_JQ1 | 36.9262 | 35.4736 | −0.0579 |
| Ptprs | Day_1 | Day_6 | OK | 3.04775 | 26.6387 | 3.12771 | Ptprs | Day_6 | Day_6_JQ1 | 26.6387 | 25.6268 | −0.0559 |
| Morc4 | Day_1 | Day_6 | OK | 3.16557 | 8.83166 | 1.48022 | Morc4 | Day_6 | Day_6_JQ1 | 8.83166 | 8.50409 | −0.0545 |
| Gucy1b3 | Day_1 | Day_6 | OK | 14.2167 | 36.4578 | 1.35864 | Gucy1b3 | Day_6 | Day_6_JQ1 | 36.4578 | 35.4413 | −0.0408 |
| Aurka | Day_1 | Day_6 | OK | 1.12757 | 17.7402 | 3.97574 | Aurka | Day_6 | Day_6_JQ1 | 17.7402 | 17.2799 | −0.0379 |
| Prr11 | Day_1 | Day_6 | OK | 0.30032 | 6.98773 | 4.54026 | Prr11 | Day_6 | Day_6_JQ1 | 6.98773 | 6.83147 | −0.0326 |
| Gtse1 | Day_1 | Day_6 | OK | 5.76201 | 16.8384 | 1.54711 | Gtse1 | Day_6 | Day_6_JQ1 | 16.8384 | 16.4741 | −0.0316 |
| Acadsb | Day_1 | Day_6 | OK | 13.246 | 27.0576 | 1.03048 | Acadsb | Day_6 | Day_6_JQ1 | 27.0576 | 26.5141 | −0.0293 |
| Gpnmb | Day_1 | Day_6 | OK | 40.7427 | 102.959 | 1.33746 | Gpnmb | Day_6 | Day_6_JQ1 | 102.959 | 100.919 | −0.0289 |
| Cpxm1 | Day_1 | Day_6 | OK | 6.94307 | 121.636 | 4.13085 | Cpxm1 | Day_6 | Day_6_JQ1 | 121.636 | 119.242 | −0.0287 |
| 2210013O21Rik | Day_1 | Day_6 | OK | 14.1199 | 38.8005 | 1.45835 | 2210013O21Rik | Day_6 | Day_6_JQ1 | 38.8005 | 38.055 | −0.028 |
| Rbfox2 | Day_1 | Day_6 | OK | 16.1046 | 31.9806 | 0.98973 | Rbfox2 | Day_6 | Day_6_JQ1 | 31.9806 | 31.4181 | −0.0256 |
| Map1lc3a | Day_1 | Day_6 | OK | 25.7814 | 85.1635 | 1.72391 | Map1lc3a | Day_6 | Day_6_JQ1 | 85.1635 | 83.7075 | −0.0249 |
| Htra1 | Day_1 | Day_6 | OK | 8.21405 | 130.908 | 3.99432 | Htra1 | Day_6 | Day_6_JQ1 | 130.908 | 128.76 | −0.0239 |
| Olfml3 | Day_1 | Day_6 | OK | 16.5839 | 171.962 | 3.37424 | Olfml3 | Day_6 | Day_6_JQ1 | 171.962 | 169.404 | −0.0216 |
| Itgb5 | Day_1 | Day_6 | OK | 22.1763 | 209.128 | 3.2373 | Itgb5 | Day_6 | Day_6_JQ1 | 209.128 | 206.046 | −0.0214 |
| Fkbp9 | Day_1 | Day_6 | OK | 56.7025 | 155.563 | 1.45602 | Fkbp9 | Day_6 | Day_6_JQ1 | 155.563 | 153.585 | −0.0185 |
| Sil1 | Day_1 | Day_6 | OK | 11.5426 | 25.2668 | 1.13028 | Sil1 | Day_6 | Day_6_JQ1 | 25.2668 | 25.0211 | −0.0141 |
| Pqlc3 | Day_1 | Day_6 | OK | 3.7794 | 21.4219 | 2.50286 | Pqlc3 | Day_6 | Day_6_JQ1 | 21.4219 | 21.232 | −0.0129 |
| Calm3 | Day_1 | Day_6 | OK | 91.5672 | 187.128 | 1.03113 | Calm3 | Day_6 | Day_6_JQ1 | 187.128 | 185.514 | −0.0125 |
| Pcbp4 | Day_1 | Day_6 | OK | 13.373 | 31.3499 | 1.22913 | Pcbp4 | Day_6 | Day_6_JQ1 | 31.3499 | 31.1679 | −0.0084 |
| Mmab | Day_1 | Day_6 | OK | 2.99822 | 8.16979 | 1.44619 | Mmab | Day_6 | Day_6_JQ1 | 8.16979 | 8.1326 | −0.0066 |
| Aplp1 | Day_1 | Day_6 | OK | 3.64225 | 20.8112 | 2.51446 | Aplp1 | Day_6 | Day_6_JQ1 | 20.8112 | 20.7172 | −0.0065 |
| Prkab2 | Day_1 | Day_6 | OK | 8.61577 | 26.1525 | 1.6019 | Prkab2 | Day_6 | Day_6_JQ1 | 26.1525 | 26.0507 | −0.0056 |
| Ccdc3 | Day_1 | Day_6 | OK | 5.02688 | 44.0227 | 3.13051 | Ccdc3 | Day_6 | Day_6_JQ1 | 44.0227 | 44.0572 | 0.00113 |
| Palm | Day_1 | Day_6 | OK | 10.5422 | 43.3866 | 2.04108 | Palm | Day_6 | Day_6_JQ1 | 43.3866 | 43.4603 | 0.00245 |
| Vcl | Day_1 | Day_6 | OK | 62.6748 | 187.3 | 1.57939 | Vcl | Day_6 | Day_6_JQ1 | 187.3 | 188.104 | 0.00618 |
| Tyms | Day_1 | Day_6 | OK | 2.83264 | 9.75153 | 1.78348 | Tyms | Day_6 | Day_6_JQ1 | 9.75153 | 9.79997 | 0.00715 |
| Fat1 | Day_1 | Day_6 | OK | 8.95306 | 67.2531 | 2.90915 | Fat1 | Day_6 | Day_6_JQ1 | 67.2531 | 68.0733 | 0.01749 |
| Cpeb2 | Day_1 | Day_6 | OK | 3.53574 | 7.53841 | 1.09225 | Cpeb2 | Day_6 | Day_6_JQ1 | 7.53841 | 7.63939 | 0.0192 |
| Hic1 | Day_1 | Day_6 | OK | 17.539 | 40.0108 | 1.18982 | Hic1 | Day_6 | Day_6_JQ1 | 40.0108 | 40.7263 | 0.02557 |
| Marveld1 | Day_1 | Day_6 | OK | 13.1707 | 49.0861 | 1.89798 | Marveld1 | Day_6 | Day_6_JQ1 | 49.0861 | 50.0393 | 0.02774 |
| Gjc2 | Day_1 | Day_6 | OK | 0.60112 | 5.0473 | 3.06979 | Gjc2 | Day_6 | Day_6_JQ1 | 5.0473 | 5.14754 | 0.02837 |
| Lrp1 | Day_1 | Day_6 | OK | 55.6211 | 242.535 | 2.12449 | Lrp1 | Day_6 | Day_6_JQ1 | 242.535 | 247.494 | 0.0292 |
| Prrx1 | Day_1 | Day_6 | OK | 11.2209 | 33.0027 | 1.5564 | Prrx1 | Day_6 | Day_6_JQ1 | 33.0027 | 33.6888 | 0.02969 |
| Grb10 | Day_1 | Day_6 | OK | 32.2316 | 114.2 | 1.82502 | Grb10 | Day_6 | Day_6_JQ1 | 114.2 | 117.152 | 0.03682 |
| Irs2 | Day_1 | Day_6 | OK | 3.79341 | 11.3494 | 1.58106 | Irs2 | Day_6 | Day_6_JQ1 | 11.3494 | 11.6492 | 0.03761 |
| Bace1 | Day_1 | Day_6 | OK | 9.4944 | 39.0624 | 2.04063 | Bace1 | Day_6 | Day_6_JQ1 | 39.0624 | 40.101 | 0.03786 |
| Kidins220 | Day_1 | Day_6 | OK | 16.9808 | 33.7795 | 0.99225 | Kidins220 | Day_6 | Day_6_JQ1 | 33.7795 | 34.7188 | 0.03957 |
| Large | Day_1 | Day_6 | OK | 14.8486 | 39.4129 | 1.40834 | Large | Day_6 | Day_6_JQ1 | 39.4129 | 40.5667 | 0.04163 |
| Ctsf | Day_1 | Day_6 | OK | 2.9745 | 19.0014 | 2.67538 | Ctsf | Day_6 | Day_6_JQ1 | 19.0014 | 19.6316 | 0.04707 |
| Smpd1 | Day_1 | Day_6 | OK | 15.9566 | 39.5415 | 1.30922 | Smpd1 | Day_6 | Day_6_JQ1 | 39.5415 | 40.8765 | 0.0479 |
| Nans | Day_1 | Day_6 | OK | 28.6808 | 57.0825 | 0.99296 | Nans | Day_6 | Day_6_JQ1 | 57.0825 | 59.0594 | 0.04912 |
| Cenpa | Day_1 | Day_6 | OK | 1.32078 | 17.5445 | 3.73156 | Cenpa | Day_6 | Day_6_JQ1 | 17.5445 | 18.2009 | 0.05299 |
| Gstm4 | Day_1 | Day_6 | OK | 3.54117 | 10.9909 | 1.63401 | Gstm4 | Day_6 | Day_6_JQ1 | 10.9909 | 11.4343 | 0.05705 |
| Lynx1 | Day_1 | Day_6 | OK | 3.01312 | 15.6798 | 2.37958 | Lynx1 | Day_6 | Day_6_JQ1 | 15.6798 | 16.3298 | 0.05859 |
| Snx7 | Day_1 | Day_6 | OK | 12.6322 | 30.3182 | 1.26307 | Snx7 | Day_6 | Day_6_JQ1 | 30.3182 | 31.6545 | 0.06223 |
| Lbh | Day_1 | Day_6 | OK | 14.2342 | 298.151 | 4.38861 | Lbh | Day_6 | Day_6_JQ1 | 298.151 | 311.939 | 0.06522 |
| Ggcx | Day_1 | Day_6 | OK | 7.06915 | 21.6084 | 1.61199 | Ggcx | Day_6 | Day_6_JQ1 | 21.6084 | 22.6363 | 0.06704 |
| Mypop | Day_1 | Day_6 | OK | 2.15018 | 8.05002 | 1.90453 | Mypop | Day_6 | Day_6_JQ1 | 8.05002 | 8.4789 | 0.07488 |
| Bmpr1a | Day_1 | Day_6 | OK | 15.2713 | 31.8013 | 1.05826 | Bmpr1a | Day_6 | Day_6_JQ1 | 31.8013 | 33.5187 | 0.07588 |
| Ptk7 | Day_1 | Day_6 | OK | 3.39928 | 31.4179 | 3.20829 | Ptk7 | Day_6 | Day_6_JQ1 | 31.4179 | 33.1681 | 0.07821 |
| Rhou | Day_1 | Day_6 | OK | 6.33704 | 25.8342 | 2.0274 | Rhou | Day_6 | Day_6_JQ1 | 25.8342 | 27.2789 | 0.0785 |
| Plk4 | Day_1 | Day_6 | OK | 1.92427 | 6.8776 | 1.8376 | Plk4 | Day_6 | Day_6_JQ1 | 6.8776 | 7.27035 | 0.08012 |
| AI464131 | Day_1 | Day_6 | OK | 0.5864 | 6.03443 | 3.36325 | AI464131 | Day_6 | Day_6_JQ1 | 6.03443 | 6.39302 | 0.08328 |
| Mpp6 | Day_1 | Day_6 | OK | 15.9083 | 70.8782 | 2.15556 | Mpp6 | Day_6 | Day_6_JQ1 | 70.8782 | 75.1224 | 0.0839 |
| Fam168a | Day_1 | Day_6 | OK | 10.575 | 22.2089 | 1.07048 | Fam168a | Day_6 | Day_6_JQ1 | 22.2089 | 23.5828 | 0.0866 |
| Lass6 | Day_1 | Day_6 | OK | 5.4331 | 15.8531 | 1.54492 | Lass6 | Day_6 | Day_6_JQ1 | 15.8531 | 16.8557 | 0.08847 |
| Suox | Day_1 | Day_6 | OK | 4.09633 | 12.9377 | 1.65918 | Suox | Day_6 | Day_6_JQ1 | 12.9377 | 13.7986 | 0.09294 |
| Ttc28 | Day_1 | Day_6 | OK | 3.68214 | 10.1406 | 1.46153 | Ttc28 | Day_6 | Day_6_JQ1 | 10.1406 | 10.8173 | 0.09319 |
| Irs1 | Day_1 | Day_6 | OK | 0.30182 | 6.56451 | 4.44295 | Irs1 | Day_6 | Day_6_JQ1 | 6.56451 | 7.0255 | 0.09791 |
| Efna5 | Day_1 | Day_6 | OK | 2.75323 | 9.41345 | 1.7736 | Efna5 | Day_6 | Day_6_JQ1 | 9.41345 | 10.088 | 0.09985 |
| Etv5 | Day_1 | Day_6 | OK | 4.79976 | 10.9307 | 1.18736 | Etv5 | Day_6 | Day_6_JQ1 | 10.9307 | 11.7278 | 0.10154 |
| Sspn | Day_1 | Day_6 | OK | 0.99829 | 6.31088 | 2.66031 | Sspn | Day_6 | Day_6_JQ1 | 6.31088 | 6.80518 | 0.10879 |
| Bbx | Day_1 | Day_6 | OK | 4.86173 | 9.76604 | 1.0063 | Bbx | Day_6 | Day_6_JQ1 | 9.76604 | 10.5491 | 0.11127 |
| Smo | Day_1 | Day_6 | OK | 9.19825 | 26.7294 | 1.539 | Smo | Day_6 | Day_6_JQ1 | 26.7294 | 28.9251 | 0.11389 |
| Ugp2 | Day_1 | Day_6 | OK | 16.9989 | 42.8931 | 1.3353 | Ugp2 | Day_6 | Day_6_JQ1 | 42.8931 | 46.5523 | 0.11811 |
| Cacnb2 | Day_1 | Day_6 | OK | 1.49137 | 5.96794 | 2.00059 | Cacnb2 | Day_6 | Day_6_JQ1 | 5.96794 | 6.48021 | 0.11881 |
| Glis2 | Day_1 | Day_6 | OK | 8.99835 | 18.382 | 1.03056 | Glis2 | Day_6 | Day_6_JQ1 | 18.382 | 19.9703 | 0.11956 |
| Nid1 | Day_1 | Day_6 | OK | 89.9739 | 246.93 | 1.45653 | Nid1 | Day_6 | Day_6_JQ1 | 246.93 | 268.392 | 0.12024 |
| Magee1 | Day_1 | Day_6 | OK | 2.0759 | 16.1185 | 2.95691 | Magee1 | Day_6 | Day_6_JQ1 | 16.1185 | 17.7101 | 0.13586 |
| Upk3b | Day_1 | Day_6 | OK | 0.16926 | 34.0351 | 7.65161 | Upk3b | Day_6 | Day_6_JQ1 | 34.0351 | 37.4728 | 0.13882 |
| Fam115a | Day_1 | Day_6 | OK | 10.6952 | 26.8101 | 1.32582 | Fam115a | Day_6 | Day_6_JQ1 | 26.8101 | 29.5331 | 0.13955 |
| Sod3 | Day_1 | Day_6 | OK | 22.4937 | 100.332 | 2.15719 | Sod3 | Day_6 | Day_6_JQ1 | 100.332 | 110.53 | 0.13965 |
| Adcy3 | Day_1 | Day_6 | OK | 3.5908 | 13.78 | 1.9402 | Adcy3 | Day_6 | Day_6_JQ1 | 13.78 | 15.2041 | 0.14189 |
| Plcd1 | Day_1 | Day_6 | OK | 3.01086 | 14.4171 | 2.25953 | Plcd1 | Day_6 | Day_6_JQ1 | 14.4171 | 15.9407 | 0.14494 |
| Plk1 | Day_1 | Day_6 | OK | 0.43154 | 18.4389 | 5.41712 | Plk1 | Day_6 | Day_6_JQ1 | 18.4389 | 20.3959 | 0.14552 |
| Adc | Day_1 | Day_6 | OK | 0.45692 | 25.6497 | 5.81086 | Adc | Day_6 | Day_6_JQ1 | 25.6497 | 28.4163 | 0.14778 |
| Asap3 | Day_1 | Day_6 | OK | 0.26729 | 4.78313 | 4.16148 | Asap3 | Day_6 | Day_6_JQ1 | 4.78313 | 5.3032 | 0.14891 |
| Fads2 | Day_1 | Day_6 | OK | 9.27193 | 46.9107 | 2.33898 | Fads2 | Day_6 | Day_6_JQ1 | 46.9107 | 52.0843 | 0.15093 |
| Tuba1a | Day_1 | Day_6 | OK | 101.599 | 316.54 | 1.6395 | Tuba1a | Day_6 | Day_6_JQ1 | 316.54 | 351.493 | 0.15111 |
| Vkorc1 | Day_1 | Day_6 | OK | 29.8715 | 99.456 | 1.73529 | Vkorc1 | Day_6 | Day_6_JQ1 | 99.456 | 110.469 | 0.15152 |
| Antxr1 | Day_1 | Day_6 | OK | 8.52716 | 37.107 | 2.12156 | Antxr1 | Day_6 | Day_6_JQ1 | 37.107 | 41.3219 | 0.15521 |
| Myo5a | Day_1 | Day_6 | OK | 8.18157 | 17.244 | 1.07564 | Myo5a | Day_6 | Day_6_JQ1 | 17.244 | 19.236 | 0.15771 |
| Cdca8 | Day_1 | Day_6 | OK | 1.46758 | 16.298 | 3.47318 | Cdca8 | Day_6 | Day_6_JQ1 | 16.298 | 18.1888 | 0.15836 |
| Rhod | Day_1 | Day_6 | OK | 17.7082 | 38.8044 | 1.1318 | Rhod | Day_6 | Day_6_JQ1 | 38.8044 | 43.311 | 0.15852 |
| Thnsl2 | Day_1 | Day_6 | OK | 1.49179 | 5.55805 | 1.89753 | Thnsl2 | Day_6 | Day_6_JQ1 | 5.55805 | 6.2203 | 0.16241 |
| Nov | Day_1 | Day_6 | OK | 1.13142 | 7.56343 | 2.74091 | Nov | Day_6 | Day_6_JQ1 | 7.56343 | 8.55233 | 0.17728 |
| Gm98 | Day_1 | Day_6 | OK | 6.32011 | 19.8369 | 1.65016 | Gm98 | Day_6 | Day_6_JQ1 | 19.8369 | 22.4723 | 0.17996 |
| Tpbg | Day_1 | Day_6 | OK | 0.8725 | 9.20024 | 3.39845 | Tpbg | Day_6 | Day_6_JQ1 | 9.20024 | 10.4275 | 0.18065 |
| Agtr1a | Day_1 | Day_6 | OK | 6.0381 | 59.6555 | 3.30449 | Agtr1a | Day_6 | Day_6_JQ1 | 59.6555 | 67.6328 | 0.18107 |
| Ehd2 | Day_1 | Day_6 | OK | 51.4602 | 199.504 | 1.95489 | Ehd2 | Day_6 | Day_6_JQ1 | 199.504 | 226.424 | 0.18261 |
| Wipi1 | Day_1 | Day_6 | OK | 11.0468 | 33.4039 | 1.59639 | Wipi1 | Day_6 | Day_6_JQ1 | 33.4039 | 37.9118 | 0.18263 |
| Plod2 | Day_1 | Day_6 | OK | 11.2897 | 188.135 | 4.05869 | Plod2 | Day_6 | Day_6_JQ1 | 188.135 | 213.631 | 0.18335 |
| Lrat | Day_1 | Day_6 | OK | 1.0492 | 131.373 | 6.96824 | Lrat | Day_6 | Day_6_JQ1 | 131.373 | 149.73 | 0.18869 |
| Chek2 | Day_1 | Day_6 | OK | 1.89608 | 6.65961 | 1.81242 | Chek2 | Day_6 | Day_6_JQ1 | 6.65961 | 7.60109 | 0.19077 |
| Tusc3 | Day_1 | Day_6 | OK | 29.3435 | 61.3958 | 1.0651 | Tusc3 | Day_6 | Day_6_JQ1 | 61.3958 | 70.401 | 0.19746 |
| Adk | Day_1 | Day_6 | OK | 17.7375 | 37.121 | 1.06543 | Adk | Day_6 | Day_6_JQ1 | 37.121 | 42.5802 | 0.19795 |
| Dlg3 | Day_1 | Day_6 | OK | 2.92286 | 14.8995 | 2.34981 | Dlg3 | Day_6 | Day_6_JQ1 | 14.8995 | 17.133 | 0.20152 |
| 1190002F15Rik | Day_1 | Day_6 | OK | 0 | 5.36311 | inf | 1190002F15Rik | Day_6 | Day_6_JQ1 | 5.36311 | 6.16927 | 0.20203 |
| Lrrk1 | Day_1 | Day_6 | OK | 9.55454 | 21.2662 | 1.1543 | Lrrk1 | Day_6 | Day_6_JQ1 | 21.2662 | 24.4921 | 0.20376 |
| Ppp1r12b | Day_1 | Day_6 | OK | 2.91538 | 8.0199 | 1.4599 | Ppp1r12b | Day_6 | Day_6_JQ1 | 8.0199 | 9.27335 | 0.20951 |
| Pbk | Day_1 | Day_6 | OK | 0.34325 | 10.7188 | 4.96476 | Pbk | Day_6 | Day_6_JQ1 | 10.7188 | 12.4065 | 0.21094 |
| Tsc22d1 | Day_1 | Day_6 | OK | 76.8564 | 288.262 | 1.90714 | Tsc22d1 | Day_6 | Day_6_JQ1 | 288.262 | 334.274 | 0.21365 |
| Fads3 | Day_1 | Day_6 | OK | 15.0044 | 51.8643 | 1.78936 | Fads3 | Day_6 | Day_6_JQ1 | 51.8643 | 60.2646 | 0.21657 |
| Flrt2 | Day_1 | Day_6 | OK | 4.08691 | 22.2263 | 2.44319 | Flrt2 | Day_6 | Day_6_JQ1 | 22.2263 | 25.9183 | 0.2217 |
| Cdc25b | Day_1 | Day_6 | OK | 1.51434 | 6.54224 | 2.1111 | Cdc25b | Day_6 | Day_6_JQ1 | 6.54224 | 7.66351 | 0.22822 |
| Ctxn1 | Day_1 | Day_6 | OK | 3.91402 | 21.1213 | 2.43198 | Ctxn1 | Day_6 | Day_6_JQ1 | 21.1213 | 24.7491 | 0.22867 |
| Aph1b | Day_1 | Day_6 | OK | 1.94091 | 5.20821 | 1.42405 | Aph1b | Day_6 | Day_6_JQ1 | 5.20821 | 6.10496 | 0.2292 |
| Smad6 | Day_1 | Day_6 | OK | 22.9769 | 57.9806 | 1.33538 | Smad6 | Day_6 | Day_6_JQ1 | 57.9806 | 67.9958 | 0.22988 |
| Raph1 | Day_1 | Day_6 | OK | 19.9191 | 57.6427 | 1.53298 | Raph1 | Day_6 | Day_6_JQ1 | 57.6427 | 67.6351 | 0.23063 |
| Adamts2 | Day_1 | Day_6 | OK | 13.0984 | 104.393 | 2.99457 | Adamts2 | Day_6 | Day_6_JQ1 | 104.393 | 122.525 | 0.23105 |
| Slc25a4 | Day_1 | Day_6 | OK | 155.352 | 475.612 | 1.61425 | Slc25a4 | Day_6 | Day_6_JQ1 | 475.612 | 559.358 | 0.23399 |
| Tpx2 | Day_1 | Day_6 | OK | 0.52619 | 17.121 | 5.02404 | Tpx2 | Day_6 | Day_6_JQ1 | 17.121 | 20.1374 | 0.2341 |
| Pfn2 | Day_1 | Day_6 | OK | 12.2657 | 29.5923 | 1.27059 | Pfn2 | Day_6 | Day_6_JQ1 | 29.5923 | 34.8317 | 0.23518 |
| Pdlim2 | Day_1 | Day_6 | OK | 4.80891 | 35.3275 | 2.87701 | Pdlim2 | Day_6 | Day_6_JQ1 | 35.3275 | 41.6397 | 0.23717 |
| Fdxr | Day_1 | Day_6 | OK | 3.55409 | 11.9364 | 1.74782 | Fdxr | Day_6 | Day_6_JQ1 | 11.9364 | 14.1099 | 0.24134 |
| Fgfrl1 | Day_1 | Day_6 | OK | 6.26687 | 15.8272 | 1.33659 | Fgfrl1 | Day_6 | Day_6_JQ1 | 15.8272 | 18.734 | 0.24325 |
| Vcan | Day_1 | Day_6 | OK | 2.87191 | 22.0326 | 2.93956 | Vcan | Day_6 | Day_6_JQ1 | 22.0326 | 26.1044 | 0.24465 |
| Ptprg | Day_1 | Day_6 | OK | 9.33083 | 19.1139 | 1.03455 | Ptprg | Day_6 | Day_6_JQ1 | 19.1139 | 22.6896 | 0.2474 |
| Tk1 | Day_1 | Day_6 | OK | 1.65438 | 18.6209 | 3.49256 | Tk1 | Day_6 | Day_6_JQ1 | 18.6209 | 22.1517 | 0.2505 |
| Bcl9l | Day_1 | Day_6 | OK | 6.52466 | 17.2048 | 1.39884 | Bcl9l | Day_6 | Day_6_JQ1 | 17.2048 | 20.5171 | 0.25401 |
| Ccnb1 | Day_1 | Day_6 | OK | 0.21547 | 11.9279 | 5.79072 | Ccnb1 | Day_6 | Day_6_JQ1 | 11.9279 | 14.2269 | 0.25428 |
| Gnb5 | Day_1 | Day_6 | OK | 4.19129 | 17.1664 | 2.03412 | Gnb5 | Day_6 | Day_6_JQ1 | 17.1664 | 20.4898 | 0.25532 |
| Bnc1 | Day_1 | Day_6 | OK | 0.26366 | 5.63494 | 4.41764 | Bnc1 | Day_6 | Day_6_JQ1 | 5.63494 | 6.72972 | 0.25615 |
| Scd2 | Day_1 | Day_6 | OK | 13.0693 | 85.613 | 2.71165 | Scd2 | Day_6 | Day_6_JQ1 | 85.613 | 102.379 | 0.25802 |
| Tspan3 | Day_1 | Day_6 | OK | 49.8098 | 110.213 | 1.1458 | Tspan3 | Day_6 | Day_6_JQ1 | 110.213 | 131.995 | 0.26018 |
| Ndufv3 | Day_1 | Day_6 | OK | 43.3067 | 114.765 | 1.40602 | Ndufv3 | Day_6 | Day_6_JQ1 | 114.765 | 137.536 | 0.26112 |
| Ccnd3 | Day_1 | Day_6 | OK | 25.2785 | 89.8993 | 1.8304 | Ccnd3 | Day_6 | Day_6_JQ1 | 89.8993 | 107.985 | 0.26445 |
| Dkk3 | Day_1 | Day_6 | OK | 16.944 | 49.7269 | 1.55325 | Dkk3 | Day_6 | Day_6_JQ1 | 49.7269 | 59.7496 | 0.2649 |
| Sdc2 | Day_1 | Day_6 | OK | 7.7981 | 74.6344 | 3.25864 | Sdc2 | Day_6 | Day_6_JQ1 | 74.6344 | 89.6847 | 0.26502 |
| Cd81 | Day_1 | Day_6 | OK | 152.97 | 331.487 | 1.1157 | Cd81 | Day_6 | Day_6_JQ1 | 331.487 | 398.479 | 0.26555 |
| Mkl1 | Day_1 | Day_6 | OK | 11.6292 | 36.615 | 1.65468 | Mkl1 | Day_6 | Day_6_JQ1 | 36.615 | 44.1353 | 0.2695 |
| Rcn2 | Day_1 | Day_6 | OK | 33.4559 | 89.2621 | 1.41579 | Rcn2 | Day_6 | Day_6_JQ1 | 89.2621 | 107.612 | 0.26972 |
| Fzd7 | Day_1 | Day_6 | OK | 5.18896 | 37.0523 | 2.83605 | Fzd7 | Day_6 | Day_6_JQ1 | 37.0523 | 44.7243 | 0.27149 |
| Nsg1 | Day_1 | Day_6 | OK | 4.74356 | 21.9505 | 2.21021 | Nsg1 | Day_6 | Day_6_JQ1 | 21.9505 | 26.5272 | 0.27322 |
| Aebp1 | Day_1 | Day_6 | OK | 39.7483 | 218.695 | 2.45996 | Aebp1 | Day_6 | Day_6_JQ1 | 218.695 | 264.901 | 0.27653 |
| Gulp1 | Day_1 | Day_6 | OK | 0.96606 | 10.3018 | 3.41463 | Gulp1 | Day_6 | Day_6_JQ1 | 10.3018 | 12.4818 | 0.27694 |
| Pkd1 | Day_1 | Day_6 | OK | 10.529 | 27.741 | 1.39765 | Pkd1 | Day_6 | Day_6_JQ1 | 27.741 | 33.6568 | 0.27888 |
| Fam102b | Day_1 | Day_6 | OK | 7.92854 | 16.2425 | 1.03465 | Fam102b | Day_6 | Day_6_JQ1 | 16.2425 | 19.8193 | 0.28713 |
| Wls | Day_1 | Day_6 | OK | 61.1644 | 220.882 | 1.85252 | Wls | Day_6 | Day_6_JQ1 | 220.882 | 269.873 | 0.289 |
| Ccnyl1 | Day_1 | Day_6 | OK | 8.40387 | 18.1803 | 1.11325 | Ccnyl1 | Day_6 | Day_6_JQ1 | 18.1803 | 22.2687 | 0.29264 |
| Amotl2 | Day_1 | Day_6 | OK | 34.3719 | 146.696 | 2.09353 | Amotl2 | Day_6 | Day_6_JQ1 | 146.696 | 180.707 | 0.30083 |
| Fzd1 | Day_1 | Day_6 | OK | 7.51 | 45.5577 | 2.60081 | Fzd1 | Day_6 | Day_6_JQ1 | 45.5577 | 56.322 | 0.306 |
| Pcdh18 | Day_1 | Day_6 | OK | 2.3605 | 10.0512 | 2.09021 | Pcdh18 | Day_6 | Day_6_JQ1 | 10.0512 | 12.4277 | 0.30619 |
| Klhl22 | Day_1 | Day_6 | OK | 16.1934 | 39.8741 | 1.30005 | Klhl22 | Day_6 | Day_6_JQ1 | 39.8741 | 49.3373 | 0.30723 |
| Masp1 | Day_1 | Day_6 | OK | 24.6899 | 101.462 | 2.03895 | Masp1 | Day_6 | Day_6_JQ1 | 101.462 | 125.598 | 0.30786 |
| Kirrel | Day_1 | Day_6 | OK | 17.2616 | 43.8672 | 1.34558 | Kirrel | Day_6 | Day_6_JQ1 | 43.8672 | 54.3631 | 0.30949 |
| Colec12 | Day_1 | Day_6 | OK | 13.8387 | 67.6124 | 2.28858 | Colec12 | Day_6 | Day_6_JQ1 | 67.6124 | 83.8876 | 0.31117 |
| Pdgfrb | Day_1 | Day_6 | OK | 27.3596 | 131.107 | 2.26063 | Pdgfrb | Day_6 | Day_6_JQ1 | 131.107 | 163.464 | 0.31823 |
| Fbn1 | Day_1 | Day_6 | OK | 15.1034 | 159.051 | 3.39654 | Fbn1 | Day_6 | Day_6_JQ1 | 159.051 | 198.339 | 0.31848 |
| Rtn2 | Day_1 | Day_6 | OK | 0.98853 | 12.3608 | 3.64435 | Rtn2 | Day_6 | Day_6_JQ1 | 12.3608 | 15.4223 | 0.31924 |
| Odz3 | Day_1 | Day_6 | OK | 0.29315 | 6.43101 | 4.45535 | Odz3 | Day_6 | Day_6_JQ1 | 6.43101 | 8.02762 | 0.31993 |
| Ccdc99 | Day_1 | Day_6 | OK | 1.57281 | 5.62489 | 1.83848 | Ccdc99 | Day_6 | Day_6_JQ1 | 5.62489 | 7.0341 | 0.32254 |
| Rnase4 | Day_1 | Day_6 | OK | 14.6654 | 114.613 | 2.96628 | Rnase4 | Day_6 | Day_6_JQ1 | 114.613 | 143.742 | 0.32671 |
| Ect2 | Day_1 | Day_6 | OK | 0.87167 | 8.1845 | 3.23104 | Ect2 | Day_6 | Day_6_JQ1 | 8.1845 | 10.3091 | 0.33295 |
| S100a11 | Day_1 | Day_6 | OK | 264.07 | 934.601 | 1.82343 | S100a11 | Day_6 | Day_6_JQ1 | 934.601 | 1179.49 | 0.33574 |
| Kank2 | Day_1 | Day_6 | OK | 12.8483 | 43.8331 | 1.77045 | Kank2 | Day_6 | Day_6_JQ1 | 43.8331 | 55.7877 | 0.34793 |
| Pknox2 | Day_1 | Day_6 | OK | 0.95407 | 10.1366 | 3.40933 | Pknox2 | Day_6 | Day_6_JQ1 | 10.1366 | 12.9177 | 0.34978 |
| Narf | Day_1 | Day_6 | OK | 4.52876 | 12.642 | 1.48104 | Narf | Day_6 | Day_6_JQ1 | 12.642 | 16.1256 | 0.35113 |
| Anln | Day_1 | Day_6 | OK | 0.51615 | 22.0337 | 5.41577 | Anln | Day_6 | Day_6_JQ1 | 22.0337 | 28.1204 | 0.3519 |
| Gda | Day_1 | Day_6 | OK | 8.22186 | 20.3325 | 1.30625 | Gda | Day_6 | Day_6_JQ1 | 20.3325 | 26.0731 | 0.35877 |
| Anxa1 | Day_1 | Day_6 | OK | 55.4465 | 324.197 | 2.5477 | Anxa1 | Day_6 | Day_6_JQ1 | 324.197 | 417.067 | 0.36341 |
| Clybl | Day_1 | Day_6 | OK | 3.13235 | 10.2168 | 1.70563 | Clybl | Day_6 | Day_6_JQ1 | 10.2168 | 13.1715 | 0.36647 |
| Nptn | Day_1 | Day_6 | OK | 84.8882 | 170.749 | 1.00824 | Nptn | Day_6 | Day_6_JQ1 | 170.749 | 220.466 | 0.36868 |
| 4632434I11Rik | Day_1 | Day_6 | OK | 2.02206 | 7.69931 | 1.9289 | 4632434I11Rik | Day_6 | Day_6_JQ1 | 7.69931 | 9.9596 | 0.37136 |
| G0s2 | Day_1 | Day_6 | OK | 7.23516 | 29.7741 | 2.04096 | G0s2 | Day_6 | Day_6_JQ1 | 29.7741 | 38.5429 | 0.37241 |
| Gpc6 | Day_1 | Day_6 | OK | 0.99493 | 10.2853 | 3.36985 | Gpc6 | Day_6 | Day_6_JQ1 | 10.2853 | 13.3385 | 0.37501 |
| 2810408I11Rik | Day_1 | Day_6 | OK | 1.58755 | 9.353 | 2.55862 | 2810408I11Rik | Day_6 | Day_6_JQ1 | 9.353 | 12.1324 | 0.37537 |
| Pigs | Day_1 | Day_6 | OK | 17.3349 | 35.7657 | 1.0449 | Pigs | Day_6 | Day_6_JQ1 | 35.7657 | 46.4598 | 0.37741 |
| Fn1 | Day_1 | Day_6 | OK | 23.4324 | 791.46 | 5.07794 | Fn1 | Day_6 | Day_6_JQ1 | 791.46 | 1028.26 | 0.37761 |
| Bhlhb9 | Day_1 | Day_6 | OK | 4.2983 | 12.1266 | 1.49634 | Bhlhb9 | Day_6 | Day_6_JQ1 | 12.1266 | 15.7871 | 0.38057 |
| Gucy1a3 | Day_1 | Day_6 | OK | 9.97978 | 45.3184 | 2.18302 | Gucy1a3 | Day_6 | Day_6_JQ1 | 45.3184 | 59.0286 | 0.38132 |
| Bub1b | Day_1 | Day_6 | OK | 1.44425 | 9.70881 | 2.74898 | Bub1b | Day_6 | Day_6_JQ1 | 9.70881 | 12.6773 | 0.38489 |
| Fam164a | Day_1 | Day_6 | OK | 5.09112 | 15.7404 | 1.62842 | Fam164a | Day_6 | Day_6_JQ1 | 15.7404 | 20.5633 | 0.3856 |
| Mxra7 | Day_1 | Day_6 | OK | 13.7029 | 41.834 | 1.6102 | Mxra7 | Day_6 | Day_6_JQ1 | 41.834 | 54.8528 | 0.39089 |
| Plekhg2 | Day_1 | Day_6 | OK | 9.9083 | 25.8296 | 1.38231 | Plekhg2 | Day_6 | Day_6_JQ1 | 25.8296 | 33.8948 | 0.39204 |
| Ube2h | Day_1 | Day_6 | OK | 16.0023 | 34.3873 | 1.10359 | Ube2h | Day_6 | Day_6_JQ1 | 34.3873 | 45.1252 | 0.39206 |
| Arhgap24 | Day_1 | Day_6 | OK | 5.98419 | 24.334 | 2.02375 | Arhgap24 | Day_6 | Day_6_JQ1 | 24.334 | 32.005 | 0.39533 |
| Higd2a | Day_1 | Day_6 | OK | 66.4233 | 140.49 | 1.0807 | Higd2a | Day_6 | Day_6_JQ1 | 140.49 | 184.81 | 0.39558 |
| Gstm2 | Day_1 | Day_6 | OK | 23.7447 | 95.4712 | 2.00746 | Gstm2 | Day_6 | Day_6_JQ1 | 95.4712 | 125.725 | 0.39714 |
| Incenp | Day_1 | Day_6 | OK | 5.29409 | 14.4252 | 1.44614 | Incenp | Day_6 | Day_6_JQ1 | 14.4252 | 19.0259 | 0.39937 |
| Cyp27a1 | Day_1 | Day_6 | OK | 2.70186 | 12.8055 | 2.24474 | Cyp27a1 | Day_6 | Day_6_JQ1 | 12.8055 | 16.9115 | 0.40124 |
| Dut | Day_1 | Day_6 | OK | 4.48552 | 12.8074 | 1.51362 | Dut | Day_6 | Day_6_JQ1 | 12.8074 | 16.9152 | 0.40135 |
| Pik3r2 | Day_1 | Day_6 | OK | 9.76669 | 29.4016 | 1.58995 | Pik3r2 | Day_6 | Day_6_JQ1 | 29.4016 | 38.9832 | 0.40696 |
| Cyp1b1 | Day_1 | Day_6 | OK | 15.2673 | 75.6563 | 2.30902 | Cyp1b1 | Day_6 | Day_6_JQ1 | 75.6563 | 100.635 | 0.4116 |
| Mfge8 | Day_1 | Day_6 | OK | 113.787 | 284.582 | 1.32251 | Mfge8 | Day_6 | Day_6_JQ1 | 284.582 | 379.144 | 0.4139 |
| Lhfp | Day_1 | Day_6 | OK | 80.006 | 277.689 | 1.79529 | Lhfp | Day_6 | Day_6_JQ1 | 277.689 | 371.966 | 0.4217 |
| Lamb2 | Day_1 | Day_6 | OK | 10.6079 | 30.7012 | 1.53316 | Lamb2 | Day_6 | Day_6_JQ1 | 30.7012 | 41.1365 | 0.42212 |
| Dzip3 | Day_1 | Day_6 | OK | 2.60919 | 6.2619 | 1.263 | Dzip3 | Day_6 | Day_6_JQ1 | 6.2619 | 8.39884 | 0.42359 |
| Uhrf1 | Day_1 | Day_6 | OK | 4.87265 | 12.4813 | 1.35699 | Uhrf1 | Day_6 | Day_6_JQ1 | 12.4813 | 16.7743 | 0.42648 |
| Wdr35 | Day_1 | Day_6 | OK | 2.61254 | 7.32896 | 1.48816 | Wdr35 | Day_6 | Day_6_JQ1 | 7.32896 | 9.87411 | 0.43004 |
| Cbx5 | Day_1 | Day_6 | OK | 10.0186 | 21.4308 | 1.09701 | Cbx5 | Day_6 | Day_6_JQ1 | 21.4308 | 28.9059 | 0.43168 |
| Cdkn2c | Day_1 | Day_6 | OK | 3.00086 | 11.9132 | 1.98911 | Cdkn2c | Day_6 | Day_6_JQ1 | 11.9132 | 16.1063 | 0.43507 |
| Eno2 | Day_1 | Day_6 | OK | 2.58974 | 7.14956 | 1.46505 | Eno2 | Day_6 | Day_6_JQ1 | 7.14956 | 9.67986 | 0.43713 |
| Gpx1 | Day_1 | Day_6 | OK | 102.55 | 245.702 | 1.26058 | Gpx1 | Day_6 | Day_6_JQ1 | 245.702 | 332.885 | 0.43812 |
| Plxdc2 | Day_1 | Day_6 | OK | 2.04979 | 19.3096 | 3.23577 | Plxdc2 | Day_6 | Day_6_JQ1 | 19.3096 | 26.1768 | 0.43897 |
| Trim59 | Day_1 | Day_6 | OK | 1.77049 | 20.0859 | 3.50396 | Trim59 | Day_6 | Day_6_JQ1 | 20.0859 | 27.2401 | 0.43955 |
| Osbpl5 | Day_1 | Day_6 | OK | 13.6193 | 30.4829 | 1.16235 | Osbpl5 | Day_6 | Day_6_JQ1 | 30.4829 | 41.3593 | 0.44021 |
| Spc24 | Day_1 | Day_6 | OK | 0.22087 | 6.22016 | 4.81569 | Spc24 | Day_6 | Day_6_JQ1 | 6.22016 | 8.49353 | 0.44941 |
| C330027C09Rik | Day_1 | Day_6 | OK | 1.07071 | 7.42951 | 2.7947 | C330027C09Rik | Day_6 | Day_6_JQ1 | 7.42951 | 10.1673 | 0.4526 |
| Ildr2 | Day_1 | Day_6 | OK | 2.09611 | 8.35201 | 1.99441 | Ildr2 | Day_6 | Day_6_JQ1 | 8.35201 | 11.4416 | 0.45409 |
| Fam171b | Day_1 | Day_6 | OK | 2.31133 | 9.40175 | 2.0242 | Fam171b | Day_6 | Day_6_JQ1 | 9.40175 | 12.8947 | 0.45578 |
| Ext2 | Day_1 | Day_6 | OK | 30.2979 | 63.6742 | 1.07149 | Ext2 | Day_6 | Day_6_JQ1 | 63.6742 | 87.8458 | 0.46426 |
| Pld3 | Day_1 | Day_6 | OK | 62.9899 | 137.922 | 1.13066 | Pld3 | Day_6 | Day_6_JQ1 | 137.922 | 190.597 | 0.46668 |
| Racgap1 | Day_1 | Day_6 | OK | 0.84944 | 16.5034 | 4.2801 | Racgap1 | Day_6 | Day_6_JQ1 | 16.5034 | 22.85 | 0.46943 |
| Mras | Day_1 | Day_6 | OK | 2.40608 | 11.1516 | 2.2125 | Mras | Day_6 | Day_6_JQ1 | 11.1516 | 15.4481 | 0.47018 |
| Dact3 | Day_1 | Day_6 | OK | 1.59911 | 19.016 | 3.57188 | Dact3 | Day_6 | Day_6_JQ1 | 19.016 | 26.3749 | 0.47195 |
| Trp53inp2 | Day_1 | Day_6 | OK | 26.0154 | 71.8603 | 1.46583 | Trp53inp2 | Day_6 | Day_6_JQ1 | 71.8603 | 99.8045 | 0.47391 |
| Cdc45 | Day_1 | Day_6 | OK | 1.28152 | 5.33452 | 2.05751 | Cdc45 | Day_6 | Day_6_JQ1 | 5.33452 | 7.41885 | 0.47584 |
| Basp1 | Day_1 | Day_6 | OK | 28.202 | 124.37 | 2.14077 | Basp1 | Day_6 | Day_6_JQ1 | 124.37 | 173.108 | 0.47703 |
| Wt1 | Day_1 | Day_6 | OK | 1.42473 | 20.8041 | 3.86811 | Wt1 | Day_6 | Day_6_JQ1 | 20.8041 | 29.0084 | 0.4796 |
| Ctnnbip1 | Day_1 | Day_6 | OK | 3.82287 | 10.2471 | 1.42249 | Ctnnbip1 | Day_6 | Day_6_JQ1 | 10.2471 | 14.2922 | 0.48002 |
| Ckap2l | Day_1 | Day_6 | OK | 0.42608 | 13.2798 | 4.96197 | Ckap2l | Day_6 | Day_6_JQ1 | 13.2798 | 18.5683 | 0.48361 |
| 2310022B05Rik | Day_1 | Day_6 | OK | 21.4357 | 94.4617 | 2.13971 | 2310022B05Rik | Day_6 | Day_6_JQ1 | 94.4617 | 132.474 | 0.48791 |
| 1110003E01Rik | Day_1 | Day_6 | OK | 25.2143 | 49.2227 | 0.96508 | 1110003E01Rik | Day_6 | Day_6_JQ1 | 49.2227 | 69.1288 | 0.48997 |
| Tacc3 | Day_1 | Day_6 | OK | 1.75713 | 12.8006 | 2.86492 | Tacc3 | Day_6 | Day_6_JQ1 | 12.8006 | 18.1794 | 0.50609 |
| St5 | Day_1 | Day_6 | OK | 7.70436 | 24.7489 | 1.68362 | St5 | Day_6 | Day_6_JQ1 | 24.7489 | 35.2195 | 0.50901 |
| Vgll3 | Day_1 | Day_6 | OK | 8.47913 | 66.9426 | 2.98094 | Vgll3 | Day_6 | Day_6_JQ1 | 66.9426 | 95.3643 | 0.51053 |
| Bphl | Day_1 | Day_6 | OK | 3.33116 | 12.9312 | 1.95676 | Bphl | Day_6 | Day_6_JQ1 | 12.9312 | 18.4476 | 0.51258 |
| Evc | Day_1 | Day_6 | OK | 1.68535 | 7.29207 | 2.11328 | Evc | Day_6 | Day_6_JQ1 | 7.29207 | 10.4125 | 0.51392 |
| Dcxr | Day_1 | Day_6 | OK | 7.03746 | 25.2345 | 1.84227 | Dcxr | Day_6 | Day_6_JQ1 | 25.2345 | 36.0469 | 0.51448 |
| Msx1 | Day_1 | Day_6 | OK | 0.18607 | 4.15055 | 4.47935 | Msx1 | Day_6 | Day_6_JQ1 | 4.15055 | 5.96237 | 0.52259 |
| Dcbld2 | Day_1 | Day_6 | OK | 17.2916 | 51.0491 | 1.56181 | Dcbld2 | Day_6 | Day_6_JQ1 | 51.0491 | 73.5335 | 0.52652 |
| Oat | Day_1 | Day_6 | OK | 58.4558 | 147.721 | 1.33745 | Oat | Day_6 | Day_6_JQ1 | 147.721 | 213.454 | 0.53106 |
| Ahdc1 | Day_1 | Day_6 | OK | 4.23238 | 9.13 | 1.10915 | Ahdc1 | Day_6 | Day_6_JQ1 | 9.13 | 13.24 | 0.53622 |
| Dag1 | Day_1 | Day_6 | OK | 41.6218 | 112.258 | 1.43141 | Dag1 | Day_6 | Day_6_JQ1 | 112.258 | 162.916 | 0.53731 |
| Mrc2 | Day_1 | Day_6 | OK | 2.27393 | 76.5869 | 5.07384 | Mrc2 | Day_6 | Day_6_JQ1 | 76.5869 | 111.626 | 0.54351 |
| Rarb | Day_1 | Day_6 | OK | 18.859 | 48.4333 | 1.36075 | Rarb | Day_6 | Day_6_JQ1 | 48.4333 | 70.6683 | 0.54506 |
| Tinagl1 | Day_1 | Day_6 | OK | 92.7868 | 221.772 | 1.25709 | Tinagl1 | Day_6 | Day_6_JQ1 | 221.772 | 326.013 | 0.55585 |
| Nuf2 | Day_1 | Day_6 | OK | 0.16616 | 6.26115 | 5.23583 | Nuf2 | Day_6 | Day_6_JQ1 | 6.26115 | 9.22711 | 0.55945 |
| Arhgef17 | Day_1 | Day_6 | OK | 6.9294 | 23.7663 | 1.77811 | Arhgef17 | Day_6 | Day_6_JQ1 | 23.7663 | 35.1441 | 0.56437 |
| Adam15 | Day_1 | Day_6 | OK | 13.2573 | 52.9726 | 1.99846 | Adam15 | Day_6 | Day_6_JQ1 | 52.9726 | 78.5951 | 0.56919 |
| Tro | Day_1 | Day_6 | OK | 0.35325 | 4.92171 | 3.80039 | Tro | Day_6 | Day_6_JQ1 | 4.92171 | 7.30735 | 0.57019 |
| Cyb5r3 | Day_1 | Day_6 | OK | 85.2756 | 172.182 | 1.01373 | Cyb5r3 | Day_6 | Day_6_JQ1 | 172.182 | 256.005 | 0.57224 |
| Ckb | Day_1 | Day_6 | OK | 26.6799 | 208.452 | 2.96589 | Ckb | Day_6 | Day_6_JQ1 | 208.452 | 310.111 | 0.57307 |
| Snap47 | Day_1 | Day_6 | OK | 19.6018 | 69.4621 | 1.82524 | Snap47 | Day_6 | Day_6_JQ1 | 69.4621 | 103.523 | 0.57565 |
| Ndc80 | Day_1 | Day_6 | OK | 0.32489 | 5.76587 | 4.14951 | Ndc80 | Day_6 | Day_6_JQ1 | 5.76587 | 8.60445 | 0.57754 |
| BC018242 | Day_1 | Day_6 | OK | 3.40851 | 11.5402 | 1.75945 | BC018242 | Day_6 | Day_6_JQ1 | 11.5402 | 17.3751 | 0.59036 |
| Zbtb7c | Day_1 | Day_6 | OK | 1.02532 | 5.24073 | 2.3537 | Zbtb7c | Day_6 | Day_6_JQ1 | 5.24073 | 7.89481 | 0.59114 |
| Gstt3 | Day_1 | Day_6 | OK | 2.90241 | 9.5465 | 1.71772 | Gstt3 | Day_6 | Day_6_JQ1 | 9.5465 | 14.4369 | 0.59672 |
| Scrn1 | Day_1 | Day_6 | OK | 0.34413 | 5.22481 | 3.92438 | Scrn1 | Day_6 | Day_6_JQ1 | 5.22481 | 7.91099 | 0.59848 |
| Kif22 | Day_1 | Day_6 | OK | 1.06984 | 9.87145 | 3.20587 | Kif22 | Day_6 | Day_6_JQ1 | 9.87145 | 14.9542 | 0.59921 |
| Lphn1 | Day_1 | Day_6 | OK | 6.46648 | 13.2649 | 1.03656 | Lphn1 | Day_6 | Day_6_JQ1 | 13.2649 | 20.1469 | 0.60295 |
| Sipa111 | Day_1 | Day_6 | OK | 5.08935 | 18.8508 | 1.88907 | Sipa111 | Day_6 | Day_6_JQ1 | 18.8508 | 28.6555 | 0.60419 |
| Twist1 | Day_1 | Day_6 | OK | 4.02431 | 13.135 | 1.70661 | Twist1 | Day_6 | Day_6_JQ1 | 13.135 | 19.9784 | 0.60502 |
| Kif4 | Day_1 | Day_6 | OK | 0.1042 | 4.82026 | 5.53164 | Kif4 | Day_6 | Day_6_JQ1 | 4.82026 | 7.33178 | 0.60505 |
| Kifl1 | Day_1 | Day_6 | OK | 0.20403 | 7.09124 | 5.11919 | Kifl1 | Day_6 | Day_6_JQ1 | 7.09124 | 10.8132 | 0.60868 |
| Mmp11 | Day_1 | Day_6 | OK | 2.19593 | 23.6416 | 3.42842 | Mmp11 | Day_6 | Day_6_JQ1 | 23.6416 | 36.1215 | 0.61153 |
| BC031353 | Day_1 | Day_6 | OK | 2.62297 | 6.54601 | 1.31942 | BC031353 | Day_6 | Day_6_JQ1 | 6.54601 | 10.0028 | 0.61172 |
| S100a6 | Day_1 | Day_6 | OK | 249.02 | 1051.65 | 2.07832 | S100a6 | Day_6 | Day_6_JQ1 | 1051.65 | 1608.42 | 0.61299 |
| Elovl5 | Day_1 | Day_6 | OK | 20.7934 | 42.7846 | 1.04097 | Elovl5 | Day_6 | Day_6_JQ1 | 42.7846 | 65.5058 | 0.61453 |
| 1600014C10Rik | Day_1 | Day_6 | OK | 6.37767 | 13.9803 | 1.1323 | 1600014C10Rik | Day_6 | Day_6_JQ1 | 13.9803 | 21.437 | 0.6167 |
| Tmem53 | Day_1 | Day_6 | OK | 0.66088 | 11.3331 | 4.10002 | Tmem53 | Day_6 | Day_6_JQ1 | 11.3331 | 17.4035 | 0.61883 |
| Smc4 | Day_1 | Day_6 | OK | 7.69177 | 22.0455 | 1.51909 | Smc4 | Day_6 | Day_6_JQ1 | 22.0455 | 33.8803 | 0.61997 |
| Pygb | Day_1 | Day_6 | OK | 12.7507 | 27.3579 | 1.10138 | Pygb | Day_6 | Day_6_JQ1 | 27.3579 | 42.1403 | 0.62325 |
| Pbx1 | Day_1 | Day_6 | OK | 9.71944 | 31.4665 | 1.69487 | Pbx1 | Day_6 | Day_6_JQ1 | 31.4665 | 48.4716 | 0.62332 |
| Melk | Day_1 | Day_6 | OK | 0.20231 | 4.84753 | 4.58261 | Melk | Day_6 | Day_6_JQ1 | 4.84753 | 7.46724 | 0.62333 |
| Prc1 | Day_1 | Day_6 | OK | 0.84883 | 19.8871 | 4.55022 | Prc1 | Day_6 | Day_6_JQ1 | 19.8871 | 30.6759 | 0.62527 |
| Foxm1 | Day_1 | Day_6 | OK | 1.12178 | 10.1786 | 3.18167 | Foxm1 | Day_6 | Day_6_JQ1 | 10.1786 | 15.7609 | 0.63081 |
| Apln | Day_1 | Day_6 | OK | 1.44594 | 10.3508 | 2.83966 | Apln | Day_6 | Day_6_JQ1 | 10.3508 | 16.0329 | 0.6313 |
| Cks1b | Day_1 | Day_6 | OK | 18.0279 | 73.4148 | 2.02584 | Cks1b | Day_6 | Day_6_JQ1 | 73.4148 | 113.98 | 0.63464 |
| Dlgap5 | Day_1 | Day_6 | OK | 0.2621 | 7.03835 | 4.74707 | Dlgap5 | Day_6 | Day_6_JQ1 | 7.03835 | 10.9428 | 0.63668 |
| Cdk1 | Day_1 | Day_6 | OK | 0.62317 | 18.5089 | 4.89244 | Cdk1 | Day_6 | Day_6_JQ1 | 18.5089 | 28.779 | 0.6368 |
| Ccnb2 | Day_1 | Day_6 | OK | 0.59555 | 14.5655 | 4.61219 | Ccnb2 | Day_6 | Day_6_JQ1 | 14.5655 | 22.7753 | 0.64491 |
| Gas1 | Day_1 | Day_6 | OK | 1.58789 | 51.3516 | 5.01522 | Gas1 | Day_6 | Day_6_JQ1 | 51.3516 | 80.3077 | 0.64513 |
| Gmnn | Day_1 | Day_6 | OK | 3.43057 | 12.6804 | 1.88608 | Gmnn | Day_6 | Day_6_JQ1 | 12.6804 | 19.8433 | 0.64605 |
| Ddr2 | Day_1 | Day_6 | OK | 17.9982 | 40.3519 | 1.16478 | Ddr2 | Day_6 | Day_6_JQ1 | 40.3519 | 63.2059 | 0.64742 |
| Tfdp2 | Day_1 | Day_6 | OK | 2.8816 | 6.75582 | 1.22926 | Tfdp2 | Day_6 | Day_6_JQ1 | 6.75582 | 10.5854 | 0.64788 |
| Osr1 | Day_1 | Day_6 | OK | 1.41811 | 10.5979 | 2.90174 | Osr1 | Day_6 | Day_6_JQ1 | 10.5979 | 16.6329 | 0.65026 |
| Ephb3 | Day_1 | Day_6 | OK | 1.34668 | 10.5143 | 2.96488 | Ephb3 | Day_6 | Day_6_JQ1 | 10.5143 | 16.6667 | 0.66461 |
| Arhgap11a | Day_1 | Day_6 | OK | 1.87972 | 8.16252 | 2.1185 | Arhgap11a | Day_6 | Day_6_JQ1 | 8.16252 | 12.9839 | 0.66964 |
| Smc2 | Day_1 | Day_6 | OK | 5.13697 | 12.2769 | 1.25696 | Smc2 | Day_6 | Day_6_JQ1 | 12.2769 | 19.557 | 0.67174 |
| Hand2 | Day_1 | Day_6 | OK | 7.88479 | 69.2278 | 3.13421 | Hand2 | Day_6 | Day_6_JQ1 | 69.2278 | 110.598 | 0.6759 |
| Hmmr | Day_1 | Day_6 | OK | 0.14398 | 10.6929 | 6.21466 | Hmmr | Day_6 | Day_6_JQ1 | 10.6929 | 17.0843 | 0.67602 |
| Ptgr1 | Day_1 | Day_6 | OK | 4.81003 | 15.6172 | 1.69902 | Ptgr1 | Day_6 | Day_6_JQ1 | 15.6172 | 25.0296 | 0.6805 |
| Cadm4 | Day_1 | Day_6 | OK | 1.31914 | 14.4092 | 3.44932 | Cadm4 | Day_6 | Day_6_JQ1 | 14.4092 | 23.19 | 0.68651 |
| Slit2 | Day_1 | Day_6 | OK | 0.62565 | 9.23408 | 3.88355 | Slit2 | Day_6 | Day_6_JQ1 | 9.23408 | 14.8622 | 0.68661 |
| Colec11 | Day_1 | Day_6 | OK | 7.38427 | 42.7652 | 2.53391 | Colec11 | Day_6 | Day_6_JQ1 | 42.7652 | 68.8593 | 0.68722 |
| Cpt1c | Day_1 | Day_6 | OK | 3.45645 | 18.9279 | 2.45315 | Cpt1c | Day_6 | Day_6_JQ1 | 18.9279 | 30.5415 | 0.69026 |
| Pxmp4 | Day_1 | Day_6 | OK | 2.6137 | 10.9554 | 2.06748 | Pxmp4 | Day_6 | Day_6_JQ1 | 10.9554 | 17.7124 | 0.69312 |
| Afap1l2 | Day_1 | Day_6 | OK | 2.00581 | 36.3858 | 4.18112 | Afap1l2 | Day_6 | Day_6_JQ1 | 36.3858 | 58.9193 | 0.69537 |
| Cend1 | Day_1 | Day_6 | OK | 0.24235 | 4.69509 | 4.27599 | Cend1 | Day_6 | Day_6_JQ1 | 4.69509 | 7.61491 | 0.69768 |
| Nkd1 | Day_1 | Day_6 | OK | 3.21698 | 10.4236 | 1.69607 | Nkd1 | Day_6 | Day_6_JQ1 | 10.4236 | 16.9471 | 0.70119 |
| Id3 | Day_1 | Day_6 | OK | 182.706 | 651.261 | 1.83371 | Id3 | Day_6 | Day_6_JQ1 | 651.261 | 1063.75 | 0.70785 |
| Ephx1 | Day_1 | Day_6 | OK | 14.1851 | 80.7597 | 2.50926 | Ephx1 | Day_6 | Day_6_JQ1 | 80.7597 | 132.832 | 0.7179 |
| Hadh | Day_1 | Day_6 | OK | 15.7722 | 33.9076 | 1.10423 | Hadh | Day_6 | Day_6_JQ1 | 33.9076 | 56.1898 | 0.7287 |
| Mki67 | Day_1 | Day_6 | OK | 0.57478 | 16.7576 | 4.86568 | Mki67 | Day_6 | Day_6_JQ1 | 16.7576 | 27.8603 | 0.73339 |
| Kif2c | Day_1 | Day_6 | OK | 0.08168 | 7.07907 | 6.43743 | Kif2c | Day_6 | Day_6_JQ1 | 7.07907 | 11.8118 | 0.7386 |
| Cenph | Day_1 | Day_6 | OK | 0.48507 | 5.33307 | 3.4587 | Cenph | Day_6 | Day_6_JQ1 | 5.33307 | 8.89881 | 0.73865 |
| Tmsb10 | Day_1 | Day_6 | OK | 264.817 | 668.693 | 1.33635 | Tmsb10 | Day_6 | Day_6_JQ1 | 668.693 | 1117.74 | 0.74117 |
| BC046404 | Day_1 | Day_6 | OK | 3.56512 | 16.3393 | 2.19633 | BC046404 | Day_6 | Day_6_JQ1 | 16.3393 | 27.3155 | 0.74137 |
| E2f1 | Day_1 | Day_6 | OK | 2.33339 | 12.5964 | 2.43251 | E2f1 | Day_6 | Day_6_JQ1 | 12.5964 | 21.0746 | 0.7425 |
| D2Ertd750e | Day_1 | Day_6 | OK | 0.74234 | 6.14865 | 3.05013 | D2Ertd750e | Day_6 | Day_6_JQ1 | 6.14865 | 10.3027 | 0.74469 |
| Spry1 | Day_1 | Day_6 | OK | 7.45186 | 26.4358 | 1.82682 | Spry1 | Day_6 | Day_6_JQ1 | 26.4358 | 44.5222 | 0.75203 |
| Kif20b | Day_1 | Day_6 | OK | 0.7828 | 4.3217 | 2.46488 | Kif20b | Day_6 | Day_6_JQ1 | 4.3217 | 7.29913 | 0.75613 |
| Diap3 | Day_1 | Day_6 | OK | 0.23656 | 6.33959 | 4.74413 | Diap3 | Day_6 | Day_6_JQ1 | 6.33959 | 10.7413 | 0.76071 |
| Aurkb | Day_1 | Day_6 | OK | 0.43067 | 10.7428 | 4.64064 | Aurkb | Day_6 | Day_6_JQ1 | 10.7428 | 18.2306 | 0.76299 |
| Gyg | Day_1 | Day_6 | OK | 23.9741 | 48.3962 | 1.01342 | Gyg | Day_6 | Day_6_JQ1 | 48.3962 | 82.2854 | 0.76574 |
| Bambi | Day_1 | Day_6 | OK | 2.98618 | 11.8601 | 1.98974 | Bambi | Day_6 | Day_6_JQ1 | 11.8601 | 20.2742 | 0.77353 |
| Bicc1 | Day_1 | Day_6 | OK | 5.88022 | 50.5532 | 3.10386 | Bicc1 | Day_6 | Day_6_JQ1 | 50.5532 | 86.5801 | 0.77623 |
| Gpm6b | Day_1 | Day_6 | OK | 2.69918 | 11.5364 | 2.0956 | Gpm6b | Day_6 | Day_6_JQ1 | 11.5364 | 19.7607 | 0.77643 |
| Fignl1 | Day_1 | Day_6 | OK | 1.9668 | 7.3073 | 1.89349 | Fignl1 | Day_6 | Day_6_JQ1 | 7.3073 | 12.5326 | 0.77828 |
| Fscn1 | Day_1 | Day_6 | OK | 99.7474 | 224.268 | 1.16887 | Fscn1 | Day_6 | Day_6_JQ1 | 224.268 | 385.037 | 0.77977 |
| Cenpe | Day_1 | Day_6 | OK | 0.20724 | 5.31127 | 4.67967 | Cenpe | Day_6 | Day_6_JQ1 | 5.31127 | 9.12855 | 0.78133 |
| Cdca3 | Day_1 | Day_6 | OK | 0.50015 | 18.2337 | 5.18809 | Cdca3 | Day_6 | Day_6_JQ1 | 18.2337 | 31.4087 | 0.78456 |
| Ccnf | Day_1 | Day_6 | OK | 1.03748 | 6.84076 | 2.72108 | Ccnf | Day_6 | Day_6_JQ1 | 6.84076 | 11.7934 | 0.78576 |
| Fbxo5 | Day_1 | Day_6 | OK | 1.58829 | 6.87728 | 2.11436 | Fbxo5 | Day_6 | Day_6_JQ1 | 6.87728 | 11.9069 | 0.79189 |
| Chaf1a | Day_1 | Day_6 | OK | 2.84919 | 9.72837 | 1.77164 | Chaf1a | Day_6 | Day_6_JQ1 | 9.72837 | 16.8473 | 0.79225 |
| Rad51 | Day_1 | Day_6 | OK | 0.71715 | 6.95591 | 3.2779 | Rad51 | Day_6 | Day_6_JQ1 | 6.95591 | 12.1115 | 0.80007 |
| Mxd4 | Day_1 | Day_6 | OK | 20.0829 | 43.5914 | 1.11808 | Mxd4 | Day_6 | Day_6_JQ1 | 43.5914 | 76.146 | 0.80472 |
| Slc2a8 | Day_1 | Day_6 | OK | 3.05054 | 9.19196 | 1.59131 | Slc2a8 | Day_6 | Day_6_JQ1 | 9.19196 | 16.1545 | 0.8135 |
| Ptrf | Day_1 | Day_6 | OK | 45.433 | 147.463 | 1.69854 | Ptrf | Day_6 | Day_6_JQ1 | 147.463 | 259.645 | 0.81618 |
| Top2a | Day_1 | Day_6 | OK | 0.66522 | 23.1815 | 5.12299 | Top2a | Day_6 | Day_6_JQ1 | 23.1815 | 41.0029 | 0.82275 |
| Meis1 | Day_1 | Day_6 | OK | 4.50446 | 13.9908 | 1.63505 | Meis1 | Day_6 | Day_6_JQ1 | 13.9908 | 24.7836 | 0.8249 |
| Barx1 | Day_1 | Day_6 | OK | 0.80072 | 6.44345 | 3.00847 | Barx1 | Day_6 | Day_6_JQ1 | 6.44345 | 11.4288 | 0.82677 |
| Nek2 | Day_1 | Day_6 | OK | 0.18185 | 6.46598 | 5.15203 | Nek2 | Day_6 | Day_6_JQ1 | 6.46598 | 11.522 | 0.83345 |
| Aug-00 | Day_1 | Day_6 | OK | 2.18631 | 63.7502 | 4.86586 | Aug-00 | Day_6 | Day_6_JQ1 | 63.7502 | 113.948 | 0.83787 |
| Bub1 | Day_1 | Day_6 | OK | 0.06873 | 5.0278 | 6.19288 | Bub1 | Day_6 | Day_6_JQ1 | 5.0278 | 8.99346 | 0.83895 |
| Cdon | Day_1 | Day_6 | OK | 2.00259 | 9.37626 | 2.22715 | Cdon | Day_6 | Day_6_JQ1 | 9.37626 | 16.8708 | 0.84744 |
| Birc5 | Day_1 | Day_6 | OK | 0.87651 | 28.0229 | 4.99869 | Birc5 | Day_6 | Day_6_JQ1 | 28.0229 | 50.4974 | 0.8496 |
| Fam123b | Day_1 | Day_6 | OK | 2.07636 | 5.2225 | 1.33068 | Fam123b | Day_6 | Day_6_JQ1 | 5.2225 | 9.43046 | 0.85259 |
| Kifc1 | Day_1 | Day_6 | OK | 0.4616 | 8.77026 | 4.24789 | Kifc1 | Day_6 | Day_6_JQ1 | 8.77026 | 15.9311 | 0.86115 |
| Cep55 | Day_1 | Day_6 | OK | 0.71923 | 7.23919 | 3.3313 | Cep55 | Day_6 | Day_6_JQ1 | 7.23919 | 13.1668 | 0.86301 |
| Ddr1 | Day_1 | Day_6 | OK | 11.4971 | 58.3424 | 2.34328 | Ddr1 | Day_6 | Day_6_JQ1 | 58.3424 | 106.289 | 0.86537 |
| Mad2l1 | Day_1 | Day_6 | OK | 2.98757 | 17.4725 | 2.54805 | Mad2l1 | Day_6 | Day_6_JQ1 | 17.4725 | 31.9743 | 0.87182 |
| Ghr | Day_1 | Day_6 | OK | 8.91835 | 33.0628 | 1.89036 | Ghr | Day_6 | Day_6_JQ1 | 33.0628 | 60.5749 | 0.87351 |
| Rbp1 | Day_1 | Day_6 | OK | 10.8502 | 119.075 | 3.45608 | Rbp1 | Day_6 | Day_6_JQ1 | 119.075 | 218.378 | 0.87496 |
| Casc4 | Day_1 | Day_6 | OK | 8.96553 | 20.03 | 1.1597 | Casc4 | Day_6 | Day_6_JQ1 | 20.03 | 36.8307 | 0.87875 |
| Kif23 | Day_1 | Day_6 | OK | 0.77133 | 6.45484 | 3.06497 | Kif23 | Day_6 | Day_6_JQ1 | 6.45484 | 11.9374 | 0.88703 |
| Shcbp1 | Day_1 | Day_6 | OK | 0.20221 | 6.48548 | 5.00327 | Shcbp1 | Day_6 | Day_6_JQ1 | 6.48548 | 11.9996 | 0.8877 |
| Ncapd2 | Day_1 | Day_6 | OK | 3.2224 | 10.0027 | 1.63418 | Ncapd2 | Day_6 | Day_6_JQ1 | 10.0027 | 18.5667 | 0.89233 |
| Nusap1 | Day_1 | Day_6 | OK | 0.22436 | 5.38904 | 4.58612 | Nusap1 | Day_6 | Day_6_JQ1 | 5.38904 | 10.0613 | 0.90071 |
| Timp2 | Day_1 | Day_6 | OK | 30.5458 | 176.05 | 2.52694 | Timp2 | Day_6 | Day_6_JQ1 | 176.05 | 329.559 | 0.90455 |
| Cdca5 | Day_1 | Day_6 | OK | 0.40063 | 5.38662 | 3.74903 | Cdca5 | Day_6 | Day_6_JQ1 | 5.38662 | 10.0872 | 0.90508 |
| Spag5 | Day_1 | Day_6 | OK | 0.10357 | 5.21072 | 5.65285 | Spag5 | Day_6 | Day_6_JQ1 | 5.21072 | 9.80302 | 0.91174 |
| Ncapg2 | Day_1 | Day_6 | OK | 1.61099 | 6.09607 | 1.91993 | Ncapg2 | Day_6 | Day_6_JQ1 | 6.09607 | 11.6063 | 0.92896 |
| Bcmo1 | Day_1 | Day_6 | OK | 24.8771 | 83.7036 | 1.75047 | Bcmo1 | Day_6 | Day_6_JQ1 | 83.7036 | 159.902 | 0.93383 |
| Ckap2 | Day_1 | Day_6 | OK | 2.71366 | 17.3132 | 2.67356 | Ckap2 | Day_6 | Day_6_JQ1 | 17.3132 | 33.1117 | 0.93547 |
| Clca1 | Day_1 | Day_6 | OK | 1.25274 | 26.7946 | 4.41879 | Clca1 | Day_6 | Day_6_JQ1 | 26.7946 | 52.2679 | 0.96398 |
| Klhl13 | Day_1 | Day_6 | OK | 2.2679 | 50.9016 | 4.48828 | Klhl13 | Day_6 | Day_6_JQ1 | 50.9016 | 100 | 0.97423 |
| Lama1 | Day_1 | Day_6 | OK | 1.17131 | 8.49297 | 2.85814 | Lama1 | Day_6 | Day_6_JQ1 | 8.49297 | 16.8277 | 0.98649 |
| Wdr6 | Day_1 | Day_6 | OK | 10.7278 | 28.5176 | 1.4105 | Wdr6 | Day_6 | Day_6_JQ1 | 28.5176 | 56.5942 | 0.9888 |
| Reck | Day_1 | Day_6 | OK | 3.73372 | 8.74178 | 1.22731 | Reck | Day_6 | Day_6_JQ1 | 8.74178 | 17.3878 | 0.99207 |
| Rrm2 | Day_1 | Day_6 | OK | 2.39336 | 34.2032 | 3.83702 | Rrm2 | Day_6 | Day_6_JQ1 | 34.2032 | 68.04 | 0.99225 |
| Pla2r1 | Day_1 | Day_6 | OK | 1.92972 | 13.362 | 2.79168 | Pla2r1 | Day_6 | Day_6_JQ1 | 13.362 | 26.6778 | 0.9975 |
| Igf2r | Day_1 | Day_6 | OK | 12.7405 | 33.831 | 1.40892 | Igf2r | Day_6 | Day_6_JQ1 | 33.831 | 67.7147 | 1.00112 |
| Cd248 | Day_1 | Day_6 | OK | 0.99919 | 58.5667 | 5.87317 | Cd248 | Day_6 | Day_6_JQ1 | 58.5667 | 118.878 | 1.02133 |
| Fbln1 | Day_1 | Day_6 | OK | 0.9594 | 6.62881 | 2.78855 | Fbln1 | Day_6 | Day_6_JQ1 | 6.62881 | 13.4635 | 1.02224 |
| Shisa2 | Day_1 | Day_6 | OK | 0.45469 | 71.1475 | 7.28978 | Shisa2 | Day_6 | Day_6_JQ1 | 71.1475 | 144.995 | 1.02712 |
| Sbsn | Day_1 | Day_6 | OK | 1.3781 | 7.98866 | 2.53528 | Sbsn | Day_6 | Day_6_JQ1 | 7.98866 | 16.3231 | 1.03089 |
| Peg10 | Day_1 | Day_6 | OK | 1.30056 | 59.2925 | 5.51065 | Peg10 | Day_6 | Day_6_JQ1 | 59.2925 | 121.813 | 1.03874 |
| Egr1 | Day_1 | Day_6 | OK | 16.8092 | 67.8666 | 2.01345 | Egr1 | Day_6 | Day_6_JQ1 | 67.8666 | 141.166 | 1.05662 |
| Ncaph | Day_1 | Day_6 | OK | 2.36158 | 7.72022 | 1.70889 | Ncaph | Day_6 | Day_6_JQ1 | 7.72022 | 16.1078 | 1.06105 |
| Igfbp6 | Day_1 | Day_6 | OK | 3.51816 | 25.1305 | 2.83655 | Igfbp6 | Day_6 | Day_6_JQ1 | 25.1305 | 52.4488 | 1.06147 |
| Scara3 | Day_1 | Day_6 | OK | 8.36565 | 20.633 | 1.3024 | Scara3 | Day_6 | Day_6_JQ1 | 20.633 | 43.4366 | 1.07396 |
| Atoh8 | Day_1 | Day_6 | OK | 2.61153 | 34.6452 | 3.72969 | Atoh8 | Day_6 | Day_6_JQ1 | 34.6452 | 73.1716 | 1.07863 |
| Gamt | Day_1 | Day_6 | OK | 0.85939 | 6.04897 | 2.81531 | Gamt | Day_6 | Day_6_JQ1 | 6.04897 | 12.8445 | 1.08639 |
| Prelp | Day_1 | Day_6 | OK | 14.0376 | 574.066 | 5.35385 | Prelp | Day_6 | Day_6_JQ1 | 574.066 | 1220.99 | 1.08877 |
| Mapk8ip1 | Day_1 | Day_6 | OK | 2.90214 | 7.61518 | 1.39176 | Mapk8ip1 | Day_6 | Day_6_JQ1 | 7.61518 | 16.3375 | 1.10123 |
| Plat | Day_1 | Day_6 | OK | 16.9511 | 65.2445 | 1.94448 | Plat | Day_6 | Day_6_JQ1 | 65.2445 | 141.636 | 1.11826 |
| Pcsk6 | Day_1 | Day_6 | OK | 1.62232 | 7.28721 | 2.1673 | Pcsk6 | Day_6 | Day_6_JQ1 | 7.28721 | 16.0741 | 1.1413 |
| S100a4 | Day_1 | Day_6 | OK | 8.52213 | 113.335 | 3.73323 | S100a4 | Day_6 | Day_6_JQ1 | 113.335 | 250.226 | 1.14264 |
| Fhl1 | Day_1 | Day_6 | OK | 38.0191 | 226.187 | 2.57272 | Fhl1 | Day_6 | Day_6_JQ1 | 226.187 | 503.063 | 1.15322 |
| Emilin2 | Day_1 | Day_6 | OK | 7.2383 | 39.2756 | 2.43991 | Emilin2 | Day_6 | Day_6_JQ1 | 39.2756 | 88.1199 | 1.16583 |
| Asf1b | Day_1 | Day_6 | OK | 2.65062 | 10.1429 | 1.93607 | Asf1b | Day_6 | Day_6_JQ1 | 10.1429 | 22.9334 | 1.17697 |
| Pcyox1 | Day_1 | Day_6 | OK | 21.9975 | 59.619 | 1.43843 | Pcyox1 | Day_6 | Day_6_JQ1 | 59.619 | 135.704 | 1.18662 |
| Fam117a | Day_1 | Day_6 | OK | 1.51063 | 6.83129 | 2.177 | Fam117a | Day_6 | Day_6_JQ1 | 6.83129 | 15.5563 | 1.18727 |
| Zwilch | Day_1 | Day_6 | OK | 0.91737 | 5.87362 | 2.67867 | Zwilch | Day_6 | Day_6_JQ1 | 5.87362 | 13.4294 | 1.19307 |
| Itm2a | Day_1 | Day_6 | OK | 1.53022 | 25.9026 | 4.08129 | Itm2a | Day_6 | Day_6_JQ1 | 25.9026 | 59.8717 | 1.20878 |
| Spc25 | Day_1 | Day_6 | OK | 1.24637 | 11.7693 | 3.23923 | Spc25 | Day_6 | Day_6_JQ1 | 11.7693 | 27.2563 | 1.21156 |
| Itga1 | Day_1 | Day_6 | OK | 11.1952 | 42.8116 | 1.93512 | Itga1 | Day_6 | Day_6_JQ1 | 42.8116 | 101.182 | 1.24087 |
| Epha7 | Day_1 | Day_6 | OK | 1.22068 | 4.81249 | 1.97909 | Epha7 | Day_6 | Day_6_JQ1 | 4.81249 | 11.3808 | 1.24175 |
| Hspa2 | Day_1 | Day_6 | OK | 2.76943 | 8.25548 | 1.57576 | Hspa2 | Day_6 | Day_6_JQ1 | 8.25548 | 19.5751 | 1.24559 |
| Hist1h1c | Day_1 | Day_6 | OK | 42.2787 | 101.881 | 1.26888 | Hist1h1c | Day_6 | Day_6_JQ1 | 101.881 | 246.293 | 1.27349 |
| Figf | Day_1 | Day_6 | OK | 0.38804 | 11.1285 | 4.84192 | Figf | Day_6 | Day_6_JQ1 | 11.1285 | 27.0541 | 1.28158 |
| Qpct | Day_1 | Day_6 | OK | 9.06177 | 34.6815 | 1.9363 | Qpct | Day_6 | Day_6_JQ1 | 34.6815 | 87.1308 | 1.32901 |
| Serpini1 | Day_1 | Day_6 | OK | 0.91764 | 4.84342 | 2.40003 | Serpini1 | Day_6 | Day_6_JQ1 | 4.84342 | 12.3234 | 1.3473 |
| Pkia | Day_1 | Day_6 | OK | 2.10564 | 10.9865 | 2.38341 | Pkia | Day_6 | Day_6_JQ1 | 10.9865 | 28.0014 | 1.34976 |
| Ptms | Day_1 | Day_6 | OK | 104.072 | 246.064 | 1.24145 | Ptms | Day_6 | Day_6_JQ1 | 246.064 | 627.919 | 1.35154 |
| Hmgn3 | Day_1 | Day_6 | OK | 5.24461 | 23.3317 | 2.15338 | Hmgn3 | Day_6 | Day_6_JQ1 | 23.3317 | 59.5606 | 1.35207 |
| Prss35 | Day_1 | Day_6 | OK | 0.32611 | 9.28011 | 4.83072 | Prss35 | Day_6 | Day_6_JQ1 | 9.28011 | 23.844 | 1.36141 |
| Snai2 | Day_1 | Day_6 | OK | 1.44675 | 8.90852 | 2.62237 | Snai2 | Day_6 | Day_6_JQ1 | 8.90852 | 23.1888 | 1.38017 |
| Slc9a3r1 | Day_1 | Day_6 | OK | 30.6589 | 61.6482 | 1.00775 | Slc9a3r1 | Day_6 | Day_6_JQ1 | 61.6482 | 161.158 | 1.38634 |
| Tbx20 | Day_1 | Day_6 | OK | 5.71776 | 20.8407 | 1.86588 | Tbx20 | Day_6 | Day_6_JQ1 | 20.8407 | 54.5866 | 1.38914 |
| Gdf11 | Day_1 | Day_6 | OK | 1.50056 | 8.7254 | 2.53972 | Gdf11 | Day_6 | Day_6_JQ1 | 8.7254 | 22.9531 | 1.39539 |
| Lum | Day_1 | Day_6 | OK | 18.2755 | 180.682 | 3.30547 | Lum | Day_6 | Day_6_JQ1 | 180.682 | 485.984 | 1.42745 |
| Fgfr2 | Day_1 | Day_6 | OK | 7.17234 | 31.2281 | 2.12233 | Fgfr2 | Day_6 | Day_6_JQ1 | 31.2281 | 86.0077 | 1.46162 |
| Isyna1 | Day_1 | Day_6 | OK | 16.3615 | 41.1524 | 1.33067 | Isyna1 | Day_6 | Day_6_JQ1 | 41.1524 | 113.425 | 1.46269 |
| Fam83d | Day_1 | Day_6 | OK | 1.50658 | 8.52721 | 2.5008 | Fam83d | Day_6 | Day_6_JQ1 | 8.52721 | 24.1741 | 1.50332 |
| Hmcn1 | Day_1 | Day_6 | OK | 0.59801 | 4.70187 | 2.97498 | Hmcn1 | Day_6 | Day_6_JQ1 | 4.70187 | 13.3966 | 1.51056 |
| Arxes2 | Day_1 | Day_6 | OK | 0.32244 | 9.31609 | 4.85261 | Arxes2 | Day_6 | Day_6_JQ1 | 9.31609 | 27.1896 | 1.54526 |
| Mmp17 | Day_1 | Day_6 | OK | 0.89231 | 8.60914 | 3.27025 | Mmp17 | Day_6 | Day_6_JQ1 | 8.60914 | 25.3895 | 1.56029 |
| Sesn3 | Day_1 | Day_6 | OK | 2.90474 | 9.04681 | 1.639 | Sesn3 | Day_6 | Day_6_JQ1 | 9.04681 | 27.3512 | 1.59612 |
| Upk1b | Day_1 | Day_6 | OK | 1.07085 | 25.0577 | 4.54842 | Upk1b | Day_6 | Day_6_JQ1 | 25.0577 | 78.8086 | 1.6531 |
| Tcf19 | Day_1 | Day_6 | OK | 2.37714 | 13.6858 | 2.52539 | Tcf19 | Day_6 | Day_6_JQ1 | 13.6858 | 43.53 | 1.66932 |
| Sdpr | Day_1 | Day_6 | OK | 3.87385 | 27.4875 | 2.82694 | Sdpr | Day_6 | Day_6_JQ1 | 27.4875 | 94.9074 | 1.78774 |
| Col14a1 | Day_1 | Day_6 | OK | 5.80191 | 52.3728 | 3.17422 | Col14a1 | Day_6 | Day_6_JQ1 | 52.3728 | 181.433 | 1.79255 |
| Fbln5 | Day_1 | Day_6 | OK | 4.40396 | 22.6672 | 2.36373 | Fbln5 | Day_6 | Day_6_JQ1 | 22.6672 | 79.5082 | 1.8105 |
| H2afx | Day_1 | Day_6 | OK | 14.969 | 43.7663 | 1.54784 | H2afx | Day_6 | Day_6_JQ1 | 43.7663 | 155.278 | 1.82696 |
| Abat | Day_1 | Day_6 | OK | 3.2569 | 9.85593 | 1.59749 | Abat | Day_6 | Day_6_JQ1 | 9.85593 | 35.0761 | 1.83142 |
| H1fx | Day_1 | Day_6 | OK | 0.47677 | 14.1724 | 4.89365 | H1fx | Day_6 | Day_6_JQ1 | 14.1724 | 50.515 | 1.83363 |
| Peg3 | Day_1 | Day_6 | OK | 0.37894 | 9.11016 | 4.58744 | Peg3 | Day_6 | Day_6_JQ1 | 9.11016 | 37.0421 | 2.02362 |
| Dzip1 | Day_1 | Day_6 | OK | 1.06203 | 7.4541 | 2.81121 | Dzip1 | Day_6 | Day_6_JQ1 | 7.4541 | 31.5814 | 2.08297 |
| H19 | Day_1 | Day_6 | OK | 0.70481 | 19.2203 | 4.76926 | H19 | Day_6 | Day_6_JQ1 | 19.2203 | 82.2113 | 2.0967 |
| Adamts5 | Day_1 | Day_6 | OK | 0.66941 | 20.6435 | 4.94666 | Adamts5 | Day_6 | Day_6_JQ1 | 20.6435 | 91.9646 | 2.15539 |
| Rtn1 | Day_1 | Day_6 | OK | 1.38392 | 10.0727 | 2.86361 | Rtn1 | Day_6 | Day_6_JQ1 | 10.0727 | 47.263 | 2.23026 |
| Svep1 | Day_1 | Day_6 | OK | 0.85337 | 17.4189 | 4.35133 | Svep1 | Day_6 | Day_6_JQ1 | 17.4189 | 86.2113 | 2.30723 |
| 1190002N15Rik | Day_1 | Day_6 | OK | 7.94395 | 21.8157 | 1.45744 | 1190002N15Rik | Day_6 | Day_6_JQ1 | 21.8157 | 132.567 | 2.60328 |
| Efemp1 | Day_1 | Day_6 | OK | 12.3716 | 29.4527 | 1.25137 | Efemp1 | Day_6 | Day_6_JQ1 | 29.4527 | 205.387 | 2.80187 |
| Aldh1a1 | Day_1 | Day_6 | OK | 5.0413 | 39.4483 | 2.9681 | Aldh1a1 | Day_6 | Day_6_JQ1 | 39.4483 | 311.654 | 2.98191 |
| Aard | Day_1 | Day_6 | OK | 2.08082 | 9.44578 | 2.18251 | Aard | Day_6 | Day_6_JQ1 | 9.44578 | 83.973 | 3.15218 |

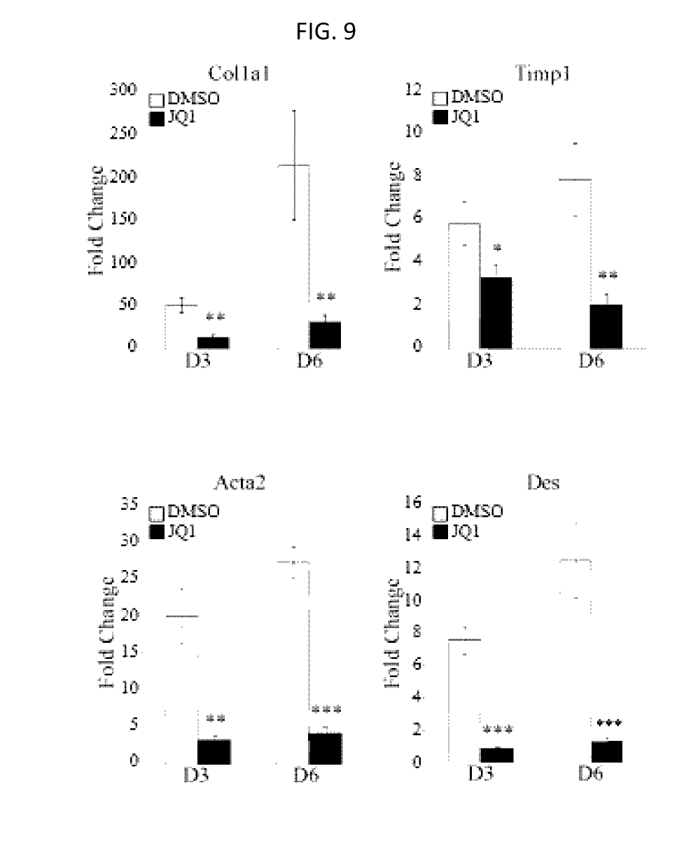

A heatmap of genes selected based on the highest magnitude of JQ1-mediated suppression illustrated an important role of BETs in regulating inducible gene expression during HSC activation into myofibroblasts (FIG. 8A). GO analysis of activation-induced genes that were suppressed by JQ1 revealed that BETs facilitate expression of a wide spectrum of biological processes and cellular components known to play critical roles in HSC transdifferentiation into myofibroblasts and liver fibrosis, including extracellular region, extracellular matrix (ECM), collagens, integrins, muscle contraction and focal adhesion (FIG. 8D), which is highly consistent with the GO analysis of putative BET target genes in LX-2 cells (FIG. 2B). Notably, during activation of HSCs into myofibroblasts, induction of key marker genes such as Col1a1, Acta2, Col1a2 and Des is drastically abolished by JQ1 (FIG. 8A & FIG. 9), indicating that BETs are essential for myofibroblast activation. Indeed, JQ1 treatment dramatically arrested HSCs transdifferentation into myofibroblasts phenotypically, such that the morphology, Acta2 distribution and lipid content of treated cells closely resembled quiescent (day 1) cells (FIG. 8F-8G).

### Example 6

### BET Inhibition Blocks Proliferation Underlying HSC Activation into Myofbibroblasts

The pathological relevance of HSC activation into myofibroblasts in liver fibrosis is not only established by induction of pro-fibrotic gene expression in individual cells but also manifested by acquired proliferative potential^{1, 2, 4}.

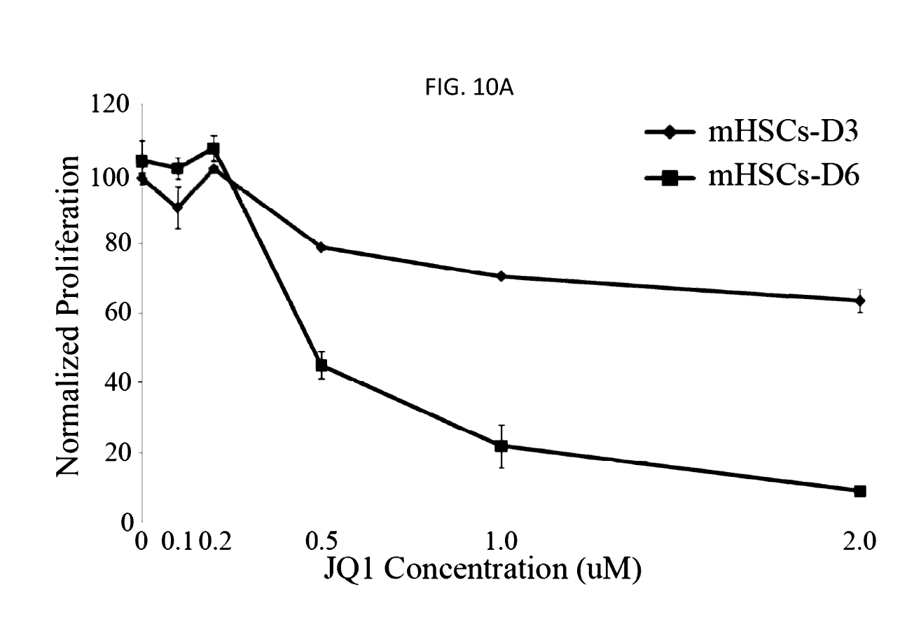

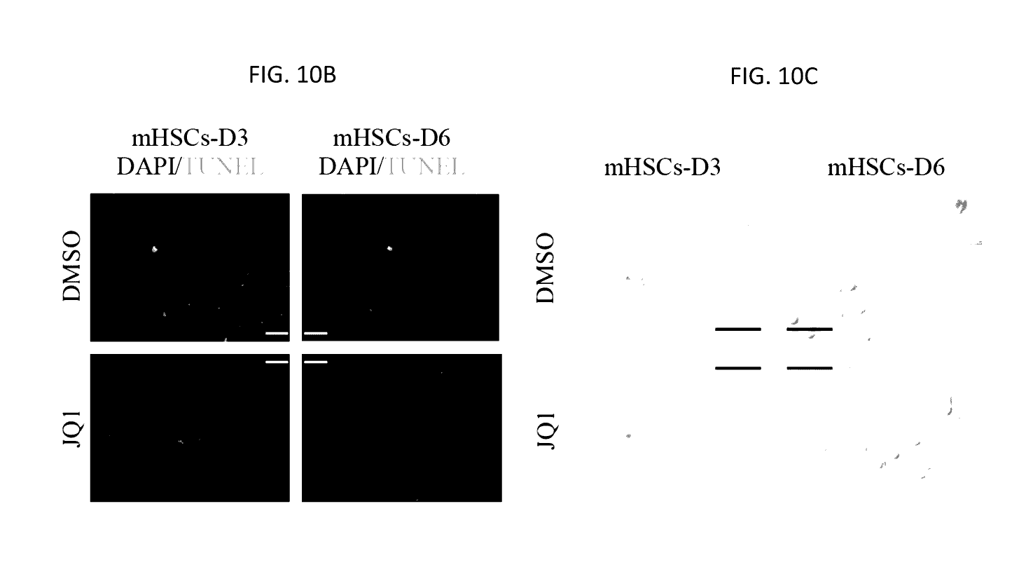

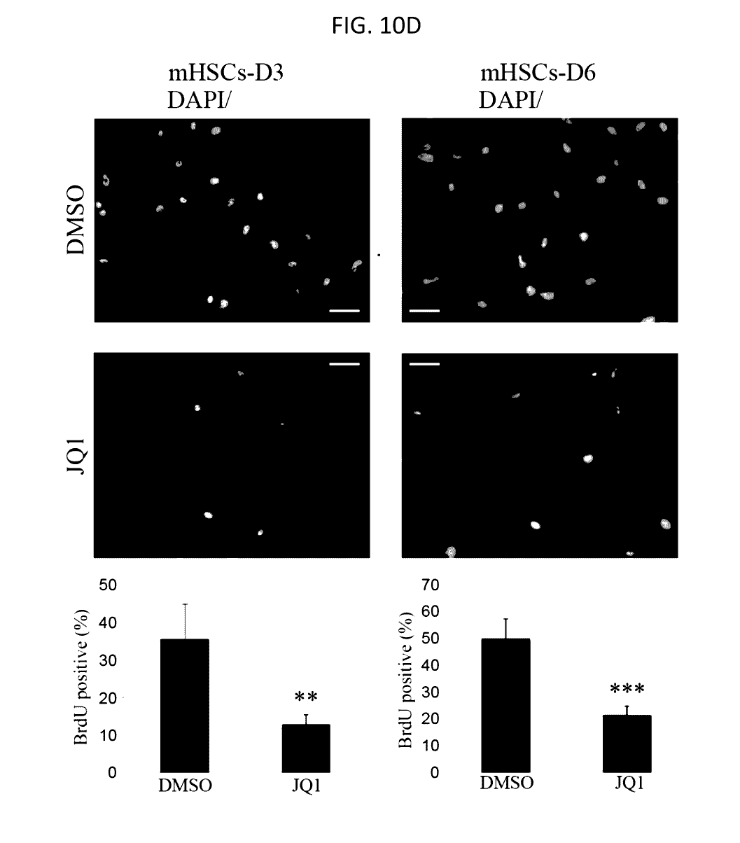

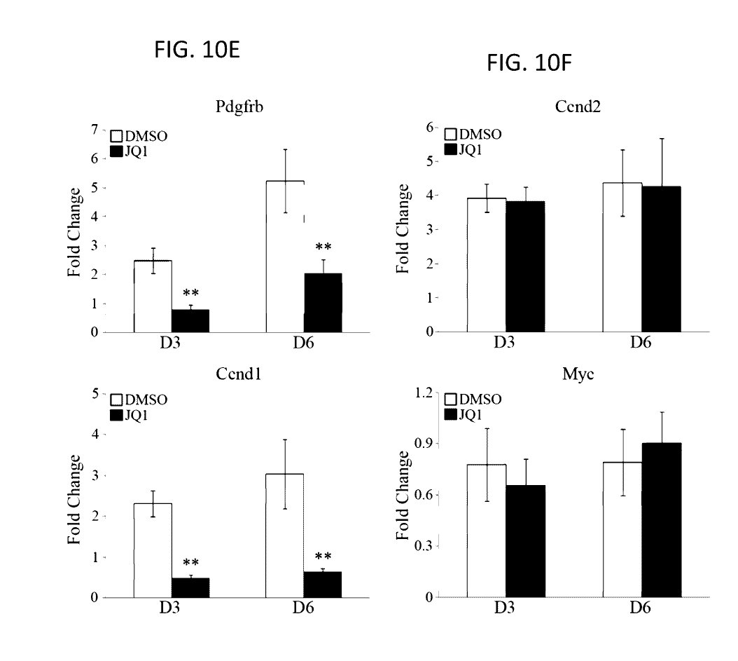

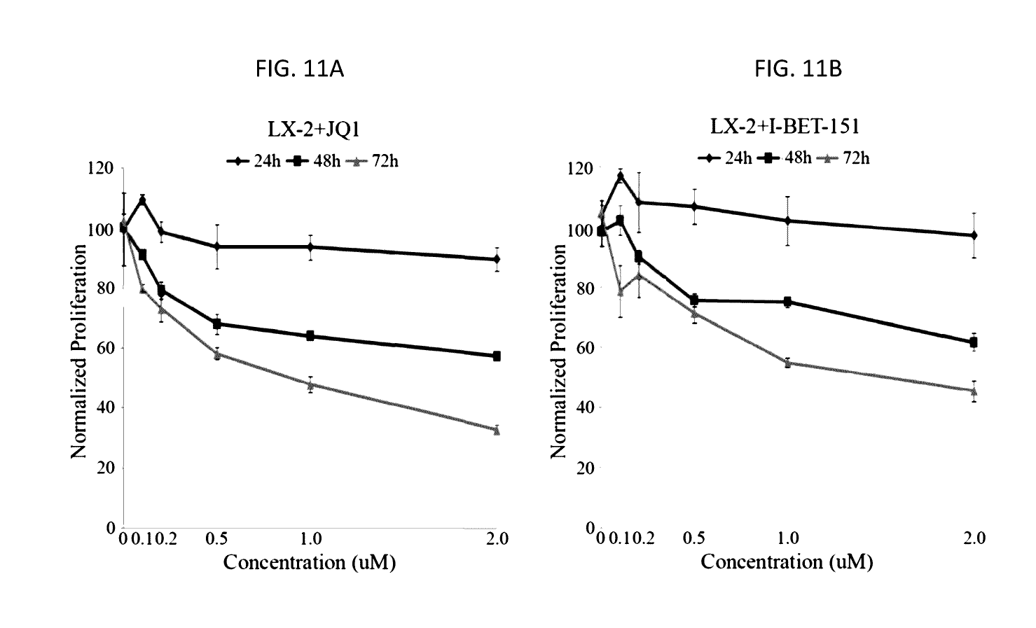

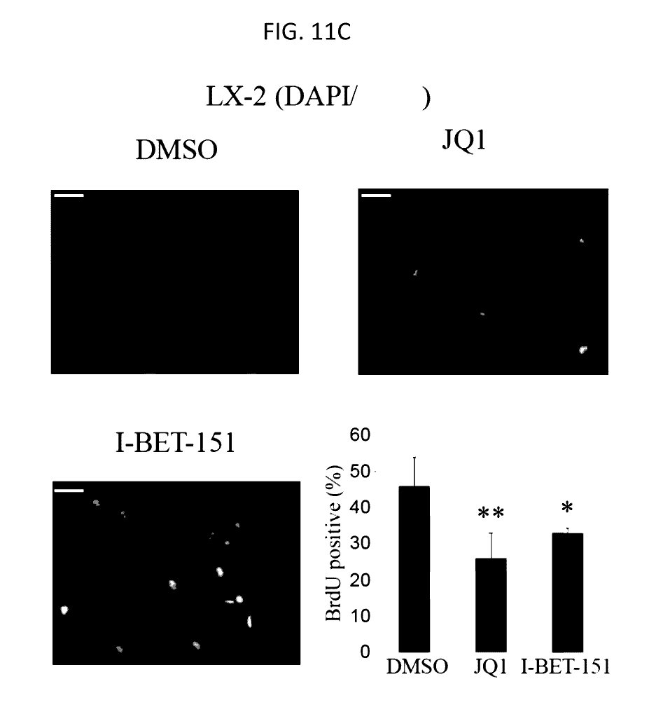

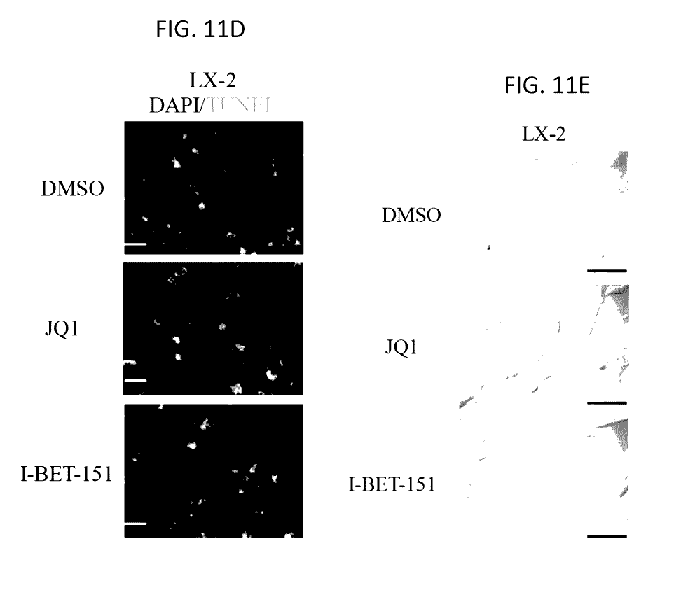

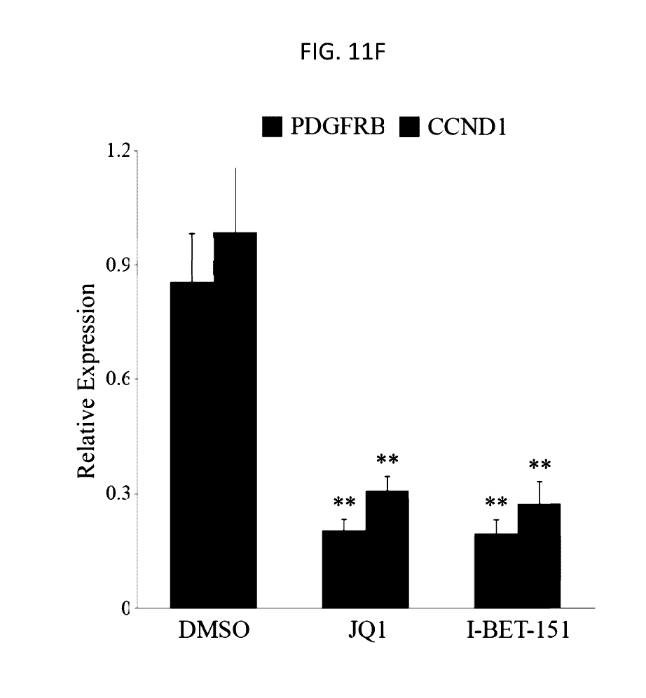

Interestingly, JQ1 exhibited significant anti-proliferative activity against activated HSCs in a dose-dependent manner (FIG. 10A) an effect that was not due to either apoptosis (FIG. 10B) or cellular senescence (FIG. 10C). Cell proliferation assays using BrdU revealed that JQ1 caused a significant decrease in BrdU incorporation into activated HSCs (FIG. 10D), indicating that BETs are important for proliferation of activated HSCs. Since platelet-derived growth factor (PDGF) signaling is a potent mitogenic pathway in myofibroblasts²¹ and this pathway is directly targeted by BET-loaded super-enhancers (FIGS. 2D & 2H), we speculated that BETs might functionally communicate with this pathway to regulate proliferation in activated HSCs. Gene expression analysis revealed that JQ1 disrupted induction of key PDGF pathway components such as Pdgfrb and downstream mitogenic targets, such as Ccnd1²², during HSC activation (FIG. 10E) without perturbing Ccnd2 and Myc expression (FIG. 10F). Similar findings were obtained in LX-2 cells (FIGS. 11A-11F). Thus, in addition to their role in controlling pro-fibrotic gene expression, these results support BETs as critical mitogenic regulators of myofibroblasts.

### Example 7

### BET Inhibition Ameliorates Liver Fibrosis In Vivo

The ability of BET inhibition as a pharmacological approach to attenuate liver fibrosis in vivo was examined, based on the role of BETs and pro-fibrotic super-enhancers in governing myofibroblast activation. The ability of JQ1 to prevent liver fibrosis in a standard mouse model was tested, where liver injury and an associated wound healing response is induced by carbon tetrachloride (CCl₄).

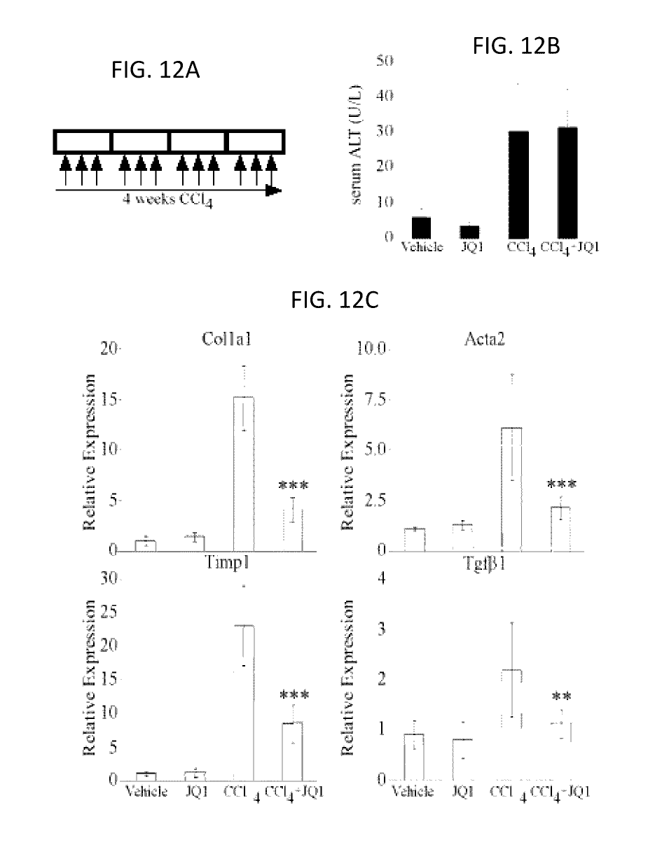

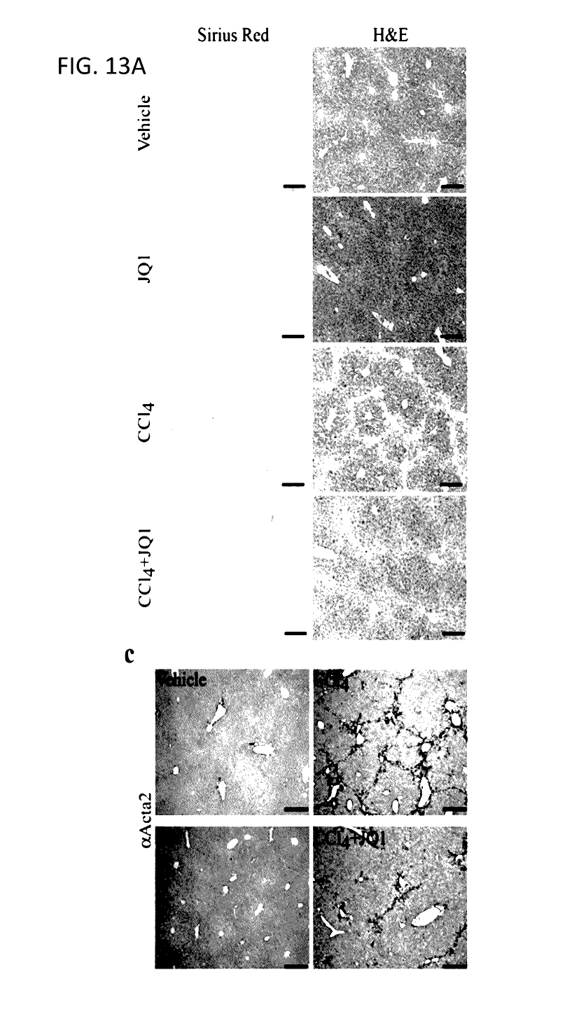

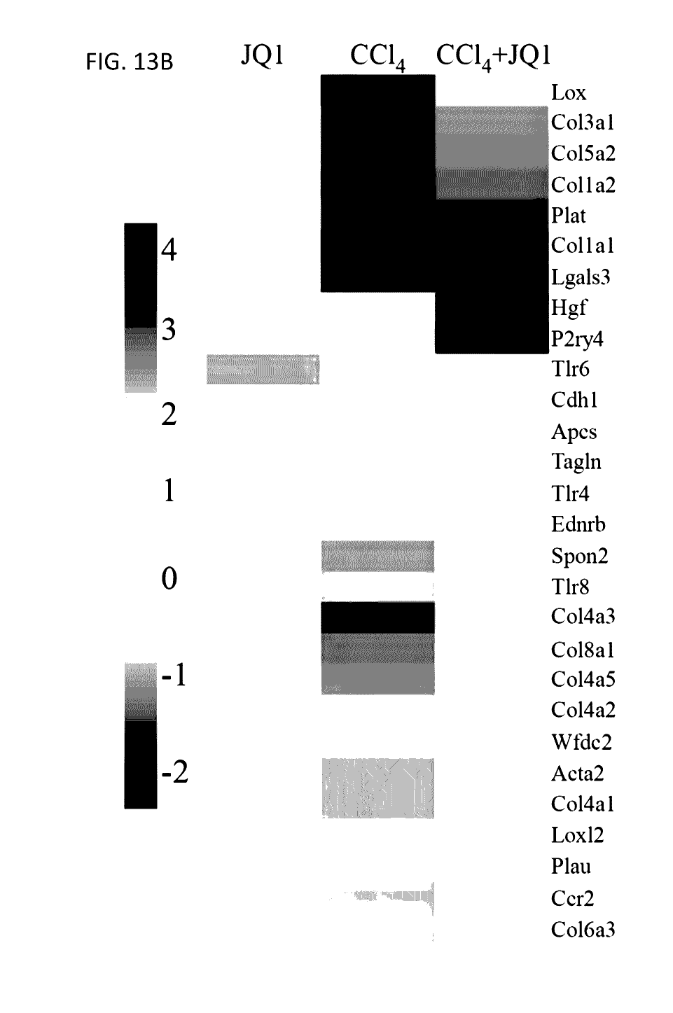

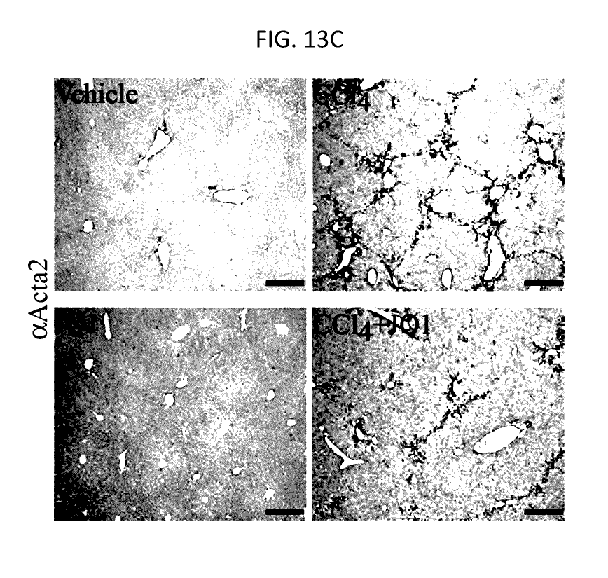

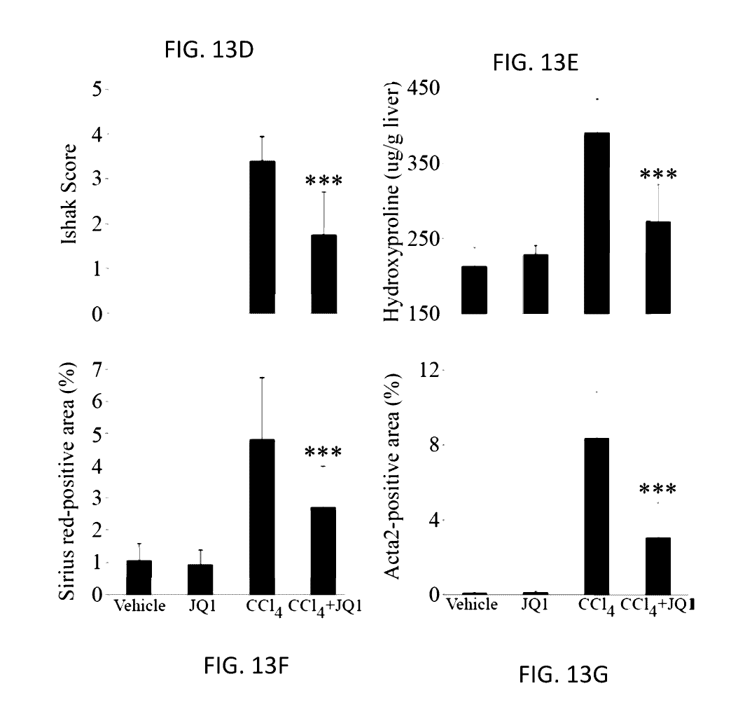

By four weeks, the livers of CCl₄-treated C57BL/6J mice exhibited extensive bridging fibrosis and substantial collagen deposition, whereas CCl₄/JQ1-co-treated mice demonstrated a dramatic reduction fibrosis as well as markers of HSC activation (FIG. 12A and FIG. 13A). These results were confirmed by quantitation of Sirius red staining, hepatic hydroxyproline content, Acta2 expression and histological fibrotic scoring (FIGS. 13C-13G). Liver injury due to CCl₄ was not significantly impacted upon by JQ1 as assessed by serum alanine aminotransferase (ALT) (FIG. 12B). At the molecular level, mRNA-Seq analysis confirmed significant suppression of CCl₄-induced key fibrotic marker genes by JQ1 treatment in liver (FIG. 13B and FIG. 12C).

### Example 8

### Therapeutic Effects of BET Inhibition Against Liver Fibrosis

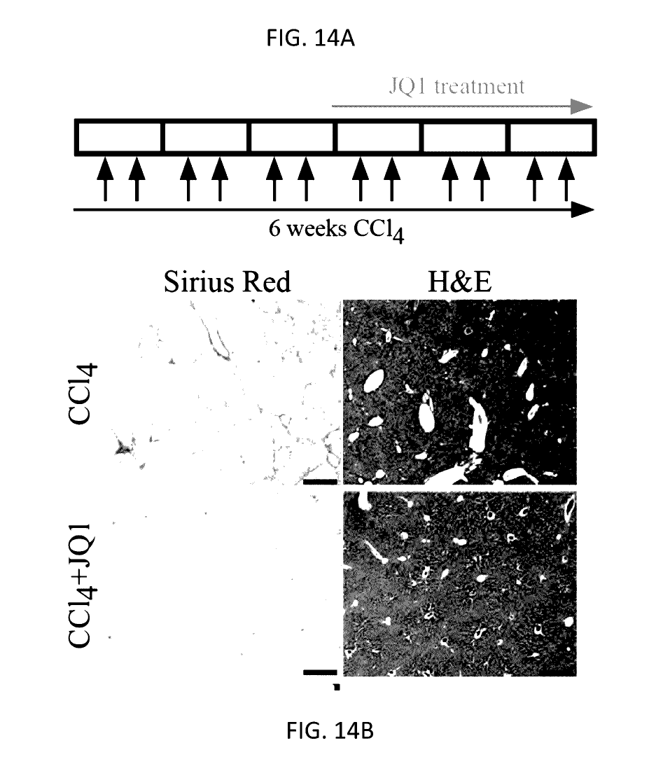

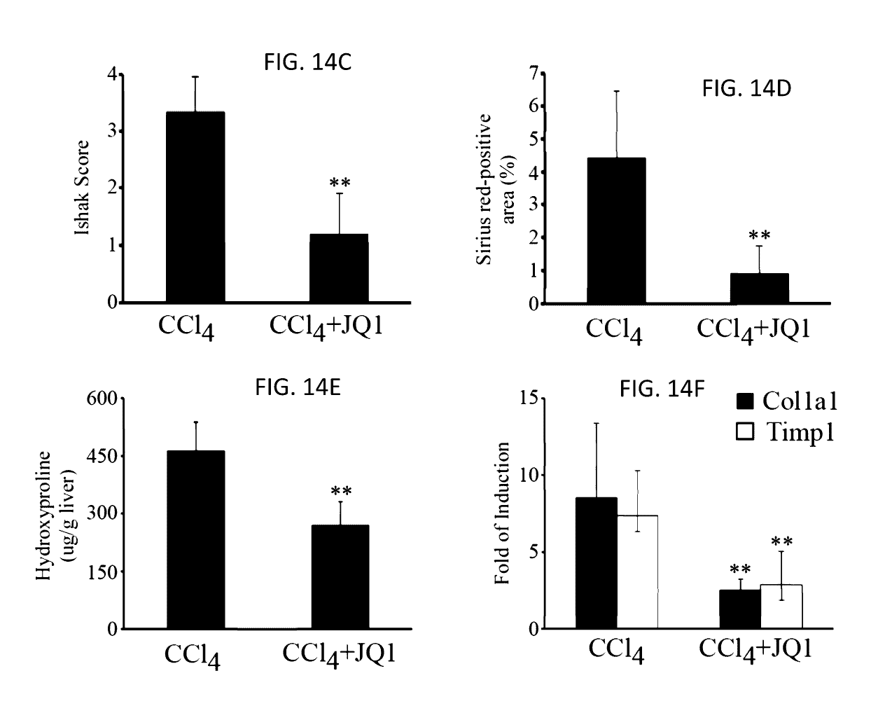

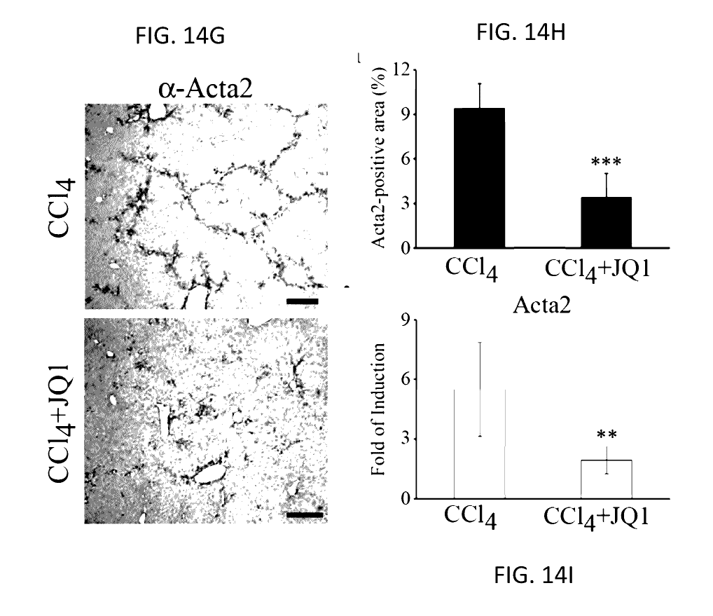

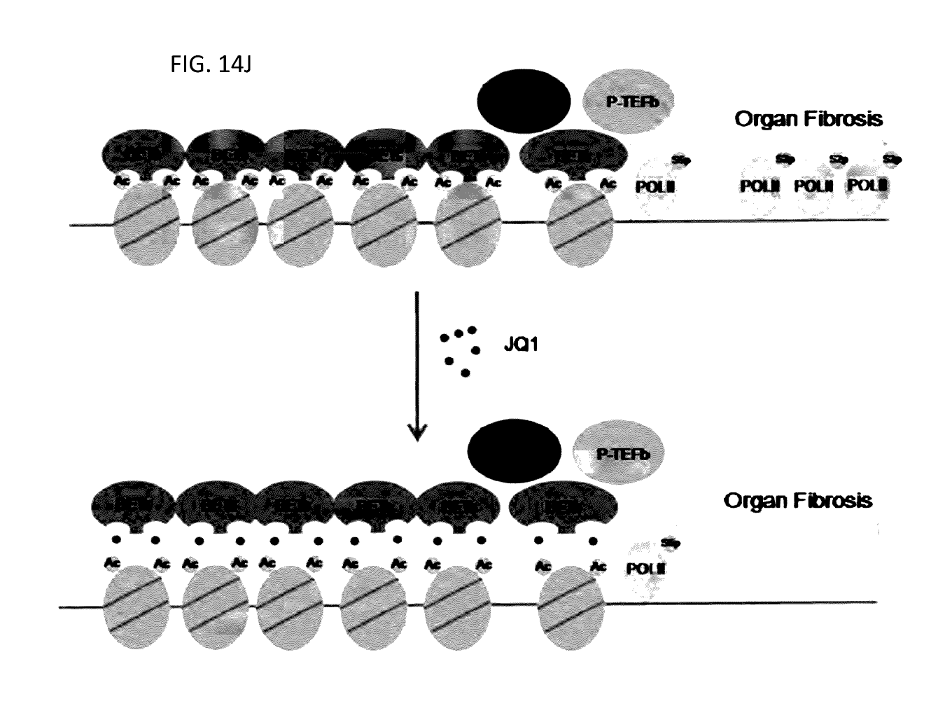

The dramatic anti-fibrotic properties of JQ1 in vitro and in vivo raised an intriguing and more clinically relevant question about the therapeutic effects of BET inhibition against liver fibrosis. Therefore, the ability of JQ1 to prevent progression of ongoing liver fibrosis was determined. In this regard, liver fibrosis was first initiated in C57BL/6J mice by CCl₄ treatment for three weeks prior to CCl₄/JQ1-co-treatment for an additional three weeks (FIG. 14A).

Remarkably, JQ1 blocked the progression of liver fibrosis as determined by histological scoring, quantitation of Sirius red staining, hepatic hydroxyproline content and pro-fibrotic marker gene expression (FIGS. 14B-14F). In addition, HSC activation in livers was examined from control and JQ1-treated mice following chronic CCl₄ administration using quantitative Acta2 immunohistochemistry and it was observed that CCl₄-induced HSC activation was dramatically reduced by JQ1 treatment (FIGS. 14G-14J). This data demonstrates that small molecule-mediated BET inhibition has the capability to ameliorate liver fibrosis.

### Example 9

### Therapeutic Effects of BET Inhibition Against Pancreatic Cancer Cell Lines

Pancreatic cancer cell lines (AsPc1, MIAPaCa2, PancT and P53 2.1.1) were embedded in HyStem®-C hydrogels (ESI-BIO) at a concentration of 5×10⁵ cells/ml. For astromal hydrogels, Glycosil® and Extralink™ components were resuspended in degassed H₂O per manufacturer's procotol, and Glycosil®+degassed H2O+Extralink™ were combined in a 1:1:1 ratio. For the stromal hydrogels, Glycosil®, Gelin-S® (denatured collagens), and Extralink™ components were resuspended in degassed H₂O per manufacturer's procotol, and Glycosil®+Gelin-S®+Extralink™ were combined in a 1:1:1 ratio. Pancreatic cancer cells were resuspended in the hydrogels and seeded into 96-well plates, 100 μl/well (5×10⁴ cells/well). Triplicate wells were seeded for each condition to be tested. After hydrogel polymerization was evident (30-45 minutes), DMEM+10% FBS was added to astromal wells and conditioned media from cancer-associated PSCs was added to stromal wells. Conditioned media was prepared by growing primary cancer-associated PSCs (grown out of human pancreatic tumors) to confluency, changing to fresh DMEM+10% FBS, then collecting the media after 48 hours and passing through a 0.45 um filter to clear debris. Vehicle (DMSO) was added to control wells for both astromal and stromal conditions, and 500 nM JQ1 was added to experimental wells for both conditions. Viability assays were performed after 72 hours using the Cell TiterGlo reagent (Promega) according to the manufacturer's instructions, but with a 45 minute incubation to ensure efficient lysis in 3D cultures.

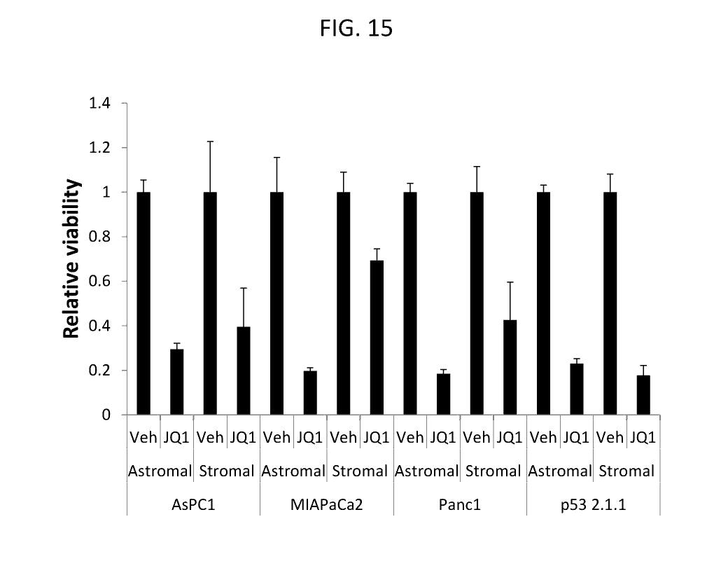

As shown in FIG. 15, JQ1 significantly reduced growth of pancreatic cancer cell lines in vitro. Thus JQ1 and other BET inhibitors as part of the compositions provided herein can be used to reduce pancreatic fibrosis and/or pancreatic cancer, such as a reduction of growth or viability of such cells by at least 20%, at least 40%, at least 50%, at least 70%, at least 75%, at least 80%, as compared to an absence of such treatment.

### Example 10

### Therapeutic Effects of BET Inhibition Against Orthotopic Allografts

The p53 2.1.1 cell line, a luciferase-expressing cell line derived from autochthonous pancreatic cancer in pure FVB/n mice, was used for orthotopic transplantation into pancreata of immune-competent FVB/n hosts. Cells were resuspended in 50% DMEM+10% FBS, 50% Matrigel and 1,000 cells per mouse were injected into the body of the pancreas. One week after transplantation, transplanted mice were subject to bioluminescence imaging to measure luciferase activity and, thus, tumor burden. Mice were randomized and treated with vehicle (10% β-cyclodextrin in sterile saline) or 75 mg/kg JQ1 i.p. daily for 14 days. Mice were imaged again to measure bioluminescence signal (tumor burden) at experimental endpoint; pancreata were removed, weighed, and fixed or flash-frozen for further analysis.

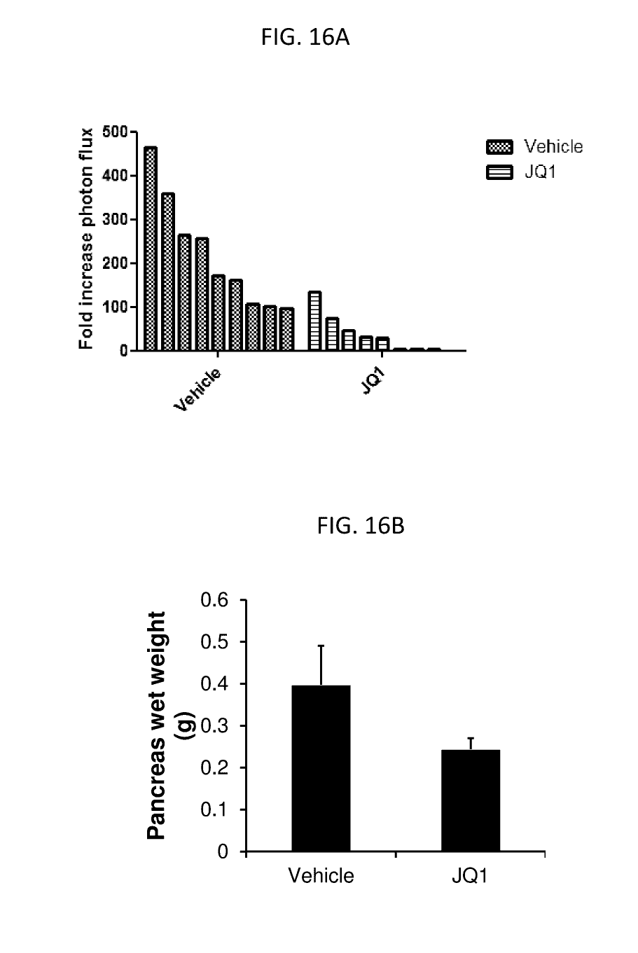

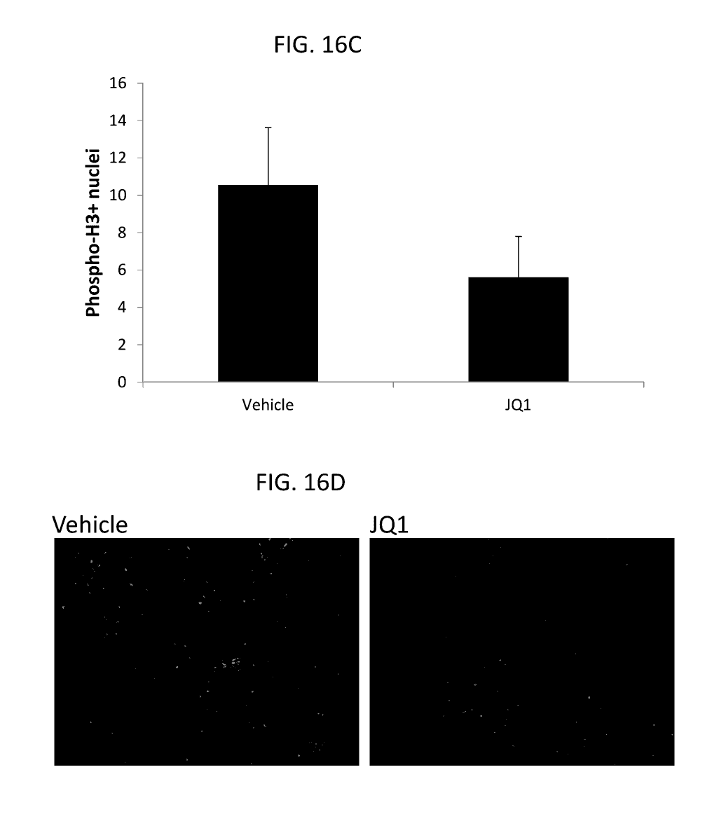

As shown in FIGS. 16A and 16B, JQ1 significantly reduced BLI and pancreas weight in vivo. The pancreatic tumor cells express luciferase, which is quantified by light units, and thus is used as a measure of the tumor cell count. A decrease of about 33% was observed. Thus, a composition provided herein that includes one or more BET inhibitors can be used to decrease pancreatic tumor burden.

As shown in FIGS. 16C and 16D, JQ1 significantly reduced the number of phospho-H3+ tumor cells, and the number of CD45 and DAPI containing cells, in vivo. Phospho-histone H3 (PHH3) is an immunomarker specific for cells undergoing mitoses. A decrease in cell proliferation of about 50% was observed. CD45 is a lymphocyte common antigen, and DAPI stains nuclei. A decrease in leukocyte recruitment of about 70% was observed. Thus, a composition provided herein that includes one or more BET inhibitors can be used to decrease actively dividing cells and leukocyte recruitment. Thus, inhibition of acetyl-lysine sensing by the BET bromodomain family blocks subset of stroma-inducible expression changes, significantly reduces or inhibits tumor growth and associated inflammation in vivo.

### REFERENCES

¹Bataller, R. & Brenner, D. A. Liver fibrosis. J Clin Invest 115, 209-218, doi:10.1172/JCl24282 (2005).
²Hernandez-Gea, V. & Friedman, S. L. Pathogenesis of liver fibrosis. Annu Rev Pathol 6, 425-456, (2011).
³Cohen-Naftaly, M. & Friedman, S. L. Current status of novel antifibrotic therapies in patients with chronic liver disease. Therap Adv Gastroenterol 4, 391-417, doi:10.1177/1756283X11413002 [doi] 10.1177_1756283 X11413002 [pii] (2011).
⁴Friedman, S. L. Hepatic stellate cells: protean, multifunctional, and enigmatic cells of the liver. Physiol Rev 88, 125-172, doi:88/1/125 [pii] 10.1152/physrev.00013.2007 [doi] (2008).
⁵Ding, N. et al. A Vitamin D Receptor/SMAD Genomic Circuit Gates Hepatic Fibrotic Response. Cell 153, 601-613 (2013).
⁶Helin, K. & Dhanak, D. Chromatin proteins and modifications as drug targets. Nature 502, 480-488 (2013).
⁷Filippakopoulos, P. et al. Histone recognition and large-scale structural analysis of the human bromodomain family. Cell 149, 214-231 (2012).
⁸Zuber, J. et al. RNAi screen identifies Brd4 as a therapeutic target in acute myeloid leukaemia. Nature 478, 524-528 (2011).
⁹Delmore, J. E. et al. BET bromodomain inhibition as a therapeutic strategy to target c-Myc. Cell 146, 904-917 (2011).
¹⁰Filippakopoulos, P. et al. Selective inhibition of BET bromodomains. Nature 468, 1067-1073 (2010).
¹¹Dawson, M. A. et al. Inhibition of BET recruitment to chromatin as an effective treatment for MLL-fusion leukaemia. Nature 478, 529-533 (2011).
¹²Banerjee, C. et al. BET bromodomain inhibition as a novel strategy for reactivation of HIV-1. J Leukoc Biol 92, 1147-1154 (2012).
¹³Nicodeme, E. et al. Suppression of inflammation by a synthetic histone mimic. Nature 468, 1119-1123 (2010).
¹⁴Anand, P. et al. BET bromodomains mediate transcriptional pause release in heart failure. Cell 154, 569-582 (2013).
¹⁵Spiltoir, J. I. et al. BET acetyl-lysine binding proteins control pathological cardiac hypertrophy. J Mol Cell Cardiol 63, 175-179 (2013).
¹⁶Bachem, M. G., Meyer, D., Melchior, R., Sell, K. M. & Gressner, A. M. Activation of rat liver perisinusoidal lipocytes by transforming growth factors derived from myofibroblastlike cells. A potential mechanism of self perpetuation in liver fibrogenesis. J Clin Invest. 89, 19-27. (1992).
¹⁷Friedman, S. L., Roll, F. J., Boyles, J., Arenson, D. M. & Bissell, D. M. Maintenance of differentiated phenotype of cultured rat hepatic lipocytes by basement membrane matrix. J Biol Chem. 264, 10756-10762. (1989).
¹⁸Geerts, A. et al. In vitro differentiation of fat-storing cells parallels marked increase of collagen synthesis and secretion. J Hepatol. 9, 59-68. (1989).
¹⁹Fish, P. V. et al. Identification of a chemical probe for bromo and extra C-terminal bromodomain inhibition through optimization of a fragment-derived hit. J Med Chem 55, 9831-9837 (2012).
²⁰Xu, L. et al. Human hepatic stellate cell lines, LX-1 and LX-2: new tools for analysis of hepatic fibrosis. Gut 54, 142-151, doi:54/1/142 [pii] 10.1136/gut.2004.042127 [doi] (2005).
²¹Wong, L., Yamasaki, G., Johnson, R. J. & Friedman, S. L. Induction of beta-platelet-derived growth factor receptor in rat hepatic lipocytes during cellular activation in vivo and in culture. J Clin Invest. 94, 1563-1569. (1994).
²²Chen, H. et al. PDGF signalling controls age-dependent proliferation in pancreatic beta-cells. Nature 478, 349-355 (2011).
²³Trojanowska, M. Ets factors and regulation of the extracellular matrix. Oncogene. 19, 6464-6471. (2000).
²⁴Small, E. M. The actin-MRTF-SRF gene regulatory axis and myofibroblast differentiation. J Cardiovasc Transl Res 5, 794-804 (2012).
²⁵Vincent, K. J. et al. Regulation of E-box DNA binding during in vivo and in vitro activation of rat and human hepatic stellate cells. Gut. 49, 713-719. (2001).
²⁶Schnabl, B. et al. The role of Smad3 in mediating mouse hepatic stellate cell activation. Hepatology. 34, 89-100. (2001).
²⁷Seki, E. et al. TLR4 enhances TGF-beta signaling and hepatic fibrosis. Nat Med 13, 1324-1332, doi:nm1663 [pii] 10.1038/nm1663 (2007).
²⁸Loven, J. et al. Selective inhibition of tumor oncogenes by disruption of super-enhancers. Cell 153, 320-334 (2013).
²⁹Dobin, A. et al. STAR: ultrafast universal RNA-seq aligner. Bioinformatics 29, 15-21 (2013).
³⁰Trapnell, C. et al. Differential analysis of gene regulation at transcript resolution with RNA-seq. Nat Biotechnol 31, 46-53 (2013).
³¹Barish, G. D. et al. The Bcl6-SMRT/NCoR cistrome represses inflammation to attenuate atherosclerosis. Cell Metab 15, 554-562 (2012).
³²Chapuy, B. et al. Discovery and characterization of super-enhancer-associated dependencies in diffuse large B cell lymphoma. Cancer Cell. 24, 777-790. (2013)

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A composition comprising:
a nanoparticle; and
a compound that reduces the biological activity of one or more bromodomain and extra-terminal family member (BET) proteins.

2. The composition of claim 1, wherein the nanoparticle comprises a lipid nanoparticle or polymeric nanoparticle.

3. The composition of claim 1, wherein the one or more BET proteins comprise one or more of human bromodomain-containing protein 2 (Brd2), Brd3, and Brd4.

4. The composition of claim 1, wherein the biological activity of one or more BET proteins comprises one or more of release of vitamin A, vitamin D and/or lipids from a cell.

5. The composition of claim 1, wherein the compound reduces the biological activity of one or more BET proteins by at least 25% as compared to the biological activity in the absence of the compound.

6. The composition of claim 1, wherein the compound reduces the biological activity of one or more BET proteins in a stellate cell, an epithelial cell, or both.

7. The composition of claim 1, wherein the compound reduces the biological activity of one or more BET proteins in a pancreatic, kidney or hepatic stellate cell.

8. The composition of claim 1, wherein the compound that reduces the biological activity of one or more BET proteins comprises:
(a) JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate)
(b) LY294002 (2-Morpholin-4-yl-8-phenylchromen-4-one)
(c) a combination of (a) and (b);
(d) (S)-2-(6-(4-chlorophenyl)-8-methoxy-1-methyl-4H-benzo[f][1,2,4]triazolo[4,3-a][1,4]diazepin-4-yl)-N-ethylacetamide
(e) (6S)-4-(4-chlorophenyl)-N-(4-hydroxyphenyl)-2,3,9-trimethyl-6H-thieno[3,2-f][1,2,4]triazolo[4,3-a][1,4]diazepine-6-acetamide
(f) a combination of two or more of (a), (b), (d), and (e).

9. The composition of claim 1, wherein the composition further comprises a chemotherapeutic, a biologic, a vitamin D receptor (VDR) agonist, or combinations thereof.

10. The composition of claim 9, wherein the chemotherapeutic comprises gemcitabine.

11. The composition of claim 9, wherein the VDR agonist is vitamin D, a vitamin D precursor, a vitamin D analog, a vitamin D receptor ligand, a vitamin D receptor agonist precursor, or combinations thereof.

12. The composition of claim 9, wherein the VDR agonist is calcipotriol, 25-hydroxy-D₃ (25-OH-D₃) (calcidiol); vitamin D3 (cholecalciferol); vitamin D2 (ergocalciferol), 1,α25-dihydroxyvitamin D₃ (calcitriol), or combinations thereof.

13. A method for increasing or retaining vitamin A, vitamin D, and/or lipid in an epithelial or stellate cell, comprising:
contacting a therapeutically effective amount of the composition of claim 1 with the epithelial or stellate cell, thereby increasing or retaining vitamin A, vitamin D, and/or lipid in the epithelial or stellate cell.

14. The method of claim 13, wherein the epithelial or stellate cell is in a subject, and wherein contacting comprises administering a therapeutically effective amount of the composition to the subject, thereby increasing or retaining vitamin A, vitamin D, and/or lipid in the epithelial or stellate cell.

15. The method of claim 14, wherein the subject has a liver disease.

16. The method of claim 15, wherein the liver disease is one or more of alcohol liver disease, fatty liver disease, liver fibrosis/cirrhosis, biliary fibrosis/cirrhosis, liver cancer, hepatitis, sclerosing cholangitis, Budd-Chiari syndrome, jaundice, hemochromatosis, or Wilson's disease.

17. The method of claim 16, wherein the liver cancer is a hepatocellular carcinoma, cholangiocarcinoma, angiosarcoma, or hemangiosarcoma.

18. The method of claim 14, wherein the subject has a pancreatic disease.

19. The method of claim 18, wherein the pancreatic disease is pancreatic fibrosis, pancreatic ductal adenocarcinoma (PDA).

20. The method of claim 14, wherein the subject has fibrosis of the kidney.

21. The method of claim 14, wherein the subject has pancreatic cancer.

22. A method of treating pancreatic cancer in a subject, comprising administering to the subject a therapeutically effective amount of the composition of claim 1, thereby treating the pancreatic cancer.

23. The method of claim 22, wherein the pancreatic cancer is an adenocarcinoma.

24. The method of claim 22, wherein the pancreatic cancer is a ductal adenocarcinoma.


SEQUENCE LISTING (32 sequences)

SEQ ID NO: 1 - DNA, 24 bp, Mus musculus
tcaacagcaactcccactcttcca

SEQ ID NO: 2 - DNA, 24 bp, Mus musculus
ttgtcattgagagcaatgccagcc

SEQ ID NO: 3 - DNA, 20 bp, Mus musculus
gctgaacctccatgaggaag

SEQ ID NO: 4 - DNA, 20 bp, Mus musculus
ggatcatcttggcgtagagc

SEQ ID NO: 5 - DNA, 24 bp, Mus musculus
gaccgcaaacagcttgatgtggat

SEQ ID NO: 6 - DNA, 24 bp, Mus musculus
atattcctcacatcttccgcccgt

SEQ ID NO: 7 - DNA, 24 bp, Mus musculus
actgcaacatggagacaggtcaga

SEQ ID NO: 8 - DNA, 24 bp, Mus musculus
atcggtcatgctctctccaaacca

SEQ ID NO: 9 - DNA, 24 bp, Mus musculus
tttggagcctggacacacagtaca

SEQ ID NO: 10 - DNA, 24 bp, Mus musculus
tgtgttggttgtagagggcaagga

SEQ ID NO: 11 - DNA, 24 bp, Mus musculus
ggtgtgcacagtgtttccctgttt

SEQ ID NO: 12 - DNA, 24 bp, Mus musculus
tccgtccacaaacagtgagtgtca

SEQ ID NO: 13 - DNA, 22 bp, Homo sapiens
gcagtgatgtaaaatttcttgg

SEQ ID NO: 14 - DNA, 23 bp, Homo sapiens
aaagctcggttttactcttcaca

SEQ ID NO: 15 - DNA, 20 bp, Homo sapiens
ctgtggcaaccaagactaca

SEQ ID NO: 16 - DNA, 20 bp, Homo sapiens
cccacctggaacttgatgag

SEQ ID NO: 17 - DNA, 21 bp, Homo sapiens
cctgctgcagattctctggaa

SEQ ID NO: 18 - DNA, 21 bp, Homo sapiens
agggtgtcgtgctgtttcttg

SEQ ID NO: 19 - DNA, 24 bp, Homo sapiens
accgaaggcagacggtaacaagta

SEQ ID NO: 20 - DNA, 24 bp, Homo sapiens
gacatgcttgagcaacgcactgaa

SEQ ID NO: 21 - DNA, 24 bp, Homo sapiens
atgtcaacaggaatgcagcagtgg

SEQ ID NO: 22 - DNA, 24 bp, Homo sapiens
atagcgctggttacagttgggaga

SEQ ID NO: 23 - DNA, 18 bp, Homo sapiens
cggtgtgactcgtgcagc

SEQ ID NO: 24 - DNA, 20 bp, Homo sapiens
acagccgcttcacctacagc

SEQ ID NO: 25 - DNA, 19 bp, Homo sapiens
tcaaactggctgccagcat

SEQ ID NO: 26 - DNA, 21 bp, Homo sapiens
caagaaacacgtctggctagg

SEQ ID NO: 27 - DNA, 24 bp, Homo sapiens
cgctaaggcgaaagccctcaattt

SEQ ID NO: 28 - DNA, 24 bp, Homo sapiens
acaattcctggcgatacctcagca

SEQ ID NO: 29 - DNA, 24 bp, Homo sapiens
tctgcaattccgacctcgtcatca

SEQ ID NO: 30 - DNA, 24 bp, Homo sapiens
aaggtggtctggttgacttctggt

SEQ ID NO: 31 - DNA, 20 bp, Homo sapiens
cattcccagctcccctctct

SEQ ID NO: 32 - DNA, 20 bp, Homo sapiens
agtctacgtggcaggcaagg